(19) United States
(12) Patent Application Publication
(10) Pub. No.: US 2025/0161425 A1
(43) Pub. Date: May 22, 2025

(54) SIGNAL SEQUENCES FOR NUCLEIC ACID VACCINES

(71) Applicant: SANOFI, Paris (FR)

(72) Inventors: Yves GIRERD-CHAMBAZ, Paris (FR); Vincent PAVOT, Paris (FR); Céline CIVAT, Paris (FR); Corinne PION, Paris (FR)

(21) Appl. No.: 18/929,923

(22) Filed: Oct. 29, 2024

Related U.S. Application Data
(63) Continuation of application No. PCT/EP2023/062066, filed on May 5, 2023.
(60) Provisional application No. 63/449,573, filed on Mar. 2, 2023.

(30) Foreign Application Priority Data
May 6, 2022 (EP) 22305680.5
Aug. 16, 2022 (EP) 22306227.4

(51) Int. Cl. A61K 39/02; A61K 9/1272; A61K 9/51; A61K 39/00; A61K 39/145; A61K 39/165; A61K 39/20; A61K 39/205; A61K 39/215; A61K 39/25; A61K 39/285; A61P 31/04; C12N 7/00; C12N 15/88

(52) U.S. Cl. CPC A61K 39/0225; A61K 9/1272; A61K 9/5123; A61K 39/145; A61K 39/165; A61K 39/20; A61K 39/205; A61K 39/215; A61K 39/25; A61K 39/285; A61P 31/04; C12N 7/00; C12N 15/88; A61K 2039/53; A61K 2039/55555; A61K 2039/70; C12N 2710/16734; C12N 2710/24134; C12N 2760/14134; C12N 2760/16134; C12N 2760/16234; C12N 2760/18434; C12N 2760/18734; C12N 2760/20134; C12N 2770/20034; C12N 2770/36234; C12N 2830/50

(57) ABSTRACT

Provided herein is a nucleic acid (e.g., messenger RNA) vaccine encoding at least one antigenic prokaryotic polypeptide linked to one or both of a viral secretion signal peptide and a transmembrane domain. Also provided are methods of vaccination against a prokaryotic infection with the nucleic acid described herein.

### RELATED APPLICATIONS

This application is a continuation of International Patent Application No. PCT/EP2023/062066, filed May 5, 2023, which claims priority to European Patent Application Nos. 22305680.5, filed May 6, 2022, and 22306227.4, filed Aug. 16, 2022, and U.S. Provisional Patent Application No. 63/449,573, filed Mar. 2, 2023, the entire contents of which are hereby incorporated herein by reference.

### SEQUENCE LISTING

The instant application contains a Sequence Listing which has been submitted electronically in XML format and is hereby incorporated by reference in its entirety. Said XML file, created on Oct. 29, 2024, is named 758218_SA9-326PCCON_ST26.xml and is 275,797 bytes in size.

### BACKGROUND OF THE DISCLOSURE

Prokaryotic infections (e.g., bacterial infections) represent a significant threat to human health globally. An estimated 5 million people die annually from antibiotic resistant bacterial infections, along with an increased burden on the healthcare system (Antimicrobial Resistance Collaborators. The Lancet. 399(10325): 629-655. 2022). Although vaccines against prokaryotic infections exist, they are fewer in number compared to more common anti-viral vaccines.

Nucleic acid-based vaccines, and more particularly mRNA vaccines, have recently emerged as an additional vaccine type with a rapid, safe, and cost-effective production process, in particular against viral pathogens. mRNA vaccines against severe acute respiratory syndrome coronavirus 2 (SARS CoV-2) mostly use the spike viral protein, as antigen. Often combined with a delivery vehicle, such as a lipid nanoparticle (LNP), COVID-19 mRNA vaccines may achieve high efficacy. None-the-less, there exists a need for more effective RNA-based vaccines against prokaryotic infections.

Generating nucleic acid-based vaccines, such as RNA (e.g., mRNA)-based vaccines, comprising prokaryotic antigens (i.e., antigens derived from antigens in a prokaryotic cell) presents a challenge because prokaryotic antigens are not naturally expressed by eukaryotic cells. In particular, prokaryotic cells and eukaryotic cells have different secretion systems. Without proper engineering, the prokaryotic antigens used in these vaccines would accumulate in the intracellular compartment of eukaryotic (e.g., human) cells, which may reduce the immunogenicity of these vaccines.

This invention aims to address this issue by improving the immunogenicity of prokaryotic antigens expressed through nucleic acid (e.g., mRNA)-based vaccines. As described herein, this is achieved by adding a secretion signal to allow the prokaryotic antigen to be secreted in the extracellular compartment and/or a transmembrane domain to allow expression at the cell surface. By doing so, immune cells can better access the antigen, ultimately improving the immunogenicity of the nucleic acid (e.g., mRNA)-based vaccine.

### SUMMARY OF THE DISCLOSURE

The present disclosure provides a nucleic acid comprising an open reading frame (ORF), wherein the ORF comprises:—a polynucleotide sequence encoding at least one antigenic prokaryotic polypeptide and—a polynucleotide sequence encoding at least one viral secretion signal peptide.

In certain embodiments, the ORF further comprises a polynucleotide sequence encoding at least one transmembrane domain (TMB).

In certain embodiments, the viral secretion signal peptide is derived from a viral sequence in a virus able to infect humans.

In certain embodiments, the viral secretion signal peptide is derived from a viral sequence selected from the group consisting of: an influenza secretion signal peptide sequence, and a non-influenza secretion signal peptide sequence selected from the group consisting of a SARS CoV-2 secretion signal peptide sequence, a varicella-zoster virus (VZV) secretion signal peptide sequence, a measles secretion signal peptide sequence, a rubella secretion signal peptide sequence, a mumps secretion signal peptide sequence, an Ebola secretion signal peptide sequence, a smallpox secretion signal peptide sequence, and a rabies secretion signal peptide sequence.

In certain embodiments, the viral secretion signal peptide is selected from the group consisting of: an influenza hemagglutinin (HA) secretion signal peptide sequence, a SARS CoV-2 spike secretion signal peptide sequence, a VZV gB secretion signal peptide sequence, a VZV gE secretion signal peptide sequence, a VZV gI secretion signal peptide sequence, a VZV gK secretion signal peptide sequence, a measles F-protein secretion signal peptide sequence, a rubella E1 protein secretion signal peptide sequence, a rubella E2 protein secretion signal peptide sequence, a mumps F-protein secretion signal peptide sequence, an Ebola GP protein secretion signal peptide sequence, a smallpox 6 kDa IC protein secretion signal peptide sequence, and a rabies G protein secretion signal peptide sequence, preferably wherein the viral secretion signal peptide comprises an HA secretion signal peptide sequence from influenza A or influenza B, more preferably from influenza A.

In certain embodiments, the HA secretion signal peptide sequence comprises an amino acid sequence MKX₁X₂LX₃VX₄LX₅TFX₆X₇X₅X₉A (SEQ ID NO: 145) wherein X₁ is selected from A and V; X₂ is selected from I and K; X₃ is selected from V and L; X₄ is selected from L and M; Xₛ is selected from Y and C; X₆ is selected from T and A X₇ is selected from T and A; Xs is selected from A and T; and X₉ is selected from N and Y.

In certain embodiments, the HA secretion signal peptide sequence comprises an amino acid sequence selected from SEQ ID NO: 95-109.

In certain embodiments, the HA secretion signal peptide sequence comprises an amino acid sequence MKX₁IIALSX₂ILCLVFX₃ (SEQ ID NO: 146) wherein X₁ is selected from T and A; X₂ is selected from Y, N, C, and H; and X₃ is selected from T and A.

In certain embodiments, the HA secretion signal peptide sequence comprises an amino acid sequence selected from SEQ ID NOs: 110-131.

In certain embodiments, the HA secretion signal peptide sequence comprises an amino acid sequence MKAIIVLLMVVTSX₁A (SEQ ID NO: 147) wherein X₁ is selected from S and N.

In certain embodiments, the HA secretion signal peptide sequence comprises an amino acid sequence MX₁AIIVLLMVVTSNA (SEQ ID NO: 148) wherein X₁ is selected from K and E.

In certain embodiments, the HA secretion signal peptide sequence comprises an amino acid sequence selected from SEQ ID NOs: 132-144.

In certain embodiments, the viral secretion signal peptide comprises an amino acid sequence selected from the group consisting of: MKAKLLVLLCTFTATYA (SEQ ID NO: 1); MKAILVVLLYTFATANA (SEQ ID NO: 2); MKTIIALSYILCLVFA (SEQ ID NO: 3); MKAIIVLLMVVTSNA (SEQ ID NO: 4); MFVFLVLLPLVS (SEQ ID NO: 5); MFLLTTKRTMFVFLVLLPLVS (SEQ ID NO: 6) MSPCGYYSKWRNRDRPEYRRNLRFRRFFSSIHPNAAAGSGFNGPGVFITSVTGVWLCFL CIFSMFVTAVVS (SEQ ID NO: 7); MGTVNKPVVGVLMGFGIITGTLRITNPVRA (SEQ ID NO: 8); MFLIQCLISAVIFYIQVTNA (SEQ ID NO: 9); MQALGIKTEHFIIMCLLSGHA (SEQ ID NO: 10); MGLKVNVSAIFMAVLLTLQTPTG (SEQ ID NO: 11); MGAAAALTAVVLQGYNPPAYG (SEQ ID NO: 12); MGAPQAFLAGLLLAAVAVGTARA (SEQ ID NO: 13); MKVFLVTCLGFAVFSSSVC (SEQ ID NO: 14); MGVTGILQLPRDRFKRTSFFLWVIILFQRTFS (SEQ ID NO: 15); MRSLIIFLLFPSIIYS (SEQ ID NO: 16); and MVPQALLFVPLLVFPLCFG (SEQ ID NO: 184).

In certain embodiments, the viral secretion signal peptide comprises an amino acid sequence of MKAKLLVLLCTFTATYA (SEQ ID NO: 1).

In certain embodiments, the viral secretion signal peptide is positioned at the N-terminus of the antigenic prokaryotic polypeptide.

In certain embodiments, the viral secretion signal peptide is positioned at the C-terminus of the antigenic prokaryotic polypeptide.

In certain embodiments, the viral secretion signal peptide is attached to the antigenic prokaryotic polypeptide with a linker.

In certain embodiments the TMB: (a) comprises or consists of 15 to 50 amino acid residues, preferably 15 to 30 amino acid residues, more preferably 18 to 25 amino acid residues; and/or (b) comprises at least 50% of hydrophobic amino acid residues, preferably selected in the group consisting of: alanine, isoleucine, leucine, valine, phenylalanine, tryptophane and tyrosine ; and/or (c) comprises at least one alpha helix.

In certain embodiments the TMB is derived from an integral membrane protein, preferably from a single pass membrane protein, more preferably from a bitopic membrane protein, even more preferably from a bitopic membrane protein of Type I.

In certain embodiments the TMB is derived from a non-human sequence.

In certain embodiments, the antigenic prokaryotic polypeptide is derived from a prokaryotic transmembrane protein, and wherein the TMB is the TMB of the prokaryotic transmembrane protein.

In certain embodiments, the antigenic prokaryotic polypeptide is not derived from a prokaryotic transmembrane protein.

In certain embodiments, the TMB is derived from a viral sequence.

In certain embodiments, the TMB is derived from a viral transmembrane domain sequence selected from the group consisting of: an influenza transmembrane domain sequence, and a non-influenza transmembrane domain sequence selected from the group consisting of a SARS CoV-2 transmembrane domain sequence, a varicella-zoster virus (VZV) transmembrane domain sequence, a measles transmembrane domain sequence, a rubella transmembrane domain sequence, a mumps transmembrane domain sequence, an Ebola transmembrane domain sequence, and a rabies transmembrane domain sequence.

In certain embodiments, the TMB is selected from the group consisting of: an influenza hemagglutinin (HA) transmembrane domain sequence, a SARS CoV-2 spike transmembrane domain sequence, a VZV gB transmembrane domain sequence, a VZV gE transmembrane domain sequence, a VZV gI transmembrane domain sequence, a VZV gK transmembrane domain sequence, a measles F-protein transmembrane domain sequence, a rubella E1 protein transmembrane domain sequence, a rubella E2 protein transmembrane domain sequence, a mumps F-protein transmembrane domain sequence, an Ebola GP protein transmembrane domain sequence and a rabies G protein transmembrane domain sequence, preferably wherein the TMB comprises an HA transmembrane domain sequence from influenza A or influenza B, more preferably from influenza A.

In certain embodiments, the TMB comprises an amino acid sequence selected from the group consisting of: ILAIYSTVASSLVLLVSLGAISF (SEQ ID NO: 17); ILAIYSTVASSLVLVVSLGAISF (SEQ ID NO: 18); ILWISFAISCFLLCVVLLGFI (SEQ ID NO: 19); STAASSLAVTLMLAIFIVYMV (SEQ ID NO: 20); WYIWLGFIAGLIAIVMVTIML (SEQ ID NO: 21); FGALAVGLLVLAGLVAAFFAY (SEQ ID NO: 22); AAWTGGLAAVVLLCLVIFLIC (SEQ ID NO: 23); IIIPIVASVMILTAMVIVIVI (SEQ ID NO: 24); YFWCVQLKMIFFAWFVYGMYL (SEQ ID NO: 25); IVYILIAVCLGGLIGIPALIC (SEQ ID NO: 26); LDHAFAAFVLLVPWVLIFMVC (SEQ ID NO: 27); WWQLTLGAICALLLAGLLACC (SEQ ID NO: 28); IVAALVLSILSIIISLLFCCW (SEQ ID NO: 29); WIPAGIGVTGVIIAVIALFCI (SEQ ID NO: 30); and VLLSAGALTALMLIIFLMTCW (SEQ ID NO: 185).

In certain embodiments, the TMB comprises an amino acid sequence of ILAIYSTVASSLVLLVSLGAISF (SEQ ID NO: 17).

In certain embodiments, the TMB is attached to the antigenic prokaryotic polypeptide with a linker.

In certain embodiments, the TMB is positioned at the N-terminus of the antigenic prokaryotic polypeptide.

In certain embodiments, the TMB is positioned at the C-terminus of the antigenic prokaryotic polypeptide.

In another aspect, the disclosure provides a nucleic acid comprising an open reading frame (ORF), wherein the ORF comprises:—a polynucleotide sequence encoding at least one antigenic polypeptide, preferably an antigenic prokaryotic polypeptide, and—a polynucleotide sequence encoding at least one transmembrane domain (TMB), wherein the TMB is heterologous to the antigenic polypeptide,

wherein optionally the ORF further comprises a polynucleotide sequence encoding at least one secretion signal peptide, preferably a viral secretion signal peptide, more preferably a viral secretion signal peptide as described in any one of the preceding claims.

In another aspect, the disclosure provides a nucleic acid comprising an open reading frame (ORF), wherein the ORF comprises:—a polynucleotide sequence encoding at least one antigenic prokaryotic polypeptide and—a polynucleotide sequence encoding at least one transmembrane domain (TMB).

In certain embodiments the TMB: (a) comprises or consists of 15 to 50 amino acid residues, preferably 15 to 30 amino acid residues, more preferably 18 to 25 amino acid residues; and/or (b) comprises at least 50% of hydrophobic amino acid residues, preferably selected in the group consisting of: alanine, isoleucine, leucine, valine, phenylalanine, tryptophane and tyrosine; and/or (c) comprises at least one alpha helix.

In certain embodiments, the TMB is derived from an integral membrane protein, preferably from a single pass membrane protein, more preferably from a bitopic membrane protein, even more preferably from a bitopic membrane protein of Type I.

In certain embodiments, the TMB is derived from a non-human sequence.

In certain embodiments, the antigenic polypeptide is not derived from a transmembrane protein.

In certain embodiments, the TMB is derived from a viral sequence.

In certain embodiments, the TMB is derived from a viral transmembrane domain sequence selected from the group consisting of: an influenza transmembrane domain sequence, and a non-influenza transmembrane domain sequence selected from the group consisting of a SARS CoV-2 transmembrane domain sequence, a varicella-zoster virus (VZV) transmembrane domain sequence, a measles transmembrane domain sequence, a rubella transmembrane domain sequence, a mumps transmembrane domain sequence, an Ebola transmembrane domain sequence, and a rabies transmembrane domain sequence.

In certain embodiments, the TMB is selected from the group consisting of: an influenza hemagglutinin (HA) transmembrane domain sequence, a SARS CoV-2 spike transmembrane domain sequence, a VZV gB transmembrane domain sequence, a VZV gE transmembrane domain sequence, a VZV gI transmembrane domain sequence, a VZV gK transmembrane domain sequence, a measles F-protein transmembrane domain sequence, a rubella E1 protein transmembrane domain sequence, a rubella E2 protein transmembrane domain sequence, a mumps F-protein transmembrane domain sequence, an Ebola GP protein transmembrane domain sequence and a rabies G protein transmembrane domain sequence, preferably wherein the TMB comprises an HA transmembrane domain sequence from influenza A or influenza B, more preferably from influenza A.

In certain embodiments, the TMB comprises an amino acid sequence selected from the group consisting of: ILAIYSTVASSLVLLVSLGAISF (SEQ ID NO: 17); ILAIYSTVASSLVLVVSLGAISF (SEQ ID NO: 18); ILWISFAISCFLLCVVLLGFI (SEQ ID NO: 19); STAASSLAVTLMLAIFIVYMV (SEQ ID NO: 20); WYIWLGFIAGLIAIVMVTIML (SEQ ID NO: 21); FGALAVGLLVLAGLVAAFFAY (SEQ ID NO: 22); AAWTGGLAAVVLLCLVIFLIC (SEQ ID NO: 23); IIIPIVASVMILTAMVIVIVI (SEQ ID NO: 24); YFWCVQLKMIFFAWFVYGMYL (SEQ ID NO: 25); IVYILIAVCLGGLIGIPALIC (SEQ ID NO: 26); LDHAFAAFVLLVPWVLIFMVC (SEQ ID NO: 27); WWQLTLGAICALLLAGLLACC (SEQ ID NO: 28); IVAALVLSILSIIISLLFCCW (SEQ ID NO: 29); WIPAGIGVTGVIIAVIALFCI (SEQ ID NO: 30); and VLLSAGALTALMLIIFLMTCW (SEQ ID NO: 185).

In certain embodiments, the TMB comprises an amino acid sequence of ILAIYSTVASSLVLLVSLGAISF (SEQ ID NO: 17).

In certain embodiments, the TMB is attached to the antigenic prokaryotic polypeptide with a linker.

In certain embodiments, the TMB is positioned at the N-terminus of the antigenic prokaryotic polypeptide.

In certain embodiments, the TMB is positioned at the C-terminus of the antigenic prokaryotic polypeptide.

In certain embodiments, the antigenic prokaryotic polypeptide is derived from a bacteria of a genus selected from the group consisting of Acetobacter, Acinetobacter, Actinomyces, Aerococcus, Agrobacterium, Anaplasma, Azorhizobia, Azotobacter, Bacillus, Bacteroides, Bartonella, Bordetella, Borrelia, Brucella, Burkkolderia, Calymmatobacterium, Campylobacter, Chlamydia, Chlamydophila, Clostridium, Corynebacterium, Coxiella, Cutibacterium, Ehrlichia, Enterobacter, Enterococcus, Escherichia, Francisella, Fusobacterium, Gardnerella, Haemophilus, Helicobacter, Klebsiella, Lactobacillus, Lactococcus, Legionella, Listeria, Methanobacterium, Microbacterium, Micrococcus, Moraxella, Mycobacterium, Mycoplasma, Neisseria, Pasteurella, Pediococcus, Peptostreptococcus, Porphyromonas, Prevotella, Propionibacterium, Pseudomonas, Rhizobium, Rickettsia, Rochalimaea, Rothia, Salmonella, Serratia, Shigella, Sarcina, Spirillum, Spirochaetes, Staphylococcus, Stenotrophomonas, Streptobacillus, Streptococcus, Tetragenococcus, Treponema, Vibrio, Viridans, Walbachia, and Yersinia, preferably from a bacteria of a species selected from the group consisting of Acetobacter aurantius, Acinetobacter baumannii, Actinomyces israelii, Agrobacterium radiobacter, Agrobacterium tumefaciens, Anaplasma phagocytophilum, Azorhizobium caulinodans, Azotobacter vinelandii, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus Thuringiensis, Bacteroides fragilis, Bacteroides gingivalis, Bacteroides melaninogenicus, Bartonella henselae, Bartonella Quintana, Bordetella bronchiseptica, Bordetella pertussis, Borrelia burgdorferi. Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Campylobacter pylori, Chlamydia trachomatis, Chlamydophila pneumoniae, Chlamydophila psittaci, Clostridium botulinum, Clostridium difficile, Clostridium perfringens, Clostridium tetani, Corynebacterium diphtheriae, Corynebacterium fusiforme, Coxiella burnetii, Cutibacterium acnes, Cutibacterium avidum, Cutibacterium granulosum, Cutibacterium namnetense, Cutibacterium humerusii, Ehrlichia chaffeensis, Enterobacter cloacae, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus bulgaricus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Neisseria gonorrhoeae, Neisseria meningitidis, Pasteurella multocida, Pasteurella tularensis, Peptostreptococcus, Porphyromonas gingivalis, Prevotella melaninogenica, Propionibacterium acnes, Pseudomonas aeruginosa, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus, Staphylococcus epidermidis, Stenotrophomonas maltophilia, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Viridans streptococci, Wolbachia, Yersinia enterocolitica, Yersinia pestis and Yersinia pseudotuberculosis.

In certain embodiments, the antigenic prokaryotic polypeptide is derived from a bacteria of the genus Borrelia, preferably selected from the species B. burgdorferi, afzelii, garinii, bavariensis, mayonii, spielmanii, lusitaniae, bissettii and/or valaisiana.

In certain embodiments, the antigenic prokaryotic polypeptide is OspA or a fragment or variant thereof, wherein the OspA or fragment or variant thereof comprises at least 5 amino acids, preferably wherein the antigenic prokaryotic polypeptide comprises: (a) an amino acid sequence derived from OspA ST1, preferably with at least 85% identity to the sequence KQNVSSLDEKNSVSVDLPGEMKVLVSKEKNKDGKYDLIATVDKLELKGTSDKNNGSGV LEGVKADKSKVKLTISDDLGQTTLEVFKEDGKTLVSKKVTSKDKSSTEEKFNEKGEVSE KIITRADGTRLEYTGIKSDGSGKAKEVLKGYDLKGELSSEKTTLVVKEGTVTLSKNISKS GEVSVELNDTDSSAATKKTAAWNSGTSTLTITVNSKKTKDLVFTKENTITVQQYDSNGT KLEGSAVEITKLDEIKNALK (SEQ ID NO: 31); (b) an amino acid sequence derived from OspA ST2, preferably with at least 85% identity to the sequence KQNVSSLDEKNSASVDLPGEMKVLVSKEKDKDGKYSLKATVDKIELKGTSDKDNGSGV LEGTKDDKSKAKLTIADDLSKTTFELFKEDGKTLVSRKVSSKDKTSTDEMFNEKGELSA KTMTRENGTKLEYTEMKSDGTGKAKEVLKNFTLEGKVANDKVTLEVKEGTVTLSKEIA KSGEVTVALNDTNTTQATKKTGAWDSKTSTLTISVNSKKTTQLVFTKQDTITVQKYDSA GTNLEGTAVEIKTLDELKNALK (SEQ ID NO: 32); (c) an amino acid sequence derived from OspA ST3, preferably with at least 85% identity to the sequence KQNVSSLDEKNSVSVDLPGGMKVLVSKEKDKDGKYSLMATVEKLELKGTSDKSNGSG VLEGEKADKSKAKLTISQDLNQTTFEIFKEDGKTLVSRKVNSKDKSSTEEKFNDKGKLSE KVVTRANGTRLEYTEIKNDGSGKAKEVLKGFALEGTLTDGGETKLTVTEGTVTLSKNIS KSGEITVALNDTETTPADKKTGEWKSDTSTLTISKNSQKPKQLVFTKENTITVQNYNRAG NALEGSPAEIKDLAELKAALK (SEQ ID NO: 186); (d) an amino acid sequence derived from OspA ST4, preferably with at least 85% identity to the sequence KQNVSSLDEKNSVSVDLPGEMKVLVSKEKDKDGKYSLMATVDKLELKGTSDKSNGSG TLEGEKSDKSKAKLTISEDLSKTTFEIFKEDGKTLVSKKVNSKDKSSIEEKFNAKGELSEK TILRANGTRLEYTEIKSDGTGKAKEVLKDFALEGTLAADKTTLKVTEGTVVLSKHIPNSG EITVELNDSNSTQATKKTGKWDSNTSTLTISVNSKKTKNIVFTKEDTITVQKYDSAGTNL EGNAVEIKTLDELKNALK (SEQ ID NO: 187); (e) an amino acid sequence derived from OspA ST5, preferably with at least 85% identity to the sequence KQNVSSLDEKNSVSVDLPGGMKVLVSKEKDKDGKYSLMATVEKLELKGTSDKNNGSG TLEGEKTDKSKVKLTIAEDLSKTTFEIFKEDGKTLVSKKVTLKDKSSTEEKFNEKGEISEK TIVRANGTRLEYTDIKSDGSGKAKEVLKDFTLEGTLAADGKTTLKVTEGTVVLSKNILKS GEITVALDDSDTTQATKKTGKWDSKTSTLTISVNSQKTKNLVFTKEDTITVQKYDSAGT NLEGKAVEITTLEKLKDALK (SEQ ID NO: 188); (f) an amino acid sequence derived from OspA ST6, preferably with at least 85% identity to the sequence KQNVSSLDEKNSVSVDLPGGMTVLVSKEKDKDGKYSLEATVDKLELKGTSDKNNGSGT LEGEKTDKSKVKSTIADDLSQTKFEIFKEDGKTLVSKKVTLKDKSSTEEKFNGKGETSEK TIVRANGTRLEYTDIKSDGSGKAKEVLKDFTLEGTLAADGKTTLKVTEGTVVLSKNILKS GEITAALDDSDTTRATKKTGKWDSKTSTLTISVNSQKTKNLVFTKEDTITVQRYDSAGT NLEGKAVEITTLKELKNALK (SEQ ID NO: 189); (g) an amino acid sequence derived from OspA ST7, preferably with at least 85% identity to the sequence KQNVSSLDEKNSVSVDLPGEMKVLVSKEKDKDGKYSLEATVDKLELKGTSDKNNGSG VLEGVKAAKSKAKLTIADDLSQTKFEIFKEDGKTLVSKKVTLKDKSSTEEKFNDKGKLS EKVVTRANGTRLEYTEIQNDGSGKAKEVLKSLTLEGTLTADGETKLTVEAGTVTLSKNI SESGEITVELKDTETTPADKKSGTWDSKTSTLTISKNSQKTKQLVFTKENTITVQKYNTA GTKLEGSPAEIKDLEALKAALK (SEQ ID NO: 190); (h) any combination of (a)-(g); (i) a sequence derived from OspA ST1 according to (a) and a sequence derived from OspA ST2 according to (b), or (j) a sequence derived from OspA ST1 according to (a), a sequence derived from OspA ST2 according to (b), a sequence derived from OspA ST3 according to (c), a sequence derived from OspA ST4 according to (d), a sequence derived from OspA ST5 according to (e), a sequence derived from OspA ST6 according to (f) and a sequence derived from OspA ST7 according to (g).

In certain embodiments, the antigenic prokaryotic polypeptide comprises at least one mutated glycosylation site, preferably at least one mutated N-linked glycosylation site.

In certain embodiments, the polynucleotide sequence of the nucleic acid is codon optimized.

In certain embodiments, the polynucleotide sequence of the ORF is codon optimized.

In certain embodiments, the polynucleotide sequence encoding the at least one viral secretion signal peptide is codon optimized.

In certain embodiments, the polynucleotide sequence encoding the at least one TMB is codon optimized.

In certain embodiments, the nucleic acid is DNA.

In certain embodiments, the nucleic acid is messenger RNA (mRNA), wherein in particular the mRNA may be non-replicating mRNA, self-replicating mRNA or trans-replicating mRNA.

In certain embodiments, the mRNA comprises at least one 5′ untranslated region (5′ UTR), at least one 3′ untranslated region (3′ UTR), and/or at least one polyadenylation (poly(A)) sequence.

In certain embodiments, the mRNA comprises at least one chemical modification.

In certain embodiments, at least 20%, at least 30%, at least 40%, at least 50%, at least 60%, at least 70%, at least 80%, at least 85%, at least 90%, at least 95%, or 100% of the uracil nucleotides in the mRNA are chemically modified.

In certain embodiments, at least 20%, at least 30%, at least 40%, at least 50%, at least 60%, at least 70%, at least 80%, at least 85%, at least 90%, at least 95%, or 100% of the uracil nucleotides in the ORF are chemically modified.

In certain embodiments, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In certain embodiments, the chemical modification is selected from the group consisting of pseudouridine, N1-methylpseudouridine, 5-methylcytosine, 5-methoxyuridine, and a combination thereof.

In certain embodiments, the chemical modification is N1-methylpseudouridine.

In one aspect, the disclosure provides a composition comprising at least one nucleic acid described above.

In certain embodiments, the composition further comprises a lipid nanoparticle (LNP). In certain embodiments, the nucleic acid is encapsulated in the LNP.

In certain embodiments, the LNP comprises at least one cationic lipid. In certain embodiments the cationic lipid is biodegradable. In certain embodiments, the cationic lipid is not biodegradable. In certain embodiments, the cationic lipid is cleavable. In certain embodiments, the cationic lipid is not cleavable. In certain embodiments, the cationic lipid is selected from the group consisting of OF-02, cKK-E10, OF-Deg-Lin, GL-HEPES-E3-E10-DS-3-E18-1, GL-HEPES-E3-E12-DS-4-E10, GL-HEPES-E3-E12-DS-3-E14, SM-102, and ALC-0315.

In certain embodiments, the LNP further comprises a polyethylene glycol (PEG) conjugated (PEGylated) lipid, a cholesterol-based lipid, and a helper lipid.

In certain embodiments, the LNP comprises:—a cationic lipid at a molar ratio of 35% to 55%;—a polyethylene glycol (PEG) conjugated (PEGylated) lipid at a molar ratio of 0.25% to 2.75%;—a cholesterol-based lipid at a molar ratio of 20% to 45%; and—a helper lipid at a molar ratio of 5% to 35%, wherein all of the molar ratios are relative to the total lipid content of the LNP.

In certain embodiments, the LNP comprises:—a cationic lipid at a molar ratio of 40%;-a PEGylated lipid at a molar ratio of 1.5%;—a cholesterol-based lipid at a molar ratio of 28.5%; and—a helper lipid at a molar ratio of 30%.

In certain embodiments, the the LNP comprises:—a cationic lipid at a molar ratio of 45 to 50%;—a PEGylated lipid at a molar ratio of 1.5 to 1.7%;—a cholesterol-based lipid at a molar ratio of 38 to 43%; and—a helper lipid at a molar ratio of 9 to 10%.

In certain embodiments, the PEGylated lipid is dimyristoyl-PEG2000 (DMG-PEG2000) or 2-[(polyethylene glycol)-2000]-N,N-ditetradecylacetamide (ALC-0159).

In certain embodiments, the cholesterol-based lipid is cholesterol.

In certain embodiments, the helper lipid is 1,2-dioleoyl-SN-glycero-3-phosphoethanolamine (DOPE) or 1,2-distearoyl-sn-glycero-3-phosphocholine (DSPC).

In certain embodiments, the LNP comprises:—a cationic lipid selected from the group consisting of OF-02, cKK-E10, GL-HEPES-E3-E10-DS-3-E18-1, GL-HEPES-E3-E12-DS-4-E10, and GL-HEPES-E3-E12-DS-3-E14, at a molar ratio of 40%;—DMG-PEG2000 at a molar ratio of 1.5%;—cholesterol at a molar ratio of 28.5%; and—DOPE at a molar ratio of 30%.

In certain embodiments, the LNP comprises:—SM-102 at a molar ratio of 50%;—DMG-PEG2000 at a molar ratio of 1.5%;—cholesterol at a molar ratio of 38.5%; and—DSPC at a molar ratio of 10%.

In certain embodiments, the LNP comprises:—ALC-0315 at a molar ratio of 46.3%;-ALC-0159 at a molar ratio of 1.6%;—cholesterol at a molar ratio of 42.7%; and—DSPC at a molar ratio of 9.4%.

In certain embodiments, the LNP comprises:—ALC-0315 at a molar ratio of 47.4%;-ALC-0159 at a molar ratio of 1.7%;—cholesterol at a molar ratio of 40.9%; and—DSPC at a molar ratio of 10%.

In certain embodiments, the LNP has an average diameter of 30 nm to 200 nm.

In certain embodiments, the LNP has an average diameter of 80 nm to 150 nm.

In certain embodiments, the composition comprises between 1 mg/mL to 10 mg/mL of the LNP.

In certain embodiments, the LNP comprises between 1 and 20 nucleic acid molecules, preferably mRNA molecules.

In certain embodiments, the composition is formulated for administration intramuscularly, intranasally, intravenously, subcutaneously, or intradermally.

In certain embodiments, the composition comprises a phosphate-buffer saline.

In certain embodiments, the composition is a pharmaceutical composition, for example an immunogenic composition or a vaccine, in particular an mRNA vaccine.

In another aspect, the disclosure provides a nucleic acid or a composition for use in eliciting an immune response in a subject in need thereof.

In another aspect, the disclosure provides a nucleic acid or a composition for use in treating or preventing a prokaryotic infection in a subject in need thereof.

In another aspect, the disclosure provides a method of secreting an antigenic prokaryotic polypeptide in a host cell, the method comprising administering to the host cell the nucleic acid or composition described above.

In another aspect, the disclosure provides a method of displaying an antigenic prokaryotic polypeptide on the surface of a host cell, the method comprising administering to the host cell the nucleic acid or composition described above.

In another aspect, the disclosure provides a kit comprising a container comprising a single-use or multi-use dosage of the nucleic acid or composition described above, optionally wherein the container is a vial or a pre-filled syringe or injector.

### BRIEF DESCRIPTION OF THE DRAWINGS

FIG. 1 depicts the structure of the Outer Surface Protein A (OspA) serotype 1 (ST1) and serotype 2 (ST2) mRNA constructs. Different mRNA sequences were designed to direct the expression of OspA intracellularly, secreted, or transmembrane using the OspA sequence without or with fusion to hemagglutinin secretion signal (HA SS) and/or HA transmembrane domain (HA TMB). OspA native sequence was used as well as sequences with glycosylation site mutations (Gly-) to avoid glycosylation of the protein encoded by the mRNA.

FIG. 2A—FIG. 2B depict Western blot images showing the in vitro expression of OspA ST1 and ST2 mRNA in HEK293T cell's supernatants. mRNA-OspA ST1 (FIG. 2A) and mRNA-OspA ST2 (FIG. 2B) without or with an HA secretion signal, and without or with glycosylation site mutations, are depicted. A negative control (buffer) and a positive control (recombinant OspA) were also used. HEK293 cells were transfected with vaccine mRNA. After 48 hours, supernatants were collected and run on a Western blot. Blotted proteins were detected with polyclonal rabbit antibodies that recognize OspA.

FIG. 3A—FIG. 3C depict Western blot images showing the in vitro expression of OspA ST1 mRNA containing an HA SS and an HA TMB, and without or with glycosylation site mutations, in HEK293 cells. Cell's supernatants (FIG. 3A), crude extracts (FIG. 3B) and intracellular compartments (FIG. 3C) are shown. After 48 or 72 hours, supernatants and cells were collected and run on a Western blot. Blotted proteins were detected with polyclonal rabbit antibodies that recognize OspA.

FIG. 4A—FIG. 4C depict Western blot images showing the in vitro expression of OspA ST2 mRNA containing an HA SS and an HA TMB, and without or with glycosylation site mutations, in HEK293 cells. Cell's supernatants (FIG. 4A), crude extracts (FIG. 4B) and intracellular compartments (FIG. 4C) are shown. After 48 or 72 hours, supernatants and cells were collected and run on a Western blot. Blotted proteins were detected with polyclonal rabbit antibodies that recognize OspA.

FIG. 5 depicts antigenicity of OspA ST1 antigen delivered by mRNA in HEK293 cells. Transfected cell supernatants were used to perform sandwich ELISAs with functional monoclonal antibodies LA-2, 857-2 and 221-7.

FIG. 6 depicts antigenicity of OspA ST2 antigen delivered by mRNA in HEK293 cells. Transfected cell supernatants were used to perform sandwich ELISAs with functional monoclonal antibodies 857-2 and 221-7.

FIG. 7A—FIG. 7B depict IgG titer values from an anti-OspA ST1 IgG ELISA post-dose 1 (day 20) (FIG. 7A) and post-dose 2 (day 35) (FIG. 7B). HA-SS=Secretion signal Hemagglutinin; TMB=Transmembrane domain; Gly(-)=glycosylation sites mutations; Dotted line=limit of quantification.

FIG. 8 is a schematic representation of the elements included in an expanded panel of mRNA constructs. The panel consisted of three different prokaryotic antigens: OspA ST1, CAMP2, and PITP as displayed on the left side of the schematic. The right side of the schematic shows that the antigens were fused to signal sequences (labeled “SS”) of glycoproteins derived from the following viral families: Influenza (subtypes A or B), Rabies, Varicella (VZV), or Ebola viruses. Some of the panel constructs further contained the respective glycoprotein transmembrane domain (labeled “TMB”). “Flu” stands for “influenza” virus.

FIG. 9 depicts Western blot images showing the in vitro protein expression of OspA ST1 mRNA containing a SS from glycoprotein derived from Influenza (subtypes A or B), Rabies, Varicella (VZV), or Ebola viruses, with or without their respective glycoprotein TMB domain post transfection in HEK Expi293F cells. OspA ST1 (expected size about 28-34 kDa) was detected using a rabbit polyclonal antibody to OspA at a 1:2000 dilution (ABCAM ab106081). Controls included an OspA ST1 construct without any SS or any lipidation sequence (first lane on all gels, labeled “No SS”) as well as recombinant OspA ST1 (last lane on all gels). Samples were collected at a 48-hour time point from crude extracts (total lysate), cell supernatants, and fractionated cell samples containing either intracellular or transmembrane compartments.

FIG. 10 depicts Western blot images showing the in vitro protein expression of OspA ST1 mRNA containing a SS from glycoprotein derived from Influenza (subtypes A or B) and Rabies (left gel) and Varicella (VZV) and Ebola (right gel) viruses with or without their respective glycoprotein TMB domain post transfection in HEK Expi293F cells. The cell supernatants were collected at a 48-hour time point and were concentrated 7-fold. Half of the resulting sample volume was deglycosylated (represented by a “D” on the gels) to analyze the protein by Western blot before and after enzymatic treatment. OspA ST1 (expected size about 28-34 kDa) was detected using a rabbit polyclonal antibody to OspA at a 1:2000 dilution (ABCAM ab106081). Controls included a transfection control (cells transfected without mRNA), an OspA ST1 construct without any SS (labeled “No SS”) as well as recombinant OspA ST1 (last lane on all gels).

FIG. 11 depicts Western blot images showing the in vitro protein expression of CAMP2 mRNA containing a SS from glycoprotein derived from Influenza (subtypes A or B), Rabies, Varicella (VZV), or Ebola viruses with or without their respective glycoprotein TMB domain post transfection in HEK Expi293F cells. CAMP2 (expected size about 26-32 kDa) was detected using a rabbit polyclonal antibody to CAMP2 at a 1:1500 dilution. Controls included a CAMP2 construct without any SS (first lane on all gels, labeled “No SS”) as well as recombinant CAMP2 (last lane on all gels). Samples were collected at either a 48-hour time point from crude extracts (total lysate), cell supernatants, and fractionated cell samples containing either intracellular or transmembrane compartments.

FIG. 12 depicts Western blot images showing the in vitro protein expression of PITP mRNA containing a SS from glycoprotein derived from Influenza (subtypes A or B), Rabies, Varicella (VZV), or Ebola viruses with or without their respective glycoprotein TMB domain post transfection in HEK Expi293F cells. PITP (expected size about 42-48 kDa) was detected using a mouse polyclonal antibody to PITP at a 1:1000 dilution. Controls included an PITP construct without any SS or any TMB (first lane on all gels, labeled “No SS”) as well as recombinant PITP (last lane on all gels). Samples were collected at a 48-hour time point from crude extracts (total lysate), cell supernatants, and fractionated cell samples containing either intracellular or transmembrane compartments.

FIG. 13 left side of the table summarizes the Western blot analysis of protein expression and localization from OspA ST1, CAMP2, and PITP mRNA constructs containing a SS from glycoprotein derived from Influenza (subtypes A or B), Rabies, Varicella (VZV), or Ebola viruses, with or without their respective glycoprotein TMB domain post transfection in HEK Expi293F cells. FIG. 13 right side of the table is the in-silico signal sequence prediction scores derived from SignalP.

### DETAILED DESCRIPTION OF THE DISCLOSURE

The present disclosure is directed to, inter alia, nucleic acid (e.g., mRNA) compositions encoding an antigenic prokaryotic polypeptide linked to one or both of a viral secretion signal peptide sequence and transmembrane domain (TMB), and methods of vaccination with the same.

### I. Definitions

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art.

Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

It is to be noted that the term “a” or “an” entity refers to one or more of that entity; for example, “a nucleotide sequence,” is understood to represent one or more nucleotide sequences.

As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, may provide one of skill with a general dictionary of many of the terms used in this disclosure.

Units, prefixes, and symbols are denoted in their International System of Units (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

The term “approximately” or “about” is used herein to mean approximately, roughly, around, or in the regions of. When the term “about” is used in conjunction with a numerical range, it modifies that range by extending the boundaries above and below the numerical values set forth.

In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or ±0.01%. In some embodiments, “about” indicates deviation from the indicated numerical value by ±10%. In some embodiments, “about” indicates deviation from the indicated numerical value by +5%. In some embodiments, “about” indicates deviation from the indicated numerical value by +4%. In some embodiments, “about” indicates deviation from the indicated numerical value by +3%. In some embodiments, “about” indicates deviation from the indicated numerical value by +2%. In some embodiments, “about” indicates deviation from the indicated numerical value by +1%. In some embodiments, “about” indicates deviation from the indicated numerical value by +0.9%. In some embodiments, “about” indicates deviation from the indicated numerical value by +0.8%. In some embodiments, “about” indicates deviation from the indicated numerical value by ±0.7%. In some embodiments, “about” indicates deviation from the indicated numerical value by +0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by +0.3%. In some embodiments, “about” indicates deviation from the indicated numerical value by +0.1%. In some embodiments, “about” indicates deviation from the indicated numerical value by +0.05%. In some embodiments, “about” indicates deviation from the indicated numerical value by ±0.01%.

As used herein, the term “messenger RNA” or “mRNA” refers to a polynucleotide that encodes at least one polypeptide. mRNA as used herein encompasses both modified and unmodified RNA. mRNA may contain one or more coding and non-coding regions. A coding region is alternatively referred to as an open reading frame (ORF). Non-coding regions in mRNA include the 5′ cap, 5′ untranslated region (UTR), 3′ UTR, and a polyA tail. mRNA can be purified from natural sources, produced using recombinant expression systems (e.g., in vitro transcription) and optionally purified, or chemically synthesized.

As used herein, the term “open reading frame”, “ORF”, or “coding region” refers to a polynucleotide sequence beginning with a start codon (e.g. ATG) and ending with a stop codon (e.g. TAA, TAG or TGA), without any other stop codon in between, and that encodes a protein (e.g., an antigenic prokaryotic polypeptide).

As used herein, the term “viral secretion signal peptide” or “SS” refers to an amino acid sequence derived from a virus that directs a polypeptide sequence to which it is attached through the cellular secretory pathway. Polypeptides with SS sequences are transited through one or more organelles in the cell until secretion outside of the cell through a secretory vesicle.

As used herein, the term “transmembrane domain” or “TMB” refers to an amino acid sequence that possesses a cell membrane-spanning property. A TMB triggers anchoring of a polypeptide to which it is attached to the cell membrane.

Also included in the present disclosure are fragments or variants of polypeptides, and any combination thereof. The term “fragment” or “variant” when referring to the antigenic prokaryotic polypeptides of the present disclosure include any polypeptides which retain at least some of the properties (e.g., specific antigenic property of the polypeptide or the ability of polypeptide to contribute to the induction of antibody binding) of the reference polypeptide. Fragments of polypeptides include N-terminally and/or C-terminally truncated fragments, e.g., C-terminal fragments and N-terminal fragments, as well as deletion fragments but do not include the naturally occurring full-length polypeptide (or mature polypeptide). A deletion fragment refers to a polypeptide with 1 or more internal amino acids deleted from the full-length polypeptide. Variants of polypeptides include fragments as described above, and also polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions or additions. Such variations (i.e. truncations and/or amino acid substitutions, deletions, or insertions) may occur either on the amino acid level or correspondingly on the nucleic acid level.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another embodiment, a string of amino acids can be conservatively replaced with a structurally similar string that differs in order and/or composition of side chain family members.

The term “linked” or “attached” as used herein refers to a first amino acid sequence or nucleotide sequence covalently or non-covalently joined to a second amino acid sequence or nucleotide sequence, respectively (e.g., a secretion signal amino acid sequence and/or a transmembrane domain amino acid sequence linked to an antigenic prokaryotic polypeptide amino acid sequence). The first amino acid or nucleotide sequence can be directly joined or juxtaposed to the second amino acid or nucleotide sequence or alternatively an intervening sequence can covalently join the first sequence to the second sequence. The term “linked” means not only a fusion of a first amino acid sequence to a second amino acid sequence at the C-terminus or the N-terminus, but also includes insertion of the whole first amino acid sequence (or the second amino acid sequence) into any two amino acids in the second amino acid sequence (or the first amino acid sequence, respectively). In one embodiment, the first amino acid sequence can be linked to a second amino acid sequence by a peptide bond or a linker. The first nucleotide sequence can be linked to a second nucleotide sequence by a phosphodiester bond or a linker. The linker can be a peptide or a polypeptide (for polypeptide chains) or a nucleotide or a nucleotide chain (for nucleotide chains) or any chemical moiety (for both polypeptide and polynucleotide chains). The term “linked” is also indicated by a hyphen (-).

As used herein, the term “glycosylation” refers to the addition of a saccharide unit to a protein.

As used herein, the term “N-glycan” refers to a saccharide chain attached to a protein at the amide nitrogen of an N (asparagine) residue of the protein. As such, an N-glycan is formed by the process of N-glycosylation. This glycan may be a polysaccharide.

As used herein, the term “immune response” refers to a response of a cell of the immune system, such as a B cell, T cell, dendritic cell, macrophage or polymorphonucleocyte, to a stimulus such as an antigen or vaccine. An immune response can include any cell of the body involved in a host defense response, including for example, an epithelial cell that secretes an interferon or a cytokine. An immune response includes, but is not limited to, an innate and/or adaptive immune response.

As used herein, a “protective immune response” refers to an immune response that protects a subject from infection (e.g., prevents infection or prevents the development of disease associated with infection). Methods of measuring immune responses are well known in the art and include, for example, by measuring proliferation and/or activity of lymphocytes (such as B or T cells), secretion of cytokines or chemokines, inflammation, antibody production and the like.

As used herein, an “antibody response” is an immune response in which antibodies are produced.

As used herein, an “antigen” refers to an agent that elicits an immune response, and/or an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism. Alternatively, or additionally, in some embodiments, an antigen elicits a cellular response (e.g., involving T-cells whose receptors specifically interact with the antigen) in an organism. A particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor, and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity. Antigens include prokaryotic antigen polypeptides (e.g., OspA ST1 and ST2) encoded by the mRNA as described herein. A “prokaryotic antigen” or “antigenic prokaryotic polypeptide” includes any antigenic polypeptide derived from a prokaryotic organism that is capable of eliciting an immune response.

As used herein, an “adjuvant” refers to a substance or vehicle that enhances the immune response to an antigen. Adjuvants can include, without limitation, a suspension of minerals (e.g., alum, aluminum hydroxide, or phosphate) on which antigen is adsorbed; a water-in-oil or oil-in-water emulsion in which antigen solution is emulsified in mineral oil or in water (e.g., Freund's incomplete adjuvant). Sometimes killed mycobacteria is included (e.g., Freund's complete adjuvant) to further enhance antigenicity. Immuno-stimulatory oligonucleotides (e.g., a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants can also include biological molecules, such as Toll-Like Receptor (TLR) agonists and costimulatory molecules. As used herein, a “subject” refers to any member of the animal kingdom. In some embodiments, “subject” refers to humans. In some embodiments, “subject” refers to non-human animals. In certain embodiments, the non-human subject is a mammal, e.g., a rodent, a mouse, a rat, a rabbit, a monkey, a llama, a horse, a dog, a cat, a bovine, a sheep, a goat, a primate, a pig.

In some embodiments, when the subject is a human, the terms “individual” or “patient” are used and are intended to be interchangeable with “subject”.

As used herein, the terms “prevent”, “preventing”, “prevention” or “prophylaxis” (and grammatical variants thereof) refer to partially or completely inhibiting the onset of one or more symptoms or features of a particular infection, disease, disorder, and/or condition.

As used herein, the terms “treat”, “treating”, “treatment”, “therapy” or “therapeutic” (and grammatical variants thereof) refer to partially or completely alleviating, ameliorating, improving, relieving, inhibiting progression of, and/or reducing severity of one or more symptoms or features of an infection, disease, disorder, and/or condition.

As used herein, the term “effective amount” refers to an amount (e.g., of a nucleic acid or composition) sufficient to effect beneficial or desired results. An effective amount can be administered in one or more administrations, applications or dosages, and is not intended to be limited to a particular formulation or administration route.

The term “effective amount” includes, e.g., “therapeutically effective amount” and/or “prophylactically effective amount”.

The phrase “therapeutically effective amount” as used herein refers to an amount (e.g., of a nucleic acid or composition) which is effective for producing some desired therapeutic effects in the treatment of an infection, disease, disorder and/or condition at a reasonable benefit/risk ratio applicable to any medical treatment.

The phrase “prophylactically effective amount” as used herein refers to an amount (e.g., of a nucleic acid or composition) which is effective for producing some desired prophylactic effects in the prevention of an infection, disease, disorder and/or condition at a reasonable benefit/risk ratio applicable to any medical treatment.

As used herein, the term “vaccination” or “vaccinate” refers to the administration of a composition intended to generate an immune response, for example to a disease-causing agent.

Vaccination can be administered before, during, and/or after exposure to a disease-causing agent, and/or to the development of one or more symptoms, and in some embodiments, before, during, and/or shortly after exposure to the agent. In some embodiments, vaccination includes multiple administrations, appropriately spaced in time, of a vaccine composition.

The disclosure describes nucleic acid sequences (e.g., DNA and RNA sequences) and amino acid sequences having a certain degree of identity to a given nucleic acid sequence or amino acid sequence, respectively (a reference sequence).

“Sequence identity” between two nucleic acid sequences indicates the percentage of nucleotides that are identical between the sequences. “Sequence identity” between two amino acid sequences indicates the percentage of amino acids that are identical between the sequences.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional property of said given sequence, e.g., and in some instances, are functionally equivalent to said given sequence. In some embodiments, a nucleic acid sequence or amino acid sequence having a particular degree of identity to a given nucleic acid sequence or amino acid sequence is functionally equivalent to said given sequence.

As used herein, the term “kit” refers to a packaged set of related components, such as one or more compounds or compositions and one or more related materials such as solvents, solutions, buffers, instructions, or desiccants.

### II. Secretion Signal Sequences

The use of viral secretion signal peptide (SS) sequences attached to antigenic prokaryotic polypeptides may offer numerous advantages for vaccination. When expressed from an mRNA, especially in a eukaryotic cell, the SS-prokaryotic antigen fusion protein may have increased extracellular expression relative to the prokaryotic antigen without the SS sequence. The increased extracellular expression may promote higher immunogenicity and by extension, better vaccine efficacy.

Viral SS sequences may be found in publicly accessible databases (e.g., the NCBI or UniProt databases) which include an annotated viral polypeptide sequence and identify the start and end position of an experimentally validated SS.

In certain embodiments, the SS sequence as well as the location of the SS sequence cleavage site for a given known input polypeptide sequence may be predicted by using the SignalP algorithm. The SignalP algorithm (and more particularly SignalP v6.0) is described in further detail in Armenteros et al. (Nature Biotechnology. 37: 420-423. 2019), Teufel et al. (Nature Biotechnology. 40: 1023-1025. 2022), and https:/services.healthtech.dtu.dk/services/SignalP-6.0/, each of which is incorporated herein by reference in their entirety. The strength of the prediction is assessed based on a cumulative rank score that considers the likelihood of detecting canonical features of the signal sequence (SS likelihood score) and the probability of cleavage at the cleavage site (cleavage probability score).

In certain embodiments, the viral secretion signal peptide is derived from a viral sequence in a virus able to infect humans. The phrase “influenza”, “SARS CoV-2”, “varicella-zoster virus (VZV)”, “measles”, “rubella”, “rabies,” “Ebola,” and “smallpox” preceding the phrase “secretion signal peptide sequence” indicates that the secretion signal peptide was derived from the virus corresponding to that name.

In certain embodiments, the viral secretion signal peptide is derived from a viral sequence selected from the group consisting of: an influenza secretion signal peptide sequence, a SARS CoV-2 secretion signal peptide sequence, a varicella-zoster virus (VZV) secretion signal peptide sequence, a measles secretion signal peptide sequence, a rubella secretion signal peptide sequence, a mumps secretion signal peptide sequence, an Ebola secretion signal peptide sequence, a rabies secretion signal peptide sequence, and a smallpox secretion signal peptide sequence. These particular signal peptides are derived from viral sequences in viruses which have been administered to humans as vaccines (live-attenuated, inactivated or mRNA), with demonstrated strong safety profiles.

In certain embodiments, the viral secretion signal peptide is selected from the group consisting of: an influenza hemagglutinin (HA) secretion signal peptide sequence, a SARS CoV-2 spike secretion signal peptide sequence, a VZV gB secretion signal peptide sequence, a VZV gE secretion signal peptide sequence, a VZV gI secretion signal peptide sequence, a VZV gK secretion signal peptide sequence, a measles F-protein secretion signal peptide sequence, a rubella E1 protein secretion signal peptide sequence, a rubella E2 protein secretion signal peptide sequence, a mumps F-protein secretion signal peptide sequence, an Ebola GP protein secretion signal peptide sequence, a rabies virus glycoprotein (Rabies G) secretion signal peptide sequence, and a smallpox 6 kDa IC protein secretion signal peptide sequence.

In certain embodiments, the viral secretion signal peptide comprises an HA secretion signal peptide sequence from influenza A or influenza B, preferably from influenza A.

Exemplary viral secretion signal peptide amino acid sequences of the disclosure are shown below in Table 1. Exemplary viral secretion signal peptide amino acid sequences derived from Influenza A or B of the disclosure are shown below in Table 2.

TABLE 1
Viral Secretion Signal Peptide (SS) Amino Acid Sequences

| NAME OF | | | |
|---|---|---|---|
| THE | | | |
| PROTEIN | ORGANISM | STRAIN | SEQUENCE OF THE SS |
| HA (H1N1) | Influenza virus | A/New Caledonia/20/1999 | MKAKLLVLLCTFTATYA |
| | | | (SEQ ID NO: 1) |
| HA (H1N1pdm) | Influenza virus | A/California/7/2009 | MKAILVVLLYTFATANA |
| | | | (SEQ ID NO: 2) |
| HA (H3N2) | Influenza virus | A/Moscow/10/1999 | MKTIIALSYILCLVFA |
| | | | (SEQ ID NO: 3) |
| HA B | Influenza virus | B/Phuket/3073/2013 | MKAIIVLLMVVTSNA |
| | | | (SEQ ID NO: 4) |
| Spike | SARS CoV-2 | Wuhan-1 | MFVFLVLLPLVS |
| | | | (SEQ ID NO: 5) |
| Spike | SARS CoV-2 | Wuhan-1 (long version) | MFLLTTKRTMFVFLVLLPLV |
| | | | S |
| | | | (SEQ ID NO: 6) |
| gB | VZV | Oka strain | MSPCGYYSKWRNRDRPEYR |
| | | | RNLRFRRFFSSIHPNAAAGSG |
| | | | FNGPGVFITSVTGVWLCFLCI |
| | | | FSMFVTAVVS |
| | | | (SEQ ID NO: 7) |
| gE | VZV | Oka strain | MGTVNKPVVGVLMGFGIIT |
| | | | GTLRITNPVRA |
| | | | (SEQ ID NO: 8) |
| gI | VZV | Oka strain | MFLIQCLISAVIFYIQVTNA |
| | | | (SEQ ID NO: 9) |
| gK | VZV | Oka strain | MQALGIKTEHFIIMCLLSGH |
| | | | A |
| | | | (SEQ ID NO: 10) |
| F | Measles | Edmonston-Zagreb strain | MGLKVNVSAIFMAVLLTLQ |
| | | | TPTG |
| | | | (SEQ ID NO: 11) |
| E1 | Rubella | RA27/3 strain | MGAAAALTAVVLQGYNPPA |
| | | | YG |
| | | | (SEQ ID NO: 12) |
| E2 | Rubella | RA27/3 strain | MGAPQAFLAGLLLAAVAVG |
| | | | TARA |
| | | | (SEQ ID NO: 13) |
| F | Mumps | Miyahara strain | MKVFLVTCLGFAVFSSSVC |
| | | | (SEQ ID NO: 14) |
| GP | Ebola virus | Mayinga-76 strain | MGVTGILQLPRDRFKRTSFF |
| | | | LWVIILFQRTES |
| | | | (SEQ ID NO: 15) |
| 6 kDa IC | Smallpox | Germany 91-3 strain | MRSLIIFLLFPSIIYS |
| | | | (SEQ ID NO: 16) |
| Rabies G | Rabies | Rabies Pasteur strain | MVPQALLFVPLLVFPLCFG |
| | | | (SEQ ID NO: 184) |

TABLE 2
Influenza virus A and B Specific Viral Secretion Signal Peptide (SS) Amino Acid
Sequences

| STRAIN NAME OR ID | SEQUENCE OF THE SS |
|---|---|
| A/Beijing/262/95 | MKAKLLVLLCTFTATYA |
| (H1N1)-like virus | (SEQ ID NO: 95) |
| A/Brisbane/02/2018 | MKAILVVLLYTFTTANA |
| (H1N1)pdm09-like virus | (SEQ ID NO: 96) |
| A/Brisbane/59/2007 | MKVKLLVLLCTFTATYA |
| (H1N1)-like virus | (SEQ ID NO: 97) |
| A/California/7/2009 | MKAILVVLLYTFATANA |
| (H1N1)-like virus | (SEQ ID NO: 98) |
| A/Guangdong- | MKAILVVLLYTFTTANA |
| Maonan/SWL1536/2019 | (SEQ ID NO: 99) |
| (H1N1)pdm09-like virus | |
| A/Hawaii/70/2019 | MKAILVVLLYTFTTANA |
| (H1N1)pdm09-like virus | (SEQ ID NO: 100) |
| A/Michigan/45/2015 | MKAILVVLLYTFTTANA |
| (H1N1)pdm09-like virus | (SEQ ID NO: 101) |
| A/New Caledonia/20/99 | MKAKLLVLLCTFTATYA |
| (H1N1)-like virus | (SEQ ID NO: 102) |
| A/Solomon Islands/3/2006 | MKVKLLVLLCTFTATYA |
| (H1N1)-like virus | (SEQ ID NO: 103) |
| A/Sydney/5/2021 | MKAILVVMLYTFTTANA |
| (H1N1)pdm09-like virus | (SEQ ID NO: 104) |
| A/Victoria/2570/2019 | MKAILVVMLYTFTTANA |
| (H1N1)pdm09-like virus | (SEQ ID NO: 105) |
| A/Victoria/4897/2022 | MKAILVVMLYTFTTANA |
| (H1N1)pdm09-like virus | (SEQ ID NO: 106) |
| A/Wisconsin/588/2019 | MKAILVVMLYTFTTANA |
| (H1N1)pdm09-like virus | (SEQ ID NO: 107) |
| A/Wisconsin/67/2022 | MKAILVVMLYTFTTANA |
| (H1N1)pdm09-like virus | (SEQ ID NO: 108) |
| H1N1 CONSENSUS SEQ #1 | MKAILVVLLYTFTTANA |
| (without substitutions) | (SEQ ID NO: 109) |
| A/Brisbane/10/2007 | MKTIIALSYILCLVFT |
| (H3N2)-like virus | (SEQ ID NO: 110) |
| A/California/7/2004 | MKTIIALSYILCLVFA |
| (H3N2)-like virus | (SEQ ID NO: 111) |
| A/Cambodia/e0826360/2020 | MKTIIALSYILCLVFA |
| (H3N2)-like virus | (SEQ ID NO: 112) |
| A/Darwin/6/2021 | MKTIIALSNILCLVFA |
| (H3N2)-like virus | (SEQ ID NO: 113) |
| A/Darwin/9/2021 | MKTIIALSNILCLVFA |
| (H3N2)-like virus | (SEQ ID NO: 114) |
| A/Fujian/411/2002 | MKTIIALSYILCLVFA |
| (H3N2)-like virus | (SEQ ID NO: 115) |
| A/Hong Kong/2671/2019 | MKAIIALSNILCLVFA |
| (H3N2)-like virus | (SEQ ID NO: 116) |
| A/Hong Kong/45/2019 | MKAIIALSNILCLVFA |
| (H3N2)-like virus | (SEQ ID NO: 117) |
| A/Hong Kong/4801/2014 | MKTIIALSYILCLVFA |
| (H3N2)-like virus | (SEQ ID NO: 118) |
| A/Kansas/14/2017 | MKTIIALSCILCLVFA |
| (H3N2)-like virus | (SEQ ID NO: 119) |
| A/Moscow/10/99 | MKTIIALSYILCLVFA |
| (H3N2)-like virus | (SEQ ID NO: 120) |
| A/Perth/16/2009 | MKTIIALSYILCLVFA |
| (H3N2)-like virus | (SEQ ID NO: 121) |
| A/Singapore/INFIMH-16- | MKTIIALSYILCLVFA |
| 0019/2016 | (SEQ ID NO: 122) |
| (H3N2)-likevirus | |
| A/South | MKTIIALSYILCLVFA |
| Australia/34/2019 | (SEQ ID NO: 123) |
| (H3N2)-like virus | |
| A/Switzerland/8060/2017 | MKTIIALSYILCLVFA |
| (H3N2)-like virus | (SEQ ID NO: 124) |
| A/Switzerland/9715293/2013 | MKTIIALSYILCLVFA |
| (H3N2)-like virus | (SEQ ID NO: 125) |
| A/Sydney/5/97 | MKTIALSYILCLVFA |
| (H3N2)-like virus | (SEQ ID NO: 126) |
| A/Texas/50/2012 | MKTIIALSYILCLVFA |
| (H3N2)-like virus | (SEQ ID NO: 127) |
| A/Victoria/361/2011 | MKTIIALSHILCLVFA |
| (H3N2)-like virus | (SEQ ID NO: 128) |
| A/Wellington/1/2004 | MKTIIALSYILCLVFA |
| (H3N2)-like virus | (SEQ ID NO: 129) |
| A/Wisconsin/67/2005 | MKTIIALSYILCLVFA |
| (H3N2)-like virus | (SEQ ID NO: 130) |
| H3N2 CONSENSUS SEQ #1 | MKTIIALSYILCLVFA |
| (without substitutions) | (SEQ ID NO: 131) |
| B/Austria/1359417/2021 | MKAIIVLLMVVTSNA |
| (B/Victoria lineage)-like | (SEQ ID NO: 132) |
| B/Brisbane/60/2008-like | MKAIIVLLMVVTSNA |
| virus | (SEQ ID NO: 134) |
| B/Colorado/06/2017-like | MKAIIVLLMVVTSSA |
| virus (B/Victoria/2/87 | (SEQ ID NO: 135) |
| lineage) | |
| B/Hong Kong/330/2001- | MKAIIVLLMVVTSNA |
| like virus | (SEQ ID NO: 136) |
| B/Malaysia/2506/2004- | MKAIIVLLMVVTSNA |
| like virus | (SEQ ID NO: 137) |
| B/Washington/02/2019 | MKAIIVLLMVVTSNA |
| (B/Victoria lineage)-like | (SEQ ID NO: 138) |
| virus | |
| B/Beijing/184/93-like | MKAIIVLLMVVTSNA |
| virus | (SEQ ID NO: 139) |
| B/Florida/4/2006-like | MKAIIVLLMVVTSNA |
| virus | (SEQ ID NO: 140) |
| B/Massachusetts/2/2012- | MKAIIVLLMVVTSNA |
| like virus | (SEQ ID NO: 141) |
| B/Phuket/3073/2013-like | MKAIIVLLMVVTSNA |
| virus | (SEQ ID NO: 142) |
| B/Sichuan/379/99-like | MEAIIVLLMVVTSNA |
| virus | (SEQ ID NO: 143) |
| INFLUENZA B | MKAIIVLLMVVTSNA |
| VICTORIA/YAMAGATA | (SEQ ID NO: 144) |
| CONSENSUS | |
| SEQUENCE (without | |
| substitutions) | |
| H1N1 CONSENSUS SEQ #2 | MKX₁X₂LX₃VX₄LX₅TFX₆X₇X₈X₉A |
| (with substitutions) | X₁ is selected from A and V; X₂ is selected from I and K; |
| | X₃ is selected from V and L; X₄ is selected from L and M; X₅ |
| | is selected from Y and C; X₆ is selected from T and A X₇ is |
| | selected from T and A; X₈ is selected from A and T; and X₉ |
| | is selected from N and Y (SEQ ID NO: 145) |
| H3N2 CONSENSUS SEQ #2 | MKX₁IIALSX₂ILCLVFX₃ |
| (with substitutions) | X₁ is selected from T and A; X₂ is selected from Y, N, C, |
| | and H; and X₃ is selected from T and A |
| | (SEQ ID NO: 146) |
| INFLUENZA B | MKAIIVLLMVVTSX₁A |
| VICTORIA | X₁ is selected from S and N |
| CONSENSUS SEQ (with | (SEQ ID NO: 147) |
| substitutions) | |
| INFLUENZA B | MX₁AIIVLLMVVTSNA |
| YAMAGATA | X₁ is selected from K and E |
| CONSENSUS SEQ (with | (SEQ ID NO: 148) |
| substitutions) | |

### III. Transmembrane Domains

The inclusion of a transmembrane domain (TMB) in a construct, especially an mRNA construct used as a vaccine antigen, which may also comprise a SS, thereby producing a SS-antigen-TMB fusion protein (and possibly thereafter an antigen-TMB protein, after cleavage of the SS in the mature protein), aims to localize the antigen to the cell surface, by anchoring it at the membrane. This may reduce antigen intracellular localization and further promote higher immunogenicity relative to the antigen without the TMB sequence. The addition of a TMB may allow in particular to increase the humoral (B-cell) response against the antigen. This may be useful for prokaryotic antigens, but may also be used for other antigens (e.g., viral antigens). The addition of a TMB may be used with an antigen derived from a membrane protein or with an antigen derived from a protein that is not a membrane protein (e.g., secreted protein or intracellular protein). The addition of any of the more specific TMBs as described herein may particularly be useful for antigens that are derived from a protein that is not a membrane protein, i.e., a protein which does not naturally contain a TMB (or similar).

The TMB may be from any known TMB in the art, including but not limited to, TMBs from eukaryotic transmembrane proteins (e.g., mammalian transmembrane proteins, such as human transmembrane proteins), TMBs from prokaryotic transmembrane proteins, and TMBs from viral transmembrane proteins. TMBs may further be identified through in silico prediction algorithms, for example, in the TMHMM prediction method described in Krogh et al. (J Mol Biol. 305(3): 567-580. 2001) and https://services.healthtech.dtu.dk/services/TMHMM-2.0/, each of which is incorporated herein by reference in their entirety. Some features of TMBs are described in further detail in Albers et al. (Chapter 2 —cell membrane structures and functions. Basic Neurochemistry eighth edition. Pages 26-39. 2012), incorporated herein by reference. TMBs are typically, but not exclusively, comprised predominantly of nonpolar (hydrophobic) amino acid residues and may traverse a lipid bilayer once or several times. The skilled person knows well methods to determine the hydrophobicity of an amino acid. See Simm et al. (2016), Biol Res., 49(1):31; Wimlet and White (1996), Nat Struct Biol., 3(10): 842-848; https://blanco.biomol.uci.edu/hydrophobicity_scales.html; and https://www.cgl.ucsf.edu/chimera/docs/UsersGuide/midas/hydrophob.html.

The TMBs usually comprise alpha helices, each helix containing 18-21 amino acids, which is sufficient to span the lipid bilayer. Accordingly, in certain embodiments, the transmembrane domain comprises one or more alpha helices.

In certain embodiments, the transmembrane domain is derived from an integral membrane protein, as further defined hereafter and in Albers et al., An “integral membrane protein” (also known as an intrinsic membrane protein) is a membrane protein that is permanently attached to the lipid membrane. In certain embodiments, the transmembrane domain is derived from an integral polytopic protein. An integral polytopic protein is one that spans the entire membrane. In certain embodiments, the transmembrane domain is derived from a single pass (trans)membrane protein, more particularly a bitopic membrane protein, e.g., of Type I or Type II.

Single-pass membrane proteins cross the membrane only once (i.e., a bitopic membrane protein), while multi-pass membrane proteins weave in and out, crossing several times. Single pass transmembrane proteins can be categorized as Type I, which are positioned such that their carboxyl-terminus is towards the cytosol, or Type II, which have their amino-terminus towards the cytosol. In certain embodiments, the transmembrane domain is derived from an integral monotopic protein. An integral monotopic protein is one that is associated with the membrane from only one side and does not span the lipid bilayer completely.

In certain embodiments, the transmembrane domain is derived from a non-human sequence. In certain embodiments, the antigenic prokaryotic polypeptide is derived from a prokaryotic transmembrane protein, and the transmembrane domain is the transmembrane domain of the prokaryotic transmembrane protein.

In certain embodiments, the transmembrane domain is derived from a viral sequence. The phrase “influenza”, “SARS CoV-2”, “varicella-zoster virus (VZV)”, “measles”, “rubella”, “rabies,” “Ebola,” and “smallpox” preceding the phrase “transmembrane domain sequence” indicates that the transmembrane domain sequence was derived from the virus corresponding to that name.

In certain embodiments, the transmembrane domain is derived from a viral transmembrane domain sequence selected from the group consisting of: an influenza transmembrane domain sequence, a SARS CoV-2 transmembrane domain sequence, a varicella-zoster virus (VZV) transmembrane domain sequence, a measles transmembrane domain sequence, a rubella transmembrane domain sequence, a mumps transmembrane domain sequence, a rabies transmembrane domain sequence, and an Ebola transmembrane domain sequence. These particular transmembrane domains are derived from viral sequences in viruses which have been administered to humans as vaccines (live-attenuated, inactivated or mRNA), with demonstrated strong safety profiles.

In certain embodiments, the transmembrane domain is selected from the group consisting of: an influenza hemagglutinin (HA) transmembrane domain sequence, a SARS CoV-2 spike transmembrane domain sequence, a VZV gB transmembrane domain sequence, a VZV gE transmembrane domain sequence, a VZV gI transmembrane domain sequence, a VZV gK transmembrane domain sequence, a measles F-protein transmembrane domain sequence, a rubella E1 protein transmembrane domain sequence, a rubella E2 protein transmembrane domain sequence, a mumps F-protein transmembrane domain sequence, a rabies virus glycoprotein (Rabies G) transmembrane domain sequence, and an Ebola GP protein transmembrane domain sequence.

In certain embodiments, the transmembrane domain comprises an HA transmembrane domain sequence from influenza A or influenza B, preferably from influenza A.

Exemplary viral transmembrane domain amino acid sequences of the disclosure are shown below in Table 3.

TABLE 3
Viral Transmembrane Domain (TMB) Signal Amino Acid Sequences

| NAME OF | | | |
|---|---|---|---|
| THE | | | |
| PROTEIN | ORGANISM | STRAIN | SEQUENCE OF THE TMB |
| HA (H1N1) | Influenza virus | A/New Caledonia/20/1999 | ILAIYSTVASSLVLLVSLGAIS |
| | | | F |
| | | | (SEQ ID NO: 17) |
| HA (H1N1pdm) | Influenza virus | A/California/7/2009 | ILAIYSTVASSLVLVVSLGAIS |
| | | | F |
| | | | (SEQ ID NO: 18) |
| HA (H3N2) | Influenza virus | A/Moscow/10/1999 | ILWISFAISCFLLCVVLLGFI |
| | | | (SEQ ID NO: 19) |
| HA B | Influenza virus | B/Phuket/3073/2013 | STAASSLAVTLMLAIFIVYM |
| | | | V |
| | | | (SEQ ID NO: 20) |
| Spike | SARS CoV-2 | Wuhan-1 | WYIWLGFIAGLIAIVMVTIM |
| | | | L |
| | | | (SEQ ID NO: 21) |
| gB | VZV | Oka strain | FGALAVGLLVLAGLVAAFF |
| | | | AY |
| | | | (SEQ ID NO: 22) |
| gE | VZV | Oka strain | AAWTGGLAAVVLLCLVIFLI |
| | | | C |
| | | | (SEQ ID NO: 23) |
| gI | VZV | Oka strain | IIIPIVASVMILTAMVIVIVI |
| | | | (SEQ ID NO: 24) |
| gK | VZV | Oka strain | YFWCVQLKMIFFAWFVYGM |
| | | | YL |
| | | | (SEQ ID NO: 25) |
| F | Measles | Edmonston-Zagreb strain | IVYILIAVCLGGLIGIPALIC |
| | | | (SEQ ID NO: 26) |
| E1 | Rubella | RA27/3 strain | LDHAFAAFVLLVPWVLIFMV |
| | | | C |
| | | | (SEQ ID NO: 27) |
| E2 | Rubella | RA27/3 strain | WWQLTLGAICALLLAGLLA |
| | | | CC |
| | | | (SEQ ID NO: 28) |
| F | Mumps | Miyahara strain | IVAALVLSILSIIISLLFCCW |
| | | | (SEQ ID NO: 29) |
| GP | Ebola | Mayinga-76 strain | WIPAGIGVTGVIIAVIALFCI |
| | | | (SEQ ID NO: 30) |
| Rabies G | Rabies | Rabies Pasteur strain | VLLSAGALTALMLIIFLMTC |
| | | | W (SEQ ID NO: 185) |

In certain embodiments, the SS sequence is positioned at the N-terminus of the antigenic prokaryotic polypeptide.

In certain embodiments, the SS sequence is positioned at the C-terminus of the antigenic prokaryotic polypeptide.

In certain embodiments, the TMB sequence is positioned at the N-terminus of the antigenic prokaryotic polypeptide.

In certain embodiments, the TMB sequence is positioned at the C-terminus of the antigenic prokaryotic polypeptide.

In certain embodiments, the SS amino acid sequence is encoded by a codon-optimized polynucleotide sequence.

In certain embodiments, the TMB amino acid sequence is encoded by a codon-optimized polynucleotide sequence.

### IV. Linkers

In certain embodiments of the disclosure, the viral secretion signal peptide (SS) sequence or transmembrane domain (TMB) are directly fused to the antigenic prokaryotic polypeptide (i.e., there is no linker, such as an amino acid linker, connecting the SS sequence or TMB to the antigenic prokaryotic polypeptide).

In other embodiments, the SS sequences and TMBs of the disclosure are optionally attached to an antigenic prokaryotic polypeptide with a linker. In certain embodiments, the linker is an amino acid linker. In the certain embodiments, the amino acid linker is 1-10 amino acids in length (e.g., the amino acid linker has a length of 1 amino acid, 2 amino acids, 3 amino acids, 4 amino acids, 5 amino acids, 6 amino acids, 7 amino acids, 8 amino acids, 9 amino acids, or 10 amino acids).

Illustrative examples of linkers include glycine polymers (Gly)n(SEQ ID NO: 191), where n is an integer of at least one, two, three, four, five, six, seven, or eight; glycine-serine polymers (GlySer)n(SEQ ID NO: 192), where n is an integer of at least one, two, three, four, five, six, seven, or eight; glycine-alanine polymers; alanine-serine polymers; and other flexible linkers known in the art.

Glycine and glycine-serine polymers are relatively unstructured and flexible, and therefore may be able to serve as a neutral tether between the SS sequence and/or TMB and the antigenic prokaryotic polypeptide. In certain embodiments, the linker is SGS or GSG.

Other exemplary linkers include, but are not limited to, the following amino acid sequences: GGG; DGGGS (SEQ ID NO: 81); TGEKP (SEQ ID NO: 82) (Liu et al. Proc. Natl. Acad. Sci. 94: 5525-5530. 1997); GGRR (SEQ ID NO: 92); (GGGGS)n (SEQ ID NO: 93), wherein n=1, 2, 3, 4 or 5 (Kim et al. Proc. Natl. Acad. Sci. 93: 1156-1160. 1996); EGKSSGSGSESKVD (SEQ ID NO: 83) (Chaudhary et al. Proc. Natl. Acad. Sci. 87: 1066-1070. 1990); KESGSVSSEQLAQFRSLD (SEQ ID NO: 84) (Bird et al. Science. 242:423-426. 1988), GGRRGGGS (SEQ ID NO: 85); LRQRDGERP (SEQ ID NO: 86); LRQKDGGGSERP (SEQ ID NO: 87); and GSTSGSGKPGSGEGSTKG (SEQ ID NO: 88) (Cooper et al. Blood. 101(4): 1637-1644. 2003). Preferred linkers are shorter, e.g., consisting of 3, 4 or 5 amino acids.

Additional examples of linkers are provided in Chen et al. (Adv Drug Deliv Rev. 65(10): 1357-1369. 2013), incorporated herein by reference.

### V. Antigenic Prokaryotic Polypeptides

The viral secretion signal peptides (SS) and/or transmembrane domains (TMB) of the disclosure are linked to antigenic prokaryotic polypeptides.

### A. Prokaryotic Genera

In certain embodiments, the antigenic prokaryotic polypeptide is derived from a bacteria of a genus selected from the group consisting of Acetobacter, Acinetobacter, Actinomyces, Aerococcus, Agrobacterium, Anaplasma, Azorhizobia, Azotobacter, Bacillus, Bacteroides, Bartonella, Bordetella, Borrelia, Brucella, Burkkolderia, Calymmatobacterium, Campylobacter, Chlamydia, Chlamydophila, Clostridium, Corynebacterium, Coxiella, Cutibacterium, Ehrlichia, Enterobacter, Enterococcus, Escherichia, Francisella, Fusobacterium, Gardnerella, Haemophilus, Helicobacter, Klebsiella, Lactobacillus, Lactococcus, Legionella, Listeria, Methanobacterium, Microbacterium, Micrococcus, Moraxella, Mycobacterium, Mycoplasma, Neisseria, Pasteurella, Pediococcus, Peptostreptococcus, Porphyromonas, Prevotella, Propionibacterium, Pseudomonas, Rhizobium, Rickettsia, Rochalimaea, Rothia, Salmonella, Serratia, Shigella, Sarcina, Spirillum, Spirochaetes, Staphylococcus, Stenotrophomonas, Streptobacillus, Streptococcus, Tetragenococcus, Treponema, Vibrio, Viridans, Walbachia, and Yersinia. In certain embodiments, the antigenic prokaryotic polypeptide is derived from a bacteria of a species selected from the group consisting of Acetobacter aurantius, Acinetobacter baumannii, Actinomyces israelii, Agrobacterium radiobacter, Agrobacterium tumefaciens, Anaplasma phagocytophilum, Azorhizobium caulinodans, Azotobacter vinelandii, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus Thuringiensis, Bacteroides fragilis, Bacteroides gingivalis, Bacteroides melaninogenicus, Bartonella henselae, Bartonella Quintana, Bordetella bronchiseptica, Bordetella pertussis, Borrelia burgdorferi. Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Campylobacter pylori, Chlamydia trachomatis, Chlamydophila pneumoniae, Chlamydophila psittaci, Clostridium botulinum, Clostridium difficile, Clostridium perfringens, Clostridium tetani, Corynebacterium diphtheriae, Corynebacterium fusiforme, Coxiella burnetii, Cutibacterium acnes, Cutibacterium avidum, Cutibacterium granulosum, Cutibacterium namnetense, Cutibacterium humerusii, Ehrlichia chaffeensis, Enterobacter cloacae, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus bulgaricus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Neisseria gonorrhoeae, Neisseria meningitidis, Pasteurella multocida, Pasteurella tularensis, Peptostreptococcus, Porphyromonas gingivalis, Prevotella melaninogenica, Propionibacterium acnes, Pseudomonas aeruginosa, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus, Staphylococcus epidermidis, Stenotrophomonas maltophilia, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Viridans streptococci, Wolbachia, Yersinia enterocolitica, Yersinia pestis and Yersinia pseudotuberculosis. In certain embodiments, the antigenic prokaryotic polypeptide is derived from a bacteria of the genus Borrelia, preferably selected from the species B. burgdorferi, afzelii, garinii, bavariensis, mayonii, spielmanii, lusitaniae, bissettii and/or valaisiana.

### B. Glycosylation

Glycosylation may occur in eukaryotic cells (but not in prokaryotic cells). In particular, N-linked glycosylation is the attachment of glycan to an amide nitrogen of an asparagine (Asn; N) residue of a protein. The process of attachment results in a glycosylated protein. Glycosylation can occur at any asparagine residue in a protein that is accessible to and recognized by glycosylating enzymes following translation of the protein, and is most common at accessible asparagines that are part of an NXS/T motif, wherein the first amino acid residue following the asparagine (X) is any amino acid except proline, and the second amino acid residue following the asparagine is a serine or threonine. A non-human glycosylation pattern can render a polypeptide undesirably reactogenic when used to elicit antibodies. Additionally, glycosylation of a polypeptide that is not normally glycosylated (such as an antigenic prokaryotic polypeptide) may alter its immunogenicity. For example, glycosylation can mask important immunogenic epitopes within a protein. Thus, to reduce or eliminate glycosylation, either asparagine residues or serine/threonine residues can be modified, for example, by substitution to another amino acid.

In certain embodiments, the antigenic prokaryotic polypeptide comprises at least one mutated glycosylation site, preferably at least one mutated N-linked glycosylation site and/or at least one O-linked glycosylation site. In some embodiments, one or more N-glycosylation sites in an antigenic prokaryotic polypeptide are removed. In some embodiments, the removal of an N-glycosylation site decreases glycosylation of an antigenic prokaryotic polypeptide. In some embodiments, the antigenic prokaryotic polypeptide has decreased glycosylation relative to a native antigenic prokaryotic polypeptide. In some embodiments, the removal of N-glycosylation sites eliminates N-glycosylation of an antigenic prokaryotic polypeptide.

In certain embodiments, the modification comprises a substitution of one or more of an N, S, and T amino acid in an NXS/T sequence motif, wherein X corresponds to any amino acid except Proline (P). In some embodiments, an N, S, or T amino acid is substituted with a conservative amino acid substitution. In certain embodiments, the polynucleotide sequences encoding the antigenic prokaryotic polypeptide is codon optimized.

### C. OspA

In certain embodiments, the antigenic prokaryotic polypeptide is OspA (Outer Surface Protein A). In certain embodiments, the OspA is preferably derived from OspA serotype (ST) 1, 2, 3, 4, 5, 6, and/or 7, more preferably from Borrelia burgdorferi strain B31 of Serotype 1, Borrelia afzelii strain PKO of Serotype 2, Borrelia garinii strain PBr of Serotype 3, Borrelia bavariensis of Serotype 4, Borrelia garinii of Serotype 5, Borrelia garinii of serotype 6, or Borrelia garinii of Serotype 7.

Exemplary amino acid sequences encoding an OspA proteins of the disclosure are shown below in Table 4.

### D. CAMP2

In certain embodiments, the antigenic prokaryotic polypeptide is a pore-forming toxin, preferably CAMP2 (Christie-Atkins-Munch-Peterson factor 2).

In certain embodiments, the CAMP2 is preferably derived from a bacterium of the genus Cutibacterium, more preferably of the species Cutibacterium acnes (formally known as Propionibacterium acnes).

In certain embodiments, the CAMP2 polypeptide comprises the amino acid sequence

(SEQ ID NO: 149)
MVEPTTTISATSTHELSASDARNSIQLLNAHIATLQSVQKSVPGSDYSD
QIRDLLKAAFDLRGLIETLAHGGIPFYDPSTIMPRIKLVATTIDTIHTA
TTTLQNKVRPAHVELGLEVTKAVLLTANPASTAKELDAEGAALKARLEK
VSQYPDLTPNDVATVYVRTNFSKTIWQVRANRDRYILGHKSAAVYKTLN
HAITKAVGVRLNPKTTVGNIQAARTELLAAYQTAFNSPDVKKAA.

Exemplary amino acid sequences encoding a C. acnes CAMP2 factor protein of the disclosure is shown below in Table 10.

E. PITP

In certain embodiments, the antigenic prokaryotic polypeptide is a putative iron-transport protein (PITP).

In certain embodiments, the PITP is preferably derived from a bacterium of the genus Cutibacterium, more preferably of the species Cutibacterium acnes (formally known as Propionibacterium acnes).

In certain embodiments, the PITP polypeptide comprises the amino acid sequence

(SEQ ID NO: 150)
MAGPTVTVIPVGREGGDITISGKGFSTTGFGVYVAVAPASVPEFYGNSD
KFYGYDPSKDTTESPSTIWVYTPSQKAIGSRFAQGRPMNNDGSFTITMK
APPFEQGKDFVVLTTKAHGVGKTDHSDDTRTPVTYREATPAPTGPKTPI
APSKQPSKQAAPSKQVKPSKQAGPNKQSTTPQQKTAEHRSQTPAAHRTM
TKQVCTIGASKVTSGSLTWGIRTSFTSYLRGPIANGSWKLSGGANWNGS
AFTFPLTSGSFDPATKSGSLKYSGSVHMTGHHGILDMTLAEPSLQIKGS
TGHLYLDVKSSSMDGKKTNYGRVDFATFGVSVSGNAAIKGSPVKLTATG
AKAFAGFYRAGEPMNPLSTNLTLSAEKVCHNVTVDAVTGKVIGDDSGKG
AGRGLPVT.

Exemplary amino acid sequences encoding a C. acnes PITP factor protein of the disclosure is shown below in Table 10.

### VI. Lipid Nanoparticle (LNP)

The LNPs of the disclosure comprise four categories of lipids: (i) an ionizable lipid (e.g., a cationic lipid); (ii) a PEGylated lipid; (iii) a cholesterol-based lipid, and (iv) a helper lipid.

### A. Ionizable Lipids

An ionizable lipid facilitates mRNA encapsulation and may be a cationic lipid. A cationic lipid affords a positively charged environment at low pH to facilitate efficient encapsulation of the negatively charged mRNA drug substance.

In some embodiments, the cationic lipid is OF-02:

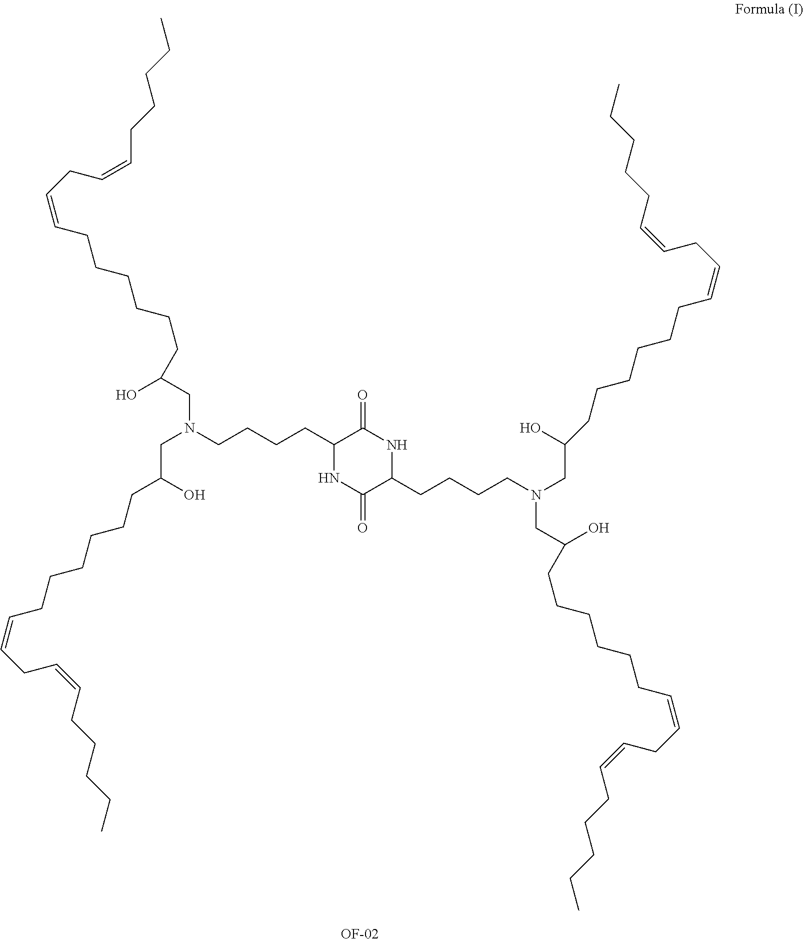 [SMILES: CCCCC/C=C\C/C=C\CCCCCCC(O)CN(CCCCC1NC(=O)C(CCCCN(CC(O)CCCCCC/C=C\C/C=C\CCCCC)CC(O)CCCCCC/C=C\C/C=C\CCCCC)NC1=O)CC(O)CCCCCC/C=C\C/C=C\CCCCC]

OF-02 is a non-degradable structural analog of OF-Deg-Lin. OF-Deg-Lin contains degradable ester linkages to attach the diketopiperazine core and the doubly-unsaturated tails, whereas OF-02 contains non-degradable 1,2-amino-alcohol linkages to attach the same diketopiperazine core and the doubly-unsaturated tails (Fenton et al., Adv Mater. (2016) 28:2939; U.S. Pat. No. 10,201,618). An exemplary LNP formulation herein, Lipid A, contains OF-2.

In some embodiments, the cationic lipid is cKK-E10 (Dong et al., PNAS (2014) 111(11):3955-60; U.S. Pat. No. 9,512,073):

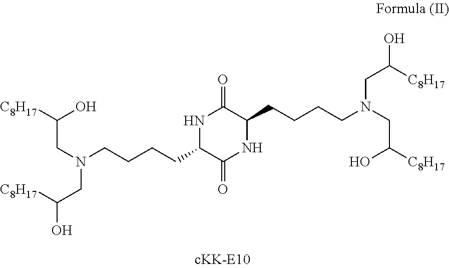

An exemplary LNP formulation herein, Lipid B, contains cKK-E10.

In some embodiments, the cationic lipid is GL-HEPES-E3-E10-DS-3-E18-1 (2-(4-(2-((3-(Bis((Z)-2-hydroxyoctadec-9-en-1-yl)amino)propyl)disulfaneyl)ethyl)piperazin-1-yl)ethyl 4-(bis(2-hydroxydecyl)amino)butanoate), which is a HEPES-based disulfide cationic lipid with a piperazine core, having the Formula III:

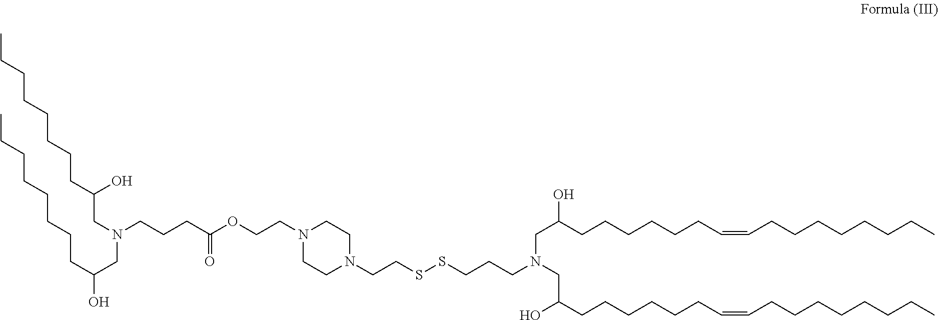 [SMILES: CCCCCCCC/C=C\CCCCCCC(O)CN(CCCSSCCN1CCN(CCOC(=O)CCCN(CC(O)CCCCCCCC)CC(O)CCCCCCCC)CC1)CC(O)CCCCCC/C=C\CCCCCCCC]

An exemplary LNP formulation herein, Lipid C, contains GL-HEPES-E3-E10-DS-3-E18-1. Lipid C has the same composition as Lipid A or Lipid B but for the difference in the cationic lipid.

In some embodiments, the cationic lipid is GL-HEPES-E3-E12-DS-4-E10 (2-(4-(2-((3-(bis(2-hydroxydecyl)amino)butyl)disulfaneyl)ethyl)piperazin-1-yl)ethyl 4-(bis(2-hydroxydodecyl)amino)butanoate), which is a HEPES-based disulfide cationic lipid with a piperazine core, having the Formula IV:

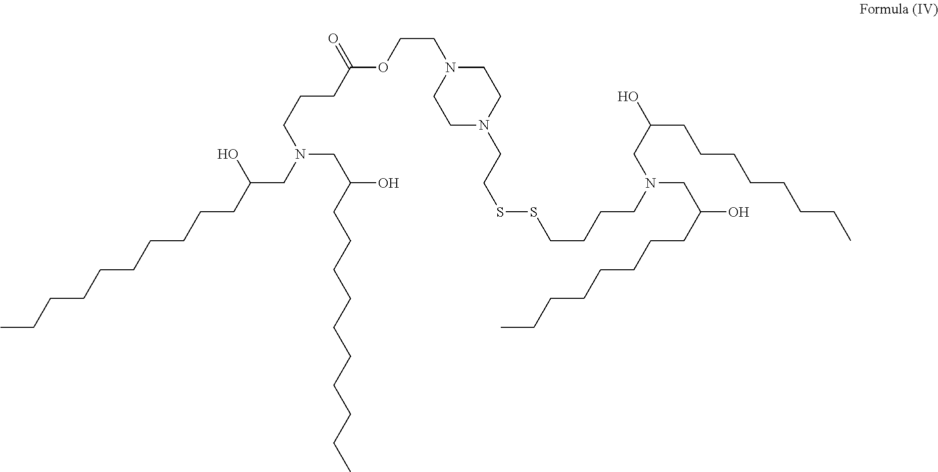 [SMILES: CCCCCCCCCCC(O)CN(CCCC(=O)OCCN1CCN(CCSSCCCCN(CC(O)CCCCCCCC)CC(O)CCCCCCCC)CC1)CC(O)CCCCCCCCCC]

An exemplary LNP formulation herein, Lipid D, contains GL-HEPES-E3-E12-DS-4-E10. Lipid D has the same composition as Lipid A or Lipid B but for the difference in the cationic lipid.

In some embodiments, the cationic lipid is GL-HEPES-E3-E12-DS-3-E14 (2-(4-(2-((3-(Bis(2-hydroxytetradecyl)amino)propyl)disulfaneyl)ethyl)piperazin-1-yl)ethyl 4-(bis(2-hydroxydodecyl)amino)butanoate), which is a HEPES-based disulfide cationic lipid with a piperazine core, having the Formula V:

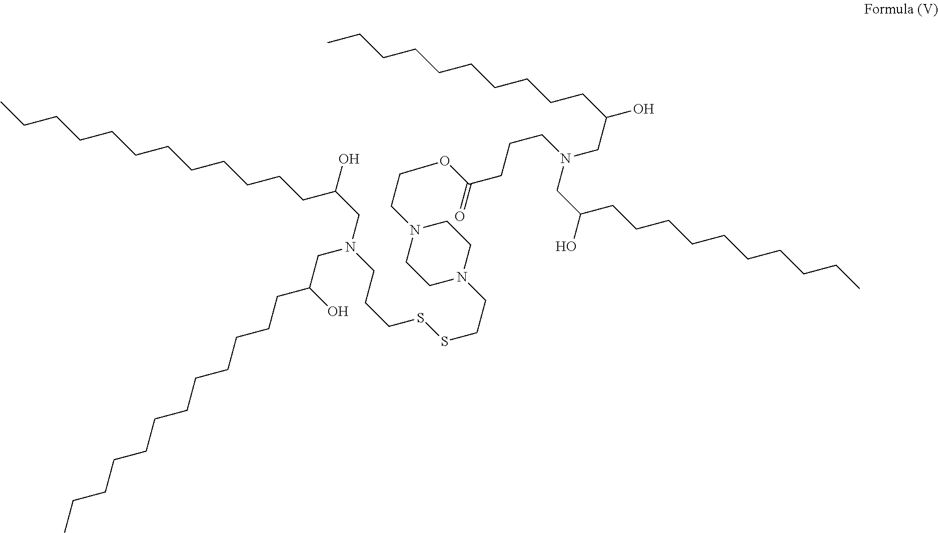 [SMILES: CCCCCCCCCCCCC(O)CN(CCCSSCCN1CCN(CCOC(=O)CCCN(CC(O)CCCCCCCCCC)CC(O)CCCCCCCCCC)CC1)CC(O)CCCCCCCCCCCC]

An exemplary LNP formulation herein, Lipid E, contains GL-HEPES-E3-E12-DS-3-E14. Lipid E has the same composition as Lipid A or Lipid B but for the difference in the cationic lipid.

The cationic lipids GL-HEPES-E3-E10-DS-3-E18-1 (III), GL-HEPES-E3-E12-DS-4-E10 (IV), and GL-HEPES-E3-E12-DS-3-E14 (V) can be synthesized according to the general procedure set out in Scheme 1:

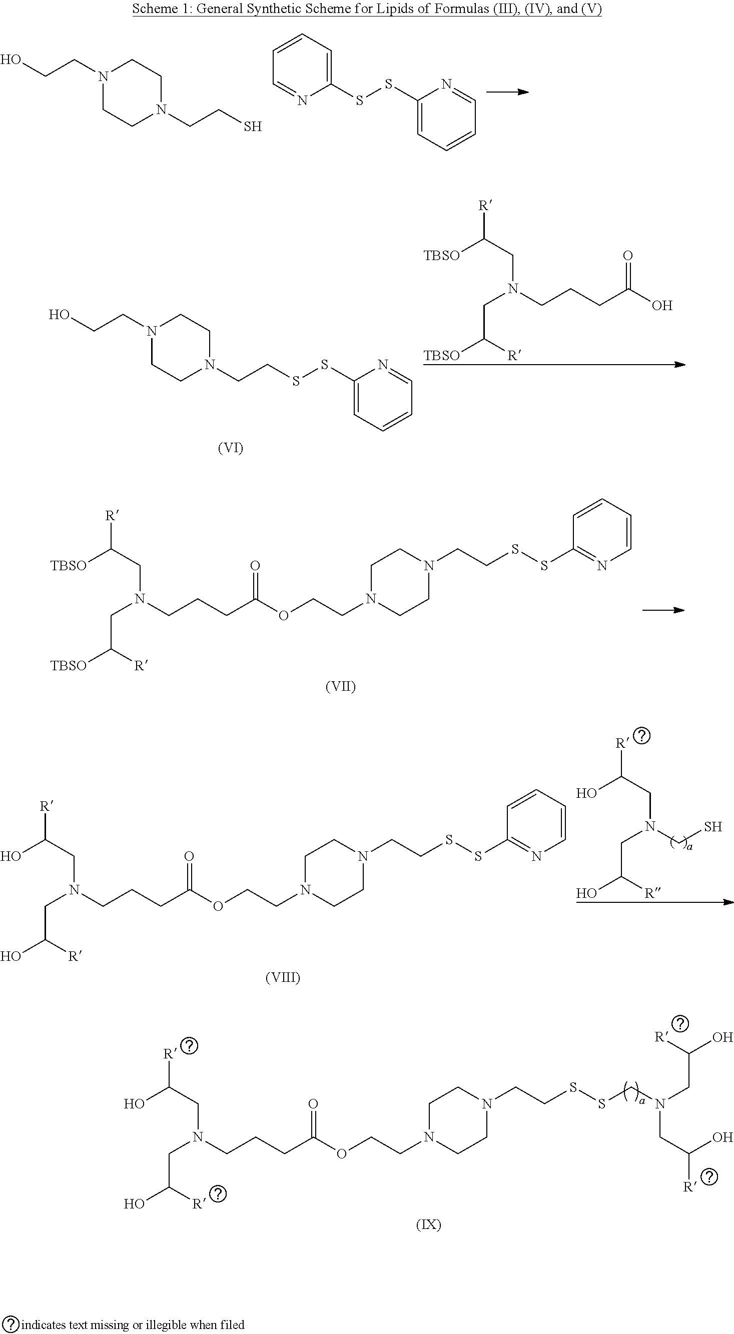

In some embodiments, the cationic lipid is MC3, having the Formula VI:

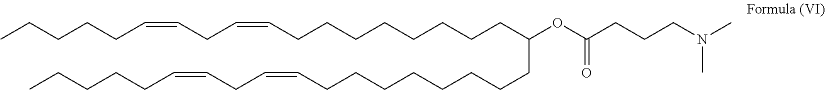 [SMILES: CCCCC/C=C\C/C=C\CCCCCCCCC(CCCCCCCC/C=C\C/C=C\CCCCC)OC(=O)CCCN(C)C]

In some embodiments, the cationic lipid is SM-102 (9-heptadecanyl 8-{(2-hydroxyethyl)[6-oxo-6-(undecyloxy)hexyl]amino}octanoate), having the Formula VII:

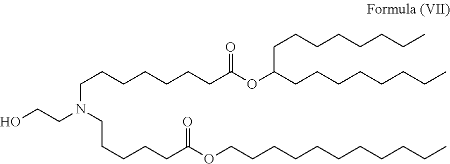 [SMILES: CCCCCCCCCCCOC(=O)CCCCCN(CCO)CCCCCCCC(=O)OC(CCCCCCCC)CCCCCCCC]

In some embodiments, the cationic lipid is ALC-0315 [(4-hydroxybutyl)azanediyl] di(hexane-6,1-diyl) bis(2-hexyldecanoate), having the Formula VIII:

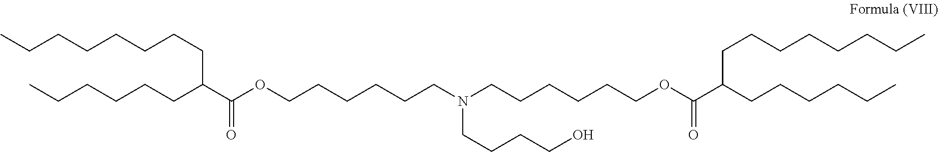 [SMILES: CCCCCCCCC(CCCCCC)C(=O)OCCCCCCN(CCCCO)CCCCCCOC(=O)C(CCCCCC)CCCCCCCC]

In some embodiments, the cationic lipid is cOrn-EE1, having the Formula IX:

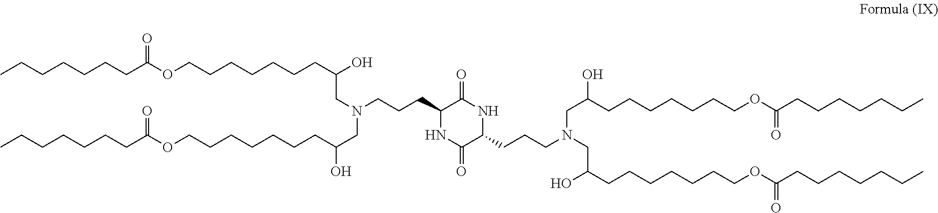 [SMILES: CCCCCCCC(=O)OCCCCCCCC(O)CN(CCC[C@@H]1NC(=O)[C@@H](CCCN(CC(O)CCCCCCCOC(=O)CCCCCCC)CC(O)CCCCCCCOC(=O)CCCCCCC)NC1=O)CC(O)CCCCCCCOC(=O)CCCCCCC]

In some embodiments, the cationic lipid may be selected from the group comprising cKK-E10; OF-02; [(6Z,9Z,28Z,31Z)-heptatriaconta-6,9,28,31-tetraen-19-yl]4-(dimethylamino)butanoate (D-Lin-MC3-DMA); 2,2-dilinoleyl-4-dimethylaminoethyl-[1,3]-dioxolane (DLin-KC2-DMA); 1,2-dilinoleyloxy-N,N-dimethyl-3-aminopropane (DLin-DMA); di((Z)-non-2-en-1-yl) 9-((4-(dimethylamino)butanoyl)oxy)heptadecanedioate (L319); 9-heptadecanyl 8-{(2-hydroxyethyl)[6-oxo-6-(undecyloxy)hexyl]amino}octanoate (SM-102); [(4-hydroxybutyl)azanediyl] di(hexane-6,1-diyl) bis(2-hexyldecanoate) (ALC-0315); [3-(dimethylamino)-2-[(Z)-octadec-9-enoyl]oxypropyl](Z)-octadec-9-enoate (DODAP); 2,5-bis(3-aminopropylamino)-N-[2—[di(heptadecyl)amino]-2-oxoethyl]pentanamide (DOGS); [(3S,8S,9S,10R,13R,14S,17R)-10,13-dimethyl-17-[(2R)-6-methylheptan-2-yl]-2,3,4,7,8,9,11,12,14,15,16,17-dodecahydro-1H-cyclopenta[a]phenanthren-3-yl]N-[2-(dimethylamino)ethyl]carbamate (DC-Chol); tetrakis(8-methylnonyl) 3,3′,3″,3″′-(((methylazanediyl) bis(propane-3,1 diyl))bis (azanetriyl))tetrapropionate (3060i10); decyl (2-(dioctylammonio)ethyl) phosphate (9A1P9); ethyl 5,5-di((Z)-heptadec-8-en-1-yl)-1-(3-(pyrrolidin-1-yl)propyl)-2,5-dihydro-1H-imidazole-2-carboxylate (A2-Iso5-2DC18); bis(2-(dodecyldisulfanyl)ethyl) 3,3′-((3-methyl-9-oxo-10-oxa-13,14-dithia-3,6-diazahexacosyl)azanediyl)dipropionate (BAME-016B); 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol) (C12-200); 3,6-bis(4-(bis(2-hydroxydodecyl)amino)butyl)piperazine-2,5-dione (cKK-E12); hexa(octan-3-yl) 9,9′,9″,9″′,9” ”,9″″′—((((benzene-1,3,5-tricarbonyl)yris(azanediyl)) tris (propane-3,1-diyl)) tris(azanetriyl))hexanonanoate (FTT5); (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis (octadeca-9,12-dienoate) (OF-Deg-Lin); TT3; N¹,N³,N⁵-tris(3-(didodecylamino)propyl)benzene-1,3,5-tricarboxamide; N1-[2-((1S)-1-[(3-aminopropyl)amino]-4—[di(3-aminopropyl)amino]butylcarboxamido)ethyl]-3,4-di[oleyloxy]-benzamide (MVL5); heptadecan-9-yl 8-((2-hydroxyethyl)(8-(nonyloxy)-8-oxooctyl)amino)octanoate (Lipid 5); GL-HEPES-E3-E10-DS-3-E18-1; GL-HEPES-E3-E12-DS-4-E10; GL-HEPES-E3-E12-DS-3-E14; and combinations thereof.

In some embodiments, the cationic lipid is biodegradable.

In some embodiments, the cationic lipid is not biodegradable.

In some embodiments, the cationic lipid is cleavable.

In some embodiments, the cationic lipid is not cleavable.

Cationic lipids are described in further detail in Dong et al. (PNAS. 111(11):3955-60. 2014); Fenton et al. (Adv Mater. 28:2939. 2016); U.S. Pat. Nos. 9,512,073; and 10,201,618, each of which is incorporated herein by reference.

### B. PEGylated Lipids

The PEGylated lipid component provides control over particle size and stability of the nanoparticle. The addition of such components may prevent complex aggregation and provide a means for increasing circulation lifetime and increasing the delivery of the lipid-nucleic acid pharmaceutical composition to target tissues (Klibanov et al. FEBS Letters 268(1):235-7. 1990).

These components may be selected to rapidly exchange out of the pharmaceutical composition in vivo (see, e.g., U.S. Pat. No. 5,885,613).

Contemplated PEGylated lipids include, but are not limited to, a polyethylene glycol (PEG) chain of up to 5 kDa in length covalently attached to a lipid with alkyl chain(s) of C₆-C₂₀ (e.g., C₈, C₁₀, C₁₂, C₁₄, C₁₆, or C₁₈) length, such as a derivatized ceramide (e.g., N-octanoyl-sphingosine-1-[succinyl(methoxypolyethylene glycol)](C8 PEG ceramide)). In some embodiments, the PEGylated lipid is 1,2-dimyristoyl-rac-glycero-3-methoxypolyethylene glycol (DMG-PEG); 1,2-distearoyl-sn-glycero-3-phosphoethanolamine-polyethylene glycol (DSPE-PEG); 1,2-dilauroyl-sn-glycero-3-phosphoethanolamine-polyethylene glycol (DLPE-PEG); or 1,2-distearoyl-rac-glycero-polyethelene glycol (DSG-PEG), PEG-DAG; PEG-PE; PEG-S-DAG; PEG-S-DMG; PEG-cer; a PEG-dialkyoxypropylcarbamate; 2-[(polyethylene glycol)-2000]-N,N-ditetradecylacetamide (ALC-0159); and combinations thereof.

In certain embodiments, the PEG has a high molecular weight, e.g., 2000-2400 g/mol. In certain embodiments, the PEG is PEG2000 (or PEG-2K). In certain embodiments, the PEGylated lipid herein is DMG-PEG2000, DSPE-PEG2000, DLPE-PEG2000, DSG-PEG2000, C8 PEG2000, or ALC-0159 (2-[(polyethylene glycol)-2000]-N,N-ditetradecylacetamide). In certain embodiments, the PEGylated lipid herein is DMG-PEG2000.

### C. Cholesterol-Based Lipids

The cholesterol component provides stability to the lipid bilayer structure within the nanoparticle. In some embodiments, the LNPs comprise one or more cholesterol-based lipids. Suitable cholesterol-based lipids include, for example: DC-Choi (N,N-dimethyl-N-ethylcarboxamidocholesterol), 1,4-bis(3-N-oleylamino-propyl)piperazine (Gao et al., Biochem Biophys Res Comm. (1991) 179:280; Wolf et al., BioTechniques (1997) 23:139; U.S. Pat. No. 5,744,335), imidazole cholesterol ester (“ICE”; WO2011/068810), sitosterol (22,23-dihydrostigmasterol), 0-sitosterol, sitostanol, fucosterol, stigmasterol (stigmasta-5,22-dien-3-ol), ergosterol; desmosterol (3B-hydroxy-5,24-cholestadiene); lanosterol (8,24-lanostadien-3b-ol); 7-dehydrocholesterol (A5,7-cholesterol); dihydrolanosterol (24,25-dihydrolanosterol); zymosterol (5a-cholesta-8,24-dien-3M-ol); lathosterol (5a-cholest-7-en-3M-ol); diosgenin ((30,25R)-spirost-5-en-3-ol); campesterol (campest-5-en-3M-ol); campestanol (5a-campestan-3b-ol); 24-methylene cholesterol (5,24(28)-cholestadien-24-methylen-3B-ol); cholesteryl margarate (cholest-5-en-3M-yl heptadecanoate); cholesteryl oleate; cholesteryl stearate and other modified forms of cholesterol. In some embodiments, the cholesterol-based lipid used in the LNPs is cholesterol.

### D. Helper Lipids

A helper lipid enhances the structural stability of the LNP and helps the LNP in endosome escape. It improves uptake and release of the mRNA drug payload. In some embodiments, the helper lipid is a zwitterionic lipid, which has fusogenic properties for enhancing uptake and release of the drug payload. Examples of helper lipids are 1,2-dioleoyl-SN-glycero-3-phosphoethanolamine (DOPE); 1,2-distearoyl-sn-glycero-3-phosphocholine (DSPC); 1,2-dioleoyl-sn-glycero-3-phospho-L-serine (DOPS); 1,2-dielaidoyl-sn-glycero-3-phosphoethanolamine (DEPE); and 1,2-dioleoyl-sn-glycero-3-phosphocholine (DPOC), dipalmitoylphosphatidylcholine (DPPC), DMPC, 1,2-dilauroyl-sn-glycero-3-phosphocholine (DLPC), 1,2-Distearoylphosphatidylethanolamine (DSPE), and 1,2-dilauroyl-sn-glycero-3-phosphoethanolamine (DLPE).

Other exemplary helper lipids are dioleoylphosphatidylcholine (DOPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), phosphatidylserine, sphingolipids, sphingomyelins, ceramides, cerebrosides, gangliosides, 16-O-monomethyl PE, 16-O-dimethyl PE, 18-1-trans PE, 1-stearoyl-2-oleoyl-phosphatidyethanolamine (SOPE), or a combination thereof. In certain embodiments, the helper lipid is DOPE. In certain embodiments, the helper lipid is DSPC.

In various embodiments, the present LNPs comprise (i) a cationic lipid selected from OF-02, cKK-E10, GL-HEPES-E3-E10-DS-3-E18-1, GL-HEPES-E3-E12-DS-4-E10, or GL-HEPES-E3-E12-DS-3-E14; (ii) DMG-PEG2000; (iii) cholesterol; and (iv) DOPE.

In other embodiments, the present LNPs comprise (i) SM-102; (ii) DMG-PEG2000; (iii) cholesterol; and (iv) DSPC.

In yet other embodiments, the present LNPs comprise (i) ALC-0315; (ii) ALC-0159; (iii) cholesterol; and (iv) DSPC.

### E. Molar Ratios of the Lipid Components

The molar ratios of the above components are important for the LNPs' effectiveness in delivering mRNA. The molar ratio of the cationic lipid, the PEGylated lipid, the cholesterol-based lipid, and the helper lipid is A: B: C: D, where A +B+C+D=100%. In some embodiments, the molar ratio of the cationic lipid in the LNPs relative to the total lipids (i.e., A) is 35-55%, such as 35-50% (e.g., 38-42% such as 40%, or 45-50%). In some embodiments, the molar ratio of the PEGylated lipid component relative to the total lipids (i.e., B) is 0.25-2.75% (e.g., 1-2% such as 1.5%). In some embodiments, the molar ratio of the cholesterol-based lipid relative to the total lipids (i.e., C) is 20-50% (e.g., 27-30% such as 28.5%, or 38-43%). In some embodiments, the molar ratio of the helper lipid relative to the total lipids (i.e., D) is 5-35% (e.g., 28-32% such as 30%, or 8-12%, such as 10%). In some embodiments, the (PEGylated lipid+cholesterol) components have the same molar amount as the helper lipid. In some embodiments, the LNPs contain a molar ratio of the cationic lipid to the helper lipid that is more than 1.

In certain embodiments, the LNP of the disclosure comprises:

a cationic lipid at a molar ratio of 35% to 55% or 40% to 50% (e.g., a cationic lipid at a molar ratio of 35%, 36%, 37%, 38%, 39%, 40%, 41% 42%, 43%, 44%, 45%, 46%, 47%, 48%, 49%, 50%, 51%, 52%, 53%, 54%, or 55%);

a polyethylene glycol (PEG) conjugated (PEGylated) lipid at a molar ratio of 0.25% to 2.75% or 1.00% to 2.00% (e.g., a PEGylated lipid at a molar ratio of 0.25%, 0.50%, 0.75%, 1.00%, 1.25%, 1.50%, 1.75%, 2.00%, 2.25%, 2.50%, or 2.75%);

a cholesterol-based lipid at a molar ratio of 20% to 50%, 25% to 45%, or 28.5% to 43% (e.g., a cholesterol-based lipid at a molar ratio of 20%, 21%, 22%, 23%, 24%, 25%, 26%, 27%, 28%, 29%, 30%, 31%, 32%, 33%, 34%, 35%, 36%, 37%, 38%, 39%, 40%, 41% 42%, 43%, 44%, 45%, 46%, 47%, 48%, 49%, or 50%); and

a helper lipid at a molar ratio of 5% to 35%, 8% to 30%, or 10% to 30% (e.g., a helper lipid at a molar ratio of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35%),

wherein all of the molar ratios are relative to the total lipid content of the LNP.

In certain embodiments, the LNP comprises: a cationic lipid at a molar ratio of 40%; a PEGylated lipid at a molar ratio of 1.5%; a cholesterol-based lipid at a molar ratio of 28.5%; and a helper lipid at a molar ratio of 30%.

In certain embodiments, the LNP of the disclosure comprises: a cationic lipid at a molar ratio of 45 to 50%; a PEGylated lipid at a molar ratio of 1.5 to 1.7%; a cholesterol-based lipid at a molar ratio of 38 to 43%; and a helper lipid at a molar ratio of 9 to 10%.

In certain embodiments, the PEGylated lipid is dimyristoyl-PEG2000 (DMG-PEG2000).

In various embodiments, the cholesterol-based lipid is cholesterol.

In some embodiments, the helper lipid is 1,2-dioleoyl-SN-glycero-3-phosphoethanolamine (DOPE).

In certain embodiments, the LNP comprises: OF-02 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: cKK-E10 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E10-DS-3-E18-1 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-4-E10 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-3-E14at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: SM-102 at a molar ratio of 35% to 55%; DMG-PEG2000 at a molar ratio of 0.25% to 2.75%; cholesterol at a molar ratio of 20% to 50%; and DSPC at a molar ratio of 5% to 35%.

In certain embodiments, the LNP comprises: ALC-0315 at a molar ratio of 35% to 55%; ALC-0159 at a molar ratio of 0.25% to 2.75%; cholesterol at a molar ratio of 20% to 50%; and DSPC at a molar ratio of 5% to 35%.

In certain embodiments, the LNP comprises: OF-02 at a molar ratio of 40%; DMG-PEG2000 at a molar ratio of 1.5%; cholesterol at a molar ratio of 28.5%; and DOPE at a molar ratio of 30%. This LNP formulation is designated “Lipid A” herein.

In certain embodiments, the LNP comprises: cKK-E10 at a molar ratio of 40%; DMG-PEG2000 at a molar ratio of 1.5%; cholesterol at a molar ratio of 28.5%; and DOPE at a molar ratio of 30%. This LNP formulation is designated “Lipid B” herein.

In certain embodiments, the LNP comprises: GL-HEPES-E3-E10-DS-3-E18-1 at a molar ratio of 40%; DMG-PEG2000 at a molar ratio of 1.5%; cholesterol at a molar ratio of 28.5%; and DOPE at a molar ratio of 30%. This LNP formulation is designated “Lipid C” herein.

In certain embodiments, the LNP comprises: GL-HEPES-E3-E12-DS-4-E10 (at a molar ratio of 40%; DMG-PEG2000 at a molar ratio of 1.5%; cholesterol at a molar ratio of 28.5%; and DOPE at a molar ratio of 30%. This LNP formulation is designated “Lipid D” herein.

In certain embodiments, the LNP comprises: GL-HEPES-E3-E12-DS-3-E14at a molar ratio of 40%; DMG-PEG2000 at a molar ratio of 1.5%; cholesterol at a molar ratio of 28.5%; and DOPE at a molar ratio of 30%. This LNP formulation is designated “Lipid E” herein.

In certain embodiments, the LNP comprises: 9-heptadecanyl 8-{(2-hydroxyethyl)[6-oxo-6-(undecyloxy)hexyl]amino}octanoate (SM-102) at a molar ratio of 50%; 1,2-distearoyl-sn-glycero-3-phosphocholine (DSPC) at a molar ratio of 10%; cholesterol at a molar ratio of 38.5%; and 1,2-dimyristoyl-rac-glycero-3-methoxypolyethylene glycol-2000 (DMG-PEG2000) at a molar ratio of 1.5%.

In certain embodiments, the LNP comprises: (4-hydroxybutyl)azanediyl] di(hexane-6,1-diyl) bis(2-hexyldecanoate) (ALC-0315) at a molar ratio of 46.3%; 1,2-distearoyl-sn-glycero-3-phosphocholine (DSPC) at a molar ratio of 9.4%; cholesterol at a molar ratio of 42.7%; and 2-[(polyethylene glycol)-2000]-N,N-ditetradecylacetamide (ALC-0159) at a molar ratio of 1.6%.

In certain embodiments, the LNP comprises: (4-hydroxybutyl)azanediyl] di(hexane-6,1-diyl) bis(2-hexyldecanoate) (ALC-0315) at a molar ratio of 47.4%; 1,2-distearoyl-sn-glycero-3-phosphocholine (DSPC) at a molar ratio of 10%; cholesterol at a molar ratio of 40.9%; and 2-[(polyethylene glycol)-2000]-N,N-ditetradecylacetamide (ALC-0159) at a molar ratio of 1.7%.

To calculate the actual amount of each lipid to be put into an LNP formulation, the molar amount of the cationic lipid is first determined based on a desired N/P ratio, where N is the number of nitrogen atoms in the cationic lipid and P is the number of phosphate groups in the mRNA to be transported by the LNP. Next, the molar amount of each of the other lipids is calculated based on the molar amount of the cationic lipid and the molar ratio selected. These molar amounts are then converted to weights using the molecular weight of each lipid

### F. Active Ingredients of the LNPs

The active ingredient of the present LNP vaccine composition is a nucleic acid (e.g., a mRNA) that encodes an antigenic prokaryotic polypeptide.

Where desired, the LNP may be multi-valent. In some embodiments, the LNP may carry nucleic acids, such as mRNAs, that encode more than one antigenic prokaryotic polypeptide, such as two, three, four, five, six, seven, or eight antigens. For example, the LNP may carry multiple nucleic acids (e.g., mRNA), each encoding a different antigenic prokaryotic polypeptide; or carry a polycistronic mRNA that can be translated into more than one antigenic prokaryotic polypeptide (e.g., each antigen-coding sequence is separated by a nucleotide linker encoding a self-cleaving peptide such as a 2A peptide). An LNP carrying different nucleic acids (e.g., mRNA) typically comprises (encapsulate) multiple copies of each nucleic acid. For example, an LNP carrying or encapsulating two different nucleic acids typically carries multiple copies of each of the two different nucleic acids.

In some embodiments, a single LNP formulation may comprise multiple kinds (e.g., two, three, four, five, six, seven, eight, nine, ten, or more) of LNPs, each kind carrying a different nucleic acid (e.g., mRNA).

When the nucleic acid is mRNA, the mRNA may be unmodified (i.e., containing only natural ribonucleotides A, U, C, and/or G linked by phosphodiester bonds), or chemically modified (e.g., including nucleotide analogs such as pseudouridines (e.g., N-1-methyl pseudouridine), 2′-fluoro ribonucleotides, and 2′-methoxy ribonucleotides, and/or phosphorothioate bonds). The mRNA molecule may comprise a 5′ cap and a polyA tail.

### G. Buffer and Other Components

To stabilize the nucleic acid and/or LNPs (e.g., to prolong the shelf-life of the vaccine product), to facilitate administration of the LNP pharmaceutical composition, and/or to enhance in vivo expression of the nucleic acid, the nucleic acid and/or LNP can be formulated in combination with one or more carriers, targeting ligands, stabilizing reagents (e.g., preservatives and antioxidants), and/or other pharmaceutically acceptable excipients. Examples of such excipients are parabens, thimerosal, thiomersal, chlorobutanol, benzalkonium chloride, and chelators (e.g., EDTA).

The LNP compositions of the present disclosure can be provided as a frozen liquid form or a lyophilized form. A variety of cryoprotectants may be used, including, without limitations, sucrose, trehalose, glucose, mannitol, mannose, dextrose, and the like. The cryoprotectant may constitute 5-30% (w/v) of the LNP composition. In some embodiments, the LNP composition comprises trehalose, e.g., at 5-30% (e.g., 10%) (w/v). Once formulated with the cryoprotectant, the LNP compositions may be frozen (or lyophilized and cryopreserved) at −20° C. to −80° C.

The LNP compositions may be provided to a patient in an aqueous buffered solution-thawed if previously frozen, or if previously lyophilized, reconstituted in an aqueous buffered solution at bedside. The buffered solution preferably is isotonic and suitable for e.g., intramuscular or intradermal injection. In some embodiments, the buffered solution is a phosphate-buffered saline (PBS).

### VII. RNA

The present vaccine compositions of the disclosure may comprise an RNA molecule (e.g., mRNA) that encodes an antigen of interest (e.g., an antigenic prokaryotic polypeptide). The RNA molecule of the present disclosure may comprise at least one ribonucleic acid (RNA) comprising an ORF encoding an antigen of interest. In certain embodiments, the RNA is a messenger RNA (mRNA) comprising an ORF encoding an antigen of interest. In certain embodiments, the RNA (e.g., mRNA) further comprises at least one 5′ UTR, 3′ UTR, a poly(A) tail, and/or a 5′ cap.

### A. 5′ Cap

An mRNA 5′ cap can provide resistance to nucleases found in most eukaryotic cells and promote translation efficiency. Several types of 5′ caps are known. A 7-methylguanosine cap (also referred to as “m⁷G” or “Cap-0”), comprises a guanosine that is linked through a 5′—5′-triphosphate bond to the first transcribed nucleotide.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp, (5′(A,G(5′)ppp(5′)A, and G(5′)ppp(5′)G. Additional cap structures are described in U.S.

Publication No. US 2016/0032356 and U.S. Publication No. US 2018/0125989, which are incorporated herein by reference.

5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′)G (the ARCA cap); G(5′)ppp(5′)A; G(5′)ppp(5′)G; m7G(5′)ppp(5′)A; m7G(5′)ppp(5′)G; m7G(5′)ppp(5′)(2′OMeA)pG; m7G(5′)ppp(5′)(2′OMeA)pU; m7G(5′)ppp(5′)(2′OMeG)pG (New England BioLabs, Ipswich, MA; TriLink Biotechnologies). 5′-capping of modified RNA may be completed post-transcriptionally using a vaccinia virus capping enzyme to generate the Cap 0 structure: m7G(5′)ppp(5′)G. Cap 1 structure may be generated using both vaccinia virus capping enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase.

In certain embodiments, the mRNA of the disclosure comprises a 5′ cap selected from the group consisting of 3′-O-Me-m7G(5′)ppp(5′)G (the ARCA cap), G(5′)ppp(5′)A, G(5′)ppp(5′)G, m7G(5′)ppp(5′)A, m7G(5′)ppp(5′)G, m7G(5′)ppp(5′)(2′OMeA)pG, m7G(5′)ppp(5′)(2′OMeA)pU, and m7G(5′)ppp(5′)(2′OMeG)pG.

In certain embodiments, the mRNA of the disclosure comprises a 5′ cap of:

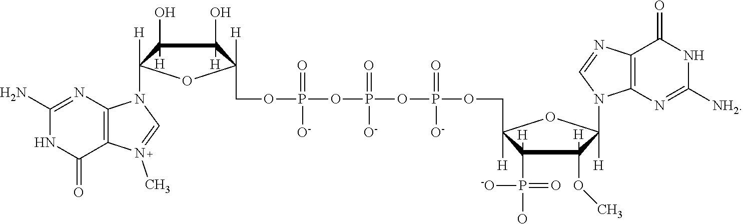

### B. Untranslated Region (UTR)

In some embodiments, the mRNA of the disclosure includes a 5′ and/or 3′ untranslated region (UTR). In mRNA, the 5′ UTR starts at the transcription start site and continues to the start codon but does not include the start codon. The 3′ UTR starts immediately following the stop codon and continues until the transcriptional termination signal.

In some embodiments, the mRNA disclosed herein may comprise a 5′ UTR that includes one or more elements that affect an mRNA's stability or translation. In some embodiments, a 5′ UTR may be about 10 to 5,000 nucleotides in length. In some embodiments, a 5′ UTR may be about 50 to 500 nucleotides in length. In some embodiments, the 5′ UTR is at least about 10 nucleotides in length, about 20 nucleotides in length, about 30 nucleotides in length, about 40 nucleotides in length, about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length or about 5,000 nucleotides in length.

In some embodiments, the mRNA disclosed herein may comprise a 3′ UTR comprising one or more of a polyadenylation signal, a binding site for proteins that affect an mRNA's stability of location in a cell, or one or more binding sites for miRNAs. In some embodiments, a 3′ UTR may be 50 to 5,000 nucleotides in length or longer. In some embodiments, a 3′ UTR may be 50 to 1,000 nucleotides in length or longer. In some embodiments, the 3′ UTR is at least about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length.

In some embodiments, the mRNA disclosed herein may comprise a 5′ or 3′ UTR that is derived from a gene distinct from the one encoded by the mRNA transcript (i.e., the UTR is a heterologous UTR).

In certain embodiments, the 5′ and/or 3′ UTR sequences can be derived from mRNA which are stable (e.g., globin, actin, GAPDH, tubulin, histone, or citric acid cycle enzymes) to increase the stability of the mRNA. For example, a 5′ UTR sequence may include a partial sequence of a CMV immediate-early 1 (IE1) gene, or a fragment thereof, to improve the nuclease resistance and/or improve the half-life of the mRNA. Also contemplated is the inclusion of a sequence encoding human growth hormone (hGH), or a fragment thereof, to the 3′ end or untranslated region of the mRNA. Generally, these modifications improve the stability and/or pharmacokinetic properties (e.g., half-life) of the mRNA relative to their unmodified counterparts, and include, for example, modifications made to improve such mRNA resistance to in vivo nuclease digestion.

Exemplary 5′ UTRs include a sequence derived from a CMV immediate-early 1 (IE1) gene (U.S. Publication Nos. 2014/0206753 and 2015/0157565, each of which is incorporated herein by reference), or the sequence GGGAUCCUACC (SEQ ID NO: 94) (U.S. Publication No. 2016/0151409, incorporated herein by reference).

In various embodiments, the 5′ UTR may be derived from the 5′ UTR of a TOP gene. TOP genes are typically characterized by the presence of a 5′-terminal oligopyrimidine (TOP) tract. Furthermore, most TOP genes are characterized by growth-associated translational regulation. However, TOP genes with a tissue specific translational regulation are also known. In certain embodiments, the 5′ UTR derived from the 5′ UTR of a TOP gene lacks the 5′ TOP motif (the oligopyrimidine tract) (e.g., U.S. Publication Nos. 2017/0029847, 2016/0304883, 2016/0235864, and 2016/0166710, each of which is incorporated herein by reference).

In certain embodiments, the 5′ UTR is derived from a ribosomal protein Large 32 (L32) gene (U.S. Publication No. 2017/0029847, supra).

In certain embodiments, the 5′ UTR is derived from the 5′ UTR of an hydroxysteroid (17-b) dehydrogenase 4 gene (HSD17B4) (U.S. Publication No. 2016/0166710, supra).

In certain embodiments, the 5′ UTR is derived from the 5′ UTR of an ATP5A1 gene (U.S. Publication No. 2016/0166710, supra).

In some embodiments, an internal ribosome entry site (IRES) is used instead of a 5′ UTR.

In some embodiments, the 5′UTR comprises a nucleic acid sequence set forth in SEQ ID NO: 89 and reproduced below:

(SEQ ID NO: 89)
GGACAGAUCGCCUGGAGACGCCAUCCACGCUGUUUUGACCUCCAUAGAA
GACACCGGGACCGAUCCAGCCUCCGCGGCCGGGAACGGUGCAUUGGAAC
GCGGAUUCCCCGUGCCAAGAGUGACUCACCGUCCUUGACACG.

In some embodiments, the 3′UTR comprises a nucleic acid sequence set forth in SEQ ID NO: 90 and reproduced below:

(SEQ ID NO: 90)
CGGGUGGCAUCCCUGUGACCCCUCCCCAGUGCCUCUCCUGGCCCUGGAA
GUUGCCACUCCAGUGCCCACCAGCCUUGUCCUAAUAAAAUUAAGUUGCA
UC.

The 5′ UTR and 3′UTR are described in further detail in WO2012/075040, incorporated herein by reference.

### C. Polyadenylated Tail

As used herein, the terms “poly(A) sequence,” “poly(A) tail,” and “poly(A) region” refer to a sequence of adenosine nucleotides at the 3′ end of the mRNA molecule. The poly(A) tail may confer stability to the mRNA and protect it from exonuclease degradation. The poly(A) tail may enhance translation. In some embodiments, the poly(A) tail is essentially homopolymeric. For example, a poly(A) tail of 100 adenosine nucleotides may have essentially a length of 100 nucleotides. In certain embodiments, the poly(A) tail may be interrupted by at least one nucleotide different from an adenosine nucleotide (e.g., a nucleotide that is not an adenosine nucleotide). For example, a poly(A) tail of 100 adenosine nucleotides may have a length of more than 100 nucleotides (comprising 100 adenosine nucleotides and at least one nucleotide, or a stretch of nucleotides, that are different from an adenosine nucleotide). In certain embodiments, the poly(A) tail comprises the sequence

(SEQ ID NO: 91)
AAAAAAAAAAAAAAAAAAAAAAAAAAAAAAGCAUAUGACUAAAAAAAAA
AAAAAAAAAAAAAAAAAAAAAAAAAAAAAAAAAAAAAAAAAAAAAAAAA
AAAAAAAAAAAA.

The “poly(A) tail,” as used herein, typically relates to RNA. However, in the context of the disclosure, the term likewise relates to corresponding sequences in a DNA molecule (e.g., a “poly(T) sequence”).

The poly(A) tail may comprise about 10 to about 500 adenosine nucleotides, about 10 to about 200 adenosine nucleotides, about 40 to about 200 adenosine nucleotides, or about 40 to about 150 adenosine nucleotides. The length of the poly(A) tail may be at least about 10, 50, 75, 100, 150, 200, 250, 300, 350, 400, 450, or 500 adenosine nucleotides.

In some embodiments where the nucleic acid is an RNA, the poly(A) tail of the nucleic acid is obtained from a DNA template during RNA in vitro transcription. In certain embodiments, the poly(A) tail is obtained in vitro by common methods of chemical synthesis without being transcribed from a DNA template. In various embodiments, poly(A) tails are generated by enzymatic polyadenylation of the RNA (after RNA in vitro transcription) using commercially available polyadenylation kits and corresponding protocols, or alternatively, by using immobilized poly(A)polymerases, e.g., using methods and means as described in WO2016/174271.

The nucleic acid may comprise a poly(A) tail obtained by enzymatic polyadenylation, wherein the majority of nucleic acid molecules comprise about 100 (+/−20) to about 500 (+1-50) or about 250 (+/−20) adenosine nucleotides.

In some embodiments, the nucleic acid may comprise a poly(A) tail derived from a template DNA and may additionally comprise at least one additional poly(A) tail generated by enzymatic polyadenylation, e.g., as described in WO2016/091391.

In certain embodiments, the nucleic acid comprises at least one polyadenylation signal.

In various embodiments, the nucleic acid may comprise at least one poly(C) sequence.

The term “poly(C) sequence,” as used herein, is intended to be a sequence of cytosine nucleotides of up to about 200 cytosine nucleotides. In some embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In some embodiments, the poly(C) sequence comprises about 30 cytosine nucleotides.

### D. Chemical Modification

The mRNA disclosed herein may be modified or unmodified. In some embodiments, the mRNA may comprise at least one chemical modification. In some embodiments, the mRNA disclosed herein may contain one or more modifications that typically enhance RNA stability.

Exemplary modifications can include backbone modifications, sugar modifications, or base modifications. In some embodiments, the disclosed mRNA may be synthesized from naturally occurring nucleotides and/or nucleotide analogues (modified nucleotides) including, but not limited to, purines (adenine (A) and guanine (G)) or pyrimidines (thymine (T), cytosine (C), and uracil (U)). In certain embodiments, the disclosed mRNA may be synthesized from modified nucleotide analogues or derivatives of purines and pyrimidines, such as, e.g., 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, R-D-mannosyl-queosine, phosphoramidates, phosphorothioates, peptide nucleotides, methylphosphonates, 7-deazaguanosine, 5-methylcytosine, and inosine.

In some embodiments, the disclosed mRNA may comprise at least one chemical modification including, but not limited to, pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In some embodiments, the chemical modification is selected from the group consisting of pseudouridine, N1-methylpseudouridine, 5-methylcytosine, 5-methoxyuridine, and a combination thereof.

In some embodiments, the chemical modification comprises N1-methylpseudouridine.

In some embodiments, at least 20%, at least 30%, at least 40%, at least 50%, at least 60%, at least 70%, at least 80%, at least 85%, at least 90%, at least 95%, or 100% of the uracil nucleotides in the mRNA are chemically modified.

In some embodiments, at least 20%, at least 30%, at least 40%, at least 50%, at least 60%, at least 70%, at least 80%, at least 85%, at least 90%, at least 95%, or 100% of the uracil nucleotides in the ORF are chemically modified.

The preparation of such analogues is described, e.g., in U.S. Pat. Nos. 4,373,071, 4,401,796, 4,415,732, 4,458,066, 4,500,707, 4,668,777, 4,973,679, 5,047,524, 5,132,418, 5,153,319, 5,262,530, and 5,700,642.

E. mRNA Synthesis

The mRNAs disclosed herein may be synthesized according to any of a variety of methods. For example, mRNAs according to the present disclosure may be synthesized via in vitro transcription (IVT). Some methods for in vitro transcription are described, e.g., in Geall et al. (2013) Semin. Immunol. 25(2): 152-159; Brunelle et al. (2013) Methods Enzymol. 530:101-14. Briefly, IVT is typically performed with a linear or circular DNA template containing a promoter, a pool of ribonucleotide triphosphates, a buffer system that may include DTT and magnesium ions, an appropriate RNA polymerase (e.g., T3, T7, or SP6 RNA polymerase), DNase I, pyrophosphatase, and/or RNase inhibitor. The exact conditions may vary according to the specific application. The presence of these reagents is generally undesirable in a final mRNA product and these reagents can be considered impurities or contaminants which can be purified or removed to provide a clean and/or homogeneous mRNA that is suitable for therapeutic use. While mRNA provided from in vitro transcription reactions may be desirable in some embodiments, other sources of mRNA can be used according to the instant disclosure including wild-type mRNA produced from bacteria, fungi, plants, and/or animals.

### VIII. Processes for Making the Present LNP Vaccines

The present LNPs can be prepared by various techniques presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion that results in the formation of MLVs. Unilamellar vesicles (ULV) can then be formed by homogenization, sonication or extrusion of the multilamellar vesicles. In addition, unilamellar vesicles can be formed by detergent removal techniques.

Various methods are described in US 2011/0244026, US 2016/0038432, US 2018/0153822, US 2018/0125989, and PCT/US2020/043223 (filed Jul. 23, 2020) and can be used to practice the present disclosure. One exemplary process entails encapsulating mRNA by mixing it with a mixture of lipids, without first pre-forming the lipids into lipid nanoparticles, as described in US 2016/0038432. Another exemplary process entails encapsulating mRNA by mixing pre-formed LNPs with mRNA, as described in US 2018/0153822.

In some embodiments, the process of preparing mRNA-loaded LNPs includes a step of heating one or more of the solutions to a temperature greater than ambient temperature, the one or more solutions being the solution comprising the pre-formed lipid nanoparticles, the solution comprising the mRNA and the mixed solution comprising the LNP-encapsulated mRNA. In some embodiments, the process includes the step of heating one or both of the mRNA solution and the pre-formed LNP solution, prior to the mixing step. In some embodiments, the process includes heating one or more of the solutions comprising the pre-formed LNPs, the solution comprising the mRNA and the solution comprising the LNP-encapsulated mRNA, during the mixing step. In some embodiments, the process includes the step of heating the LNP-encapsulated mRNA, after the mixing step. In some embodiments, the temperature to which one or more of the solutions is heated is or is greater than about 30° C., 37° C., 40° C., 45° C., 50° C., 55° C., 60° C., 65° C., or 70° C. In some embodiments, the temperature to which one or more of the solutions is heated ranges from about 25-70° C., about 30-70° C., about 35-70° C., about 40-70° C., about 45-70° C., about 50-70° C., or about 60-70° C. In some embodiments, the temperature is about 65° C.

Various methods may be used to prepare an mRNA solution suitable for the present disclosure. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water or a buffer at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, or 1.6 mg/ml, 2.0 mg/ml, 2.5 mg/ml, 3.0 mg/ml, 3.5 mg/ml, 4.0 mg/ml, 4.5 mg/ml, or 5.0 mg/ml.

In some embodiments, an mRNA stock solution is mixed with a buffer solution using a pump. Exemplary pumps include but are not limited to gear pumps, peristaltic pumps and centrifugal pumps. Typically, the buffer solution is mixed at a rate greater than that of the mRNA stock solution. For example, the buffer solution may be mixed at a rate at least 1×, 2×, 3×, 4×, 5×, 6×, 7×, 8×, 9×, 10×, 15×, or 20x greater than the rate of the mRNA stock solution. In some embodiments, a buffer solution is mixed at a flow rate ranging between about 100-6000 ml/minute (e.g., about 100-300 ml/minute, 300-600 ml/minute, 600-1200 ml/minute, 1200-2400 ml/minute, 2400-3600 ml/minute, 3600-4800 ml/minute, 4800-6000 ml/minute, or 60-420 ml/minute). In some embodiments, a buffer solution is mixed at a flow rate of, or greater than, about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute.

In some embodiments, an mRNA stock solution is mixed at a flow rate ranging between about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute). In some embodiments, an mRNA stock solution is mixed at a flow rate of or greater than about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute.

The process of incorporation of a desired mRNA into a lipid nanoparticle is referred to as “loading.” Exemplary methods are described in Lasic et al., FEBS Lett. (1992) 312:255-8. The LNP-incorporated nucleic acids may be completely or partially located in the interior space of the lipid nanoparticle, within the bilayer membrane of the lipid nanoparticle, or associated with the exterior surface of the lipid nanoparticle membrane. The incorporation of an mRNA into lipid nanoparticles is also referred to herein as “encapsulation” wherein the nucleic acid is entirely or substantially contained within the interior space of the lipid nanoparticle.

Suitable LNPs may be made in various sizes. In some embodiments, decreased size of lipid nanoparticles is associated with more efficient delivery of an mRNA. Selection of an appropriate LNP size may take into consideration the site of the target cell or tissue and to some extent the application for which the lipid nanoparticle is being made.

A variety of methods known in the art are available for sizing of a population of lipid nanoparticles. Preferred methods herein utilize Zetasizer Nano ZS (Malvern Panalytical) to measure LNP particle size. In one protocol, 10 μl of an LNP sample are mixed with 990 μl of 10% trehalose. This solution is loaded into a cuvette and then put into the Zetasizer machine. The z-average diameter (nm), or cumulants mean, is regarded as the average size for the LNPs in the sample. The Zetasizer machine can also be used to measure the polydispersity index (PDI) by using dynamic light scattering (DLS) and cumulant analysis of the autocorrelation function.

Average LNP diameter may be reduced by sonication of formed LNP. Intermittent sonication cycles may be alternated with quasi-elastic light scattering (QELS) assessment to guide efficient lipid nanoparticle synthesis.

In some embodiments, the majority of purified LNPs, i.e., greater than about 50%, 55%, 60%, 65%, 70%, 75%, 80%, 85%, 90%, 95%, 96%, 97%, 98%, or 99% of the LNPs, have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). In some embodiments, substantially all (e.g., greater than 80 or 90%) of the purified lipid nanoparticles have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm).

In some embodiments, the LNPs in the present composition have an average size of less than 150 nm, less than 120 nm, less than 100 nm, less than 90 nm, less than 80 nm, less than 70 nm, less than 60 nm, less than 50 nm, less than 30 nm, or less than 20 nm.

In some embodiments, greater than about 70%, 75%, 80%, 85%, 90%, 95%, 96%, 97%, 98%, 99% of the LNPs in the present composition have a size ranging from about 40-90 nm (e.g., about 45-85 nm, about 50-80 nm, about 55-75 nm, about 60-70 nm) or about 50-70 nm (e.g., 55-65 nm) are particular suitable for pulmonary delivery via nebulization.

In some embodiments, the dispersity, or measure of heterogeneity in size of molecules (PDI), of LNPs in a pharmaceutical composition provided by the present disclosure is less than about 0.5. In some embodiments, an LNP has a PDI of less than about 0.5, less than about 0.4, less than about 0.3, less than about 0.28, less than about 0.25, less than about 0.23, less than about 0.20, less than about 0.18, less than about 0.16, less than about 0.14, less than about 0.12, less than about 0.10, or less than about 0.08. The PDI may be measured by a Zetasizer machine as described above.

In some embodiments, greater than about 75%, 80%, 85%, 90%, 95%, 96%, 97%, 98%, or 99% of the purified LNPs in a pharmaceutical composition provided herein encapsulate an mRNA within each individual particle. In some embodiments, substantially all (e.g., greater than 80% or 90%) of the purified lipid nanoparticles in a pharmaceutical composition encapsulate an mRNA within each individual particle. In some embodiments, a lipid nanoparticle has an encapsulation efficiency of between 50% and 99%; or greater than about 60, 65, 70, 75, 80, 85, 90, 92, 95, 98, or 99%. Typically, lipid nanoparticles for use herein have an encapsulation efficiency of at least 90% (e.g., at least 91, 92, 93, 94, or 95%).

In some embodiments, an LNP has a N/P ratio of between 1 and 10. In some embodiments, a lipid nanoparticle has a N/P ratio above 1, about 1, about 2, about 3, about 4, about 5, about 6, about 7, or about 8. In further embodiments, a typical LNP herein has an N/P ratio of 4.

In some embodiments, a pharmaceutical composition according to the present disclosure contains at least about 0.5 kg, 1 kg, 5 kg, 10 kg, 100 kg, 500 kg, or 1000 g of encapsulated mRNA. In some embodiments, a pharmaceutical composition contains about 0.1 g to 1000 μg, at least about 0.5 μg, at least about 0.8 μg, at least about 1 μg, at least about 5 μg, at least about 8 kg, at least about 10 kg, at least about 50 kg, at least about 100 kg, at least about 500 kg, or at least about 1000 kg of encapsulated mRNA.

In some embodiments, mRNA can be made by chemical synthesis or by in vitro transcription (IVT) of a DNA template. An exemplary process for making and purifying mRNA is described in Example 1. In this process, in an IVT process, a cDNA template is used to produce an mRNA transcript and the DNA template is degraded by a DNase. The transcript is purified by depth filtration and tangential flow filtration (TFF). The purified transcript is further modified by adding a cap and a tail, and the modified RNA is purified again by depth filtration and TFF.

The mRNA is then prepared in an aqueous buffer and mixed with an amphiphilic solution containing the lipid components of the LNPs. An amphiphilic solution for dissolving the four lipid components of the LNPs may be an alcohol solution. In some embodiments, the alcohol is ethanol.

The aqueous buffer may be, for example, a citrate, phosphate, acetate, or succinate buffer and may have a pH of about 3.0-7.0, e.g., about 3.5, about 4.0, about 4.5, about 5.0, about 5.5, about 6.0, or about 6.5. The buffer may contain other components such as a salt (e.g., sodium, potassium, and/or calcium salts). In particular embodiments, the aqueous buffer has 1 mM citrate, 150 mM NaCl, pH 4.5.

An exemplary, nonlimiting process for making an mRNA-LNP composition is described in Example 1. The process involves mixing of a buffered mRNA solution with a solution of lipids in ethanol in a controlled homogeneous manner, where the ratio of lipids:mRNA is maintained throughout the mixing process. In this illustrative example, the mRNA is presented in an aqueous buffer containing citric acid monohydrate, tri-sodium citrate dihydrate, and sodium chloride. The mRNA solution is added to the solution (1 mM citrate buffer, 150 mM NaCl, pH 4.5). The lipid mixture of four lipids (e.g., a cationic lipid, a PEGylated lipid, a cholesterol-based lipid, and a helper lipid) is dissolved in ethanol. The aqueous mRNA solution and the ethanol lipid solution are mixed at a volume ratio of 4:1 in a “T” mixer with a near “pulseless” pump system. The resultant mixture is then subjected for downstream purification and buffer exchange. The buffer exchange may be achieved using dialysis cassettes or a TFF system. TFF may be used to concentrate and buffer-exchange the resulting nascent LNP immediately after formation via the T-mix process. The diafiltration process is a continuous operation, keeping the volume constant by adding appropriate buffer at the same rate as the permeate flow.

IX. Packaging and Use of the mRNA-LNP Vaccines

The mRNA-LNP vaccines can be formulated or packaged for parenteral (e.g., intramuscular, intradermal or subcutaneous) administration or nasopharyngeal (e.g., intranasal) administration. The vaccine compositions may be in the form of an extemporaneous formulation, where the LNP composition is lyophilized and reconstituted with a physiological buffer (e.g., PBS) just before use. The vaccine compositions also may be shipped and provided in the form of an aqueous solution or a frozen aqueous solution and can be directly administered to subjects without reconstitution (after thawing, if previously frozen).

Accordingly, the present disclosure provides an article of manufacture, such as a kit, that provides the mRNA-LNP vaccine in a single container, or provides the mRNA-LNP vaccine in one container and a physiological buffer for reconstitution in another container. The container(s) may contain a single-use dosage or multi-use dosage. The containers may be pre-treated glass vials or ampules. The article of manufacture may include instructions for use as well.

In certain embodiments, the mRNA-LNP vaccine is provided for use in intramuscular (IM) injection. The vaccine can be injected to a subject at, e.g., his/her deltoid muscle in the upper arm. In some embodiments, the vaccine is provided in a pre-filled syringe or injector (e.g., single-chambered or multi-chambered). In some embodiments, the vaccine is provided for use in inhalation and is provided in a pre-filled pump, aerosolizer, or inhaler.

The mRNA-LNP vaccines can be administered to subjects in need thereof in a prophylactically effective amount, i.e., an amount that provides sufficient immune protection against a target pathogen for a sufficient amount of time (e.g., one year, two years, five years, ten years, or life-time). Sufficient immune protection may be, for example, prevention or alleviation of symptoms associated with infections by the pathogen. In some embodiments, multiple doses (e.g., two doses) of the vaccine are injected to subjects in need thereof to achieve the desired prophylactic effects. The doses (e.g., prime and booster doses) may be separated by an interval of e.g., 1 week, 2 weeks, 3 weeks, 4 weeks, one month, two months, three months, four months, five months, six months, one year, two years, five years, or ten years.

In some embodiments, a single dose of the mRNA-LNP vaccine contains 1-50 g of mRNA (e.g., monovalent or multivalent). For example, a single dose may contain about 2.5 g, about 5 g, about 7.5 g, about 10 g, about 12.5 g, or about 15 ag of the mRNA for intramuscular (IM) injection. In further embodiments, a multi-valent single dose of an LNP vaccine contains multiple (e.g., 2, 3, or 4) kinds of LNPs, each for a different antigen, and each kind of LNP has an mRNA amount of, e.g., 2.5 kg, about 5 kg, about 7.5 g, about 10 kg, about 12.5 g, or about 15 kg.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art.

Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

### X. Vectors

In one aspect, disclosed herein are vectors comprising the mRNA compositions disclosed herein. The RNA sequences encoding a protein of interest (e.g., mRNA encoding an antigenic prokaryotic polypeptide) can be cloned into a number of types of vectors. For example, the nucleic acids can be cloned into a vector including, but not limited to, a plasmid, a phagemid, a phage derivative, an animal virus, and a cosmid. Vectors of particular interest can include expression vectors, replication vectors, probe generation vectors, sequencing vectors, and vectors optimized for in vitro transcription.

In certain embodiments, the vector can be used to express mRNA in a host cell. In various embodiments, the vector can be used as a template for IVT. The construction of optimally translated IVT mRNA suitable for therapeutic use is disclosed in detail in Sahin, et al. (2014). Nat. Rev. Drug Discov. 13, 759-780; Weissman (2015). Expert Rev. Vaccines 14, 265-281.

In some embodiments, the vectors disclosed herein can comprise at least the following, from 5′ to 3′: an RNA polymerase promoter; a polynucleotide sequence encoding a 5′ UTR; a polynucleotide sequence encoding an ORF; a polynucleotide sequence encoding a 3′ UTR; and a polynucleotide sequence encoding at least one RNA aptamer. In some embodiments, the vectors disclosed herein may comprise a polynucleotide sequence encoding a poly(A) sequence and/or a polyadenylation signal.

A variety of RNA polymerase promoters are known. In some embodiments, the promoter can be a T7 RNA polymerase promoter. Other useful promoters can include, but are not limited to, T3 and SP6 RNA polymerase promoters. Consensus nucleotide sequences for T7, T3, and SP6 promoters are known.

Also disclosed herein are host cells (e.g., mammalian cells, e.g., human cells) comprising the vectors or RNA compositions disclosed herein.

Polynucleotides can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendorf, Hamburg, Germany), cationic liposome mediated transfection using lipofection, polymer encapsulation, peptide mediated transfection, biolistic particle delivery systems such as “gene guns” (see, for example, Nishikawa, et al. (2001). Hum Gene Ther. 12(8):861-70, or the TransIT-RNA transfection Kit (Mirus, Madison, WI).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes.

An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present disclosure, in order to confirm the presence of the mRNA sequence in the host cell a variety of assays may be performed.

### XI. Self-Replicating RNA, Trans-Replicating RNA and Non-Replicating RNA

### Self-replicating RNA:

Self-replicating (or self-amplifying) RNA can be produced by using replication elements derived from, e.g., alphaviruses, and substituting the structural viral proteins with a nucleotide sequence encoding a protein of interest (e.g., an antigenic prokaryotic polypeptide). A self-replicating RNA is typically a positive-strand molecule which can be directly translated after delivery to a cell, and this translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded antigen, or may be transcribed to provide further transcripts with the same sense as the delivered RNA which are translated to provide in situ expression of the antigen. The overall result of this sequence of transcriptions is a large amplification in the number of the introduced replicon RNAs and so the encoded antigen becomes a major polypeptide product of the cells.

One suitable system for achieving self-replication in this manner is to use an alphavirus-based replicon. These replicons are positive stranded (positive sense-stranded) RNAs which lead to translation of a replicase (or replicase-transcriptase) after delivery to a cell. The replicase is translated as a polyprotein which auto-cleaves to provide a replication complex which creates genomic-strand copies of the positive-strand delivered RNA. These negative (-)-stranded transcripts can themselves be transcribed to give further copies of the positive-stranded parent RNA and also to give a subgenomic transcript which encodes the antigen. Translation of the subgenomic transcript thus leads to in situ expression of the antigen by the infected cell. Suitable alphavirus replicons can use a replicase from a Sindbis virus, a Semliki forest virus, an eastern equine encephalitis virus, a Venezuelan equine encephalitis virus, etc. Mutant or wild-type virus sequences can be used, e.g., the attenuated TC83 mutant of VEEV has been used in replicons, see the following reference: WO2005/113782, incorporated herein by reference.

In one embodiment, each self-replicating RNA described herein encodes (i) an RNA-dependent RNA polymerase which can transcribe RNA from the self-replicating RNA molecule and (ii) a protein antigen. The polymerase can be an alphavirus replicase, e.g., comprising one or more of alphavirus proteins nsP1, nsP2, nsP3, and nsP4. Whereas natural alphavirus genomes encode structural virion proteins in addition to the non-structural replicase polyprotein, in certain embodiments, the self-replicating RNA molecules do not encode alphavirus structural proteins.

Thus, the self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the present disclosure and their place is taken by gene(s) encoding the immunogen of interest, such that the subgenomic transcript encodes the immunogen rather than the structural alphavirus virion proteins. Self-replicating RNA are described in further detail in WO2011005799, incorporated herein by reference.

### Trans-Replicating RNA:

Trans-replicating (or trans-amplifying) RNA possess similar elements as the self-replicating RNA described above. However, with trans replicating RNA, two separate RNA molecules are used. A first RNA molecule encodes for the RNA replicase described above (e.g., the alphavirus replicase) and a second RNA molecule encodes for the protein of interest (e.g., an antigenic prokaryotic polypeptide). The RNA replicase may replicate one or both of the first and second RNA molecule, thereby greatly increasing the copy number of RNA molecules encoding the protein of interest. Trans replicating RNA are described in further detail in WO2017162265, incorporated herein by reference.

### Non-Replicating RNA:

Non-replicating (or non-amplifying) RNA is an RNA without the ability to replicate itself.

### XI. Pharmaceutical Compositions

The pharmaceutical compositions according to this disclosure typically include a nucleic acid, in particular RNA, and more particularly mRNA, and a pharmaceutically acceptable carrier, or a pharmaceutically acceptable excipient or a pharmaceutically acceptable diluent, which makes the composition especially suitable for therapeutic use. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The pharmaceutical composition may for instance be an immunogenic composition, i.e. a composition which, when administered to a subject, elicits an immune response. It should be understood that the terms “immunogenic composition”, “vaccine composition” and “vaccine” are used interchangeably herein and are thus meant to have equivalent meanings.

A pharmaceutical composition of the present disclosure can also include one or more additional components such as small molecule immunopotentiators (e.g., TLR agonists). A pharmaceutical composition of the present disclosure can also include a delivery system for the RNA, such as a liposome, an oil-in-water emulsion, or a microparticle. In some embodiments, the pharmaceutical composition comprises a lipid nanoparticle (LNP). In certain embodiments, the composition comprises an antigen-encoding nucleic acid molecule encapsulated within an LNP.

### XII. Methods of Vaccination

The nucleic acid (e.g., mRNA) vaccines disclosed herein may be administered to a subject to induce an immune response directed against an antigenic prokaryotic polypeptide, wherein an anti-antigen antibody titer in the subject is increased following vaccination relative to an anti-antigen antibody titer in a subject that is not vaccinated with the nucleic acid vaccine disclosed herein, or relative to an alternative vaccine against the prokaryotic polypeptide. An “anti-antigen antibody” is a serum antibody that binds specifically to the antigen.

In one aspect, the disclosure provides a method of eliciting an immune response in a subject in need thereof, comprising administering to the subject, optionally intramuscularly, intranasally, intravenously, subcutaneously, or intradermally, an effective amount of the nucleic acid (e.g., mRNA) vaccine described herein.

In another aspect, the disclosure provides a method of treating or preventing a prokaryotic infection in a subject in need thereof, comprising administering to the subject, optionally intramuscularly, intranasally, intravenously, subcutaneously, or intradermally, an effective amount of the nucleic acid vaccine described herein.

In another aspect, the disclosure provides the use of the nucleic acid vaccine described herein for the manufacture of a medicament for use in eliciting an immune response in a subject in need thereof.

In another aspect, the disclosure provides the use of the nucleic acid vaccine described herein for the manufacture of a medicament for use in treating or preventing a prokaryotic infection in a subject in need thereof.

In another aspect, the disclosure provides the nucleic acid vaccine described herein for use in eliciting an immune response in a subject in need thereof.

In another aspect, the disclosure provides the nucleic acid vaccine described herein for use in treating or preventing a prokaryotic infection in a subject in need thereof.

In one aspect, the disclosure provides method of secreting an antigenic prokaryotic polypeptide in a host cell, the method comprising administering to the host cell the nucleic acid vaccine described herein.

In another aspect, the disclosure provides method of displaying an antigenic prokaryotic polypeptide on the surface of a host cell, the method comprising administering to the host cell the nucleic acid vaccine described herein.

The present invention comprises the following embodiments.
Embodiment 1. A nucleic acid comprising an open reading frame (ORF), wherein the ORF comprises:—a polynucleotide sequence encoding at least one antigenic prokaryotic polypeptide and—a polynucleotide sequence encoding at least one viral secretion signal peptide.
Embodiment 2. The nucleic acid of embodiment 1, wherein the ORF further comprises a polynucleotide sequence encoding at least one transmembrane domain (TMB).
Embodiment 3. The nucleic acid of embodiment 1 or 2, wherein the viral secretion signal peptide is derived from a viral sequence in a virus able to infect humans.
Embodiment 4. The nucleic acid of any one of embodiments 1-3, wherein the viral secretion signal peptide is derived from a viral sequence selected from the group consisting of: an influenza secretion signal peptide sequence, and a non-influenza secretion signal peptide sequence selected from the group consisting of a SARS CoV-2 secretion signal peptide sequence, a varicella-zoster virus (VZV) secretion signal peptide sequence, a measles secretion signal peptide sequence, a rubella secretion signal peptide sequence, a mumps secretion signal peptide sequence, an Ebola secretion signal peptide sequence, a smallpox secretion signal peptide sequence, and a rabies secretion signal peptide sequence.
Embodiment 5. The nucleic acid of any one of embodiments 1-4, wherein the viral secretion signal peptide is selected from the group consisting of: an influenza hemagglutinin (HA) secretion signal peptide sequence, a SARS CoV-2 spike secretion signal peptide sequence, a VZV gB secretion signal peptide sequence, a VZV gE secretion signal peptide sequence, a VZV gI secretion signal peptide sequence, a VZV gK secretion signal peptide sequence, a measles F-protein secretion signal peptide sequence, a rubella E1 protein secretion signal peptide sequence, a rubella E2 protein secretion signal peptide sequence, a mumps F-protein secretion signal peptide sequence, an Ebola GP protein secretion signal peptide sequence, a smallpox 6 kDa IC protein secretion signal peptide sequence, and a rabies G protein secretion signal peptide sequence, preferably wherein the viral secretion signal peptide comprises an HA secretion signal peptide sequence from influenza A or influenza B, more preferably from influenza A.
Embodiment 6. The nucleic acid of embodiment 5, wherein the HA secretion signal peptide sequence comprises an amino acid sequence MKX₁X₂LX₃VX₄LX₅TFX₆X₇X₅X₉A (SEQ ID NO: 145) wherein X₁ is selected from A and V; X₂ is selected from I and K; X₃ is selected from V and L; X₄ is selected from L and M; Xₛ is selected from Y and C; X₆ is selected from T and A; X₇ is selected from T and A; Xs is selected from A and T; and X₉ is selected from N and Y.
Embodiment 7. The nucleic acid of embodiment of 5 or 6, wherein the HA secretion signal peptide sequence comprises an amino acid sequence selected from SEQ ID NO: 95-109.
Embodiment 8. The nucleic acid of embodiment of 5, wherein the HA secretion signal peptide sequence comprises an amino acid sequence MKX₁IIALSX₂ILCLVFX₃ (SEQ ID NO: 146) wherein X₁ is selected from T and A; X₂ is selected from Y, N, C, and H; and X₃ is selected from T and A.
Embodiment 9. The nucleic acid of embodiment 8, wherein the HA secretion signal peptide sequence comprises an amino acid sequence selected from SEQ ID NOs: 110-131.
Embodiment 10. The nucleic acid of embodiment 5, wherein the HA secretion signal peptide sequence comprises an amino acid sequence MKAIIVLLMVVTSX₁A (SEQ ID NO: 147) wherein X₁ is selected from S and N.
Embodiment 11. The nucleic acid of embodiment 5, wherein the HA secretion signal peptide sequence comprises an amino acid sequence MX₁AIIVLLMVVTSNA (SEQ ID NO: 148) wherein X₁ is selected from K and E.
Embodiment 12. The nucleic acid of embodiment 10 or 11, wherein the HA secretion signal peptide sequence comprises an amino acid sequence selected from SEQ ID NOs: 132-144.
Embodiment 13. The nucleic acid of any one of embodiments 1-12, wherein the viral secretion signal peptide comprises an amino acid sequence selected from the group consisting of:

(SEQ ID NO: 1)
MKAKLLVLLCTFTATYA;
(SEQ ID NO: 2)
MKAILVVLLYTFATANA;
(SEQ ID NO: 3)
MKTIIALSYILCLVFA;
(SEQ ID NO: 4)
MKAIIVLLMVVTSNA;
(SEQ ID NO: 5)
MFVFLVLLPLVS;
(SEQ ID NO: 6)
MFLLTTKRTMFVFLVLLPLVS;
(SEQ ID NO: 7)
MSPCGYYSKWRNRDRPEYRRNLRFRRFFSSIHPNAAAGSGFNGPGVFIT
SVTGVWLCFLCIFSMFVTAVVS;
(SEQ ID NO: 8)
MGTVNKPVVGVLMGFGIITGTLRITNPVRA;
(SEQ ID NO: 9)
MFLIQCLISAVIFYIQVTNA;
(SEQ ID NO: 10)
MQALGIKTEHFIIMCLLSGHA;
(SEQ ID NO: 11)
MGLKVNVSAIFMAVLLTLQTPTG;
(SEQ ID NO: 12)
MGAAAALTAVVLQGYNPPAYG;
(SEQ ID NO: 13)
MGAPQAFLAGLLLAAVAVGTARA;
(SEQ ID NO: 14)
MKVFLVTCLGFAVESSSVC;
(SEQ ID NO: 15)
MGVTGILQLPRDRFKRTSFFLWVIILFQRTFS;
(SEQ ID NO: 16)
MRSLIIFLLFPSIIYS;
and
(SEQ ID NO: 184)
MVPQALLFVPLLVFPLCFG.

Embodiment 14. The nucleic acid of embodiment 13, wherein the viral secretion signal peptide comprises an amino acid sequence of MKAKLLVLLCTFTATYA (SEQ ID NO: 1).
Embodiment 15. The nucleic acid of any one of embodiments 1-14, wherein the viral secretion signal peptide is positioned at the N-terminus of the antigenic prokaryotic polypeptide.
Embodiment 16. The nucleic acid of any one of embodiments 1-14, wherein the viral secretion signal peptide is positioned at the C-terminus of the antigenic prokaryotic polypeptide.
Embodiment 17. The nucleic acid of any one of embodiments 1-16, wherein the viral secretion signal peptide is attached to the antigenic prokaryotic polypeptide with a linker.
Embodiment 18. The nucleic acid of any one of the embodiments 1-17, wherein the viral secretion signal peptide has a SignalP cleavage probability score of at least 0.8, at least 0.85, at least 0.90 or at least 0.95, as determined using SignalP 6.0.
Embodiment 19. The nucleic acid of any one of the embodiments 1-18, wherein the viral secretion signal peptide has a SignalP signal peptide likelihood score of at least 0.8, at least 0.85, at least 0.90 or at least 0.95, as determined using SignalP 6.0.
Embodiment 20. The nucleic acid of any one of embodiments 2-19, wherein the TMB: (a) comprises or consists of 15 to 50 amino acid residues, preferably 15 to 30 amino acid residues, more preferably 18 to 25 amino acid residues; and/or (b) comprises at least 50%, at least 55%, or at least 60% of hydrophobic amino acid residues, preferably selected in the group consisting of: alanine, isoleucine, leucine, valine, phenylalanine, tryptophane and tyrosine; and/or (c) comprises at least one alpha helix.
Embodiment 21. The nucleic acid of any one of embodiments 2-20, wherein the TMB is derived from an integral membrane protein, preferably from a single pass membrane protein, more preferably from a bitopic membrane protein, even more preferably from a bitopic membrane protein of Type I.
Embodiment 22. The nucleic acid of any one of embodiments 2-11, wherein the TMB is derived from a non-human sequence.
Embodiment 23. The nucleic acid of any one of embodiments 18-22, wherein the antigenic prokaryotic polypeptide is derived from a prokaryotic transmembrane protein, and wherein the TMB is the TMB of the prokaryotic transmembrane protein.
Embodiment 24. The nucleic acid of any one of embodiments 18-23, wherein the antigenic prokaryotic polypeptide is not derived from a prokaryotic transmembrane protein.
Embodiment 25. The nucleic acid of embodiment 24, wherein the TMB is derived from a viral sequence.
Embodiment 26. The nucleic acid of embodiment 25, wherein the TMB is derived from a viral transmembrane domain sequence selected from the group consisting of: an influenza transmembrane domain sequence, and a non-influenza transmembrane domain sequence selected from the group consisting of a SARS CoV-2 transmembrane domain sequence, a varicella-zoster virus (VZV) transmembrane domain sequence, a measles transmembrane domain sequence, a rubella transmembrane domain sequence, a mumps transmembrane domain sequence, an Ebola transmembrane domain sequence, and a rabies transmembrane domain sequence.
Embodiment 27. The nucleic acid of embodiment 26, wherein the TMB is selected from the group consisting of: an influenza hemagglutinin (HA) transmembrane domain sequence, a SARS CoV-2 spike transmembrane domain sequence, a VZV gB transmembrane domain sequence, a VZV gE transmembrane domain sequence, a VZV gI transmembrane domain sequence, a VZV gK transmembrane domain sequence, a measles F-protein transmembrane domain sequence, a rubella E1 protein transmembrane domain sequence, a rubella E2 protein transmembrane domain sequence, a mumps F-protein transmembrane domain sequence, an Ebola GP protein transmembrane domain sequence and a rabies G protein transmembrane domain sequence, preferably wherein the TMB comprises an HA transmembrane domain sequence from influenza A or influenza B, more preferably from influenza A.
Embodiment 28. The nucleic acid of any one of embodiments 18-22 and 24-27, wherein the TMB comprises an amino acid sequence selected from the group consisting of:

(SEQ ID NO: 17)
ILAIYSTVASSLVLLVSLGAISF;
(SEQ ID NO: 18)
ILAIYSTVASSLVLVVSLGAISF;
(SEQ ID NO: 19)
ILWISFAISCFLLCVVLLGFI;
(SEQ ID NO: 20)
STAASSLAVTLMLAIFIVYMV;
(SEQ ID NO: 21)
WYIWLGFIAGLIAIVMVTIML;
(SEQ ID NO: 22)
FGALAVGLLVLAGLVAAFFAY;
(SEQ ID NO: 23)
AAWTGGLAAVVLLCLVIFLIC;
(SEQ ID NO: 24)
IIIPIVASVMILTAMVIVIVI;
(SEQ ID NO: 25)
YFWCVQLKMIFFAWFVYGMYL;
(SEQ ID NO: 26)
IVYILIAVCLGGLIGIPALIC;
(SEQ ID NO: 27)
LDHAFAAFVLLVPWVLIFMVC;
(SEQ ID NO: 28)
WWQLTLGAICALLLAGLLACC;
(SEQ ID NO: 29)
IVAALVLSILSIIISLLFCCW;
(SEQ ID NO: 30)
WIPAGIGVTGVIIAVIALFCI;
and
(SEQ ID NO: 185)
VLLSAGALTALMLIIFLMTCW.

Embodiment 29. The nucleic acid of embodiment 28, wherein the TMB comprises an amino acid sequence of ILAIYSTVASSLVLLVSLGAISF (SEQ ID NO: 17).
Embodiment 30. The nucleic acid of embodiments 2 and 18-29, wherein the TMB is attached to the antigenic prokaryotic polypeptide with a linker.
Embodiment 31. The nucleic acid of any one of embodiments 2 and 18-30, wherein the TMB is positioned at the N-terminus of the antigenic prokaryotic polypeptide.
Embodiment 32. The nucleic acid of any one of embodiments 2 and 18-30, wherein the TMB is positioned at the C-terminus of the antigenic prokaryotic polypeptide.
Embodiment 33. A nucleic acid comprising an open reading frame (ORF), wherein the ORF comprises:—a polynucleotide sequence encoding at least one antigenic polypeptide, preferably an antigenic prokaryotic polypeptide, and—a polynucleotide sequence encoding at least one transmembrane domain (TMB), wherein the TMB is heterologous to the antigenic polypeptide, wherein optionally the ORF further comprises a polynucleotide sequence encoding at least one secretion signal peptide, preferably a viral secretion signal peptide, more preferably a viral secretion signal peptide as described in any one of the preceding claims.
Embodiment 34. A nucleic acid comprising an open reading frame (ORF), wherein the ORF comprises:—a polynucleotide sequence encoding at least one antigenic prokaryotic polypeptide and—a polynucleotide sequence encoding at least one transmembrane domain (TMB).
Embodiment 35. The nucleic acid of embodiment 34, wherein the TMB: (a) comprises or consists of 15 to 50 amino acid residues, preferably 15 to 30 amino acid residues, more preferably 18 to 25 amino acid residues; and/or (b) comprises at least 50%, at least 55%, or at least 60% of hydrophobic amino acid residues, preferably selected in the group consisting of: alanine, isoleucine, leucine, valine, phenylalanine, tryptophane and tyrosine; and/or (c) comprises at least one alpha helix.
Embodiment 36. The nucleic acid of embodiment 34 or 35, wherein the TMB is derived from an integral membrane protein, preferably from a single pass membrane protein, more preferably from a bitopic membrane protein, even more preferably from a bitopic membrane protein of Type I.
Embodiment 37. The nucleic acid of embodiment 36, wherein the TMB is derived from a non-human sequence.
Embodiment 38. The nucleic acid of any one of embodiments 1-32, wherein the antigenic prokaryotic polypeptide is derived from a prokaryotic transmembrane protein, and wherein the TMB is the transmembrane domain of the heterologous prokaryotic transmembrane protein.
Embodiment 39. The nucleic acid of any one of embodiments 35-38, wherein the antigenic polypeptide is not derived from a transmembrane protein.
Embodiment 40. The nucleic acid of any one of embodiments 35-37 and 39, wherein the TMB is derived from a viral sequence.
Embodiment 41. The nucleic acid of embodiments 33-35,39, and 40, wherein the TMB is derived from a viral transmembrane domain sequence selected from the group consisting of: an influenza transmembrane domain sequence, and a non-influenza transmembrane domain sequence selected from the group consisting of a SARS CoV-2 transmembrane domain sequence, a varicella-zoster virus (VZV) transmembrane domain sequence, a measles transmembrane domain sequence, a rubella transmembrane domain sequence, a mumps transmembrane domain sequence, an Ebola transmembrane domain sequence, and a rabies transmembrane domain sequence.
Embodiment 42. The nucleic acid of claim 41, wherein the TMB is selected from the group consisting of: an influenza hemagglutinin (HA) transmembrane domain sequence, a SARS CoV-2 spike transmembrane domain sequence, a VZV gB transmembrane domain sequence, a VZV gE transmembrane domain sequence, a VZV gI transmembrane domain sequence, a VZV gK transmembrane domain sequence, a measles F-protein transmembrane domain sequence, a rubella E1 protein transmembrane domain sequence, a rubella E2 protein transmembrane domain sequence, a mumps F-protein transmembrane domain sequence, an Ebola GP protein transmembrane domain sequence and a rabies G protein transmembrane domain sequence, preferably wherein the TMB comprises an HA transmembrane domain sequence from influenza A or influenza B, more preferably from influenza A.
Embodiment 43. The nucleic acid of embodiment 42, wherein the TMB comprises an amino acid sequence selected from the group consisting of:

(SEQ ID NO: 17)
ILAIYSTVASSLVLLVSLGAISF;
(SEQ ID NO: 18)
ILAIYSTVASSLVLVVSLGAISF;
(SEQ ID NO: 19)
ILWISFAISCFLLCVVLLGFI;
(SEQ ID NO: 20)
STAASSLAVTLMLAIFIVYMV;
(SEQ ID NO: 21)
WYIWLGFIAGLIAIVMVTIML;
(SEQ ID NO: 22)
FGALAVGLLVLAGLVAAFFAY;
(SEQ ID NO: 23)
AAWTGGLAAVVLLCLVIFLIC;
(SEQ ID NO: 24)
IIIPIVASVMILTAMVIVIVI;
(SEQ ID NO: 25)
YFWCVQLKMIFFAWFVYGMYL;
(SEQ ID NO: 26)
IVYILIAVCLGGLIGIPALIC;
(SEQ ID NO: 27)
LDHAFAAFVLLVPWVLIFMVC;
(SEQ ID NO: 28)
WWQLTLGAICALLLAGLLACC;
(SEQ ID NO: 29)
IVAALVLSILSIIISLLFCCW;
(SEQ ID NO: 30)
WIPAGIGVTGVIIAVIALFCI;
and
(SEQ ID NO: 185)
VLLSAGALTALMLIIFLMTCW.

Embodiment 44. The nucleic acid of embodiment 43, wherein the TMB comprises an amino acid sequence of ILAIYSTVASSLVLLVSLGAISF (SEQ ID NO: 17).
Embodiment 45. The nucleic acid of any one of embodiments 34-44, wherein the TMB is attached to the antigenic prokaryotic polypeptide with a linker.
Embodiment 46. The nucleic acid of any one of embodiments 34-44, wherein the TMB is positioned at the N-terminus of the antigenic prokaryotic polypeptide.
Embodiment 47. The nucleic acid of any one of embodiments 34-44, wherein the TMB is positioned at the C-terminus of the antigenic prokaryotic polypeptide.
Embodiment 48. The nucleic acid of any one of embodiments 34-47, wherein the antigenic prokaryotic polypeptide is derived from a bacteria of a genus selected from the group consisting of Acetobacter, Acinetobacter, Actinomyces, Aerococcus, Agrobacterium, Anaplasma, Azorhizobia, Azotobacter, Bacillus, Bacteroides, Bartonella, Bordetella, Borrelia, Brucella, Burkkolderia, Calymmatobacterium, Campylobacter, Chlamydia, Chlamydophila, Clostridium, Corynebacterium, Coxiella, Cutibacterium, Ehrlichia, Enterobacter, Enterococcus, Escherichia, Francisella, Fusobacterium, Gardnerella, Haemophilus, Helicobacter, Klebsiella, Lactobacillus, Lactococcus, Legionella, Listeria, Methanobacterium, Microbacterium, Micrococcus, Moraxella, Mycobacterium, Mycoplasma, Neisseria, Pasteurella, Pediococcus, Peptostreptococcus, Porphyromonas, Prevotella, Propionibacterium, Pseudomonas, Rhizobium, Rickettsia, Rochalimaea, Rothia, Salmonella, Serratia, Shigella, Sarcina, Spirillum, Spirochaetes, Staphylococcus, Stenotrophomonas, Streptobacillus, Streptococcus, Tetragenococcus, Treponema, Vibrio, Viridans, Walbachia, and Yersinia, preferably from a bacteria of a species selected from the group consisting of Acetobacter aurantius, Acinetobacter baumannii, Actinomyces israelii, Agrobacterium radiobacter, Agrobacterium tumefaciens, Anaplasma phagocytophilum, Azorhizobium caulinodans, Azotobacter vinelandii, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus Thuringiensis, Bacteroides fragilis, Bacteroides gingivalis, Bacteroides melaninogenicus, Bartonella henselae, Bartonella Quintana, Bordetella bronchiseptica, Bordetella pertussis, Borrelia burgdorferi. Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Campylobacter pylori, Chlamydia trachomatis, Chlamydophila pneumoniae, Chlamydophila psittaci, Clostridium botulinum, Clostridium difficile, Clostridium perfringens, Clostridium tetani, Corynebacterium diphtheriae, Corynebacterium fusiforme, Coxiella burnetii, Cutibacterium acnes, Cutibacterium avidum, Cutibacterium granulosum, Cutibacterium namnetense, Cutibacterium humerusii, Ehrlichia chaffeensis, Enterobacter cloacae, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus bulgaricus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Neisseria gonorrhoeae, Neisseria meningitidis, Pasteurella multocida, Pasteurella tularensis, Peptostreptococcus, Porphyromonas gingivalis, Prevotella melaninogenica, Propionibacterium acnes, Pseudomonas aeruginosa, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus, Staphylococcus epidermidis, Stenotrophomonas maltophilia, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Viridans streptococci, Wolbachia, Yersinia enterocolitica, Yersinia pestis and Yersinia pseudotuberculosis.
Embodiment 49. The nucleic acid of any one of embodiments 1-48, wherein the antigenic prokaryotic polypeptide is derived from a bacteria of the genus Borrelia, preferably selected from the species B. burgdorferi, afzelii, garinii, bavariensis, mayonii, spielmanii, lusitaniae, bissettii and/or valaisiana.
Embodiment 50. The nucleic acid of embodiment 49, wherein the antigenic prokaryotic polypeptide is a OspA ST1, a OspA ST2, a OspA ST3, a OspA ST4, a OspA ST5, a OspA ST6, a OspA ST7 or a fragment thereof.
Embodiment 51. The nucleic acid of embodiment 50, wherein the antigenic prokaryotic polypeptide is OspA or a fragment or variant thereof, wherein the OspA or fragment or variant thereof comprises at least 5, at least 10, 20, 30, 40, 50, 60, 70, 80, 90, 100, 110, 120, 130, 140, 150, 160, 170, 180, 190, 200, 210, 220, 230, 240, or 250 amino acids, preferably wherein the antigenic prokaryotic polypeptide comprises: (a) an amino acid sequence derived from OspA ST1, preferably with at least 85%, 86%, 87%, 88%, 89%, 90%, 91%, 92%, 93%, 94%, 95%, 96%, 97%, 98%, 99%, or 100% identity to the sequence KQNVSSLDEKNSVSVDLPGEMKVLVSKEKNKDGKYDLIATVDKLELKGTSDKNNGSGV LEGVKADKSKVKLTISDDLGQTTLEVFKEDGKTLVSKKVTSKDKSSTEEKFNEKGEVSE KIITRADGTRLEYTGIKSDGSGKAKEVLKGYDLKGELSSEKTTLVVKEGTVTLSKNISKS GEVSVELNDTDSSAATKKTAAWNSGTSTLTITVNSKKTKDLVFTKENTITVQQYDSNGT KLEGSAVEITKLDEIKNALK (SEQ ID NO: 31);(b) an amino acid sequence derived from OspA ST2, preferably with at least 85%, 86%, 87%, 88%, 89%, 90%, 91%, 92%, 93%, 94%, 95%, 96%, 97%, 98%, 99%, or 100% identity to the sequence KQNVSSLDEKNSASVDLPGEMKVLVSKEKDKDGKYSLKATVDKIELKGTSDKDNGSGV LEGTKDDKSKAKLTIADDLSKTTFELFKEDGKTLVSRKVSSKDKTSTDEMFNEKGELSA KTMTRENGTKLEYTEMKSDGTGKAKEVLKNFTLEGKVANDKVTLEVKEGTVTLSKEIA KSGEVTVALNDTNTTQATKKTGAWDSKTSTLTISVNSKKTTQLVFTKQDTITVQKYDSA GTNLEGTAVEIKTLDELKNALK (SEQ ID NO: 32); (c) an amino acid sequence derived from OspA ST3, preferably with at least 85%, 86%, 87%, 88%, 89%, 90%, 91%, 92%, 93%, 94%, 95%, 96%, 97%, 98%, 99%, or 100% identity to the sequence KQNVSSLDEKNSVSVDLPGGMKVLVSKEKDKDGKYSLMATVEKLELKGTSDKSNGSG VLEGEKADKSKAKLTISQDLNQTTFEIFKEDGKTLVSRKVNSKDKSSTEEKFNDKGKLSE KVVTRANGTRLEYTEIKNDGSGKAKEVLKGFALEGTLTDGGETKLTVTEGTVTLSKNIS KSGEITVALNDTETTPADKKTGEWKSDTSTLTISKNSQKPKQLVFTKENTITVQNYNRAG NALEGSPAEIKDLAELKAALK (SEQ ID NO: 186); (d) an amino acid sequence derived from OspA ST4, preferably with at least 85%, 86%, 87%, 88%, 89%, 90%, 91%, 92%, 93%, 94%, 95%, 96%, 97%, 98%, 99%, or 100% identity to the sequence KQNVSSLDEKNSVSVDLPGEMKVLVSKEKDKDGKYSLMATVDKLELKGTSDKSNGSG TLEGEKSDKSKAKLTISEDLSKTTFEIFKEDGKTLVSKKVNSKDKSSIEEKFNAKGELSEK TILRANGTRLEYTEIKSDGTGKAKEVLKDFALEGTLAADKTTLKVTEGTVVLSKHIPNSG EITVELNDSNSTQATKKTGKWDSNTSTLTISVNSKKTKNIVFTKEDTITVQKYDSAGTNL EGNAVEIKTLDELKNALK (SEQ ID NO: 187); (e) an amino acid sequence derived from OspA ST5, preferably with at least 85% identity to the sequence KQNVSSLDEKNSVSVDLPGGMKVLVSKEKDKDGKYSLMATVEKLELKGTSDKNNGSG TLEGEKTDKSKVKLTIAEDLSKTTFEIFKEDGKTLVSKKVTLKDKSSTEEKFNEKGEISEK TIVRANGTRLEYTDIKSDGSGKAKEVLKDFTLEGTLAADGKTTLKVTEGTVVLSKNILKS GEITVALDDSDTTQATKKTGKWDSKTSTLTISVNSQKTKNLVFTKEDTITVQKYDSAGT NLEGKAVEITTLEKLKDALK (SEQ ID NO: 188); (f) an amino acid sequence derived from OspA ST6, preferably with at least 85%, 86%, 87%, 88%, 89%, 90%, 91%, 92%, 93%, 94%, 95%, 96%, 97%, 98%, 99%, or 100% identity to the sequence KQNVSSLDEKNSVSVDLPGGMTVLVSKEKDKDGKYSLEATVDKLELKGTSDKNNGSGT LEGEKTDKSKVKSTIADDLSQTKFEIFKEDGKTLVSKKVTLKDKSSTEEKFNGKGETSEK TIVRANGTRLEYTDIKSDGSGKAKEVLKDFTLEGTLAADGKTTLKVTEGTVVLSKNILKS GEITAALDDSDTTRATKKTGKWDSKTSTLTISVNSQKTKNLVFTKEDTITVQRYDSAGT NLEGKAVEITTLKELKNALK (SEQ ID NO: 189); (g) an amino acid sequence derived from OspA ST7, preferably with at least 85%, 86%, 87%, 88%, 89%, 90%, 91%, 92%, 93%, 94%, 95%, 96%, 97%, 98%, 99%, or 100% identity to the sequence KQNVSSLDEKNSVSVDLPGEMKVLVSKEKDKDGKYSLEATVDKLELKGTSDKNNGSG VLEGVKAAKSKAKLTIADDLSQTKFEIFKEDGKTLVSKKVTLKDKSSTEEKFNDKGKLS EKVVTRANGTRLEYTEIQNDGSGKAKEVLKSLTLEGTLTADGETKLTVEAGTVTLSKNI SESGEITVELKDTETTPADKKSGTWDSKTSTLTISKNSQKTKQLVFTKENTITVQKYNTA GTKLEGSPAEIKDLEALKAALK (SEQ ID NO: 190); (h) any combination of (a)-(g); (i) a sequence derived from OspA ST1 according to (a) and a sequence derived from OspA ST2 according to (b), or j) a sequence derived from OspA ST1 according to (a), a sequence derived from OspA ST2 according to (b), a sequence derived from OspA ST3 according to (c), a sequence derived from OspA ST4 according to (d), a sequence derived from OspA ST5 according to (e), a sequence derived from OspA ST6 according to (f) and a sequence derived from OspA ST7 according to (g).
Embodiment 52. The nucleic acid of any one of embodiments 1-48, wherein the antigenic prokaryotic polypeptide is derived from a bacteria of the genus Cutibacterium, preferably selected from the species acnes, avidum, granulosum, namnetense, and/or humerusii.
Embodiment 53. The nucleic acid of embodiment 52, wherein the antigenic prokaryotic polypeptide is a CAMP2.
Embodiment 54. The nucleic acid of embodiment 53, wherein the amino acid sequence encoding the CAMP2 or a fragment thereof is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to an amino acid sequence comprising SEQ ID NO: 149 or SEQ ID NOs: 162-172.
Embodiment 55. The nucleic acid of embodiment 52, wherein the antigenic prokaryotic polypeptide is a PITP.
Embodiment 56. The nucleic acid of embodiment 54, wherein the amino acid sequence encoding the PITP or a fragment thereof is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to an amino acid sequence comprising SEQ ID NO: 150 or SEQ ID NOs: 173-183.
Embodiment 57. The nucleic acid of any one of embodiments 1-56, wherein the antigenic prokaryotic polypeptide comprises at least one mutated glycosylation site, preferably at least one mutated N-linked glycosylation site.
Embodiment 58. The nucleic acid of any one of embodiments 1-57, wherein the polynucleotide sequence of the nucleic acid is codon optimized.
Embodiment 59. The nucleic acid of any one of embodiments 1-58, wherein the polynucleotide sequence of the ORF is codon optimized.
Embodiment 60. The nucleic acid of any one of embodiments 1-59, wherein the polynucleotide sequence encoding the at least one viral secretion signal peptide is codon optimized.
Embodiment 61. The nucleic acid of any one of embodiments 20-60, wherein the polynucleotide sequence encoding the at least one TMB is codon optimized.
Embodiment 62. The nucleic acid of any one of embodiments 1-61, wherein the nucleic acid is DNA.
Embodiment 63. The nucleic acid of any one of embodiments 1-61, wherein the nucleic acid is messenger RNA (mRNA), wherein in particular the mRNA may be non-replicating mRNA, self-replicating mRNA or trans-replicating mRNA.
Embodiment 64. The nucleic acid of embodiment 63, wherein the mRNA comprises at least one 5′ untranslated region (5′ UTR), at least one 3′ untranslated region (3′ UTR), and/or at least one polyadenylation (poly(A)) sequence.
Embodiment 65. The nucleic acid of embodiment 63 or 64, wherein the mRNA comprises at least one chemical modification.
Embodiment 66. The nucleic acid of any one of embodiments 63-65, wherein at least 20%, at least 30%, at least 40%, at least 50%, at least 60%, at least 70%, at least 80%, at least 85%, at least 90%, at least 95%, or 100% of the uracil nucleotides in the mRNA are chemically modified.
Embodiment 67. The nucleic acid of any one of embodiments 63-66, wherein at least 20%, at least 30%, at least 40%, at least 50%, at least 60%, at least 70%, at least 80%, at least 85%, at least 90%, at least 95%, or 100% of the uracil nucleotides in the ORF are chemically modified.
Embodiment 68. The nucleic acid of any one of embodiments 65-67, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.
Embodiment 69. The nucleic acid of any one of embodiments 65-68, wherein the chemical modification is selected from the group consisting of pseudouridine, N1-methylpseudouridine, 5-methylcytosine, 5-methoxyuridine, and a combination thereof.
Embodiment 70. The nucleic acid of any one of embodiments 65-69, wherein the chemical modification is N1-methylpseudouridine.
Embodiment 71. A composition comprising at least one nucleic acid of any one of embodiments 1-70.
Embodiment 72. The composition of embodiment 71, which further comprises a lipid nanoparticle (LNP).
Embodiment 73. The composition of embodiment 72, wherein the nucleic acid is encapsulated in the LNP.
Embodiment 74. The composition of embodiment 72 or 73, wherein the LNP comprises at least one cationic lipid.
Embodiment 75. The composition of embodiment 74, wherein the cationic lipid is biodegradable.
Embodiment 76. The composition of embodiment 74, wherein the cationic lipid is not biodegradable.
Embodiment 77. The composition of any one of embodiments 74-76, wherein the cationic lipid is cleavable.
Embodiment 78. The composition of any one of embodiments 74-76, wherein the cationic lipid is not cleavable.
Embodiment 79. The composition of any one of embodiments 74-78, wherein the cationic lipid is selected from the group consisting of OF-02, cKK-E10, OF-Deg-Lin, GL-HEPES-E3-E10-DS-3-E18-1, GL-HEPES-E3-E12-DS-4-E10, GL-HEPES-E3-E12-DS-3-E14, SM-102, and ALC-0315.
Embodiment 80. The composition of any one of embodiments 74-79, wherein the LNP further comprises a polyethylene glycol (PEG) conjugated (PEGylated) lipid, a cholesterol-based lipid, and a helper lipid.
Embodiment 81. The composition of any one of embodiments 72-80, wherein the LNP comprises:—a cationic lipid at a molar ratio of 35% to 55%;—a polyethylene glycol (PEG) conjugated (PEGylated) lipid at a molar ratio of 0.25% to 2.75%;—a cholesterol-based lipid at a molar ratio of 20% to 45%; and—a helper lipid at a molar ratio of 5% to 35%, wherein all of the molar ratios are relative to the total lipid content of the LNP.
Embodiment 82. The composition of any one of embodiments 72-81, wherein the LNP comprises:—a cationic lipid at a molar ratio of 40%;—a PEGylated lipid at a molar ratio of 1.5%;—a cholesterol-based lipid at a molar ratio of 28.5%; and—a helper lipid at a molar ratio of 30%.
Embodiment 83. The composition of any one of embodiments 72-82, wherein the LNP comprises:—a cationic lipid at a molar ratio of 45 to 50%;—a PEGylated lipid at a molar ratio of 1.5 to 1.7%;—a cholesterol-based lipid at a molar ratio of 38 to 43%; and—a helper lipid at a molar ratio of 9 to 10%.
Embodiment 84. The composition of any one of embodiments 80-83, wherein the PEGylated lipid is dimyristoyl-PEG2000 (DMG-PEG2000) or 2-[(polyethylene glycol)-2000]-N,N-ditetradecylacetamide (ALC-0159).
Embodiment 85. The composition of any one of embodiments 80-84, wherein the cholesterol-based lipid is cholesterol.
Embodiment 86. The composition of any one of embodiments 80-85, wherein the helper lipid is 1,2-dioleoyl-SN-glycero-3-phosphoethanolamine (DOPE) or 1,2-distearoyl-sn-glycero-3-phosphocholine (DSPC).
Embodiment 87. The composition of any one of embodiments 72-82 and 84-86, wherein the LNP comprises:—a cationic lipid selected from the group consisting of OF-02, cKK-E10, GL-HEPES-E3-E10-DS-3-E18-1, GL-HEPES-E3-E12-DS-4-E10, and GL-HEPES-E3-E12-DS-3-E14, at a molar ratio of 40%;—DMG-PEG2000 at a molar ratio of 1.5%;-cholesterol at a molar ratio of 28.5%; and—DOPE at a molar ratio of 30%.
Embodiment 88. The composition of any one of embodiments 72-81 and 83-86, wherein the LNP comprises:—SM-102 at a molar ratio of 50%;—DMG-PEG2000 at a molar ratio of 1.5%;—cholesterol at a molar ratio of 38.5%; and—DSPC at a molar ratio of 10%.
Embodiment 89. The composition of any one of embodiments 72-81 and 83-86, wherein the LNP comprises:—ALC-0315 at a molar ratio of 46.3%;—ALC-0159 at a molar ratio of 1.6%;-cholesterol at a molar ratio of 42.7%; and—DSPC at a molar ratio of 9.4%.
Embodiment 90. The composition of any one of embodiments 72-81 and 83-86, wherein the LNP comprises:—ALC-0315 at a molar ratio of 47.4%;—ALC-0159 at a molar ratio of 1.7%;—cholesterol at a molar ratio of 40.9%; and—DSPC at a molar ratio of 10%.
Embodiment 91. The composition of any one of embodiments 72-90, wherein the LNP has an average diameter of 30 nm to 200 nm.
Embodiment 92. The composition of any one of embodiments 72-91, wherein the LNP has an average diameter of 80 nm to 150 nm.
Embodiment 93. The composition of any one of embodiments 72-92, comprising between 1 mg/mL to 10 mg/mL of the LNP.
Embodiment 94. The composition of any one of embodiments 72-93, wherein the LNP comprises between 1 and 20 nucleic acid molecules, preferably mRNA molecules.
Embodiment 95. The composition of any one of embodiments 71-94, which is formulated for administration intramuscularly, intranasally, intravenously, subcutaneously, or intradermally.
Embodiment 96. The composition of any one of embodiments 71-95, wherein the composition comprises a phosphate-buffer saline.
Embodiment 97. The composition of any one of embodiments 71-96, wherein the composition is a pharmaceutical composition, for example an immunogenic composition or a vaccine, in particular an mRNA vaccine.
Embodiment 98. The nucleic acid of any one of embodiments 1-70 or the composition of any one of embodiments 71-97, for use in eliciting an immune response in a subject in need thereof.
Embodiment 99. A method of eliciting an immune response in a subject in need thereof, comprising administering to the subject, optionally intramuscularly, intranasally, intravenously, subcutaneously, or intradermally, an effective amount of the nucleic acid of any one of embodiments 1-70 or the composition of any one of embodiments 71-97.
Embodiment 100. Use of the nucleic acid of any one of embodiments 1-70 or the composition of any one of embodiments 71-97 for the manufacture of a medicament for use in eliciting an immune response in a subject in need thereof.
Embodiment 101. The nucleic acid of any one of embodiments 1-70 or the composition of any one of embodiments 71-97 for use in treating or preventing a prokaryotic infection in a subject in need thereof.
Embodiment 102. A method of treating or preventing a prokaryotic infection in a subject in need thereof, comprising administering to the subject, optionally intramuscularly, intranasally, intravenously, subcutaneously, or intradermally, an effective amount of the nucleic acid of any one of embodiments 1-70 or the composition of any one of embodiments 71-97.
Embodiment 103. Use of the nucleic acid of any one of embodiments 1-70 or the composition of any one of embodiments 71-97 for the manufacture of a medicament for use in treating or preventing a prokaryotic infection in a subject in need thereof.
Embodiment 104. A method of secreting an antigenic prokaryotic polypeptide in a host cell, the method comprising administering to the host cell the nucleic acid of any one of embodiments 1-70 or the composition of any one of embodiments 71-97.
Embodiment 105. A method of displaying an antigenic prokaryotic polypeptide on the surface of a host cell, the method comprising administering to the host the nucleic acid of any one of embodiments 1-70 or the composition of any one of embodiments 71-97.
Embodiment 106. A kit comprising a container comprising a single-use or multi-use dosage of the nucleic acid of any one of embodiments 1-70 or the composition of any one of embodiments 71-97, optionally wherein the container is a vial or a pre-filled syringe or injector.

In order that this disclosure may be better understood, the following examples are set forth. These examples are for purposes of illustration only and are not to be construed as limiting the scope of the disclosure in any manner.

### EXAMPLES

### Example 1: Materials & Methods

mRNA production

mRNAs were produced as previously published (Kalnin et al (2021), NPJ Vaccines 6(1):61 and WO2021226436). Briefly, mRNAs incorporating coding sequences containing either the OspA ST1 or ST2 were synthesized by in vitro transcription employing RNA polymerase with a plasmid DNA template encoding the desired gene using unmodified nucleotides. The resulting purified precursor mRNA was reacted further via enzymatic addition of a 5′ cap structure (Cap 1) and a 3′ poly(A) tail of approximately 200 nucleotides in length as determined by gel electrophoresis.

For the preparation of mRNA/lipid nanoparticle (LNP) formulations, an ethanolic solution of a mixture of lipids (cationic/ionizable lipid, phosphatidylethanolamine, cholesterol and polyethylene glycol-lipid) at a fixed lipid and mRNA ratio were combined with an aqueous buffered solution of target mRNA at an acidic pH under controlled conditions to yield a suspension of uniform LNPs. Upon ultrafiltration and diafiltration into a suitable diluent system, the resulting nanoparticle suspensions were diluted to final concentration, filtered, and stored frozen at −80° C. until use.

### Production of Antigens Used as Benchmark Comparators

OspA-ferritin ST1 and ST2 antigens were produced by Sanofi Breakthrough Lab in Cambridge, MA, USA according to the Material and Method previously published (Kamp et al (2020), NPJ Vaccines 5(1):33).

For OspA fusion ST1-ST2, the in-house plasmid pSP401+LPP-chimer OspA ST1-OspA ST2, allowing the expression of the OspA fusion ST1-ST2 C-terminus domains, was introduced into the E. coli expression strain C43-(DE3) (Lucigen). After 2 to 3 hours of growth at 37° C. in a rich medium, the expression of the protein of interest was induced by the addition of an inducer and the culture was stopped 3 hours post-induction. After processing the bacterial pellets, the protein was visualized on SDS-Page gel stained Coomassie blue or by Western Blot using a specific antibody. The scale-up was done on the best expression conditions to produce enough biomass necessary for purification. Bacterial pellets were treated with lysozyme to extract membrane proteins. OspA ST1-OspA ST2 fusion protein was then extracted with Urea 2M+Triton X114 2%. After three incubation-centrifugation steps at 37° C., the lower phase was collected and subjected to a Q Sepharose chromatography in presence of Zwittergent 3.14 detergent (0.5%). The fractions eluted with 400 mM NaCl were subjected to ceramic hydroxyapatite chromatography. OspA ST1-OspA ST2 fusion protein was eluted with Tween 0.05% P04 NaNa2 180 mM pH 6,7 buffer and substituted to PBS+Tween 20 0.05% pH 7.3 as final buffer.

### Transfection of HEK Cells

HEK293T cells in suspension (5 mL at 2×10⁶ cells/mL—shaker 125 mL) were transfected with 5 μg naked mRNA at 1 μg/μL mixed with equal volume of TransIT-mRNA Reagent and mRNA Boost Reagent—TransIT-mRNA Transfection Kit Mirus (Ref MIR 2250) for 2-5 minutes. The mixture was added to the cells drop-wise and incubated at 37° C., 100 rpm, 8% CO₂ for 48 to 72 hours.

Western Blot Analysis of mRNA-Transfected Cells

After transfection, cells and medium were collected and centrifuged (500×g) to collect supernatants. The cell pellets were lysed using Lysozyme (Ready-Lyse Lysozyme Solution-Lucigen ref R1804M)+Benzonase (Sigma-ref E1014)+Protease inhibitor cocktail (Sigma-ref P8340) for 10 min at 20° C. under 800 rpm. The cell pellet lysis was then centrifuged (11,000×g) to collect supernatants and crude extracts.

Extracts from mRNA-transfected HEK293T cells were analyzed by denaturing (95° C.) PAGE using 4-12% Bis-Tris/MES gel (Invitrogen) and Western Blot. Transfer to a nitrocellulose membrane (Bio-Rad) was performed using a semi-dry transfer system (Trans-Blot Turbo Transfer System, Bio-Rad). Blotted proteins were detected with polyclonal (rabbit) antibodies that recognize OspA (anti-OspA polyclonal/Rabbit—Abcam, ref ab10608 —1:2000) and a secondary antibody (anti-rabbit IgG Goat Antibody DyLight 800 —Rockland, ref 611-145-002-1:2000). Blots were imaged with Odyssey Infrared Imager—LICOR.

Characterization of mRNA OspA ST1 or ST2 Antigenicity by Sandwich ELISA with Functional Monoclonal Antibodies (mAbs)
Characterization with ST1-Specific Functional mAb LA-2

mAb 857-2 (R&D Biotech, Internal Order) were coated at 2.5 μg/mL in PBS on microtiter plates (Greiner Bio-one). Plates were incubated overnight at 4° C. and then blocked with PBS-Tween 0.05%-milk 5% for 1 hour at RT.

Transfection supernatants were serially diluted 2-fold in dilution buffer (PBS-Tween 0.05%-milk 1%) and incubated for 1.5 h at RT. After PBS-0.05% Tween washes, detection of proteins attached to the coating were performed by incubation with the mAb LA-2 at 1:1000 (Absolute Antibody, Cat. Ab01070-3.0-BT) for 1.5 h at RT and then with a goat anti-mouse IgG HRP (Jackson Laboratories). After washes, plates were developed using 3,3,5,5-tetramethylbenzidine (Tebu-bio, cat. TMB100-1000) and stopped with 1 N HCl (VWR ProLabo). Optical densities (OD) were read at 450 nm—650 nm.

Characterization with Cross-Specific Functional mAbs 857-2 or 221-7

mAb 221-7 or 857-2 (Wang et al. J. Infect Dis. 214(2): 205-211. 2016) were coated at 5 μg/mL in PBS on microtiter plates (Greiner Bio-one, cat. 655061). Plates were incubated overnight at 4° C. and then blocked with PBS-Tween 0.05%-milk 5% for 1 hour at RT.

Transfection supernatants were serially diluted 2-fold in dilution buffer (PBS-Tween 0.05%-milk 1%) and incubated for 1.5 hours at RT. After PBS-0.05% Tween washes, detection of proteins attached to the coating were performed by incubation at RT of an anti-OspA mouse polyclonal serum (internal) for 1.5 h. Plates were washed and incubated with a goat anti-mouse IgG HRP (Jackson Laboratories, cat. 115-036-062) for 1.5 h at RT. After washes, plates were developed using 3,3,5,5-tetramethylbenzidine (Tebu-bio, cat. TMB100-1000) for 30 min in the dark at RT. Colorimetric reaction was stopped with 1 N HCl (VWR Prolabo, cat 30024290). Optical densities (OD) were read at 450 nm—650 nm.

### Antigens and Mouse Immunizations

OF-1 mice (Charles River) were randomized into immunization groups of eight animals each. Four different doses of mRNA-OspA-LNP were administered intramuscularly (50 μL) at day 0 (DO) (dose 1) and day 21 (D21) (dose 2): 0.2 μg, 1 μg, 5 μg or 10 μg. Sera were taken at baseline (DO), day 19 (D19) before dose 2, and day 35 (D35).

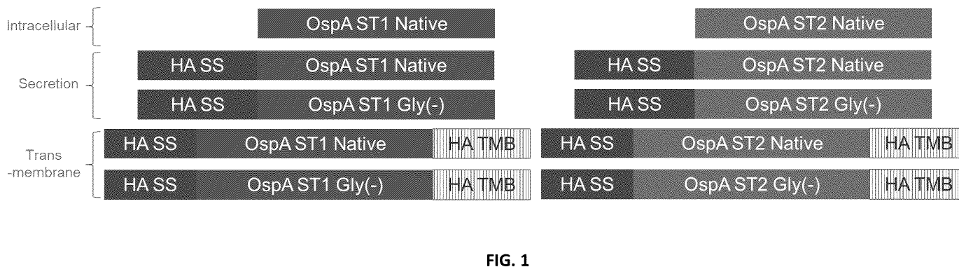

The following mRNA-OspA sequences were tested: mRNA-OspA-ST1-Native, mRNA-HA-SS-OspA-ST1-Native, mRNA-HA-SS-OspA-ST1-Gly(-), mRNA-TMB-OspA-ST1-Native, mRNA-TMB-OspA-ST1-Gly(-). The mRNA sequences with TMB also contain HA SS, as shown in FIG. 1.

Those mRNA formulations were compared to the benchmark Lyme dog vaccine Recombitek® (Merial) at 1 μg/dose (50 μL), recombinant fusion OspA ST1-ST2 (2 μg/dose)+AlOOH adjuvant and OspA-ferritin (ST1 and ST2) at 1.7 μg/dose+AF03 adjuvant.

### OspA-Specific IgG ELISA

The antibody response in mice was determined by ELISA. Briefly, 384-well microplates (Perkin Elmer #6007509) were coated with 1 μg/mL of OspA ST1-His diluted in PBS and incubated overnight at 4° C. The OspA ST1-His was removed and the plates were blocked with 5% skim milk dissolved in PBS-tween. After removing the blocking reagent, the primary serum samples were added after being serially diluted 2-fold in 1% skim milk-PBS-Tween. After a 1.5 h incubation at room temperature, with the primary serum samples, the plates were washed with PBS-Tween and incubated with Goat anti-mouse IgG-HRP (Jackson 115-036-062) for 1.5 h at room temperature. The secondary antibody was aspirated and washed, and the plates were incubated with TMB substrate (TEBU—TMB100-1000) followed by equal volume of stop solution (HCl 1N). Absorbance was measured at 450 nm—650 nm. OspA-specific IgG titers were quantified through an internal anti-OspA mouse serum reference. The titer of this reference was previously calculated as the reciprocal dilution to obtain an GD of 1.

### Statistical Analyses

A 2-way ANOVA with vaccine dose and their interactions as factors was performed (one model per time point). When necessary, the intra-group heterogeneity was taken into account.

OspA Amino Acid and mRNA Sequences

The OspA amino acid sequences and mRNA sequences used in the Examples are recited in the Tables below.

TABLE 4
Amino acid sequences of OspA proteins

| SEQ ID NO / | |
|---|---|
| Description | Amino Acid sequence |
| SEQ ID NO: 31 | MKQNVSSLDEKNSVSVDLPGEMKVLVSKEKNKDGKYDLIATVD |
| OspA ST1 Native | KLELKGTSDKNNGSGVLEGVKADKSKVKLTISDDLGQTTLEVFK |
| | EDGKTLVSKKVTSKDKSSTEEKFNEKGEVSEKIITRADGTRLEYT |
| | GIKSDGSGKAKEVLKGYDLKGELSSEKTTLVVKEGTVTLSKNISK |
| | SGEVSVELNDTDSSAATKKTAAWNSGTSTLTITVNSKKTKDLVF |
| | TKENTITVQQYDSNGTKLEGSAVEITKLDEIKNALK |
| SEQ ID NO: 32 | MKQNVSSLDEKNSASVDLPGEMKVLVSKEKDKDGKYSLKATVD |
| OspA ST2 Native | KIELKGTSDKDNGSGVLEGTKDDKSKAKLTIADDLSKTTFELFKE |
| | DGKTLVSRKVSSKDKTSTDEMFNEKGELSAKTMTRENGTKLEYT |
| | EMKSDGTGKAKEVLKNFTLEGKVANDKVTLEVKEGTVTLSKEIA |
| | KSGEVTVALNDTNTTQATKKTGAWDSKTSTLTISVNSKKTTQLV |
| | FTKQDTITVQKYDSAGTNLEGTAVEIKTLDELKNALK |
| SEQ ID NO: 33 | MKAKLLVLLCTFTATYAKQNVSSLDEKNSVSVDLPGEMKVLV |
| SS-HA-OspA ST1 | SKEKNKDGKYDLIATVDKLELKGTSDKNNGSGVLEGVKADKSK |
| Native | VKLTISDDLGQTTLEVFKEDGKTLVSKKVTSKDKSSTEEKFNEKG |
| SS bold / | EVSEKIITRADGTRLEYTGIKSDGSGKAKEVLKGYDLKGELSSEK |
| underlined | TTLVVKEGTVTLSKNISKSGEVSVELNDTDSSAATKKTAAWNSG |
| | TSTLTITVNSKKTKDLVFTKENTITVQQYDSNGTKLEGSAVEITKL |
| | DEIKNALK |
| SEQ ID NO: 34 | MKAKLLVLLCTFTATYAKQNVSSLDEKNSASVDLPGEMKVLV |
| SS-HA-OspA ST2 | SKEKDKDGKYSLKATVDKIELKGTSDKDNGSGVLEGTKDDKSK |
| Native | AKLTIADDLSKTTFELFKEDGKTLVSRKVSSKDKTSTDEMFNEKG |
| SS bold / | ELSAKTMTRENGTKLEYTEMKSDGTGKAKEVLKNFTLEGKVAN |
| underlined | DKVTLEVKEGTVTLSKEIAKSGEVTVALNDTNTTQATKKTGAW |
| | DSKTSTLTISVNSKKTTQLVFTKQDTITVQKYDSAGTNLEGTAVEI |
| | KTLDELKNALK |
| SEQ ID NO: 35 | MKAKLLVLLCTFTATYAKQNVSSLDEKNSVSVDLPGEMKVLV |
| SS-HA-OspA ST1 | SKEKNKDGKYDLIATVDKLELKGTSDKNNGSGVLEGVKADKSK |
| Native- HA-TMB | VKLTISDDLGQTTLEVFKEDGKTLVSKKVTSKDKSSTEEKFNEKG |
| SS bold / | EVSEKIITRADGTRLEYTGIKSDGSGKAKEVLKGYDLKGELSSEK |
| underlined | TTLVVKEGTVTLSKNISKSGEVSVELNDTDSSAATKKTAAWNSG |
| TMB bold / italics | TSTLTITVNSKKTKDLVFTKENTITVQQYDSNGTKLEGSAVEITKL |
| linker bold / | DEIKNALKSGSILAIYSTVASSLVLLVSLGAISFGSG |
| italics / underlined | |
| SEQ ID NO: 36 | MKAKLLVLLCTFTATYAKQNVSSLDEKNSASVDLPGEMKVLV |
| SS-HA-OspA ST2 | SKEKDKDGKYSLKATVDKIELKGTSDKDNGSGVLEGTKDDKSK |
| Native- HA-TMB | AKLTIADDLSKTTFELFKEDGKTLVSRKVSSKDKTSTDEMFNEKG |
| SS bold / | ELSAKTMTRENGTKLEYTEMKSDGTGKAKEVLKNFTLEGKVAN |
| underlined | DKVTLEVKEGTVTLSKEIAKSGEVTVALNDTNTTQATKKTGAW |
| TMB bold / italics | DSKTSTLTISVNSKKTTQLVFTKQDTITVQKYDSAGTNLEGTAVEI |
| linker bold / | KTLDELKNALKSGSILAIYSTVASSLVLLVSLGAISFGSG |
| italics / underlined | |
| SEQ ID NO: 37 | MKAKLLVLLCTFTATYAKQNVGSLDEKNSVSVDLPGEMKVLV |
| SS-HA-OspA ST1 | SKEKNKDGKYDLIATVDKLELKGTSDKNNGSGVLEGVKADKSK |
| Gly(−) | VKLTISDDLGQTTLEVFKEDGKTLVSKKVTSKDKSSTEEKFNEKG |
| SS bold / | EVSEKIITRADGTRLEYTGIKSDGSGKAKEVLKGYDLKGELSSEK |
| underlined | TTLVVKEGTVTLSKNIDKSGEVSVELDDTDSSAATKKTAAWNSG |
| | TSTLTITVNSKKTKDLVFTKENTITVQQYDSAGTKLEGSAVEITKL |
| | DEIKNALK |
| SEQ ID NO: 38 | MKAKLLVLLCTFTATYAKQNVGSLDEKNSASVDLPGEMKVLV |
| SS-HA-OspA ST2 | SKEKDKDGKYSLKATVDKIELKGTSDKDDGSGVLEGTKDDKSK |
| Gly(−) | AKLTIADDLSKTTFELFKEDGKTLVSRKVSSKDKTSTDEMFNEKG |
| SS bold / | ELSAKTMTREDGTKLEYTEMKSDGTGKAKEVLKHFTLEGKVAN |
| underlined | DKVTLEVKEGTVTLSKEIAKSGEVTVALKDTSTTQATKKTGAWD |
| | SKTSTLTISVNSKKTTQLVFTKQDTITVQKYDSAGTNLEGTAVEIK |
| | TLDELKNALK |
| SEQ ID NO: 39 | MKAKLLVLLCTFTATYAKQNVGSLDEKNSVSVDLPGEMKVLV |
| SS-HA-OspA ST1 | SKEKNKDGKYDLIATVDKLELKGTSDKNNGSGVLEGVKADKSK |
| Gly(−)- HA-TMB | VKLTISDDLGQTTLEVFKEDGKTLVSKKVTSKDKSSTEEKFNEKG |
| SS bold / | EVSEKIITRADGTRLEYTGIKSDGSGKAKEVLKGYDLKGELSSEK |
| underlined | TTLVVKEGTVTLSKNIDKSGEVSVELDDTDSSAATKKTAAWNSG |
| TMB bold / italics | TSTLTITVNSKKTKDLVFTKENTITVQQYDSAGTKLEGSAVEITKL |
| linker bold / | DEIKNALKSGSILAIYSTVASSLVLLVSLGAISFGSG |
| italics / underlined | |
| SEQ ID NO: 40 | MKAKLLVLLCTFTATYAKQNVGSLDEKNSASVDLPGEMKVLV |
| SS-HA-OspA ST2 | SKEKDKDGKYSLKATVDKIELKGTSDKDDGSGVLEGTKDDKSK |
| Gly(−)- HA-TMB | AKLTIADDLSKTTFELFKEDGKTLVSRKVSSKDKTSTDEMFNEKG |
| SS bold / | ELSAKTMTREDGTKLEYTEMKSDGTGKAKEVLKHFTLEGKVAN |
| underlined | DKVTLEVKEGTVTLSKEIAKSGEVTVALKDTSTTQATKKTGAWD |
| TMB bold / italics | SKTSTLTISVNSKKTTQLVFTKQDTITVQKYDSAGTNLEGTAVEIK |
| linker bold / | TLDELKNALKSGSILAIYSTVASSLVLLVSLGAISFGSG |
| italics / underlined | |
| SEQ ID NO: 186 | KQNVSSLDEKNSVSVDLPGGMKVLVSKEKDKDGKYSLMATVEK |
| OspA ST3 Native | LELKGTSDKSNGSGVLEGEKADKSKAKLTISQDLNQTTFEIFKED |
| | GKTLVSRKVNSKDKSSTEEKFNDKGKLSEKVVTRANGTRLEYTE |
| | IKNDGSGKAKEVLKGFALEGTLTDGGETKLTVTEGTVTLSKNISK |
| | SGEITVALNDTETTPADKKTGEWKSDTSTLTISKNSQKPKQLVFT |
| | KENTITVQNYNRAGNALEGSPAEIKDLAELKAALK |
| SEQ ID NO: 187 | KQNVSSLDEKNSVSVDLPGEMKVLVSKEKDKDGKYSLMATVDK |
| OspA ST4 Native | LELKGTSDKSNGSGTLEGEKSDKSKAKLTISEDLSKTTFEIFKEDG |
| | KTLVSKKVNSKDKSSIEEKFNAKGELSEKTILRANGTRLEYTEIKS |
| | DGTGKAKEVLKDFALEGTLAADKTTLKVTEGTVVLSKHIPNSGEI |
| | TVELNDSNSTQATKKTGKWDSNTSTLTISVNSKKTKNIVFTKEDT |
| | ITVQKYDSAGTNLEGNAVEIKTLDELKNALK |
| SEQ ID NO: 188 | KQNVSSLDEKNSVSVDLPGGMKVLVSKEKDKDGKYSLMATVEK |
| OspA ST5 Native | LELKGTSDKNNGSGTLEGEKTDKSKVKLTIAEDLSKTTFEIFKED |
| | GKTLVSKKVTLKDKSSTEEKFNEKGEISEKTIVRANGTRLEYTDI |
| | KSDGSGKAKEVLKDFTLEGTLAADGKTTLKVTEGTVVLSKNILK |
| | SGEITVALDDSDTTQATKKTGKWDSKTSTLTISVNSQKTKNLVFT |
| | KEDTITVQKYDSAGTNLEGKAVEITTLEKLKDALK |
| SEQ ID NO: 189 | KQNVSSLDEKNSVSVDLPGGMTVLVSKEKDKDGKYSLEATVDK |
| OspA ST6 Native | LELKGTSDKNNGSGTLEGEKTDKSKVKSTIADDLSQTKFEIFKED |
| | GKTLVSKKVTLKDKSSTEEKFNGKGETSEKTIVRANGTRLEYTDI |
| | KSDGSGKAKEVLKDFTLEGTLAADGKTTLKVTEGTVVLSKNILK |
| | SGEITAALDDSDTTRATKKTGKWDSKTSTLTISVNSQKTKNLVFT |
| | KEDTITVQRYDSAGTNLEGKAVEITTLKELKNALK |
| SEQ ID NO: 190 | KQNVSSLDEKNSVSVDLPGEMKVLVSKEKDKDGKYSLEATVDK |
| OspA ST7 Native | LELKGTSDKNNGSGVLEGVKAAKSKAKLTIADDLSQTKFEIFKED |
| | GKTLVSKKVTLKDKSSTEEKFNDKGKLSEKVVTRANGTRLEYTE |
| | IQNDGSGKAKEVLKSLTLEGTLTADGETKLTVEAGTVTLSKNISE |
| | SGEITVELKDTETTPADKKSGTWDSKTSTLTISKNSQKTKQLVFT |
| | KENTITVQKYNTAGTKLEGSPAEIKDLEALKAALK |

TABLE 5
mRNA sequences for OspA mRNAs, including 5′ UTR and 3′ UTR

| SEQ ID NO / | |
|---|---|
| Description | Nucleotide sequence |
| SEQ ID NO: | GGACAGAUCGCCUGGAGACGCCAUCCACGCUGUUUUGACCUCCAU |
| 41 | AGAAGACACCGGGACCGAUCCAGCCUCCGCGGCCGGGAACGGUGC |
| OspA ST1 | AUUGGAACGCGGAUUCCCCGUGCCAAGAGUGACUCACCGUCCUUG |
| Native | ACACGAUGAAGCAAAAUGUGAGUAGUCUGGACGAGAAAAAUAGC |
| | GUGAGUGUGGAUCUGCCAGGAGAGAUGAAGGUGCUGGUGUCUAA |
| | AGAGAAAAACAAGGACGGAAAGUACGACCUGAUCGCCACUGUGG |
| | ACAAGCUGGAGCUGAAAGGCACCUCUGAUAAGAACAAUGGCUCU |
| | GGCGUGCUGGAAGGGGUGAAGGCAGACAAGAGCAAAGUGAAGCU |
| | GACUAUCUCUGACGACCUGGGGCAAACCACACUGGAGGUCUUCA |
| | AGGAAGACGGCAAAACACUGGUCUCUAAGAAAGUCACCAGCAAG |
| | GAUAAGUCCUCCACAGAGGAGAAAUUCAACGAGAAAGGCGAAGU |
| | GUCUGAGAAGAUCAUCACCAGGGCCGACGGAACCAGGCUGGAGU |
| | ACACCGGCAUCAAAAGCGACGGGUCAGGGAAGGCUAAGGAAGUG |
| | CUGAAAGGAUACGAUCUGAAAGGAGAGCUGUCCUCCGAGAAGAC |
| | AACUCUGGUGGUGAAAGAGGGAACCGUUACACUGUCUAAGAACA |
| | UCUCCAAGUCUGGGGAAGUCAGCGUGGAGCUGAACGACACCGAC |
| | AGCUCUGCUGCUACAAAGAAGACUGCUGCUUGGAACAGCGGCAC |
| | CUCUACCCUGACAAUCACCGUGAAUUCUAAGAAGACCAAGGACCU |
| | GGUGUUCACUAAGGAGAAUACCAUUACUGUCCAGCAGUAUGAUU |
| | CCAAUGGGACUAAACUGGAAGGAUCCGCCGUGGAGAUUACUAAG |
| | CUGGACGAGAUCAAGAAUGCCCUGAAGUGACGGGUGGCAUCCCU |
| | GUGACCCCUCCCCAGUGCCUCUCCUGGCCCUGGAAGUUGCCACUC |
| | CAGUGCCCACCAGCCUUGUCCUAAUAAAAUUAAGUUGCAUCAAG |
| | CU |
| SEQ ID NO: | GGACAGAUCGCCUGGAGACGCCAUCCACGCUGUUUUGACCUCCAU |
| 42 | AGAAGACACCGGGACCGAUCCAGCCUCCGCGGCCGGGAACGGUGC |
| OspA ST1 | AUUGGAACGCGGAUUCCCCGUGCCAAGAGUGACUCACCGUCCUUG |
| Native | ACACGAUGAAGCAAAACGUGUCCAGCCUGGACGAGAAGAAUUCC |
| | GUUAGCGUGGAUCUGCCAGGGGAGAUGAAAGUGCUGGUGUCCAA |
| | GGAAAAGAACAAAGAUGGCAAGUAUGACCUGAUUGCAACUGUGG |
| | ACAAACUGGAGCUGAAGGGCACUUCUGAUAAAAAUAACGGCAGC |
| | GGCGUUCUGGAGGGCGUUAAGGCCGACAAGUCCAAGGUGAAGCU |
| | GACAAUCUCCGACGACCUGGGGCAGACCACACUGGAGGUGUUUA |
| | AAGAGGAUGGCAAGACCCUGGUCUCUAAAAAAGUGACUUCCAAA |
| | GACAAGUCCAGCACAGAAGAGAAAUUCAAUGAAAAGGGAGAGGU |
| | GUCCGAGAAGAUCAUCACUCGGGCCGACGGGACUAGGCUGGAGU |
| | ACACCGGGAUCAAGAGCGACGGCUCCGGAAAGGCAAAGGAGGUG |
| | CUGAAAGGCUAUGACCUGAAAGGCGAGCUGAGCAGCGAGAAAAC |
| | UACCCUGGUGGUGAAGGAAGGCACAGUGACUCUGUCAAAAAAUA |
| | UCUCUAAGUCCGGCGAGGUGAGCGUGGAACUGAAUGACACUGAC |
| | AGCUCCGCUGCAACUAAGAAAACAGCCGCUUGGAACUCUGGCACA |
| | UCUACUCUCACUAUUACCGUGAACUCCAAGAAAACCAAGGACCUG |
| | GUGUUUACCAAGGAGAAUACAAUCACAGUGCAACAGUACGACAG |
| | CAACGGCACUAAGCUGGAGGGCUCCGCCGUUGAGAUUACUAAGC |
| | UCGACGAGAUCAAAAACGCCCUGAAAUGACGGGUGGCAUCCCUG |
| | UGACCCCUCCCCAGUGCCUCUCCUGGCCCUGGAAGUUGCCACUCC |
| | AGUGCCCACCAGCCUUGUCCUAAUAAAAUUAAGUUGCAUCAAGC |
| | U |
| SEQ ID NO: | GGACAGAUCGCCUGGAGACGCCAUCCACGCUGUUUUGACCUCCAU |
| 43 | AGAAGACACCGGGACCGAUCCAGCCUCCGCGGCCGGGAACGGUGC |
| OspA ST2 | AUUGGAACGCGGAUUCCCCGUGCCAAGAGUGACUCACCGUCCUUG |
| Native | ACACGAUGAAACAGAACGUGUCUUCCCUGGACGAGAAAAAUUCU |
| | GCAAGCGUGGACCUGCCUGGCGAAAUGAAGGUGCUGGUGAGCAA |
| | GGAAAAAGACAAGGACGGCAAGUAUUCUCUGAAGGCAACAGUGG |
| | AUAAGAUUGAACUGAAGGGAACAUCUGACAAGGAUAAUGGCAGU |
| | GGGGUGCUGGAAGGGACUAAGGACGACAAGUCUAAGGCCAAGCU |
| | GACCAUCGCAGAUGACCUGUCCAAGACAACCUUUGAGCUUUUUA |
| | AAGAGGACGGCAAGACCCUGGUGAGCCGGAAAGUGUCUUCUAAG |
| | GACAAGACAUCUACUGACGAGAUGUUCAAUGAAAAAGGGGAGCU |
| | GAGCGCCAAGACAAUGACCAGAGAGAACGGAACAAAGCUGGAGU |
| | ACACUGAGAUGAAGUCCGACGGGACCGGAAAAGCUAAGGAAGUC |
| | CUGAAGAAUUUCACUCUGGAGGGCAAGGUUGCCAACGAUAAGGU |
| | GACACUGGAGGUGAAAGAGGGAACAGUGACCCUGUCCAAGGAAA |
| | UUGCCAAGAGUGGCGAAGUGACCGUGGCUCUGAACGAUACCAAU |
| | ACAACUCAGGCCACAAAGAAGACCGGAGCAUGGGACUCCAAAAC |
| | AUCCACCCUGACCAUUAGCGUUAAUUCAAAGAAAACCACCCAGCU |
| | GGUGUUUACUAAGCAGGACACCAUCACCGUGCAGAAGUAUGAUU |
| | CCGCAGGCACUAAUCUGGAAGGGACAGCUGUGGAGAUCAAGACC |
| | CUGGAUGAGCUGAAAAAUGCCCUGAAAUGACGGGUGGCAUCCCU |
| | GUGACCCCUCCCCAGUGCCUCUCCUGGCCCUGGAAGUUGCCACUC |
| | CAGUGCCCACCAGCCUUGUCCUAAUAAAAUUAAGUUGCAUCAAG |
| | CU |
| SEQ ID NO: | GGACAGAUCGCCUGGAGACGCCAUCCACGCUGUUUUGACCUCCAU |
| 44 | AGAAGACACCGGGACCGAUCCAGCCUCCGCGGCCGGGAACGGUGC |
| OspA ST2 | AUUGGAACGCGGAUUCCCCGUGCCAAGAGUGACUCACCGUCCUUG |
| Native | ACACGAUGAAGCAGAACGUGAGUAGCCUGGAUGAAAAGAAUAGC |
| | GCCUCUGUGGACCUGCCCGGGGAGAUGAAGGUGCUGGUCUCUAA |
| | AGAGAAAGAUAAGGAUGGAAAAUACAGCCUGAAGGCAACAGUGG |
| | ACAAAAUCGAGCUCAAAGGAACAUCCGAUAAGGAUAACGGGAGC |
| | GGAGUCCUGGAGGGCACCAAAGACGACAAGAGCAAGGCCAAGCU |
| | GACAAUCGCCGAUGAUCUGAGUAAGACAACUUUCGAGCUCUUUA |
| | AGGAGGACGGCAAGACCCUCGUGAGCAGAAAGGUGAGCAGCAAA |
| | GACAAGACCUCUACCGACGAGAUGUUCAAUGAGAAGGGGGAACU |
| | CUCUGCUAAGACAAUGACACGGGAGAAUGGAACUAAACUGGAGU |
| | ACACAGAAAUGAAGUCUGACGGCACUGGGAAGGCAAAAGAGGUG |
| | CUGAAGAAUUUCACACUGGAAGGGAAGGUGGCUAACGAUAAAGU |
| | GACACUGGAGGUUAAGGAGGGAACCGUGACUCUGUCUAAGGAGA |
| | UUGCAAAAAGCGGAGAGGUGACAGUCGCCCUGAACGACACUAAC |
| | ACCACCCAGGCCACUAAGAAGACAGGAGCCUGGGACAGUAAAACC |
| | UCCACACUGACCAUUUCCGUGAACAGCAAGAAGACAACCCAGCUG |
| | GUCUUCACUAAACAGGAUACUAUCACAGUCCAGAAGUACGACUC |
| | CGCCGGAACCAAUCUGGAGGGAACUGCCGUGGAGAUCAAAACCC |
| | UGGACGAGCUCAAAAACGCCCUGAAGUGACGGGUGGCAUCCCUG |
| | UGACCCCUCCCCAGUGCCUCUCCUGGCCCUGGAAGUUGCCACUCC |
| | AGUGCCCACCAGCCUUGUCCUAAUAAAAUUAAGUUGCAUCAAGC |
| | U |
| SEQ ID NO: | GGACAGAUCGCCUGGAGACGCCAUCCACGCUGUUUUGACCUCCAU |
| 45 | AGAAGACACCGGGACCGAUCCAGCCUCCGCGGCCGGGAACGGUGC |
| SS-HA-OspA | AUUGGAACGCGGAUUCCCCGUGCCAAGAGUGACUCACCGUCCUUG |
| ST1 Native | ACACGAUGAAGGCCAAGCUGCUGGUCCUGCUCUGUACCUUUAC |
| SS bold / | AGCCACUUACGCCAAGCAGAACGUGUCUUCCCUGGACGAAAAGA |
| underlined | ACAGCGUCUCUGUGGACCUGCCUGGAGAGAUGAAGGUCCUGGUG |
| | UCUAAAGAGAAGAAUAAGGACGGCAAGUACGACCUCAUUGCAAC |
| | UGUGGACAAGCUGGAGCUGAAGGGAACAUCCGACAAGAAUAAUG |
| | GCAGCGGCGUUCUGGAGGGCGUGAAGGCCGACAAAAGCAAAGUG |
| | AAGCUGACUAUCUCUGAUGACCUGGGGCAAACUACACUGGAAGU |
| | GUUCAAGGAGGAUGGGAAGACACUGGUGAGCAAAAAAGUGACCA |
| | GCAAGGACAAAUCCUCUACAGAGGAAAAGUUUAAUGAGAAAGGC |
| | GAGGUGUCUGAAAAGAUCAUCACUCGCGCCGACGGGACUCGGCU |
| | GGAGUAUACCGGGAUCAAGAGCGACGGGUCCGGGAAAGCCAAGG |
| | AGGUGCUGAAAGGAUAUGAUCUGAAGGGAGAGCUCUCUUCCGAG |
| | AAGACAACACUGGUGGUCAAGGAGGGAACAGUUACCCUGUCCAA |
| | AAACAUCAGCAAGUCCGGGGAGGUCUCCGUCGAACUGAAUGACA |
| | CCGACUCCUCUGCCGCCACCAAGAAGACCGCCGCAUGGAACUCUG |
| | GAACAAGCACACUGACAAUCACAGUGAAUUCCAAGAAGACUAAG |
| | GACCUCGUGUUCACAAAGGAGAACACAAUCACAGUGCAGCAGUA |
| | CGACAGCAACGGAACCAAACUGGAGGGCUCCGCAGUGGAAAUCA |
| | CAAAACUGGACGAGAUUAAGAACGCUCUGAAGUGACGGGUGGCA |
| | UCCCUGUGACCCCUCCCCAGUGCCUCUCCUGGCCCUGGAAGUUGC |
| | CACUCCAGUGCCCACCAGCCUUGUCCUAAUAAAAUUAAGUUGCAU |
| | CAAGCU |
| SEQ ID NO: | GGACAGAUCGCCUGGAGACGCCAUCCACGCUGUUUUGACCUCCAU |
| 46 | AGAAGACACCGGGACCGAUCCAGCCUCCGCGGCCGGGAACGGUGC |
| SS-HA-OspA | AUUGGAACGCGGAUUCCCCGUGCCAAGAGUGACUCACCGUCCUUG |
| ST1 Native | ACACGAUGAAGGCCAAACUGCUCGUGCUCUUAUGCACAUUCAC |
| SS bold / | AGCAACCUACGCCAAACAGAAUGUCAGUUCCCUCGAUGAGAAGA |
| underlined | AUUCCGUGAGCGUGGACCUGCCAGGCGAGAUGAAGGUGCUGGUG |
| | UCCAAGGAGAAGAACAAAGACGGGAAAUACGACCUGAUCGCCAC |
| | CGUGGAUAAGCUGGAGCUGAAAGGCACAAGCGACAAGAAUAACG |
| | GAUCCGGCGUGCUGGAGGGAGUCAAGGCCGAUAAGAGCAAGGUG |
| | AAGCUGACCAUCAGCGACGAUCUGGGGCAGACUACCCUGGAAGU |
| | CUUUAAGGAGGACGGGAAAACACUGGUGUCAAAAAAGGUGACAU |
| | CCAAAGAUAAAUCCAGCACCGAAGAGAAGUUUAACGAGAAGGGG |
| | GAGGUCUCCGAGAAGAUUAUUACACGCGCAGACGGCACAAGGCU |
| | GGAGUAUACCGGAAUCAAGUCCGAUGGAUCCGGCAAAGCUAAGG |
| | AGGUGCUGAAGGGAUACGAUCUGAAAGGAGAGCUGUCUUCUGAG |
| | AAGACUACUCUGGUGGUGAAAGAAGGCACUGUGACUCUGUCUAA |
| | GAACAUCUCCAAGUCCGGCGAAGUUUCCGUGGAACUUAACGACA |
| | CAGAUUCAUCCGCCGCCACAAAGAAGACCGCCGCCUGGAAUUCCG |
| | GCACUAGUACACUGACCAUUACCGUCAACAGCAAGAAGACAAAG |
| | GAUCUCGUGUUCACCAAAGAAAAUACAAUUACCGUGCAGCAGUA |
| | CGACUCCAAUGGCACAAAGCUGGAGGGCUCUGCAGUGGAGAUCA |
| | CUAAGCUGGACGAGAUCAAAAAUGCCCUGAAAUGACGGGUGGCA |
| | UCCCUGUGACCCCUCCCCAGUGCCUCUCCUGGCCCUGGAAGUUGC |
| | CACUCCAGUGCCCACCAGCCUUGUCCUAAUAAAAUUAAGUUGCAU |
| | CAAGCU |
| SEQ ID NO: | GGACAGAUCGCCUGGAGACGCCAUCCACGCUGUUUUGACCUCCAU |
| 47 | AGAAGACACCGGGACCGAUCCAGCCUCCGCGGCCGGGAACGGUGC |
| SS-HA-OspA | AUUGGAACGCGGAUUCCCCGUGCCAAGAGUGACUCACCGUCCUUG |
| ST2 Native | ACACGAUGAAGGCAAAGCUGCUGGUGCUGCUGUGUACCUUCAC |
| SS bold / | UGCCACCUACGCCAAGCAGAACGUCUCCAGCCUGGACGAGAAGA |
| underlined | AUUCUGCCUCCGUCGACCUGCCCGGCGAAAUGAAGGUCCUGGUGA |
| | GCAAGGAAAAAGACAAAGACGGCAAGUAUUCCCUGAAGGCCACA |
| | GUGGACAAAAUCGAACUGAAAGGGACCUCUGAUAAGGAUAACGG |
| | AAGCGGAGUGCUGGAAGGAACUAAGGAUGAUAAGAGCAAAGCCA |
| | AGCUGACUAUUGCUGAUGAUCUCUCUAAGACCACCUUUGAACUC |
| | UUCAAGGAGGACGGCAAGACACUGGUGUCAAGAAAGGUGUCCAG |
| | CAAGGACAAGACUUCUACUGAUGAAAUGUUUAAUGAGAAGGGAG |
| | AACUGUCCGCUAAGACCAUGACACGGGAGAACGGCACUAAGCUG |
| | GAGUAUACCGAGAUGAAAAGCGAUGGCACUGGAAAGGCCAAGGA |
| | GGUGCUGAAAAAUUUUACCCUGGAAGGAAAAGUGGCCAAUGAUA |
| | AGGUGACACUGGAGGUGAAGGAGGGCACAGUCACUCUGUCCAAA |
| | GAGAUCGCCAAGUCCGGCGAAGUGACCGUGGCUCUCAAUGACACC |
| | AACACAACUCAAGCCACAAAAAAGACCGGAGCAUGGGAUUCUAA |
| | AACAUCCACUCUGACCAUUAGCGUUAACUCCAAGAAGACAACCCA |
| | GCUGGUCUUUACAAAACAGGACACUAUCACUGUGCAGAAGUAUG |
| | AUUCUGCCGGAACCAAUCUCGAGGGCACAGCCGUGGAAAUCAAG |
| | ACCCUGGACGAGCUGAAGAACGCCCUGAAGUGACGGGUGGCAUC |
| | CCUGUGACCCCUCCCCAGUGCCUCUCCUGGCCCUGGAAGUUGCCA |
| | CUCCAGUGCCCACCAGCCUUGUCCUAAUAAAAUUAAGUUGCAUCA |
| | AGCU |
| SEQ ID NO: | GGACAGAUCGCCUGGAGACGCCAUCCACGCUGUUUUGACCUCCAU |
| 48 | AGAAGACACCGGGACCGAUCCAGCCUCCGCGGCCGGGAACGGUGC |
| SS-HA-OspA | AUUGGAACGCGGAUUCCCCGUGCCAAGAGUGACUCACCGUCCUUG |
| ST1 Native- | ACACGAUGAAGGCCAAGCUGCUGGUGCUGCUGUGUACAUUCAC |
| HA-TMB | AGCAACCUAUGCCAAGCAGAACGUGUCUAGCCUGGAUGAGAAGA |
| SS bold / | ACAGCGUGUCCGUGGACCUCCCAGGAGAGAUGAAGGUUCUGGUG |
| underlined | UCUAAGGAGAAGAACAAGGAUGGGAAGUACGACCUGAUUGCAAC |
| TMB bold / | CGUCGACAAGCUGGAGCUGAAGGGAACCAGCGACAAAAACAAUG |
| italics | GAAGCGGAGUGCUGGAGGGAGUCAAAGCAGACAAAAGCAAGGUU |
| linker bold / | AAACUGACCAUCAGCGACGACCUGGGACAGACCACACUGGAGGUC |
| italics / | UUUAAGGAAGAUGGAAAAACACUCGUGAGCAAAAAGGUGACCUC |
| underlined | CAAGGAUAAGAGCUCUACAGAGGAGAAGUUCAAUGAAAAGGGAG |
| | AGGUCUCCGAGAAGAUUAUCACAAGAGCCGACGGCACAAGACUG |
| | GAGUAUACCGGCAUUAAGUCCGACGGCUCUGGGAAGGCCAAAGA |
| | GGUCCUGAAGGGAUAUGACCUGAAGGGCGAACUGUCCUCCGAAA |
| | AGACAACCCUGGUGGUGAAAGAGGGGACCGUGACCCUGAGCAAG |
| | AACAUCUCAAAAUCUGGGGAGGUGUCCGUGGAGCUGAACGACAC |
| | UGACUCUUCUGCCGCUACUAAGAAGACCGCAGCAUGGAAUUCCG |
| | GCACCAGCACUCUGACCAUCACCGUGAACAGCAAGAAGACUAAGG |
| | ACCUGGUGUUUACUAAGGAAAAUACUAUCACCGUCCAGCAGUAC |
| | GACAGCAACGGGACCAAGCUGGAGGGCUCCGCAGUGGAAAUCAC |
| | UAAGCUGGACGAGAUCAAAAACGCUCUGAAGUCCGGGAGCAUCC |
| | UGGCAAUCUAUAGCACAGUCGCCAGCUCCCUGGUUCUCCUGGUGA |
| | GCCUGGGGGCAAUUUCCUUCGGAUCUGGCUGACGGGUGGCAUCCC |
| | UGUGACCCCUCCCCAGUGCCUCUCCUGGCCCUGGAAGUUGCCACU |
| | CCAGUGCCCACCAGCCUUGUCCUAAUAAAAUUAAGUUGCAUCAA |
| | GCU |
| SEQ ID NO: | GGACAGAUCGCCUGGAGACGCCAUCCACGCUGUUUUGACCUCCAU |
| 49 | AGAAGACACCGGGACCGAUCCAGCCUCCGCGGCCGGGAACGGUGC |
| SS-HA-OspA | AUUGGAACGCGGAUUCCCCGUGCCAAGAGUGACUCACCGUCCUUG |
| ST1 Native- | ACACGAUGAAAGCAAAGCUGCUGGUGCUGCUGUGCACAUUCAC |
| HA-TMB | CGCAACAUACGCCAAACAGAAUGUGUCUUCCCUGGACGAGAAAA |
| SS bold / | AUUCCGUCUCUGUGGACCUGCCCGGCGAAAUGAAGGUCCUGGUG |
| underlined | UCUAAGGAAAAGAACAAAGACGGGAAGUACGACCUGAUCGCAAC |
| TMB bold / | CGUGGAUAAGCUGGAACUGAAGGGGACUAGCGACAAGAAUAAUG |
| italics | GAUCCGGCGUGCUGGAAGGGGUGAAAGCUGACAAGUCCAAGGUC |
| linker bold / | AAGCUGACCAUUUCUGACGACCUGGGGCAGACAACCCUGGAGGU |
| italics / | CUUCAAAGAGGACGGAAAGACACUGGUGAGCAAGAAGGUGACAU |
| underlined | CCAAGGAUAAGUCUUCCACCGAAGAAAAAUUCAAUGAGAAAGGG |
| | GAAGUGUCCGAGAAAAUCAUUACUCGGGCCGAUGGCACAAGACU |
| | GGAGUACACAGGCAUCAAAUCCGACGGAAGCGGGAAGGCCAAGG |
| | AGGUUCUGAAGGGCUACGACCUGAAAGGGGAGCUGUCCAGCGAA |
| | AAGACAACACUGGUGGUGAAAGAAGGAACUGUCACACUGUCUAA |
| | GAAUAUCAGCAAGUCAGGCGAGGUGAGCGUGGAGCUGAACGACA |
| | CCGACUCCUCCGCUGCAACAAAAAAGACCGCCGCUUGGAACUCCG |
| | GAACUAGCACCCUGACUAUUACUGUGAAUUCCAAAAAGACCAAA |
| | GACCUGGUGUUUACUAAGGAGAACACCAUUACCGUCCAGCAGUA |
| | UGAUAGCAAUGGCACCAAACUGGAAGGAAGCGCAGUUGAGAUCA |
| | CCAAACUGGACGAGAUCAAGAACGCACUGAAAUCCGGAUCCAUCC |
| | UGGCCAUCUAUAGCACCGUCGCCAGCUCUCUGGUGCUGCUGGUGU |
| | CCCUCGGGGCUAUCUCAUUCGGGUCCGGCUGACGGGUGGCAUCCC |
| | UGUGACCCCUCCCCAGUGCCUCUCCUGGCCCUGGAAGUUGCCACU |
| | CCAGUGCCCACCAGCCUUGUCCUAAUAAAAUUAAGUUGCAUCAA |
| | GCU |
| SEQ ID NO: | GGACAGAUCGCCUGGAGACGCCAUCCACGCUGUUUUGACCUCCAU |
| 50 | AGAAGACACCGGGACCGAUCCAGCCUCCGCGGCCGGGAACGGUGC |
| SS-HA-OspA | AUUGGAACGCGGAUUCCCCGUGCCAAGAGUGACUCACCGUCCUUG |
| ST2 Native- | ACACGAUGAAGGCUAAACUGCUGGUCCUGCUGUGUACCUUCAC |
| HA-TMB | CGCUACAUACGCCAAGCAGAAUGUGAGCUCCCUGGACGAGAAAA |
| SS bold / | AUUCCGCUUCCGUGGACCUGCCCGGGGAGAUGAAGGUGCUGGUG |
| underlined | UCCAAGGAGAAGGAUAAGGACGGAAAGUACAGUCUGAAGGCCAC |
| TMB bold / | UGUGGACAAGAUCGAGCUGAAGGGGACAUCUGACAAAGAUAAUG |
| italics | GAUCAGGAGUGCUGGAAGGGACAAAGGAUGACAAGAGCAAAGCC |
| linker bold / | AAGCUGACCAUUGCCGACGACCUGAGCAAAACUACUUUUGAACU |
| italics / | CUUCAAAGAAGACGGGAAGACCCUGGUCAGCCGCAAGGUGUCCU |
| underlined | CUAAGGACAAGACUAGCACUGAUGAGAUGUUCAACGAGAAGGGG |
| | GAGCUGUCUGCAAAGACAAUGACAAGGGAGAACGGGACAAAGCU |
| | GGAGUACACUGAGAUGAAGUCCGACGGCACAGGGAAAGCAAAAG |
| | AGGUUCUGAAGAACUUCACCCUGGAAGGCAAAGUGGCAAACGAC |
| | AAGGUGACACUGGAGGUGAAGGAGGGAACAGUGACUCUGUCCAA |
| | GGAGAUCGCUAAAAGCGGCGAGGUGACUGUGGCUCUGAAUGACA |
| | CUAAUACAACCCAGGCUACAAAGAAGACCGGAGCUUGGGAUUCU |
| | AAGACUAGCACUCUGACUAUCUCCGUGAAUUCUAAAAAGACAAC |
| | CCAGCUGGUGUUCACCAAGCAGGAUACCAUUACAGUGCAGAAGU |
| | ACGAUUCCGCCGGCACUAAUCUGGAGGGGACUGCUGUGGAGAUC |
| | AAGACCCUGGACGAACUGAAGAACGCACUCAAGUCCGGAUCAAUC |
| | CUGGCUAUCUAUAGCACUGUGGCUUCCUCUCUGGUGCUGCUGGUU |
| | UCCCUGGGGGCCAUUUCCUUCGGCAGUGGCUGACGGGUGGCAUCC |
| | CUGUGACCCCUCCCCAGUGCCUCUCCUGGCCCUGGAAGUUGCCAC |
| | UCCAGUGCCCACCAGCCUUGUCCUAAUAAAAUUAAGUUGCAUCA |
| | AGCU |
| SEQ ID NO: | GGACAGAUCGCCUGGAGACGCCAUCCACGCUGUUUUGACCUCCAU |
| 51 | AGAAGACACCGGGACCGAUCCAGCCUCCGCGGCCGGGAACGGUGC |
| SS-HA-OspA | AUUGGAACGCGGAUUCCCCGUGCCAAGAGUGACUCACCGUCCUUG |
| ST2 Native- | ACACGAUGAAGGCAAAACUGCUGGUGCUGCUGUGUACAUUCAC |
| HA-TMB | AGCUACUUAUGCAAAGCAGAACGUGUCCUCUCUGGACGAGAAGA |
| SS bold / | ACUCCGCCUCCGUCGAUCUCCCAGGAGAGAUGAAAGUUCUUGUG |
| underlined | AGCAAGGAGAAGGACAAGGAUGGCAAAUAUAGCCUGAAGGCCAC |
| TMB bold / | CGUGGACAAGAUCGAGCUGAAAGGCACCUCCGACAAGGACAAUG |
| italics | GCUCCGGAGUGCUGGAAGGAACCAAGGACGAUAAGUCUAAGGCU |
| linker bold / | AAGCUGACCAUCGCUGACGACCUGAGCAAGACUACAUUUGAACU |
| italics / | CUUCAAGGAGGACGGCAAGACACUGGUGUCCAGAAAGGUGAGCU |
| underlined | CUAAGGACAAGACUAGCACUGACGAAAUGUUCAAUGAGAAGGGG |
| | GAGCUGAGCGCUAAGACAAUGACUCGCGAGAAUGGCACAAAGCU |
| | CGAAUAUACAGAGAUGAAGUCCGACGGGACUGGCAAGGCCAAGG |
| | AAGUGCUGAAGAACUUUACUCUGGAAGGGAAAGUCGCUAAUGAC |
| | AAGGUGACACUCGAGGUGAAGGAGGGCACAGUGACCCUGUCUAA |
| | AGAGAUCGCUAAGUCCGGGGAGGUGACCGUCGCCCUGAACGAUA |
| | CAAACACAACCCAGGCCACAAAGAAAACCGGCGCAUGGGAUAGCA |
| | AGACAUCCACUCUGACAAUUUCUGUGAACAGCAAGAAGACAACC |
| | CAGCUGGUCUUUACCAAGCAGGAUACAAUCACAGUCCAGAAAUA |
| | UGACUCUGCCGGAACUAAUCUGGAGGGAACUGCAGUGGAGAUCA |
| | AGACACUCGAUGAACUGAAGAAUGCACUGAAGUCCGGGAGCAUC |
| | CUGGCUAUCUAUAGCACCGUCGCCUCCAGCCUCGUUCUGCUGGUG |
| | AGCCUGGGCGCCAUUUCCUUCGGCAGCGGCUGACGGGUGGCAUCC |
| | CUGUGACCCCUCCCCAGUGCCUCUCCUGGCCCUGGAAGUUGCCAC |
| | UCCAGUGCCCACCAGCCUUGUCCUAAUAAAAUUAAGUUGCAUCA |
| | AGCU |
| SEQ ID NO: | GGACAGAUCGCCUGGAGACGCCAUCCACGCUGUUUUGACCUCCAU |
| 52 | AGAAGACACCGGGACCGAUCCAGCCUCCGCGGCCGGGAACGGUGC |
| SS-HA-OspA | AUUGGAACGCGGAUUCCCCGUGCCAAGAGUGACUCACCGUCCUUG |
| ST1 Gly(-) | ACACGAUGAAGGCUAAGCUGCUGGUUCUGCUGUGUACUUUUAC |
| SS bold / | CGCCACAUACGCUAAGCAGAACGUGGGAAGCCUGGACGAGAAAA |
| underlined | ACUCCGUGUCUGUGGAUCUGCCUGGGGAGAUGAAGGUGCUCGUU |
| | AGCAAGGAGAAGAAUAAGGACGGGAAGUAUGACCUCAUUGCCAC |
| | AGUGGACAAGCUGGAGCUGAAAGGCACCAGCGAUAAGAAUAACG |
| | GCUCCGGCGUGCUGGAGGGGGUCAAAGCCGACAAGUCCAAGGUG |
| | AAACUGACAAUUUCUGACGACCUGGGGCAGACCACUCUGGAGGU |
| | CUUUAAAGAGGACGGCAAGACACUCGUGUCCAAGAAAGUGACCU |
| | CUAAAGAUAAGUCCUCCACUGAGGAGAAGUUUAAUGAAAAGGGG |
| | GAAGUUAGCGAGAAGAUUAUUACAAGAGCUGAUGGGACUCGCCU |
| | GGAGUAUACCGGCAUCAAGUCCGACGGCUCUGGCAAAGCAAAGG |
| | AGGUGCUGAAAGGCUAUGAUCUGAAGGGAGAGCUGUCUUCCGAG |
| | AAGACCACACUGGUGGUGAAAGAGGGCACCGUGACACUGAGUAA |
| | GAACAUUGACAAGUCCGGCGAAGUGAGCGUCGAACUGGACGACA |
| | CUGAUAGCUCCGCCGCCACCAAGAAAACAGCCGCAUGGAAUAGCG |
| | GGACAAGCACUCUGACCAUCACAGUUAACUCUAAAAAGACAAAG |
| | GACCUGGUUUUUACUAAAGAGAAUACCAUUACCGUUCAGCAGUA |
| | UGACAGCGCCGGAACCAAGCUGGAGGGAAGCGCAGUGGAAAUCA |
| | CUAAGCUGGACGAGAUUAAGAACGCACUGAAGUGACGGGUGGCA |
| | UCCCUGUGACCCCUCCCCAGUGCCUCUCCUGGCCCUGGAAGUUGC |
| | CACUCCAGUGCCCACCAGCCUUGUCCUAAUAAAAUUAAGUUGCAU |
| | CAAGCU |
| SEQ ID NO: | GGACAGAUCGCCUGGAGACGCCAUCCACGCUGUUUUGACCUCCAU |
| 53 | AGAAGACACCGGGACCGAUCCAGCCUCCGCGGCCGGGAACGGUGC |
| SS-HA-OspA | AUUGGAACGCGGAUUCCCCGUGCCAAGAGUGACUCACCGUCCUUG |
| ST1 Gly(-) | ACACGAUGAAGGCCAAACUCCUGGUGCUCCUGUGUACCUUCAC |
| SS bold / | CGCUACCUACGCCAAGCAGAACGUGGGAAGCCUGGACGAAAAGA |
| underlined | AUUCCGUGAGCGUCGACCUGCCCGGCGAAAUGAAGGUGCUCGUG |
| | UCCAAGGAGAAGAAUAAGGAUGGAAAGUAUGACCUGAUUGCCAC |
| | UGUCGAUAAGCUGGAACUGAAGGGGACCUCCGACAAGAAUAACG |
| | GCUCAGGCGUGCUGGAGGGCGUUAAGGCAGAUAAGAGCAAAGUG |
| | AAGCUGACAAUUUCUGAUGAUCUGGGACAGACUACACUCGAGGU |
| | CUUUAAGGAAGACGGCAAGACCCUGGUGAGCAAGAAAGUGACUA |
| | GCAAGGAUAAGUCCUCCACAGAGGAGAAAUUCAACGAAAAAGGA |
| | GAGGUGUCUGAAAAAAUUAUUACACGGGCUGAUGGAACCAGGCU |
| | GGAGUACACAGGGAUUAAGUCCGACGGGUCUGGAAAGGCUAAAG |
| | AGGUGCUGAAAGGAUAUGACCUGAAGGGGGAACUGUCUUCCGAA |
| | AAGACCACACUGGUCGUGAAGGAGGGCACAGUGACACUGUCUAA |
| | AAACAUCGACAAGAGCGGGGAGGUGAGUGUCGAGCUGGACGACA |
| | CUGAUUCUUCCGCUGCCACAAAGAAGACCGCAGCCUGGAAUAGCG |
| | GGACCUCCACACUGACUAUCACCGUGAACUCUAAGAAGACAAAG |
| | GACCUGGUGUUUACAAAGGAGAAUACCAUCACUGUGCAGCAGUA |
| | CGAUUCCGCCGGCACUAAGCUGGAGGGAUCCGCAGUGGAAAUCA |
| | CAAAGCUGGAUGAGAUCAAGAAUGCACUAAAGUGACGGGUGGCA |
| | UCCCUGUGACCCCUCCCCAGUGCCUCUCCUGGCCCUGGAAGUUGC |
| | CACUCCAGUGCCCACCAGCCUUGUCCUAAUAAAAUUAAGUUGCAU |
| | CAAGCU |
| SEQ ID NO: | GGACAGAUCGCCUGGAGACGCCAUCCACGCUGUUUUGACCUCCAU |
| 54 | AGAAGACACCGGGACCGAUCCAGCCUCCGCGGCCGGGAACGGUGC |
| SS-HA-OspA | AUUGGAACGCGGAUUCCCCGUGCCAAGAGUGACUCACCGUCCUUG |
| ST2 Gly(-) | ACACGAUGAAGGCCAAACUGCUGGUGCUGCUGUGCACUUUCAC |
| SS bold / | UGCAACUUACGCCAAGCAGAACGUGGGAUCUCUGGAUGAAAAGA |
| underlined | AUUCUGCAAGCGUGGAUCUGCCAGGCGAGAUGAAGGUUCUGGUC |
| | UCCAAGGAGAAGGACAAGGACGGCAAAUAUUCUCUGAAAGCCAC |
| | AGUGGAUAAGAUUGAGCUGAAGGGCACAUCCGAUAAGGACGACG |
| | GCUCUGGGGUGCUGGAGGGAACCAAGGACGACAAAAGCAAGGCC |
| | AAGCUGACAAUCGCCGACGACCUGUCUAAAACUACUUUCGAACUC |
| | UUUAAGGAGGAUGGCAAAACCCUGGUGUCCAGGAAAGUGUCCUC |
| | CAAAGACAAGACCUCUACAGAUGAGAUGUUCAAUGAAAAGGGGG |
| | AGCUGUCCGCCAAAACCAUGACCAGAGAGGACGGAACCAAGCUG |
| | GAAUAUACCGAAAUGAAGUCCGACGGCACCGGAAAAGCCAAGGA |
| | GGUCCUGAAACACUUCACACUGGAGGGGAAGGUGGCCAAUGACA |
| | AGGUGACACUGGAGGUGAAAGAGGGCACAGUGACUCUGAGCAAA |
| | GAGAUCGCCAAAUCUGGCGAGGUUACUGUGGCACUGAAGGAUAC |
| | AAGCACCACACAGGCCACCAAGAAAACAGGCGCCUGGGAUAGCAA |
| | GACAUCCACUCUGACAAUUUCCGUCAAUUCUAAGAAGACCACCCA |
| | GCUCGUGUUCACUAAGCAGGACACUAUCACCGUGCAGAAGUAUG |
| | ACUCUGCAGGAACAAACCUGGAAGGCACAGCUGUGGAAAUCAAA |
| | ACUCUGGAUGAACUGAAAAACGCCCUGAAGUGACGGGUGGCAUC |
| | CCUGUGACCCCUCCCCAGUGCCUCUCCUGGCCCUGGAAGUUGCCA |
| | CUCCAGUGCCCACCAGCCUUGUCCUAAUAAAAUUAAGUUGCAUCA |
| | AGCU |
| SEQ ID NO: | GGACAGAUCGCCUGGAGACGCCAUCCACGCUGUUUUGACCUCCAU |
| 55 | AGAAGACACCGGGACCGAUCCAGCCUCCGCGGCCGGGAACGGUGC |
| SS-HA-OspA | AUUGGAACGCGGAUUCCCCGUGCCAAGAGUGACUCACCGUCCUUG |
| ST2 Gly(-) | ACACGAUGAAAGCCAAACUUCUGGUCCUGCUCUGUACCUUCAC |
| SS bold / | UGCAACCUACGCCAAGCAGAACGUGGGCUCUCUGGACGAGAAGA |
| underlined | ACAGUGCUAGCGUCGACCUGCCAGGGGAAAUGAAGGUGCUGGUC |
| | UCUAAAGAGAAGGACAAAGACGGCAAGUAUAGCCUGAAGGCCAC |
| | AGUGGACAAGAUUGAACUGAAGGGAACUUCCGACAAGGACGACG |
| | GGAGCGGCGUCCUGGAAGGCACAAAGGAUGACAAGUCCAAAGCC |
| | AAGCUGACCAUUGCUGACGACCUGUCUAAGACCACCUUCGAACUC |
| | UUCAAGGAAGAUGGAAAGACACUGGUGAGCAGGAAGGUGUCUAG |
| | CAAGGACAAGACCUCAACAGACGAAAUGUUUAAUGAAAAAGGCG |
| | AGCUGUCUGCCAAGACGAUGACCCGCGAGGACGGAACCAAGCUG |
| | GAAUACACCGAAAUGAAGUCCGAUGGGACUGGCAAGGCCAAGGA |
| | GGUCCUGAAGCACUUCACACUGGAGGGGAAGGUGGCCAAUGAUA |
| | AGGUGACUCUCGAGGUCAAGGAGGGCACAGUUACCCUGUCUAAG |
| | GAGAUCGCCAAAUCCGGCGAGGUGACCGUGGCACUGAAGGAUAC |
| | AAGCACUACUCAGGCAACUAAGAAGACAGGCGCUUGGGACUCUA |
| | AGACAAGCACACUGACUAUCUCUGUGAAUUCCAAAAAGACAACU |
| | CAGCUGGUGUUCACAAAGCAGGAUACAAUUACCGUGCAGAAGUA |
| | UGAUUCCGCUGGAACAAAUCUGGAGGGAACAGCCGUGGAGAUUA |
| | AGACACUGGACGAGCUGAAGAAUGCUCUGAAAUGACGGGUGGCA |
| | UCCCUGUGACCCCUCCCCAGUGCCUCUCCUGGCCCUGGAAGUUGC |
| | CACUCCAGUGCCCACCAGCCUUGUCCUAAUAAAAUUAAGUUGCAU |
| | CAAGCU |
| SEQ ID NO: | GGACAGAUCGCCUGGAGACGCCAUCCACGCUGUUUUGACCUCCAU |
| 56 | AGAAGACACCGGGACCGAUCCAGCCUCCGCGGCCGGGAACGGUGC |
| SS-HA-OspA | AUUGGAACGCGGAUUCCCCGUGCCAAGAGUGACUCACCGUCCUUG |
| ST1 Gly(-)- | ACACGAUGAAAGCAAAGCUGCUGGUCCUGCUGUGUACUUUCAC |
| HA-TMB | AGCAACUUAUGCAAAACAGAAUGUGGGCUCUCUGGACGAGAAAA |
| SS bold / | ACAGCGUGUCCGUGGACCUGCCCGGAGAGAUGAAAGUGCUGGUG |
| underlined | UCCAAGGAGAAGAACAAGGAUGGGAAGUACGACCUGAUCGCUAC |
| TMB bold / | CGUUGACAAGCUGGAGCUGAAGGGGACUAGCGAUAAAAACAACG |
| italics | GCUCCGGCGUCCUGGAAGGAGUGAAGGCCGACAAGAGCAAGGUG |
| linker bold / | AAACUGACUAUUUCCGACGAUCUGGGACAGACAACUCUGGAAGU |
| italics / | GUUUAAGGAAGACGGAAAAACUCUCGUUUCCAAGAAGGUGACAU |
| underlined | CCAAGGACAAGUCCUCCACCGAGGAGAAGUUCAAUGAGAAGGGA |
| | GAGGUUUCCGAGAAGAUCAUCACAAGAGCAGACGGCACCAGACU |
| | GGAGUACACCGGAAUCAAGAGCGACGGCUCUGGCAAGGCCAAAG |
| | AGGUGCUGAAGGGCUAUGACCUGAAGGGGGAACUGUCCUCUGAG |
| | AAAACCACUCUGGUGGUGAAAGAGGGCACAGUGACACUGUCCAA |
| | AAACAUCGACAAGAGUGGGGAGGUGUCCGUGGAGCUGGACGAUA |
| | CAGACAGCUCCGCCGCAACCAAAAAGACUGCAGCCUGGAACUCCG |
| | GAACAUCUACCCUGACUAUCACUGUUAACUCCAAGAAAACUAAG |
| | GACCUGGUGUUUACUAAGGAGAAUACAAUCACAGUGCAGCAGUA |
| | UGAUAGCGCCGGAACCAAGCUGGAGGGCUCCGCCGUGGAGAUCA |
| | CAAAGCUGGAUGAGAUUAAGAACGCCCUGAAGUCUGGGUCCAUU |
| | CUGGCAAUCUACAGCACAGUGGCCUCUUCUCUGGUGCUGCUGGUU |
| | UCCCUGGGCGCCAUUAGUUUUGGCUCUGGCUGACGGGUGGCAUCC |
| | CUGUGACCCCUCCCCAGUGCCUCUCCUGGCCCUGGAAGUUGCCAC |
| | UCCAGUGCCCACCAGCCUUGUCCUAAUAAAAUUAAGUUGCAUCA |
| | AGCU |
| SEQ ID NO: | GGACAGAUCGCCUGGAGACGCCAUCCACGCUGUUUUGACCUCCAU |
| 57 | AGAAGACACCGGGACCGAUCCAGCCUCCGCGGCCGGGAACGGUGC |
| SS-HA-OspA | AUUGGAACGCGGAUUCCCCGUGCCAAGAGUGACUCACCGUCCUUG |
| ST1 Gly(-)- | ACACGAUGAAAGCCAAGCUCCUGGUGCUCCUGUGCACAUUCAC |
| HA-TMB | UGCAACUUACGCCAAGCAGAACGUGGGAAGCCUGGACGAGAAGA |
| SS bold / | ACAGCGUGAGCGUGGAUCUGCCCGGAGAGAUGAAGGUCCUGGUG |
| underlined | UCCAAGGAGAAGAAUAAGGACGGAAAAUACGACCUCAUUGCUAC |
| TMB bold / | CGUCGAUAAACUGGAGCUGAAGGGAACCAGCGAUAAGAACAAUG |
| italics | GAUCUGGAGUGCUGGAGGGGGUCAAGGCUGAUAAGUCAAAGGUG |
| linker bold / | AAACUGACAAUCAGUGAUGACCUGGGCCAGACAACCCUGGAGGU |
| italics / | GUUCAAGGAAGACGGGAAAACUCUGGUGAGUAAGAAGGUCACCU |
| underlined | CCAAAGAUAAGAGCAGCACAGAAGAGAAGUUCAACGAAAAGGGA |
| | GAGGUGAGCGAGAAGAUCAUCACACGCGCUGACGGCACCCGCCUG |
| | GAGUAUACCGGCAUCAAGUCCGACGGGUCCGGCAAAGCUAAAGA |
| | GGUGCUGAAAGGCUACGAUCUGAAGGGAGAGCUGAGCUCCGAGA |
| | AAACUACCCUGGUGGUGAAGGAGGGAACAGUGACACUGUCCAAA |
| | AACAUCGAUAAGAGUGGGGAGGUGAGCGUUGAGCUGGACGACAC |
| | AGACAGCAGCGCAGCCACAAAGAAGACAGCCGCAUGGAAUAGCG |
| | GGACCAGCACACUGACCAUCACAGUGAAUUCUAAAAAGACUAAA |
| | GACCUCGUCUUUACUAAAGAAAACACUAUUACUGUCCAGCAAUA |
| | CGACUCUGCCGGAACUAAGCUGGAGGGAUCUGCCGUGGAGAUUA |
| | CCAAGCUGGACGAGAUCAAGAACGCCCUGAAGUCCGGCAGCAUCC |
| | UCGCCAUCUACUCCACCGUGGCCUCUAGCCUGGUUCUGCUGGUGA |
| | GCCUGGGCGCCAUUUCUUUUGGGAGCGGAUGACGGGUGGCAUCCC |
| | UGUGACCCCUCCCCAGUGCCUCUCCUGGCCCUGGAAGUUGCCACU |
| | CCAGUGCCCACCAGCCUUGUCCUAAUAAAAUUAAGUUGCAUCAA |
| | GCU |
| SEQ ID NO: | GGACAGAUCGCCUGGAGACGCCAUCCACGCUGUUUUGACCUCCAU |
| 58 | AGAAGACACCGGGACCGAUCCAGCCUCCGCGGCCGGGAACGGUGC |
| SS-HA-OspA | AUUGGAACGCGGAUUCCCCGUGCCAAGAGUGACUCACCGUCCUUG |
| ST2 Gly(-)- | ACACGAUGAAGGCUAAGCUGCUGGUGCUGCUGUGCACCUUCAC |
| HA-TMB | UGCAACCUACGCCAAGCAAAAUGUCGGCUCCCUGGAUGAAAAGA |
| SS bold / | AUAGCGCCUCCGUGGACCUGCCUGGGGAGAUGAAAGUGCUGGUG |
| underlined | UCCAAGGAAAAAGACAAAGACGGGAAGUACAGCCUGAAGGCCAC |
| TMB bold / | UGUGGACAAGAUUGAGCUGAAAGGGACAAGCGACAAGGAUGACG |
| italics | GGUCUGGAGUGCUGGAGGGCACUAAGGACGAUAAGAGCAAGGCU |
| linker bold / | AAGCUGACUAUCGCCGAUGACCUGAGCAAGACCACUUUUGAACU |
| italics / | CUUCAAGGAAGACGGCAAAACACUGGUAUCCCGCAAGGUGUCUA |
| underlined | GCAAGGAUAAGACAUCCACAGACGAGAUGUUCAAUGAGAAAGGA |
| | GAACUGAGCGCCAAGACCAUGACCAGGGAAGAUGGAACCAAGCU |
| | GGAAUACACCGAGAUGAAGUCUGACGGCACCGGCAAGGCCAAGG |
| | AGGUGCUGAAGCACUUUACUUUGGAGGGCAAAGUGGCCAACGAC |
| | AAGGUGACACUGGAGGUGAAAGAGGGGACCGUGACCCUGUCUAA |
| | AGAGAUUGCAAAGUCCGGCGAAGUGACCGUGGCCCUCAAGGACA |
| | CUUCUACCACCCAGGCUACAAAGAAGACCGGCGCUUGGGAUUCCA |
| | AAACUUCUACCCUGACUAUCUCCGUGAAUUCUAAGAAGACAACCC |
| | AGCUGGUGUUUACCAAGCAGGAUACCAUUACCGUGCAGAAAUAU |
| | GAUAGCGCCGGCACUAAUCUGGAGGGCACCGCUGUGGAGAUUAA |
| | GACCCUGGACGAGCUGAAAAACGCACUGAAAUCCGGCUCUAUUCU |
| | GGCAAUCUACUCCACAGUGGCUUCAAGCCUGGUGCUGCUCGUGUC |
| | CCUCGGGGCAAUCUCCUUCGGAUCUGGGUGACGGGUGGCAUCCCU |
| | GUGACCCCUCCCCAGUGCCUCUCCUGGCCCUGGAAGUUGCCACUC |
| | CAGUGCCCACCAGCCUUGUCCUAAUAAAAUUAAGUUGCAUCAAG |
| | CU |
| SEQ ID NO: | GGACAGAUCGCCUGGAGACGCCAUCCACGCUGUUUUGACCUCCAU |
| 59 | AGAAGACACCGGGACCGAUCCAGCCUCCGCGGCCGGGAACGGUGC |
| SS-HA-OspA | AUUGGAACGCGGAUUCCCCGUGCCAAGAGUGACUCACCGUCCUUG |
| ST2 Gly(-)- | ACACGAUGAAGGCAAAGCUGCUGGUGCUCCUGUGCACCUUUAC |
| HA-TMB | AGCUACAUACGCAAAACAGAACGUGGGCAGCCUGGACGAGAAGA |
| SS bold / | AUAGCGCCAGCGUGGAUCUGCCAGGCGAGAUGAAGGUGCUGGUG |
| underlined | AGCAAAGAGAAGGAUAAAGAUGGGAAGUACAGCCUGAAGGCUAC |
| TMB bold / | UGUGGACAAGAUCGAACUGAAGGGAACUAGCGAUAAAGACGAUG |
| italics | GAAGCGGCGUGCUGGAGGGAACUAAGGACGAUAAAAGCAAAGCC |
| linker bold / | AAGCUGACAAUUGCCGACGACCUGUCCAAAACUACAUUUGAACU |
| italics / | CUUUAAGGAGGAUGGAAAAACACUGGUGAGCAGAAAGGUGUCUU |
| underlined | CUAAGGACAAGACCUCCACCGAUGAGAUGUUCAACGAGAAGGGC |
| | GAGCUGUCUGCAAAAACUAUGACACGCGAGGACGGAACAAAGCU |
| | GGAGUAUACUGAGAUGAAGUCUGACGGCACCGGGAAGGCCAAAG |
| | AGGUGCUGAAACACUUCACCCUGGAGGGGAAGGUGGCUAACGAU |
| | AAGGUUACCCUGGAGGUCAAGGAGGGAACUGUGACUCUGAGCAA |
| | GGAGAUCGCCAAGUCUGGCGAGGUCACAGUGGCCCUCAAGGACA |
| | CAAGCACCACCCAGGCUACAAAGAAGACAGGCGCCUGGGAUUCUA |
| | AAACAAGCACCCUGACUAUCAGUGUCAACUCCAAGAAGACAACCC |
| | AGCUGGUGUUCACCAAGCAGGACACCAUCACCGUGCAGAAAUAU |
| | GACUCUGCUGGAACAAACCUGGAAGGGACCGCUGUCGAAAUCAA |
| | GACUCUGGACGAACUGAAGAAUGCCCUGAAGUCCGGGAGCAUCCU |
| | GGCAAUCUACUCUACAGUGGCUUCCUCCCUUGUUCUGCUGGUCAG |
| | CCUGGGCGCCAUCAGCUUUGGCUCUGGCUGACGGGUGGCAUCCCU |
| | GUGACCCCUCCCCAGUGCCUCUCCUGGCCCUGGAAGUUGCCACUC |
| | CAGUGCCCACCAGCCUUGUCCUAAUAAAAUUAAGUUGCAUCAAG |
| | CU |

The following mnRNA sequences are variants encoding the same viral secretion signal peptide, MKAKLLVLLCTFTATYA (SEQ ID NO: 1):

(SEQ ID NO: 60)
AUGAAGGCCAAGCUGCUGGUCCUGCUCUGUACCUUUACAGCCACUUACG
CC;
(SEQ ID NO: 61)
AUGAAGGCCAAACUGCUCGUGCUCUUAUGCACAUUCACAGCAACCUACG
CC;
(SEQ ID NO: 62)
AUGAAGGCUAAGCUGCUGGUUCUGCUGUGUACUUUUACCGCCACAUACG
CU;
(SEQ ID NO: 63)
AUGAAGGCCAAACUCCUGGUGCUCCUGUGUACCUUCACCGCUACCUACG
CC;
(SEQ ID NO: 64)
AUGAAGGCAAAGCUGCUGGUGCUGCUGUGUACCUUCACUGCCACCUACG
CC;
(SEQ ID NO: 65)
AUGAAGGCCAAACUGCUGGUGCUGCUGUGCACUUUCACUGCAACUUACG
CC;
(SEQ ID NO: 66)
AUGAAAGCCAAACUUCUGGUCCUGCUCUGUACCUUCACUGCAACCUACG
CC;
(SEQ ID NO: 67)
AUGAAGGCCAAGCUGCUGGUGCUGCUGUGUACAUUCACAGCAACCUAUG
CC;
(SEQ ID NO: 68)
AUGAAAGCAAAGCUGCUGGUGCUGCUGUGCACAUUCACCGCAACAUACG
CC;
(SEQ ID NO: 69)
AUGAAAGCAAAGCUGCUGGUCCUGCUGUGUACUUUCACAGCAACUUAUG
CA;
(SEQ ID NO: 70)
AUGAAAGCCAAGCUCCUGGUGCUCCUGUGCACAUUCACUGCAACUUACG
CC;
(SEQ ID NO: 71)
AUGAAGGCUAAACUGCUGGUCCUGCUGUGUACCUUCACCGCUACAUACG
CC;
(SEQ ID NO: 72)
AUGAAGGCAAAACUGCUGGUGCUGCUGUGUACAUUCACAGCUACUUAUG
CA.

The following mRNA sequences are variants encoding the same transmembrane domain,

ILAIYSTVASSLVLLVSLGAISF (SEQ ID NO: 17):
(SEQ ID NO: 73)
AUCCUGGCAAUCUAUAGCACAGUCGCCAGCUCCCUGGUUCUCCUGGUGA
GCCUGGGGGCAAUUUCCUUC;
(SEQ ID NO: 74)
AUCCUGGCCAUCUAUAGCACCGUCGCCAGCUCUCUGGUGCUGCUGGUGU
CCCUCGGGGCUAUCUCAUUC;
(SEQ ID NO: 75)
AUUCUGGCAAUCUACAGCACAGUGGCCUCUUCUCUGGUGCUGCUGGUUU
CCCUGGGCGCCAUUAGUUUU;
(SEQ ID NO: 76)
AUCCUCGCCAUCUACUCCACCGUGGCCUCUAGCCUGGUUCUGCUGGUGA
GCCUGGGCGCCAUUUCUUUU;
(SEQ ID NO: 77)
AUCCUGGCUAUCUAUAGCACUGUGGCUUCCUCUCUGGUGCUGCUGGUUU
CCCUGGGGGCCAUUUCCUUC;
(SEQ ID NO: 78)
AUCCUGGCUAUCUAUAGCACCGUCGCCUCCAGCCUCGUUCUGCUGGUGA
GCCUGGGCGCCAUUUCCUUC;
(SEQ ID NO: 79)
AUUCUGGCAAUCUACUCCACAGUGGCUUCAAGCCUGGUGCUGCUCGUGU
CCCUCGGGGCAAUCUCCUUC;
or
(SEQ ID NO: 80)
AUCCUGGCAAUCUACUCUACAGUGGCUUCCUCCCUUGUUCUGCUGGUCA
GCCUGGGCGCCAUCAGCUUU.

### Example 2: In vitro mRNA Expression & Antigenicity Of Signal Sequence-Containing Antigenic Prokaryotic Polypeptides

The Borrelia genus protein Outer surface protein A (OspA) was used as an exemplary antigenic prokaryotic polypeptide to test the effects of linking one or both of a hemagglutinin secretion signal (HA1 SS) and HA transmembrane domain (TMB) to OspA. OspA serotype 1 (ST1) and serotype 2 (ST2) were used.

mRNA expressing either OspA ST1 or ST2 were designed. Different mRNA sequences were designed to direct the expression of OspA intracellularly, secreted, or transmembrane using the OspA sequence without or with fusion to hemagglutinin secretion signal (HA1 SS) and/or HA transmembrane domain (TMB). The HA1 SS causes OspA secretion, the TMB directs OspA to the membrane, and OspA without either HA1 SS or TMB is present intracellularly.

Expression and antigenicity of the OspA ST1 and ST2 antigens delivered by mRNA was confirmed in vitro before vaccination of mice. The methods of transfection and expression of the mRNA are described above in Example 1.

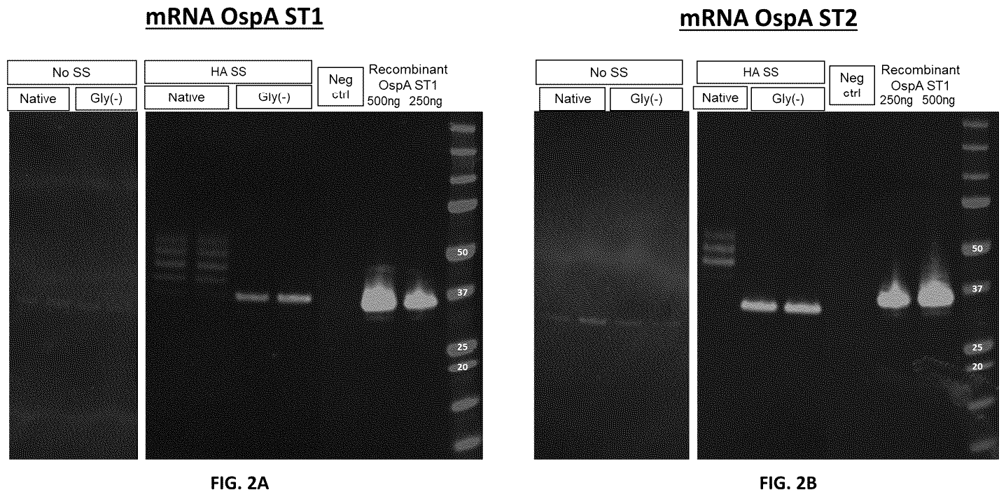

As shown in FIG. 2A and FIG. 2B, in vitro expression of OspA ST1 and ST2 mRNA in HEK293T cell supernatants was achieved. mRNA-OspA ST1 and mRNA-OspA ST2 were tested without or with an HA secretion signal, and without or with glycosylation site mutations. A negative control (buffer) and a positive control (recombinant OspA) were also used. HEK293T cells were transfected with each mRNA and after 48 hours, supernatants were collected and run on a Western Blot. Blotted proteins were detected with polyclonal rabbit antibodies that recognize OspA. The results demonstrated that adding a secretion signal increased extracellular expression of OspA. Mutation of glycosylation sites effectively avoided glycosylation of OspA, with the presence of a single spot for Gly(-), instead of multiple spots for the native (glycosylated) protein.

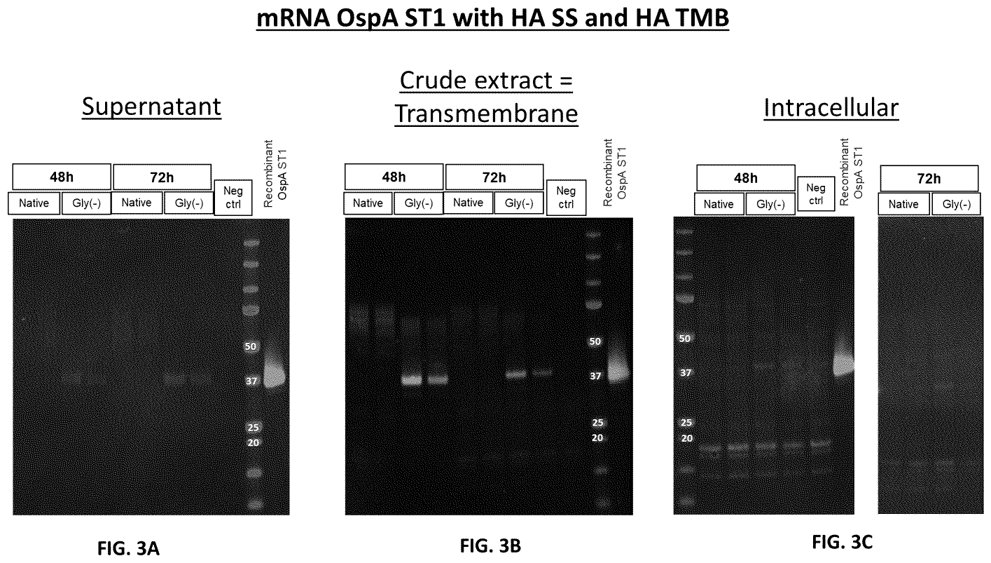

As shown in FIG. 3A—FIG. 3C, in vitro expression of mRNA-TMB-OspA ST1 in HEK293T cells was achieved. Cell supernatants, crude extracts, and intracellular compartments were tested for OspA ST1 expression. After 48-72 hours, supernatants and cells were collected and run on a Western Blot. Blotted proteins were detected with polyclonal rabbit antibodies that recognize OspA. The results demonstrate that adding a transmembrane domain induces localization of OspA ST1 at the cell membrane and reduces secretion and intracellular localization. Mutation of glycosylation sites effectively avoided glycosylation of OspA ST1.

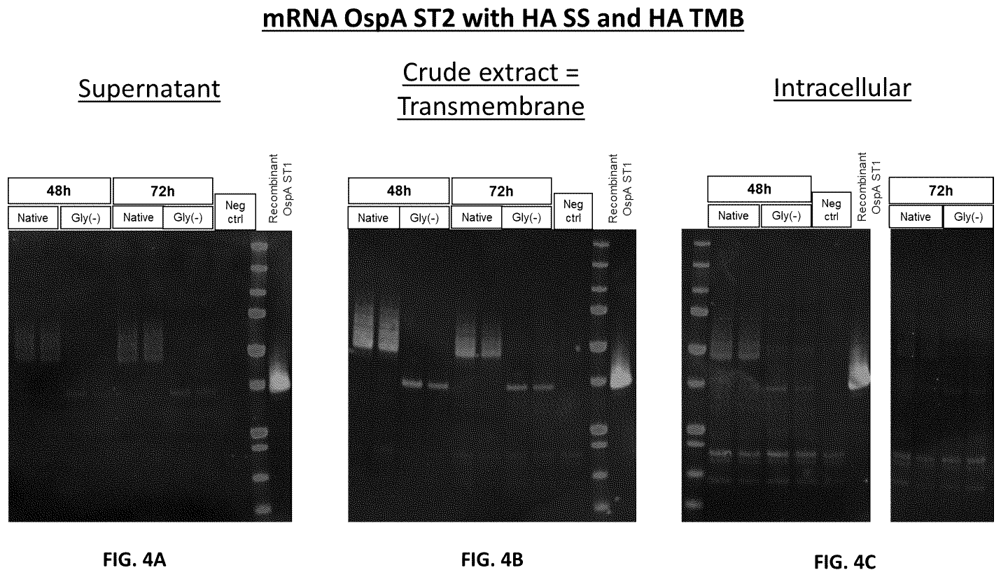

As shown in FIG. 4A—FIG. 4C, in vitro expression of mRNA-TMB-OspA ST2 in HEK293T cells was achieved. Cell supernatants, crude extracts, and intracellular compartments were tested for OspA ST2 expression. After 48-72 hours, supernatants and cells were collected and run on a Western Blot. Blotted proteins were detected with polyclonal rabbit antibodies that recognize OspA. As with OspA ST1, the results demonstrate that adding a transmembrane domain induces localization of OspA ST2 at the cell membrane and reduces secretion and intracellular localization. Mutation of glycosylation sites effectively avoided glycosylation of OspA ST2.

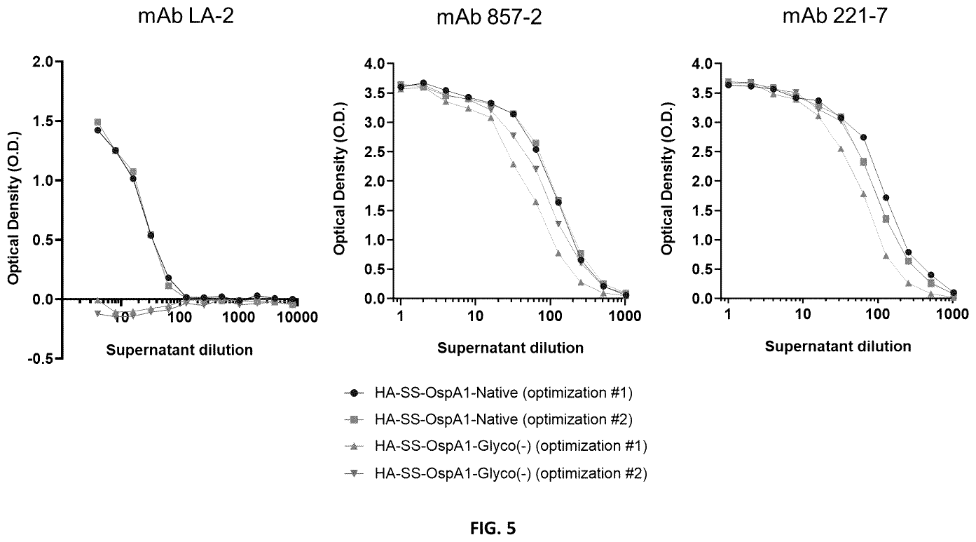

As shown in FIG. 5, the antigenicity of OspA ST1 antigen delivered by mRNA in HEK293T cells was shown. Transfected cell supernatants were used to perform sandwich ELISAs with functional monoclonal antibodies LA-2, 857-2 and 221-7.

mAb LA-2 targets the C terminus of OspA ST1 only and has been shown to correlate with protection in clinical studies (Van Hoecke, supra; Steere, supra; Embers, supra). Mabs 221-7 and 857-2 were selected as lead candidates based on borreliacidal activity and protection in mice against tick-mediated transmission of B. burgdorferi, as shown in Wang et al., supra.

Secreted OspA ST1 (HA-SS-OspA ST1) produced by mRNA was correctly recognized by the 3 functional mAbs, showing that the antigen was in the correct conformation after in vitro expression in human cells (HEK293T).

Mutation of glycosylation sites inhibited the binding of the mAb LA-2, probably due to the induction of a wrong conformation in the C terminal epitope. This was not observed with 221-7 and 857-2 antibodies.

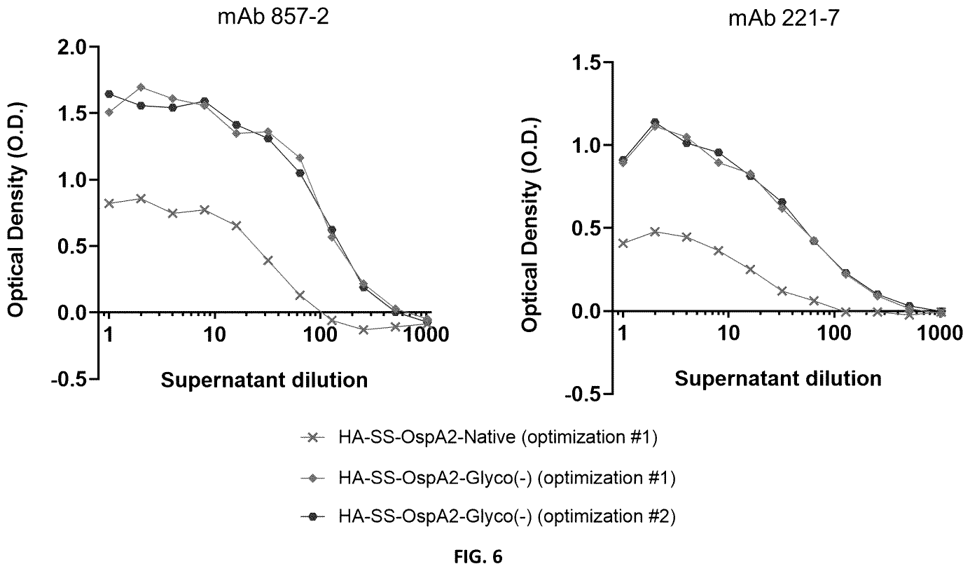

As shown in FIG. 6, the antigenicity of OspA ST2 antigen delivered by mRNA in HEK293T cells was shown. Transfected cell supernatants were used to perform sandwich ELISAs with functional monoclonal antibodies 857-2 and 221-7. The mAb LA-2 was not used with OspA ST2 as it only recognizes OspA ST1.

Secreted OspA ST2 (HA-SS-OspA ST2) produced by mRNA was correctly recognized by the functional mAbs 221-7 and 857-2, showing that the antigen was in the correct conformation after in vitro expression in human cells. Mutations of glycosylation sites improved the binding of both mAbs, probably due to lower masking of epitopes by glycosylation.

### Example 3: Immunogenicity of mRNA Encoding OspA Protein in Mice

The relative immunogenicity of the various OspA-expressing mRNA was tested in mice by measuring IgG titers against OspA, as described above in Example 1. Each mRNA was encapsulated into an LNP composed of 40% cationic lipid cKK-E10, 30% phospholipid DOPE, 1.5% PEGylated lipid DMGPEG2000, and 28.5% cholesterol. Alternatively, the LNP lipids may be recited as ratios where cationic lipid: PEGylated lipid: cholesterol: phospholipid is 40: 1.5 28.5: 30.

Each LNP-mRNA composition was administered to mice at a dose of 0.2 μg, 1 μg, 5 μg, or 10 μg. In total, 4 groups with 8 mice/group were used.

Three benchmark compositions were used: an OspA fusion with an AlOOH adjuvant (“OspA fusion ST1-ST2”) (at a 2 μg (1 μg by serotype)/dose); Lyme dog vaccine RECOMBITEK® (Merial) (at a 1 μg dose), and an OspA-ferritin fusion (ST1 or ST2) with an AF03 adjuvant (1.7 μg (of which 1 μg OspA+0.7 μg ferritin)/dose). The OspA-ferritin fusion is further described in US20210017238A1, incorporated herein by reference.

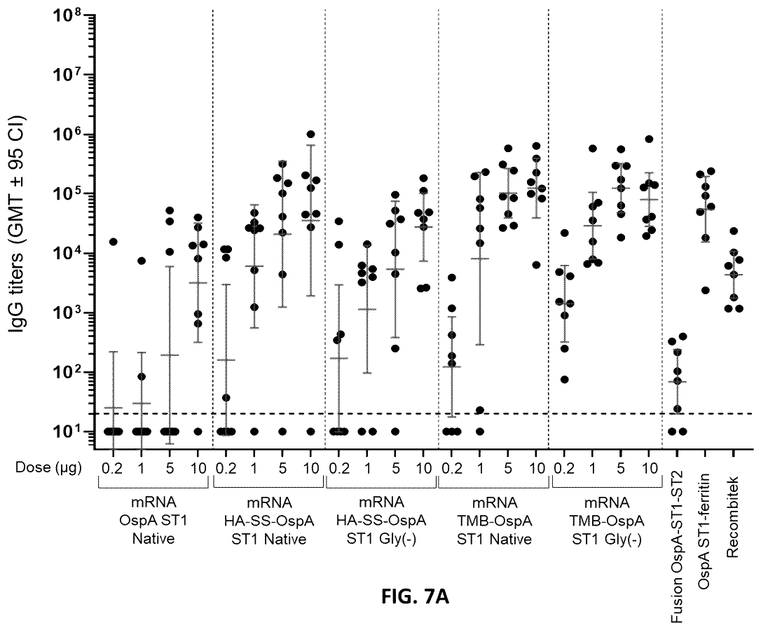

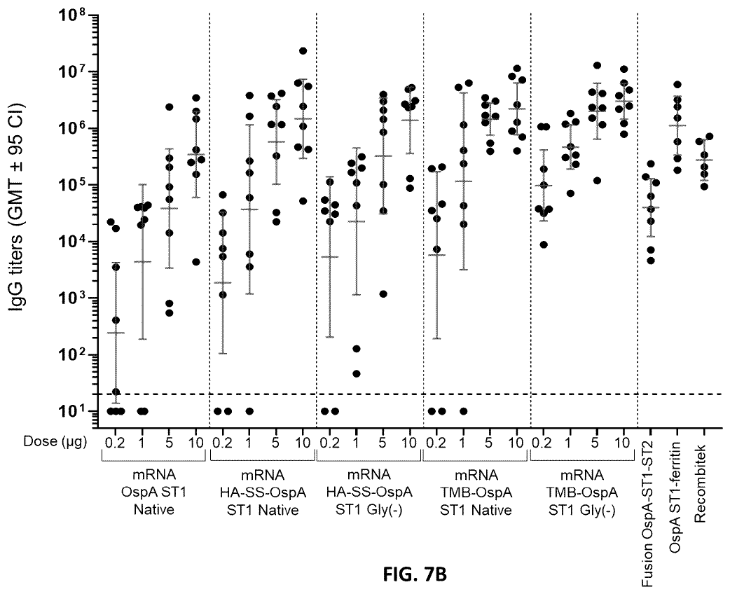

As shown in FIG. 7A, anti-OspA ST1 IgG titers were elevated post-dose 1 (day 19). A trend for HA-SS to increase IgG titers was observed. Moreover, adding a TMB domain significantly increased IgG titers.

As shown in FIG. 7B, anti-OspA ST1 IgG titers were elevated further post-dose 2 (day 35). As can be seen in Table 6, adding HA-SS and/or TMB domain significantly improved immunogenicity (p<0.05).

TABLE 6
Statistical analyses, post dose 2 (day 35), all doses combined

| Compared groups | Fold | P-value |
|---|---|---|
| SS-native = SS-gly(−) | — | P > 0.05 |
| SS-native = TMB-native | — | P > 0.05 |
| Native < SS-gly(−) | X7.9 | 0.0102 |
| Native < SS-native | X8 | 0.0088 |
| Native < TMB-gly(−) | X66.5 | P < 0.001 |
| Native < TMB-native | X19.8 | P < 0.001 |
| SS-gly(−) < TMB-gly(−) | X8.4 | P < 0.001 |
| SS-native < TMB-gly(−) | X8.3 | P < 0.001 |
| TMB-native < TMB-gly(−) | X3.4 | 0.0454 |

### Conclusions on Mouse Study:

mRNA coding for OspA ST1 with one or both of the HA-SS or TMB was immunogenic and induced strong anti-OspA IgG titers in mice both post-dose 1 and post-dose 2. It was found that adding a secretion signal (HA-SS) or a TMB domain significantly improved immunogenicity, compared to the mRNA OspA without any of HA-SS or TMB (p<0.05). A dose effect was observed (i.e., increasing IgG titers with increasing dose).

### Example 4: Expanded mRNA Design Panel

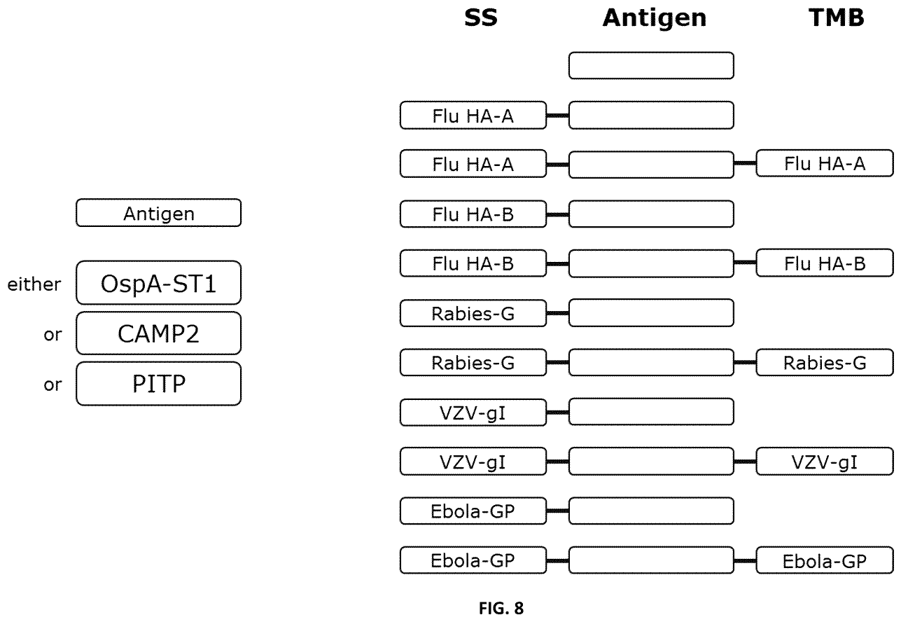

In Example 3, it was demonstrated that the addition of a hemagglutinin secretion signal (HA-SS) and/or a HA transmembrane domain (HA-TMB) enhanced the immunogenicity of the OspA ST1 target antigen. Based on these results, a more comprehensive panel of mRNA constructs were designed, which linked the secretion signal (SS) and transmembrane domains (TMB) from the glycoproteins of various viral families to different target antigens. A schematic representation of the elements included in this expanded mRNA panel is provided in FIG. 8. The panel consisted of three different prokaryotic antigens: OspA ST1, CAMP2, and PITP (the latter two derived from Cutibacterium acnes). The mRNA sequences encoding these antigens were additionally engineered to direct the localization of the antigen intracellularly, secrete it extracellularly, or expose it at the cell membrane. This was achieved by fusing the antigen sequence with or without SS and with or without TMB, derived from glycoproteins from distinct viral families such as Influenza (subtypes A or B), Rabies, Varicella (VZV), or Ebola.

Fifty-seven distinct constructs were subjected to in silico analysis using SignalP 6.0 to determine the strength of their signal peptides. SignalP is an algorithm used for signal sequence prediction and is described in further detail in Armenteros et al. (Nature Biotechnology. 37: 420-423. 2019), Teufel et al. (Nature Biotechnology. 40: 1023-1025. 2022), and https://services.healthtech.dtu.dk/services/SignalP-6.0/, each of which is incorporated herein by reference in their entirety. The strength is assessed based on a cumulative rank score that considers the likelihood of detecting canonical features of the signal sequence (SS likelihood score, also known as SP likelihood score) and the probability of cleavage at the cleavage site (cleavage probability score). The sequences used for this analysis are displayed in Table 7 and the outcome of this analysis are presented below in Table 8.

TABLE 7
Sequences Analyzed in SignalP 6.0

| NAME | SEQUENCE |
|---|---|
| >OspA | MKAKLLVLLCTFTATYAKQNVSSLDEKNSVSVDLPGEMKVLVSKEKNKDGK |
| ST1_SS_Flu- | YDLIATVDKLELKGTSDKSNGSGVLEGVKADKSKVKLTISDDLGQTTLEVFKE |
| HA_A/ | DGKTLVSKKVTSKDKSSTEEKFNEKGEVSEKIITRADGTRLEYTGIKSDGSGKA |
| NewCaledonia/ | KEVLKGYDLKGELSSEKTTLVVKEGTVTLSKNISKSGEVSVELNDTDSSAATK |
| 20/1999 | KTAAWNSGTSTLTITVNSKKTKDLVFTKENTITVQQYDSNGTKLEGSAVEITK |
| | LDEIKNALK (SEQ ID NO: 193) |
| >OspA | MKAILVVLLYTFATANAKQNVSSLDEKNSVSVDLPGEMKVLVSKEKNKDGK |
| ST1_SS_Flu- | YDLIATVDKLELKGTSDKSNGSGVLEGVKADKSKVKLTISDDLGQTTLEVFKE |
| HA_A/ | DGKTLVSKKVTSKDKSSTEEKFNEKGEVSEKIITRADGTRLEYTGIKSDGSGKA |
| California/7/ | KEVLKGYDLKGELSSEKTTLVVKEGTVTLSKNISKSGEVSVELNDTDSSAATK |
| 2009 | KTAAWNSGTSTLTITVNSKKTKDLVFTKENTITVQQYDSNGTKLEGSAVEITK |
| | LDEIKNALK (SEQ ID NO: 194) |
| >OspA | MKTIIALSYILCLVFAKQNVSSLDEKNSVSVDLPGEMKVLVSKEKNKDGKYDL |
| ST1_SS_Flu- | IATVDKLELKGTSDKSNGSGVLEGVKADKSKVKLTISDDLGQTTLEVFKEDGK |
| HA_A/Moscow/ | TLVSKKVTSKDKSSTEEKFNEKGEVSEKIITRADGTRLEYTGIKSDGSGKAKEV |
| 10/1999 | LKGYDLKGELSSEKTTLVVKEGTVTLSKNISKSGEVSVELNDTDSSAATKKTA |
| | AWNSGTSTLTITVNSKKTKDLVFTKENTITVQQYDSNGTKLEGSAVEITKLDEI |
| | KNALK |
| | (SEQ ID NO: 195) |
| >OspA | MKAIIVLLMVVTSNAKQNVSSLDEKNSVSVDLPGEMKVLVSKEKNKDGKYD |
| ST1_SS_Flu- | LIATVDKLELKGTSDKSNGSGVLEGVKADKSKVKLTISDDLGQTTLEVFKEDG |
| HA_B/ | KTLVSKKVTSKDKSSTEEKFNEKGEVSEKIITRADGTRLEYTGIKSDGSGKAKE |
| Phuket/3073/2013 | VLKGYDLKGELSSEKTTLVVKEGTVTLSKNISKSGEVSVELNDTDSSAATKKT |
| | AAWNSGTSTLTITVNSKKTKDLVFTKENTITVQQYDSNGTKLEGSAVEITKLD |
| | EIKNALK |
| | (SEQ ID NO: 196) |
| >OspA | MFVFLVLLPLVSKQNVSSLDEKNSVSVDLPGEMKVLVSKEKNKDGKYDLIAT |
| ST1_SS- | VDKLELKGTSDKSNGSGVLEGVKADKSKVKLTISDDLGQTTLEVFKEDGKTL |
| spike_CoV2_ | VSKKVTSKDKSSTEEKFNEKGEVSEKIITRADGTRLEYTGIKSDGSGKAKEVLK |
| short | GYDLKGELSSEKTTLVVKEGTVTLSKNISKSGEVSVELNDTDSSAATKKTAAW |
| | NSGTSTLTITVNSKKTKDLVFTKENTITVQQYDSNGTKLEGSAVEITKLDEIKN |
| | ALK |
| | (SEQ ID NO: 197) |
| >OspA | MFLLTTKRTMFVFLVLLPLVSKQNVSSLDEKNSVSVDLPGEMKVLVSKEKNK |
| ST1_SS- | DGKYDLIATVDKLELKGTSDKSNGSGVLEGVKADKSKVKLTISDDLGQTTLE |
| spike_CoV2_ | VFKEDGKTLVSKKVTSKDKSSTEEKFNEKGEVSEKIITRADGTRLEYTGIKSDG |
| long | SGKAKEVLKGYDLKGELSSEKTTLVVKEGTVTLSKNISKSGEVSVELNDTDSS |
| | AATKKTAAWNSGTSTLTITVNSKKTKDLVFTKENTITVQQYDSNGTKLEGSAV |
| | EITKLDEIKNALK |
| | (SEQ ID NO: 198) |
| >OspA | MVPQALLFVPLLVFPLCFGKQNVSSLDEKNSVSVDLPGEMKVLVSKEKNKDG |
| ST1_SS_ | KYDLIATVDKLELKGTSDKSNGSGVLEGVKADKSKVKLTISDDLGQTTLEVFK |
| Rabies- | EDGKTLVSKKVTSKDKSSTEEKFNEKGEVSEKIITRADGTRLEYTGIKSDGSGK |
| G_Pasteur- | AKEVLKGYDLKGELSSEKTTLVVKEGTVTLSKNISKSGEVSVELNDTDSSAAT |
| vaccins | KKTAAWNSGTSTLTITVNSKKTKDLVFTKENTITVQQYDSNGTKLEGSAVEIT |
| | KLDEIKNALK (SEQ ID NO: 199) |
| >OspA | MGTVNKPVVGVLMGFGIITGTLRITNPVRAKQNVSSLDEKNSVSVDLPGEMK |
| ST1_SS_ | VLVSKEKNKDGKYDLIATVDKLELKGTSDKSNGSGVLEGVKADKSKVKLTIS |
| VZV-gE_Oka- | DDLGQTTLEVFKEDGKTLVSKKVTSKDKSSTEEKFNEKGEVSEKIITRADGTRL |
| vaccine | EYTGIKSDGSGKAKEVLKGYDLKGELSSEKTTLVVKEGTVTLSKNISKSGEVS |
| | VELNDTDSSAATKKTAAWNSGTSTLTITVNSKKTKDLVFTKENTITVQQYDSN |
| | GTKLEGSAVEITKLDEIKNALK (SEQ ID NO: 200) |
| >OspA | MFLIQCLISAVIFYIQVTNAKQNVSSLDEKNSVSVDLPGEMKVLVSKEKNKDG |
| ST1_SS_ | KYDLIATVDKLELKGTSDKSNGSGVLEGVKADKSKVKLTISDDLGQTTLEVFK |
| VZV-gI_Oka- | EDGKTLVSKKVTSKDKSSTEEKFNEKGEVSEKIITRADGTRLEYTGIKSDGSGK |
| vaccine | AKEVLKGYDLKGELSSEKTTLVVKEGTVTLSKNISKSGEVSVELNDTDSSAAT |
| | KKTAAWNSGTSTLTITVNSKKTKDLVFTKENTITVQQYDSNGTKLEGSAVEIT |
| | KLDEIKNALK(SEQ ID NO: 201) |
| >OspA | MQALGIKTEHFIIMCLLSGHAKQNVSSLDEKNSVSVDLPGEMKVLVSKEKNK |
| ST1_SS_ | DGKYDLIATVDKLELKGTSDKSNGSGVLEGVKADKSKVKLTISDDLGQTTLE |
| VZV-gK_Oka- | VFKEDGKTLVSKKVTSKDKSSTEEKFNEKGEVSEKIITRADGTRLEYTGIKSDG |
| vaccine | SGKAKEVLKGYDLKGELSSEKTTLVVKEGTVTLSKNISKSGEVSVELNDTDSS |
| | AATKKTAAWNSGTSTLTITVNSKKTKDLVFTKENTITVQQYDSNGTKLEGSAV |
| | EITKLDEIKNALK(SEQ ID NO: 202) |
| >OspA | MGLKVNVSAIFMAVLLTLQTPTGKQNVSSLDEKNSVSVDLPGEMKVLVSKEK |
| ST1_SS_ | NKDGKYDLIATVDKLELKGTSDKSNGSGVLEGVKADKSKVKLTISDDLGQTT |
| Measles- | LEVFKEDGKTLVSKKVTSKDKSSTEEKFNEKGEVSEKIITRADGTRLEYTGIKS |
| F_Edmonston- | DGSGKAKEVLKGYDLKGELSSEKTTLVVKEGTVTLSKNISKSGEVSVELNDTD |
| Zagreb- | SSAATKKTAAWNSGTSTLTITVNSKKTKDLVFTKENTITVQQYDSNGTKLEGS |
| vacccine | AVEITKLDEIKNALK (SEQ ID NO: 203) |
| >OspA | MGAAAALTAVVLQGYNPPAYGKQNVSSLDEKNSVSVDLPGEMKVLVSKEKN |
| ST1_SS_ | KDGKYDLIATVDKLELKGTSDKSNGSGVLEGVKADKSKVKLTISDDLGQTTL |
| Rubella- | EVFKEDGKTLVSKKVTSKDKSSTEEKFNEKGEVSEKIITRADGTRLEYTGIKSD |
| E1_RA27/3- | GSGKAKEVLKGYDLKGELSSEKTTLVVKEGTVTLSKNISKSGEVSVELNDTDS |
| vaccine | SAATKKTAAWNSGTSTLTITVNSKKTKDLVFTKENTITVQQYDSNGTKLEGSA |
| | VEITKLDEIKNALK (SEQ ID NO: 204) |
| >OspA | MGAPQAFLAGLLLAAVAVGTARAKQNVSSLDEKNSVSVDLPGEMKVLVSKE |
| ST1_SS_ | KNKDGKYDLIATVDKLELKGTSDKSNGSGVLEGVKADKSKVKLTISDDLGQT |
| Rubella- | TLEVFKEDGKTLVSKKVTSKDKSSTEEKFNEKGEVSEKIITRADGTRLEYTGIK |
| E2_RA27/3- | SDGSGKAKEVLKGYDLKGELSSEKTTLVVKEGTVTLSKNISKSGEVSVELNDT |
| vaccine | DSSAATKKTAAWNSGTSTLTITVNSKKTKDLVFTKENTITVQQYDSNGTKLEG |
| | SAVEITKLDEIKNALK (SEQ ID NO: 205) |
| >OspA | MKVFLVTCLGFAVFSSSVCKQNVSSLDEKNSVSVDLPGEMKVLVSKEKNKDG |
| ST1_SS_ | KYDLIATVDKLELKGTSDKSNGSGVLEGVKADKSKVKLTISDDLGQTTLEVFK |
| Mumps- | EDGKTLVSKKVTSKDKSSTEEKFNEKGEVSEKIITRADGTRLEYTGIKSDGSGK |
| F_Miyahara- | AKEVLKGYDLKGELSSEKTTLVVKEGTVTLSKNISKSGEVSVELNDTDSSAAT |
| vaccine | KKTAAWNSGTSTLTITVNSKKTKDLVFTKENTITVQQYDSNGTKLEGSAVEIT |
| | KLDEIKNALK (SEQ ID NO: 206) |
| >OspA | MGVTGILQLPRDRFKRTSFFLWVIILFQRTFSKQNVSSLDEKNSVSVDLPGEMK |
| ST1_SS_ | VLVSKEKNKDGKYDLIATVDKLELKGTSDKSNGSGVLEGVKADKSKVKLTIS |
| Ebola- | DDLGQTTLEVFKEDGKTLVSKKVTSKDKSSTEEKFNEKGEVSEKIITRADGTRL |
| GP_Mayinga- | EYTGIKSDGSGKAKEVLKGYDLKGELSSEKTTLVVKEGTVTLSKNISKSGEVS |
| 76 | VELNDTDSSAATKKTAAWNSGTSTLTITVNSKKTKDLVFTKENTITVQQYDSN |
| | GTKLEGSAVEITKLDEIKNALK (SEQ ID NO: 207) |
| >OspA | MRSLIIFLLFPSIIYSKQNVSSLDEKNSVSVDLPGEMKVLVSKEKNKDGKYDLIA |
| ST1_SS_ | TVDKLELKGTSDKSNGSGVLEGVKADKSKVKLTISDDLGQTTLEVFKEDGKT |
| VSV-G_Indiana | LVSKKVTSKDKSSTEEKFNEKGEVSEKIITRADGTRLEYTGIKSDGSGKAKEVL |
| | KGYDLKGELSSEKTTLVVKEGTVTLSKNISKSGEVSVELNDTDSSAATKKTAA |
| | WNSGTSTLTITVNSKKTKDLVFTKENTITVQQYDSNGTKLEGSAVEITKLDEIK |
| | NALK |
| | (SEQ ID NO: 208) |
| >Camp2_SS_ | MKAKLLVLLCTFTATYAVEPTTTISATSTHELSASDARNSIQLLNAHIATLQSV |
| Flu- | QKSVPGSDYSDQIRDLLKAAFDLRGLIETLAHGGIPFYDPSTIMPRIKLVATTID |
| HA_A/ | TIHTATTTLQNKVRPAHVELGLEVTKAVLLTANPASTAKELDAEGAALKARLE |
| NewCaledonia/ | KVSQYPDLTPNDVATVYVRTNFSKTIWQVRANRDRYILGHKSAAVYKTINHA |
| 20/1999 | ITKAVGVRLNPKTTVGNIQAARTELLAAYQTAFNSPDVKKAA (SEQ ID NO: |
| | 209) |
| >Camp2_SS_ | MKAILVVLLYTFATANAVEPTTTISATSTHELSASDARNSIQLLNAHIATLQSV |
| Flu- | QKSVPGSDYSDQIRDLLKAAFDLRGLIETLAHGGIPFYDPSTIMPRIKLVATTID |
| HA_A/ | TIHTATTTLQNKVRPAHVELGLEVTKAVLLTANPASTAKELDAEGAALKARLE |
| California/7/ | KVSQYPDLTPNDVATVYVRTNFSKTIWQVRANRDRYILGHKSAAVYKTLNHA |
| 2009 | ITKAVGVRLNPKTTVGNIQAARTELLAAYQTAFNSPDVKKAA (SEQ ID NO: |
| | 210) |
| >Camp2_SS_ | MKTIIALSYILCLVFAVEPTTTISATSTHELSASDARNSIQLLNAHIATLQSVQKS |
| Flu- | VPGSDYSDQIRDLLKAAFDLRGLIETLAHGGIPFYDPSTIMPRIKLVATTIDTIHT |
| HA_A/ | ATTTLQNKVRPAHVELGLEVTKAVLLTANPASTAKELDAEGAALKARLEKVS |
| Moscow/10/1999 | QYPDLTPNDVATVYVRTNFSKTIWQVRANRDRYILGHKSAAVYKTLNHAITK |
| | AVGVRLNPKTTVGNIQAARTELLAAYQTAFNSPDVKKAA (SEQ ID NO: 211) |
| >Camp2_SS_ | MKAIIVLLMVVTSNAVEPTTTISATSTHELSASDARNSIQLLNAHIATLQSVQKS |
| Flu- | VPGSDYSDQIRDLLKAAFDLRGLIETLAHGGIPFYDPSTIMPRIKLVATTIDTIHT |
| HA B/ | ATTTLQNKVRPAHVELGLEVTKAVLLTANPASTAKELDAEGAALKARLEKVS |
| Phuket/3073/ | QYPDLTPNDVATVYVRTNFSKTIWQVRANRDRYILGHKSAAVYKTLNHAITK |
| 2013 | AVGVRLNPKTTVGNIQAARTELLAAYQTAFNSPDVKKAA (SEQ ID NO: 164) |
| >Camp2_SS- | MFVFLVLLPLVSVEPTTTISATSTHELSASDARNSIQLLNAHIATLQSVQKSVPG |
| spike_CoV2_ | SDYSDQIRDLLKAAFDLRGLIETLAHGGIPFYDPSTIMPRIKLVATTIDTIHTATT |
| short | TLQNKVRPAHVELGLEVTKAVLLTANPASTAKELDAEGAALKARLEKVSQYP |
| | DLTPNDVATVYVRTNFSKTIWQVRANRDRYILGHKSAAVYKTLNHAITKAVG |
| | VRLNPKTTVGNIQAARTELLAAYQTAFNSPDVKKAA (SEQ ID NO: 212) |
| >Camp2_SS- | MFLLTTKRTMFVFLVLLPLVSVEPTTTISATSTHELSASDARNSIQLLNAHIATL |
| spike_CoV2_ | QSVQKSVPGSDYSDQIRDLLKAAFDLRGLIETLAHGGIPFYDPSTIMPRIKLVAT |
| long | TIDTIHTATTTLQNKVRPAHVELGLEVTKAVLLTANPASTAKELDAEGAALKA |
| | RLEKVSQYPDLTPNDVATVYVRTNFSKTIWQVRANRDRYILGHKSAAVYKTL |
| | NHAITKAVGVRLNPKTTVGNIQAARTELLAAYQTAFNSPDVKKAA (SEQ ID |
| | NO: 213) |
| >Camp2_SS_ | MVPQALLFVPLLVFPLCFGVEPTTTISATSTHELSASDARNSIQLLNAHIATLQS |
| Rabies- | VQKSVPGSDYSDQIRDLLKAAFDLRGLIETLAHGGIPFYDPSTIMPRIKLVATTI |
| G_Pasteur- | DTIHTATTTLQNKVRPAHVELGLEVTKAVLLTANPASTAKELDAEGAALKAR |
| vaccine | LEKVSQYPDLTPNDVATVYVRTNFSKTIWQVRANRDRYILGHKSAAVYKTLN |
| | HAITKAVGVRLNPKTTVGNIQAARTELLAAYQTAFNSPDVKKAA (SEQ ID NO: |
| | 214) |
| >Camp2_SS_ | MGTVNKPVVGVLMGFGIITGTLRITNPVRAVEPTTTISATSTHELSASDARNSIQ |
| VZV- | LLNAHIATLQSVQKSVPGSDYSDQIRDLLKAAFDLRGLIETLAHGGIPFYDPSTI |
| gE_Oka- | MPRIKLVATTIDTIHTATTTLQNKVRPAHVELGLEVTKAVLLTANPASTAKEL |
| vaccine | DAEGAALKARLEKVSQYPDLTPNDVATVYVRTNFSKTIWQVRANRDRYILGH |
| | KSAAVYKTLNHAITKAVGVRLNPKTTVGNIQAARTELLAAYQTAFNSPDVKK |
| | AA |
| | (SEQ ID NO: 215) |
| >Camp2_SS_ | MFLIQCLISAVIFYIQVTNAVEPTTTISATSTHELSASDARNSIQLLNAHIATLQS |
| VZV- | VQKSVPGSDYSDQIRDLLKAAFDLRGLIETLAHGGIPFYDPSTIMPRIKLVATTI |
| gI_Oka- | DTIHTATTTLQNKVRPAHVELGLEVTKAVLLTANPASTAKELDAEGAALKAR |
| vaccine | LEKVSQYPDLTPNDVATVYVRTNFSKTIWQVRANRDRYILGHKSAAVYKTLN |
| | HAITKAVGVRLNPKTTVGNIQAARTELLAAYQTAFNSPDVKKAA (SEQ ID NO: |
| | 216) |
| >Camp2_SS_ | MQALGIKTEHFIIMCLLSGHAVEPTTTISATSTHELSASDARNSIQLLNAHIATL |
| VZV- | QSVQKSVPGSDYSDQIRDLLKAAFDLRGLIETLAHGGIPFYDPSTIMPRIKLVAT |
| gK_Oka- | TIDTIHTATTTLQNKVRPAHVELGLEVTKAVLLTANPASTAKELDAEGAALKA |
| vaccine | RLEKVSQYPDLTPNDVATVYVRTNFSKTIWQVRANRDRYILGHKSAAVYKTL |
| | NHAITKAVGVRLNPKTTVGNIQAARTELLAAYQTAFNSPDVKKAA (SEQ ID |
| | NO: 217) |
| >Camp2_SS_ | MGLKVNVSAIFMAVLLTLQTPTGVEPTTTISATSTHELSASDARNSIQLLNAHI |
| Measles- | ATLQSVQKSVPGSDYSDQIRDLLKAAFDLRGLIETLAHGGIPFYDPSTIMPRIKL |
| F_Edmonston- | VATTIDTIHTATTTLQNKVRPAHVELGLEVTKAVLLTANPASTAKELDAEGAA |
| Zagreb- | LKARLEKVSQYPDLTPNDVATVYVRTNFSKTIWQVRANRDRYILGHKSAAVY |
| vaccine | KTLNHAITKAVGVRLNPKTTVGNIQAARTELLAAYQTAFNSPDVKKAA |
| | (SEQ ID NO: 218) |
| >Camp2_SS_ | MGAAAALTAVVLQGYNPPAYGVEPTTTISATSTHELSASDARNSIQLLNAHIA |
| Rubella- | TLQSVQKSVPGSDYSDQIRDLLKAAFDLRGLIETLAHGGIPFYDPSTIMPRIKLV |
| E1_RA27/3- | ATTIDTIHTATTTLQNKVRPAHVELGLEVTKAVLLTANPASTAKELDAEGAAL |
| vaccine | KARLEKVSQYPDLTPNDVATVYVRTNFSKTIWQVRANRDRYILGHKSAAVYK |
| | TLNHAITKAVGVRLNPKTTVGNIQAARTELLAAYQTAFNSPDVKKAA (SEQ ID |
| | NO: 219) |
| >Camp2_SS_ | MGAPQAFLAGLLLAAVAVGTARAVEPTTTISATSTHELSASDARNSIQLLNAHI |
| Rubella- | ATLQSVQKSVPGSDYSDQIRDLLKAAFDLRGLIETLAHGGIPFYDPSTIMPRIKL |
| E2_RA27/3- | VATTIDTIHTATTTLQNKVRPAHVELGLEVTKAVLLTANPASTAKELDAEGAA |
| vaccine | LKARLEKVSQYPDLTPNDVATVYVRTNFSKTIWQVRANRDRYILGHKSAAVY |
| | KTLNHAITKAVGVRLNPKTTVGNIQAARTELLAAYQTAFNSPDVKKAA |
| | (SEQ ID NO: 220) |
| >Camp2_SS_ | MKVFLVTCLGFAVFSSSVCVEPTTTISATSTHELSASDARNSIQLLNAHIATLQS |
| Mumps- | VQKSVPGSDYSDQIRDLLKAAFDLRGLIETLAHGGIPFYDPSTIMPRIKLVATTI |
| F_Miyahara- | DTIHTATTTLQNKVRPAHVELGLEVTKAVLLTANPASTAKELDAEGAALKAR |
| vaccine | LEKVSQYPDLTPNDVATVYVRTNFSKTIWQVRANRDRYILGHKSAAVYKTLN |
| | HAITKAVGVRLNPKTTVGNIQAARTELLAAYQTAFNSPDVKKAA (SEQ ID NO: |
| | 221) |
| >Camp2_SS_ | MGVTGILQLPRDRFKRTSFFLWVIILFQRTFSVEPTTTISATSTHELSASDARNSI |
| Ebola- | QLLNAHIATLQSVQKSVPGSDYSDQIRDLLKAAFDLRGLIETLAHGGIPFYDPS |
| GP_Mayinga- | TIMPRIKLVATTIDTIHTATTTLQNKVRPAHVELGLEVTKAVLLTANPASTAKE |
| 76 | LDAEGAALKARLEKVSQYPDLTPNDVATVYVRTNFSKTIWQVRANRDRYILG |
| | HKSAAVYKTLNHAITKAVGVRLNPKTTVGNIQAARTELLAAYQTAFNSPDVK |
| | KAA |
| | (SEQ ID NO: 222) |
| >Camp2_SS_ | MRSLIIFLLFPSIIYSVEPTTTISATSTHELSASDARNSIQLLNAHIATLQSVQKSV |
| VSV- | PGSDYSDQIRDLLKAAFDLRGLIETLAHGGIPFYDPSTIMPRIKLVATTIDTIHTA |
| G_Indiana | TTTLQNKVRPAHVELGLEVTKAVLLTANPASTAKELDAEGAALKARLEKVSQ |
| | YPDLTPNDVATVYVRTNFSKTIWQVRANRDRYILGHKSAAVYKTLNHAITKA |
| | VGVRLNPKTTVGNIQAARTELLAAYQTAFNSPDVKKAA (SEQ ID NO: 223) |
| >PITP_SS_ | MKAKLLVLLCTFTATYAAGPTVTVIPVGREGGDITISGKGFSTTGFGVYVAVA |
| Flu- | PASVPEFYGNSDKFYGYDPSKDTTESPSTIWVYTPSQKAIGSRFAQGRPMNND |
| HA_A/ | GSFTITMKAPPFEQGKDFVVLTTKAHGVGKTDHSDDTRTPVTYREATPAPTGP |
| NewCaledonia/ | KTPIAPSKQPSKQAAPSKQVKPSKQAGPNKQSTTPQQKTAEHRSQTPAAHRTM |
| 20/1999 | TKQVCTIGASKVTSGSLTWGIRTSFTSYLRGPIANGSWKLSGGANWNGSAFTF |
| | PLTSGSFDPATKSGSLKYSGSVHMTGHHGILDMTLAEPSLQIKGSTGHLYLDV |
| | KSSSMDGKKTNYGRVDFATFGVSVSGNAAIKGSPVKLTATGAKAFAGFYRAG |
| | EPMNPLSTNLTLSAEKVCHNVTVDAVTGKVIGDDSGKGAGRGLPVT (SEQ ID |
| | NO: 224) |
| >PITP_SS_ | MKAILVVLLYTFATANAAGPTVTVIPVGREGGDITISGKGFSTTGFGVYVAVA |
| Flu- | PASVPEFYGNSDKFYGYDPSKDTTESPSTIWVYTPSQKAIGSRFAQGRPMNND |
| HA_A/ | GSFTITMKAPPFEQGKDFVVLTTKAHGVGKTDHSDDTRTPVTYREATPAPTGP |
| California/ | KTPIAPSKQPSKQAAPSKQVKPSKQAGPNKQSTTPQQKTAEHRSQTPAAHRTM |
| 7/2009 | TKQVCTIGASKVTSGSLTWGIRTSFTSYLRGPIANGSWKLSGGANWNGSAFTF |
| | PLTSGSFDPATKSGSLKYSGSVHMTGHHGILDMTLAEPSLQIKGSTGHLYLDV |
| | KSSSMDGKKTNYGRVDFATFGVSVSGNAAIKGSPVKLTATGAKAFAGFYRAG |
| | EPMNPLSTNLTLSAEKVCHNVTVDAVTGKVIGDDSGKGAGRGLPVT (SEQ ID |
| | NO: 225) |
| >PITP_SS_ | MKTIIALSYILCLVFAAGPTVTVIPVGREGGDITISGKGFSTTGFGVYVAVAPAS |
| Flu- | VPEFYGNSDKFYGYDPSKDTTESPSTIWVYTPSQKAIGSRFAQGRPMNNDGSF |
| HA_A/ | TITMKAPPFEQGKDFVVLTTKAHGVGKTDHSDDTRTPVTYREATPAPTGPKTP |
| Moscow/10/1999 | IAPSKQPSKQAAPSKQVKPSKQAGPNKQSTTPQQKTAEHRSQTPAAHRTMTK |
| | QVCTIGASKVTSGSLTWGIRTSFTSYLRGPIANGSWKLSGGANWNGSAFTFPLT |
| | SGSFDPATKSGSLKYSGSVHMTGHHGILDMTLAEPSLQIKGSTGHLYLDVKSS |
| | SMDGKKTNYGRVDFATFGVSVSGNAAIKGSPVKLTATGAKAFAGFYRAGEP |
| | MNPLSTNLTLSAEKVCHNVTVDAVTGKVIGDDSGKGAGRGLPVT (SEQ ID |
| | NO: 226) |
| >PITP_SS_ | MKAIIVLLMVVTSNAAGPTVTVIPVGREGGDITISGKGFSTTGFGVYVAVAPAS |
| Flu- | VPEFYGNSDKFYGYDPSKDTTESPSTIWVYTPSQKAIGSRFAQGRPMNNDGSF |
| HA_B/ | TITMKAPPFEQGKDFVVLTTKAHGVGKTDHSDDTRTPVTYREATPAPTGPKTP |
| Phuket/3073/ | IAPSKQPSKQAAPSKQVKPSKQAGPNKQSTTPQQKTAEHRSQTPAAHRTMTK |
| 2013 | QVCTIGASKVTSGSLTWGIRTSFTSYLRGPIANGSWKLSGGANWNGSAFTFPLT |
| | SGSFDPATKSGSLKYSGSVHMTGHHGILDMTLAEPSLQIKGSTGHLYLDVKSS |
| | SMDGKKTNYGRVDFATFGVSVSGNAAIKGSPVKLTATGAKAFAGFYRAGEP |
| | MNPLSTNLTLSAEKVCHNVTVDAVTGKVIGDDSGKGAGRGLPVT (SEQ ID |
| | NO: 227) |
| >PITP_SS- | MFVFLVLLPLVSAGPTVTVIPVGREGGDITISGKGFSTTGFGVYVAVAPASVPE |
| spike_CoV2_ | FYGNSDKFYGYDPSKDTTESPSTIWVYTPSQKAIGSRFAQGRPMNNDGSFTIT |
| short | MKAPPFEQGKDFVVLTTKAHGVGKTDHSDDTRTPVTYREATPAPTGPKTPIAP |
| | SKQPSKQAAPSKQVKPSKQAGPNKQSTTPQQKTAEHRSQTPAAHRTMTKQVC |
| | TIGASKVTSGSLTWGIRTSFTSYLRGPIANGSWKLSGGANWNGSAFTFPLTSGS |
| | FDPATKSGSLKYSGSVHMTGHHGILDMTLAEPSLQIKGSTGHLYLDVKSSSMD |
| | GKKTNYGRVDFATFGVSVSGNAAIKGSPVKLTATGAKAFAGFYRAGEPMNPL |
| | STNLTLSAEKVCHNVTVDAVTGKVIGDDSGKGAGRGLPVT (SEQ ID NO: 228) |
| >PITP_SS- | MFLLTTKRTMFVFLVLLPLVSAGPTVTVIPVGREGGDITISGKGFSTTGFGVYV |
| spike_CoV2_ | AVAPASVPEFYGNSDKFYGYDPSKDTTESPSTIWVYTPSQKAIGSRFAQGRPM |
| long | NNDGSFTITMKAPPFEQGKDFVVLTTKAHGVGKTDHSDDTRTPVTYREATPAP |
| | TGPKTPIAPSKQPSKQAAPSKQVKPSKQAGPNKQSTTPQQKTAEHRSQTPAAH |
| | RTMTKQVCTIGASKVTSGSLTWGIRTSFTSYLRGPIANGSWKLSGGANWNGSA |
| | FTFPLTSGSFDPATKSGSLKYSGSVHMTGHHGILDMTLAEPSLQIKGSTGHLYL |
| | DVKSSSMDGKKTNYGRVDFATFGVSVSGNAAIKGSPVKLTATGAKAFAGFYR |
| | AGEPMNPLSTNLTLSAEKVCHNVTVDAVTGKVIGDDSGKGAGRGLPVT (SEQ |
| | ID NO: 229) |
| >PITP_SS_ | MVPQALLFVPLLVFPLCFGAGPTVTVIPVGREGGDITISGKGFSTTGFGVYVAV |
| Rabies- | APASVPEFYGNSDKFYGYDPSKDTTESPSTIWVYTPSQKAIGSRFAQGRPMNN |
| G_Pasteur- | DGSFTITMKAPPFEQGKDFVVLTTKAHGVGKTDHSDDTRTPVTYREATPAPTG |
| vaccine | PKTPIAPSKQPSKQAAPSKQVKPSKQAGPNKQSTTPQQKTAEHRSQTPAAHRT |
| | MTKQVCTIGASKVTSGSLTWGIRTSFTSYLRGPIANGSWKLSGGANWNGSAFT |
| | FPLTSGSFDPATKSGSLKYSGSVHMTGHHGILDMTLAEPSLQIKGSTGHLYLDV |
| | KSSSMDGKKTNYGRVDFATFGVSVSGNAAIKGSPVKLTATGAKAFAGFYRAG |
| | EPMNPLSTNLTLSAEKVCHNVTVDAVTGKVIGDDSGKGAGRGLPVT (SEQ ID |
| | NO: 230) |
| >PITP_SS_ | MGTVNKPVVGVLMGFGIITGTLRITNPVRAAGPTVTVIPVGREGGDITISGKGF |
| VZV-gE_Oka- | STTGFGVYVAVAPASVPEFYGNSDKFYGYDPSKDTTESPSTIWVYTPSQKAIGS |
| vaccine | RFAQGRPMNNDGSFTITMKAPPFEQGKDFVVLTTKAHGVGKTDHSDDTRTPV |
| | TYREATPAPTGPKTPIAPSKQPSKQAAPSKQVKPSKQAGPNKQSTTPQQKTAE |
| | HRSQTPAAHRTMTKQVCTIGASKVTSGSLTWGIRTSFTSYLRGPIANGSWKLS |
| | GGANWNGSAFTFPLTSGSFDPATKSGSLKYSGSVHMTGHHGILDMTLAEPSLQ |
| | IKGSTGHLYLDVKSSSMDGKKTNYGRVDFATFGVSVSGNAAIKGSPVKLTAT |
| | GAKAFAGFYRAGEPMNPLSTNLTLSAEKVCHNVTVDAVTGKVIGDDSGKGA |
| | GRGLPVT |
| | (SEQ ID NO: 231) |
| >PITP_SS_ | MFLIQCLISAVIFYIQVTNAAGPTVTVIPVGREGGDITISGKGFSTTGFGVYVAV |
| VZV-gI_Oka- | APASVPEFYGNSDKFYGYDPSKDTTESPSTIWVYTPSQKAIGSRFAQGRPMNN |
| vaccine | DGSFTITMKAPPFEQGKDFVVLTTKAHGVGKTDHSDDTRTPVTYREATPAPTG |
| | PKTPIAPSKQPSKQAAPSKQVKPSKQAGPNKQSTTPQQKTAEHRSQTPAAHRT |
| | MTKQVCTIGASKVTSGSLTWGIRTSFTSYLRGPIANGSWKLSGGANWNGSAFT |
| | FPLTSGSFDPATKSGSLKYSGSVHMTGHHGILDMTLAEPSLQIKGSTGHLYLDV |
| | KSSSMDGKKTNYGRVDFATFGVSVSGNAAIKGSPVKLTATGAKAFAGFYRAG |
| | EPMNPLSTNLTLSAEKVCHNVTVDAVTGKVIGDDSGKGAGRGLPVT (SEQ ID |
| | NO: 232) |
| >PITP_SS_ | MQALGIKTEHFIIMCLLSGHAAGPTVTVIPVGREGGDITISGKGFSTTGFGVYV |
| VZV-gK_Oka- | AVAPASVPEFYGNSDKFYGYDPSKDTTESPSTIWVYTPSQKAIGSRFAQGRPM |
| vaccine | NNDGSFTITMKAPPFEQGKDFVVLTTKAHGVGKTDHSDDTRTPVTYREATPAP |
| | TGPKTPIAPSKQPSKQAAPSKQVKPSKQAGPNKQSTTPQQKTAEHRSQTPAAH |
| | RTMTKQVCTIGASKVTSGSLTWGIRTSFTSYLRGPIANGSWKLSGGANWNGSA |
| | FTFPLTSGSFDPATKSGSLKYSGSVHMTGHHGILDMTLAEPSLQIKGSTGHLYL |
| | DVKSSSMDGKKTNYGRVDFATFGVSVSGNAAIKGSPVKLTATGAKAFAGFYR |
| | AGEPMNPLSTNLTLSAEKVCHNVTVDAVTGKVIGDDSGKGAGRGLPVT (SEQ |
| | ID NO: 233) |
| >PITP_SS | MGLKVNVSAIFMAVLLTLQTPTGAGPTVTVIPVGREGGDITISGKGFSTTGFGV |
| Measles- | YVAVAPASVPEFYGNSDKFYGYDPSKDTTESPSTIWVYTPSQKAIGSRFAQGR |
| F_Edmonston- | PMNNDGSFTITMKAPPFEQGKDFVVLTTKAHGVGKTDHSDDTRTPVTYREAT |
| Zagreb- | PAPTGPKTPIAPSKQPSKQAAPSKQVKPSKQAGPNKQSTTPQQKTAEHRSQTPA |
| vaccine | AHRTMTKQVCTIGASKVTSGSLTWGIRTSFTSYLRGPIANGSWKLSGGANWN |
| | GSAFTFPLTSGSFDPATKSGSLKYSGSVHMTGHHGILDMTLAEPSLQIKGSTGH |
| | LYLDVKSSSMDGKKTNYGRVDFATFGVSVSGNAAIKGSPVKLTATGAKAFAG |
| | FYRAGEPMNPLSTNLTLSAEKVCHNVTVDAVTGKVIGDDSGKGAGRGLPVT |
| | (SEQ ID NO: 234) |
| >PITP_SS_ | MGAAAALTAVVLQGYNPPAYGAGPTVTVIPVGREGGDITISGKGFSTTGFGVY |
| Rubella- | VAVAPASVPEFYGNSDKFYGYDPSKDTTESPSTIWVYTPSQKAIGSRFAQGRP |
| E1_RA27/3- | MNNDGSFTITMKAPPFEQGKDFVVLTTKAHGVGKTDHSDDTRTPVTYREATP |
| vaccine | APTGPKTPIAPSKQPSKQAAPSKQVKPSKQAGPNKQSTTPQQKTAEHRSQTPA |
| | AHRTMTKQVCTIGASKVTSGSLTWGIRTSFTSYLRGPIANGSWKLSGGANWN |
| | GSAFTFPLTSGSFDPATKSGSLKYSGSVHMTGHHGILDMTLAEPSLQIKGSTGH |
| | LYLDVKSSSMDGKKTNYGRVDFATFGVSVSGNAAIKGSPVKLTATGAKAFAG |
| | FYRAGEPMNPLSTNLTLSAEKVCHNVTVDAVTGKVIGDDSGKGAGRGLPVT |
| | (SEQ ID NO: 235) |
| >PITP_SS_ | MGAPQAFLAGLLLAAVAVGTARAAGPTVTVIPVGREGGDITISGKGFSTTGFG |
| Rubella- | VYVAVAPASVPEFYGNSDKFYGYDPSKDTTESPSTIWVYTPSQKAIGSRFAQG |
| E2_RA27/3- | RPMNNDGSFTITMKAPPFEQGKDFVVLTTKAHGVGKTDHSDDTRTPVTYREA |
| vaccine | TPAPTGPKTPIAPSKQPSKQAAPSKQVKPSKQAGPNKQSTTPQQKTAEHRSQTP |
| | AAHRTMTKQVCTIGASKVTSGSLTWGIRTSFTSYLRGPIANGSWKLSGGANW |
| | NGSAFTFPLTSGSFDPATKSGSLKYSGSVHMTGHHGILDMTLAEPSLQIKGSTG |
| | HLYLDVKSSSMDGKKTNYGRVDFATFGVSVSGNAAIKGSPVKLTATGAKAFA |
| | GFYRAGEPMNPLSTNLTLSAEKVCHNVTVDAVTGKVIGDDSGKGAGRGLPVT |
| | (SEQ ID NO: 236) |
| >PITP_SS | MKVFLVTCLGFAVFSSSVCAGPTVTVIPVGREGGDITISGKGFSTTGFGVYVAV |
| Mumps- | APASVPEFYGNSDKFYGYDPSKDTTESPSTIWVYTPSQKAIGSRFAQGRPMNN |
| F_Miyahara- | DGSFTITMKAPPFEQGKDFVVLTTKAHGVGKTDHSDDTRTPVTYREATPAPTG |
| vaccine | PKTPIAPSKQPSKQAAPSKQVKPSKQAGPNKQSTTPQQKTAEHRSQTPAAHRT |
| | MTKQVCTIGASKVTSGSLTWGIRTSFTSYLRGPIANGSWKLSGGANWNGSAFT |
| | FPLTSGSFDPATKSGSLKYSGSVHMTGHHGILDMTLAEPSLQIKGSTGHLYLDV |
| | KSSSMDGKKTNYGRVDFATFGVSVSGNAAIKGSPVKLTATGAKAFAGFYRAG |
| | EPMNPLSTNLTLSAEKVCHNVTVDAVTGKVIGDDSGKGAGRGLPVT (SEQ ID |
| | NO: 237) |
| >PITP_SS_ | MGVTGILQLPRDRFKRTSFFLWVIILFQRTFSAGPTVTVIPVGREGGDITISGKG |
| Ebola- | FSTTGFGVYVAVAPASVPEFYGNSDKFYGYDPSKDTTESPSTIWVYTPSQKAIG |
| GP_Mayinga- | SRFAQGRPMNNDGSFTITMKAPPFEQGKDFVVLTTKAHGVGKTDHSDDTRTP |
| 76 | VTYREATPAPTGPKTPIAPSKQPSKQAAPSKQVKPSKQAGPNKQSTTPQQKTA |
| | EHRSQTPAAHRTMTKQVCTIGASKVTSGSLTWGIRTSFTSYLRGPIANGSWKL |
| | SGGANWNGSAFTFPLTSGSFDPATKSGSLKYSGSVHMTGHHGILDMTLAEPSL |
| | QIKGSTGHLYLDVKSSSMDGKKTNYGRVDFATFGVSVSGNAAIKGSPVKLTA |
| | TGAKAFAGFYRAGEPMNPLSTNLTLSAEKVCHNVTVDAVTGKVIGDDSGKGA |
| | GRGLPVT |
| | (SEQ ID NO: 238) |
| >PITP_SS_ | MRSLIIFLLFPSIIYSAGPTVTVIPVGREGGDITISGKGFSTTGFGVYVAVAPASV |
| VSV- | PEFYGNSDKFYGYDPSKDTTESPSTIWVYTPSQKAIGSRFAQGRPMNNDGSFTI |
| G_Indiana | TMKAPPFEQGKDFVVLTTKAHGVGKTDHSDDTRTPVTYREATPAPTGPKTPIA |
| | PSKQPSKQAAPSKQVKPSKQAGPNKQSTTPQQKTAEHRSQTPAAHRTMTKQV |
| | CTIGASKVTSGSLTWGIRTSFTSYLRGPIANGSWKLSGGANWNGSAFTFPLTSG |
| | SFDPATKSGSLKYSGSVHMTGHHGILDMTLAEPSLQIKGSTGHLYLDVKSSSM |
| | DGKKTNYGRVDFATFGVSVSGNAAIKGSPVKLTATGAKAFAGFYRAGEPMNP |
| | LSTNLTLSAEKVCHNVTVDAVTGKVIGDDSGKGAGRGLPVT (SEQ ID NO: 239) |

TABLE 8
Results of SignalP 6.0 Analysis From All Tested Sequences displayed in Table 7

| | | SS | SS | Cleavage | Cleavage | [E] × | |
|---|---|---|---|---|---|---|---|
| NAME | Length | Length | likelihood | post | probability | [G] | Rank |
| >OspA ST1_SS_Flu- | 273 | 17 | 0.9998 | 17 | 0.9838 | 0.984 | 2 |
| HA_A/NewCaledonia/ | | | | | | | |
| 20/1999 | | | | | | | |
| >OspA ST1_SS_Flu- | 273 | 17 | 0.9998 | 17 | 0.9823 | 0.982 | 6 |
| HA_A/California/7/ | | | | | | | |
| 2009 | | | | | | | |
| >OspA ST1_SS_Flu- | 272 | 16 | 0.9997 | 22 | 0.4879 | 0.488 | 13 |
| HA_A/Moscow/10/ | | | | | | | |
| 1999 | | | | | | | |
| >OspA ST1_SS_Flu- | 271 | 15 | 0.9998 | 15 | 0.9687 | 0.969 | 9 |
| HA_B/Phuket/3073/ | | | | | | | |
| 2013 | | | | | | | |
| >OspA ST1_SS- | 268 | 12 | 0.6664 | 16 | 0.4985 | 0.332 | 15 |
| spike_CoV2_short | | | | | | | |
| >OspA ST1_SS- | 277 | 21 | 1 | 21 | 0.9895 | 0.99 | 1 |
| spike_CoV2_long | | | | | | | |
| >OspA | 275 | 19 | 0.9998 | 19 | 0.9831 | 0.983 | 3 |
| ST1_SS_Rabies- | | | | | | | |
| G_Pasteur-vaccins | | | | | | | |
| >OspA ST1_SS_VZV- | 286 | 30 | 0.8323 | 30 | 0.6537 | 0.544 | 12 |
| gE_Oka-vaccine | | | | | | | |
| >OspA ST1_SS_VZV- | 276 | 20 | 0.9998 | 20 | 0.9831 | 0.983 | 3 |
| gI_Oka-vaccine | | | | | | | |
| >OspA ST1_SS_VZV- | 277 | 21 | 0.5 | 19 | 0.4973 | 0.249 | 16 |
| gK_Oka-vaccine | | | | | | | |
| >OspA | 279 | 23 | 0.9891 | 27 | 0.3741 | 0.37 | 14 |
| ST1_SS_Measles- | | | | | | | |
| F_Edmonston-Zagreb- | | | | | | | |
| vaccine | | | | | | | |
| >OspA | 277 | 21 | 1 | 14 | 0.9695 | 0.97 | 8 |
| ST1_SS_Rubella- | | | | | | | |
| E1_RA27/3-vaccine | | | | | | | |
| >OspA | 279 | 23 | 0.9998 | 23 | 0.9828 | 0.983 | 5 |
| ST1_SS_Rubella- | | | | | | | |
| E2_RA27/3-vaccine | | | | | | | |
| >OspA | 275 | 19 | 0.9999 | 20 | 0.8131 | 0.813 | 10 |
| ST1_SS_Mumps- | | | | | | | |
| F_Miyahara-vaccine | | | | | | | |
| >OspA ST1_SS_Ebola- | 288 | 32 | 0.832 | 32 | 0.8105 | 0.674 | 11 |
| GP_Mayinga-76 | | | | | | | |
| >OspA ST1_SS_VSV- | 272 | 16 | 0.9998 | 16 | 0.9749 | 0.975 | 7 |
| G_Indiana | | | | | | | |
| >Camp2_SS_Flu- | 256 | 17 | 0.9998 | 17 | 0.9888 | 0.989 | 1 |
| HA_A/NewCaledonia/ | | | | | | | |
| 20/1999 | | | | | | | |
| >Camp2_SS_Flu- | 256 | 17 | 0.9998 | 17 | 0.9857 | 0.986 | 4 |
| HA_A/California/7/ | | | | | | | |
| 2009 | | | | | | | |
| >Camp2_SS_Flu- | 255 | 16 | 0.9998 | 16 | 0.9722 | 0.972 | 12 |
| HA_A/Moscow/10/ | | | | | | | |
| 1999 | | | | | | | |
| >Camp2_SS_Flu- | 254 | 15 | 0.9998 | 15 | 0.9852 | 0.985 | 6 |
| HA_B/Phuket/3073/ | | | | | | | |
| 2013 | | | | | | | |
| >Camp2_SS- | 251 | 12 | 0.9998 | 13 | 0.9855 | 0.985 | 5 |
| spike_CoV2_short | | | | | | | |
| >Camp2_SS- | 260 | 21 | 0.9998 | 24 | 0.7542 | 0.754 | 13 |
| spike_CoV2_long | | | | | | | |
| >Camp2_SS_Rabies- | 258 | 19 | 0.9998 | 19 | 0.9872 | 0.987 | 2 |
| G_Pasteur-vaccins | | | | | | | |
| >Camp2_SS_VZV- | 269 | 30 | 0.9997 | 30 | 0.9743 | 0.974 | 11 |
| gE_Oka-vaccine | | | | | | | |
| >Camp2_SS_VZV- | 259 | 20 | 0.9998 | 20 | 0.984 | 0.984 | 7 |
| gI_Oka-vaccine | | | | | | | |
| >Camp2_SS_VZV- | 260 | 21 | 0.3334 | 0 | 0 | 0 | 16 |
| gK_Oka-vaccine | | | | | | | |
| >Camp2_SS_Measles- | 262 | 23 | 0.9998 | 26 | 0.9802 | 0.98 | 9 |
| F_Edmonston-Zagreb- | | | | | | | |
| vaccine | | | | | | | |
| >Camp2_SS_Rubella- | 260 | 21 | 0.6661 | 21 | 0.4954 | 0.33 | 15 |
| E1_RA27/3-vaccine | | | | | | | |
| >Camp2_SS_Rubella- | 262 | 23 | 0.9998 | 23 | 0.9805 | 0.98 | 8 |
| E2_RA27/3-vaccine | | | | | | | |
| >Camp2_SS_Mumps- | 258 | 19 | 0.9998 | 20 | 0.6496 | 0.649 | 14 |
| F_Miyahara-vaccine | | | | | | | |
| >Camp2_SS_Ebola- | 271 | 32 | 0.9996 | 32 | 0.9769 | 0.977 | 10 |
| GP_Mayinga-76 | | | | | | | |
| >Camp2_SS_VSV- | 255 | 16 | 0.9998 | 16 | 0.9858 | 0.986 | 3 |
| G_Indiana | | | | | | | |
| >PITP_SS_Flu- | 416 | 17 | 0.9999 | 17 | 0.9859 | 0.986 | 1 |
| HA_A/NewCaledonia/ | | | | | | | |
| 20/1999 | | | | | | | |
| >PITP_SS_Flu- | 416 | 17 | 0.9999 | 17 | 0.9834 | 0.983 | 5 |
| HA_A/California/7/ | | | | | | | |
| 2009 | | | | | | | |
| >PITP_SS_Flu- | 415 | 16 | 0.9998 | 16 | 0.9769 | 0.977 | 14 |
| HA_A/Moscow/10/ | | | | | | | |
| 1999 | | | | | | | |
| >PITP_SS_Flu- | 414 | 15 | 0.9999 | 15 | 0.9845 | 0.984 | 4 |
| HA_B/Phuket/3073/ | | | | | | | |
| 2013 | | | | | | | |
| >PITP_SS- | 411 | 12 | 0.9999 | 13 | 0.9811 | 0.981 | 11 |
| spike_CoV2_short | | | | | | | |
| >PITP_SS- | 420 | 21 | 0.9999 | 22 | 0.9832 | 0.983 | 8 |
| spike_CoV2_long | | | | | | | |
| >PITP_SS_Rabies- | 418 | 19 | 0.9999 | 19 | 0.9851 | 0.985 | 2 |
| G_Pasteur-vaccins | | | | | | | |
| >PITP_SS_VZV- | 429 | 30 | 0.9998 | 30 | 0.9772 | 0.977 | 13 |
| gE_Oka-vaccine | | | | | | | |
| >PITP_SS_VZV- | 419 | 20 | 0.9998 | 20 | 0.9846 | 0.984 | 3 |
| gI_Oka-vaccine | | | | | | | |
| >PITP_SS_VZV- | 420 | 21 | 0.9999 | 22 | 0.5427 | 0.543 | 16 |
| gK_Oka-vaccine | | | | | | | |
| >PITP_SS_Measles- | 422 | 23 | 0.9999 | 24 | 0.9826 | 0.983 | 9 |
| F_Edmonston-Zagreb- | | | | | | | |
| vaccine | | | | | | | |
| >PITP_SS_Rubella- | 420 | 21 | 0.9944 | 21 | 0.7335 | 0.729 | 15 |
| E1_RA27/3-vaccine | | | | | | | |
| >PITP_SS_Rubella- | 422 | 23 | 0.9998 | 23 | 0.9825 | 0.982 | 10 |
| E2_RA27/3-vaccine | | | | | | | |
| >PITP_SS_Mumps- | 418 | 19 | 0.9998 | 20 | 0.9833 | 0.983 | 6 |
| F_Miyahara-vaccine | | | | | | | |
| >PITP_SS_Ebola- | 431 | 32 | 0.9987 | 32 | 0.9817 | 0.98 | 12 |
| GP_Mayinga-76 | | | | | | | |
| >PITP_SS_VSV- | 415 | 16 | 0.9998 | 16 | 0.9833 | 0.983 | 6 |
| G_Indiana | | | | | | | |

Additionally, signal peptides and their cleavage sites are less conserved than the mature regions of protein (Nielsen et al. (2019), The Protein Journal, 38:200-216). Therefore, to understand differences amongst signal sequences at a species and subtype level, homology of several hemagglutinin signal sequences was analyzed. Table 2 displays several strains tested for the Influenza A (subtypes H1N1 and H3N2) and Influenza B (Victoria and Yamagata lineages) and the resulting consensus hemagglutinin signal sequences are shown in Table 2.

Based on these in silico results, the panel was narrowed to the constructs which were selected for downstream testing. The final combination of signal sequence and antigens selected accompanied by each construct's respective SignalP 6.0 scores is shown in Table 8 and Table 9.

The amino acid sequences corresponding to this mRNA panel design are shown in Table 7. In Table 7, the construct identification name provides information about the antigen, along with a brief representation of the viral glycoprotein signal sequence, and an indication of whether a transmembrane domain was incorporated (noted as “TMB”).

Subsequently, mRNA was produced from the constructs displayed in Table 7. The mRNA production methods were the same as described in Example 1. The parameters of the mRNA produced from the constructs in this panel including efficiency of the capping reaction and the length of the poly(A) tail were determined. All mRNA were adequately capped and polyA tailed.

TABLE 9
Results of SignalP 6.0 Analysis for the Selected Signal Sequences

| | | SS | SS | Cleavage | Cleavage | [E] × | |
|---|---|---|---|---|---|---|---|
| NAME | Length | Length | likelihood | post | probability | [G] | Rank |
| >OspA ST1_SS_Flu- | 273 | 17 | 0.9998 | 17 | 0.9823 | 0.9821 | 9 |
| HA_A/California/7/ | | | | | | | |
| 2009 | | | | | | | |
| >OspA ST1_SS_Flu- | 271 | 15 | 0.9998 | 15 | 0.9687 | 0.9685 | 12 |
| HA_B/Phuket/3073/ | | | | | | | |
| 2013 | | | | | | | |
| >OspA | 275 | 19 | 0.9998 | 19 | 0.9831 | 0.9829 | 6 |
| ST1_SS_Rabies- | | | | | | | |
| G_Pasteur-vaccins | | | | | | | |
| >OspA | 276 | 20 | 0.9998 | 20 | 0.9831 | 0.9829 | 6 |
| ST1_SS_VZV- | | | | | | | |
| gI_Oka-vaccine | | | | | | | |
| >OspA | 288 | 32 | 0.832 | 32 | 0.8105 | 0.6743 | 14 |
| ST1_SS_Ebola- | | | | | | | |
| GP_Mayinga-76 | | | | | | | |
| >Camp2_SS_Flu- | 256 | 17 | 0.9998 | 17 | 0.9857 | 0.9855 | 6 |
| HA_A/California/7/ | | | | | | | |
| 2009 | | | | | | | |
| >Camp2_SS_Flu- | 254 | 15 | 0.9998 | 15 | 0.9852 | 0.9850 | 8 |
| HA_B/Phuket/3073/ | | | | | | | |
| 2013 | | | | | | | |
| >Camp2_SS_Rabies- | 258 | 19 | 0.9998 | 19 | 0.9872 | 0.9870 | 3 |
| G_Pasteur-vaccins | | | | | | | |
| >Camp2_SS_VZV- | 259 | 20 | 0.9998 | 20 | 0.984 | 0.9838 | 9 |
| gI_Oka-vaccine | | | | | | | |
| >Camp2_SS_Ebola- | 271 | 32 | 0.9996 | 32 | 0.9769 | 0.9765 | 13 |
| GP_Mayinga-76 | | | | | | | |
| >PITP_SS_Flu- | 416 | 17 | 0.9999 | 17 | 0.9834 | 0.9833 | 8 |
| HA_A/California/7/ | | | | | | | |
| 2009 | | | | | | | |
| >PITP_SS_Flu- | 414 | 15 | 0.9999 | 15 | 0.9845 | 0.9844 | 6 |
| HA_B/Phuket/3073/ | | | | | | | |
| 2013 | | | | | | | |
| >PITP_SS_Rabies- | 418 | 19 | 0.9999 | 19 | 0.9851 | 0.9850 | 2 |
| G_Pasteur-vaccins | | | | | | | |
| >PITP_SS_VZV- | 419 | 20 | 0.9998 | 20 | 0.9846 | 0.9844 | 5 |
| gI_Oka-vaccine | | | | | | | |
| >PITP_SS_Ebola- | 431 | 32 | 0.9987 | 32 | 0.9817 | 0.9804 | 15 |
| GP_Mayinga-76 | | | | | | | |

TABLE 10
Amino Acid Sequence Designs of Expanded mRNA Design Panel

| SEQ ID NO/ | |
|---|---|
| Description | Amino Acid sequence |
| OspA ST1 | MKQNVSSLDEKNSVSVDLPGEMKVLVSKEKNKDGKYDLIATVDKLEL |
| SEQ ID NO: 151 | KGTSDKSNGSGVLEGVKADKSKVKLTISDDLGQTTLEVFKEDGKTLVS |
| | KKVTSKDKSSTEEKFNEKGEVSEKIITRADGTRLEYTGIKSDGSGKAKE |
| | VLKGYDLKGELSSEKTTLVVKEGTVTLSKNISKSGEVSVELNDTDSSAA |
| | TKKTAAWNSGTSTLTITVNSKKTKDLVFTKENTITVQQYDSNGTKLEGS |
| | AVEITKLDEIKNALK |
| OspA ST1_SS_Flu- | MKAILVVLLYTFATANAKQNVSSLDEKNSVSVDLPGEMKVLVSKEKN |
| HA_A/California/7/ | KDGKYDLIATVDKLELKGTSDKSNGSGVLEGVKADKSKVKLTISDDLG |
| 2009 | QTTLEVFKEDGKTLVSKKVTSKDKSSTEEKFNEKGEVSEKIITRADGTR |
| SEQ ID NO: 152 | LEYTGIKSDGSGKAKEVLKGYDLKGELSSEKTTLVVKEGTVTLSKNISK |
| | SGEVSVELNDTDSSAATKKTAAWNSGTSTLTITVNSKKTKDLVFTKENT |
| | ITVQQYDSNGTKLEGSAVEITKLDEIKNALK |
| OspA ST1_SS_Flu- | MKAIIVLLMVVTSNAKQNVSSLDEKNSVSVDLPGEMKVLVSKEKNKD |
| HA_B/Phuket/3073/ | GKYDLIATVDKLELKGTSDKSNGSGVLEGVKADKSKVKLTISDDLGQT |
| 2013 | TLEVFKEDGKTLVSKKVTSKDKSSTEEKFNEKGEVSEKIITRADGTRLE |
| SEQ ID NO: 153 | YTGIKSDGSGKAKEVLKGYDLKGELSSEKTTLVVKEGTVTLSKNISKSG |
| | EVSVELNDTDSSAATKKTAAWNSGTSTLTITVNSKKTKDLVFTKENTIT |
| | VQQYDSNGTKLEGSAVEITKLDEIKNALK |
| OspA | MVPQALLFVPLLVFPLCFGKQNVSSLDEKNSVSVDLPGEMKVLVSKEK |
| ST1_SS_Rabies- | NKDGKYDLIATVDKLELKGTSDKSNGSGVLEGVKADKSKVKLTISDDL |
| G_Pasteur-vaccins | GQTTLEVFKEDGKTLVSKKVTSKDKSSTEEKFNEKGEVSEKIITRADGT |
| SEQ ID NO: 154 | RLEYTGIKSDGSGKAKEVLKGYDLKGELSSEKTTLVVKEGTVTLSKNIS |
| | KSGEVSVELNDTDSSAATKKTAAWNSGTSTLTITVNSKKTKDLVFTKE |
| | NTITVQQYDSNGTKLEGSAVEITKLDEIKNALK |
| OspA | MFLIQCLISAVIFYIQVTNAKQNVSSLDEKNSVSVDLPGEMKVLVSKEK |
| ST1_SS_VZV- | NKDGKYDLIATVDKLELKGTSDKSNGSGVLEGVKADKSKVKLTISDDL |
| gI_Oka-vaccine | GQTTLEVFKEDGKTLVSKKVTSKDKSSTEEKFNEKGEVSEKIITRADGT |
| SEQ ID NO: 155 | RLEYTGIKSDGSGKAKEVLKGYDLKGELSSEKTTLVVKEGTVTLSKNIS |
| | KSGEVSVELNDTDSSAATKKTAAWNSGTSTLTITVNSKKTKDLVFTKE |
| | NTITVQQYDSNGTKLEGSAVEITKLDEIKNALK |
| OspA | MGVTGILQLPRDRFKRTSFFLWVIILFQRTFSKQNVSSLDEKNSVSVDLP |
| ST1_SS_Ebola- | GEMKVLVSKEKNKDGKYDLIATVDKLELKGTSDKSNGSGVLEGVKAD |
| GP_Mayinga-76 | KSKVKLTISDDLGQTTLEVFKEDGKTLVSKKVTSKDKSSTEEKFNEKGE |
| SEQ ID NO: 156 | VSEKIITRADGTRLEYTGIKSDGSGKAKEVLKGYDLKGELSSEKTTLVV |
| | KEGTVTLSKNISKSGEVSVELNDTDSSAATKKTAAWNSGTSTLTITVNS |
| | KKTKDLVFTKENTITVQQYDSNGTKLEGSAVEITKLDEIKNALK |
| OspA ST1_SS_Flu- | MKAILVVLLYTFATANAKQNVSSLDEKNSVSVDLPGEMKVLVSKEKN |
| HA_A/California/7/ | KDGKYDLIATVDKLELKGTSDKSNGSGVLEGVKADKSKVKLTISDDLG |
| 2009_TMB | QTTLEVFKEDGKTLVSKKVTSKDKSSTEEKFNEKGEVSEKIITRADGTR |
| SEQ ID NO: 157 | LEYTGIKSDGSGKAKEVLKGYDLKGELSSEKTTLVVKEGTVTLSKNISK |
| | SGEVSVELNDTDSSAATKKTAAWNSGTSTLTITVNSKKTKDLVFTKENT |
| | ITVQQYDSNGTKLEGSAVEITKLDEIKNALKSGSILAIYSTVASSLVLVVS |
| | LGAISFGSG |
| OspA ST1_SS_Flu- | MKAIIVLLMVVTSNAKQNVSSLDEKNSVSVDLPGEMKVLVSKEKNKD |
| HA_B/Phuket/3073/ | GKYDLIATVDKLELKGTSDKSNGSGVLEGVKADKSKVKLTISDDLGQT |
| 2013_TMB | TLEVFKEDGKTLVSKKVTSKDKSSTEEKFNEKGEVSEKIITRADGTRLE |
| SEQ ID NO: 158 | YTGIKSDGSGKAKEVLKGYDLKGELSSEKTTLVVKEGTVTLSKNISKSG |
| | EVSVELNDTDSSAATKKTAAWNSGTSTLTITVNSKKTKDLVFTKENTIT |
| | VQQYDSNGTKLEGSAVEITKLDEIKNALKSGSSTAASSLAVTLMLAIFIV |
| | YMVGSG |
| OspA | MVPQALLFVPLLVFPLCFGKQNVSSLDEKNSVSVDLPGEMKVLVSKEK |
| ST1_SS_Rabies- | NKDGKYDLIATVDKLELKGTSDKSNGSGVLEGVKADKSKVKLTISDDL |
| G_Pasteur- | GQTTLEVFKEDGKTLVSKKVTSKDKSSTEEKFNEKGEVSEKIITRADGT |
| vaccins_TMB | RLEYTGIKSDGSGKAKEVLKGYDLKGELSSEKTTLVVKEGTVTLSKNIS |
| SEQ ID NO: 159 | KSGEVSVELNDTDSSAATKKTAAWNSGTSTLTITVNSKKTKDLVFTKE |
| | NTITVQQYDSNGTKLEGSAVEITKLDEIKNALKSGSVLLSAGALTALMLI |
| | IFLMTCWGSG |
| OspA | MFLIQCLISAVIFYIQVTNAKQNVSSLDEKNSVSVDLPGEMKVLVSKEK |
| ST1_SS_VZV- | NKDGKYDLIATVDKLELKGTSDKSNGSGVLEGVKADKSKVKLTISDDL |
| gI_Oka- | GQTTLEVFKEDGKTLVSKKVTSKDKSSTEEKFNEKGEVSEKIITRADGT |
| vaccine_TMB | RLEYTGIKSDGSGKAKEVLKGYDLKGELSSEKTTLVVKEGTVTLSKNIS |
| SEQ ID NO: 160 | KSGEVSVELNDTDSSAATKKTAAWNSGTSTLTITVNSKKTKDLVFTKE |
| | NTITVQQYDSNGTKLEGSAVEITKLDEIKNALKSGSIIIPIVASVMILTAM |
| | VIVIVIGSG |
| OspA | MGVTGILQLPRDRFKRTSFFLWVIILFQRTFSKQNVSSLDEKNSVSVDLP |
| ST1_SS_Ebola- | GEMKVLVSKEKNKDGKYDLIATVDKLELKGTSDKSNGSGVLEGVKAD |
| GP_Mayinga- | KSKVKLTISDDLGQTTLEVFKEDGKTLVSKKVTSKDKSSTEEKFNEKGE |
| 76_TMB | VSEKIITRADGTRLEYTGIKSDGSGKAKEVLKGYDLKGELSSEKTTLVV |
| SEQ ID NO: 161 | KEGTVTLSKNISKSGEVSVELNDTDSSAATKKTAAWNSGTSTLTITVNS |
| | KKTKDLVFTKENTITVQQYDSNGTKLEGSAVEITKLDEIKNALKSGSWI |
| | PAGIGVTGVIIAVIALFCIGSG |
| Camp2 | MVEPTTTISATSTHELSASDARNSIQLLNAHIATLQSVQKSVPGSDYSDQ |
| SEQ ID NO: 162 | IRDLLKAAFDLRGLIETLAHGGIPFYDPSTIMPRIKLVATTIDTIHTATTTL |
| | QNKVRPAHVELGLEVTKAVLLTANPASTAKELDAEGAALKARLEKVS |
| | QYPDLTPNDVATVYVRTNFSKTIWQVRANRDRYILGHKSAAVYKTLN |
| | HAITKAVGVRLNPKTTVGNIQAARTELLAAYQTAFNSPDVKKAA |
| Camp2_SS_Flu- | MKAILVVLLYTFATANAVEPTTTISATSTHELSASDARNSIQLLNAHIAT |
| HA_A/California/7/ | LQSVQKSVPGSDYSDQIRDLLKAAFDLRGLIETLAHGGIPFYDPSTIMPRI |
| 2009 | KLVATTIDTIHTATTTLQNKVRPAHVELGLEVTKAVLLTANPASTAKEL |
| SEQ ID NO: 163 | DAEGAALKARLEKVSQYPDLTPNDVATVYVRTNFSKTIWQVRANRDR |
| | YILGHKSAAVYKTLNHAITKAVGVRLNPKTTVGNIQAARTELLAAYQT |
| | AFNSPDVKKAA |
| Camp2_SS_Flu- | MKAIIVLLMVVTSNAVEPTTTISATSTHELSASDARNSIQLLNAHIATLQ |
| HA_B/Phuket/3073/ | SVQKSVPGSDYSDQIRDLLKAAFDLRGLIETLAHGGIPFYDPSTIMPRIKL |
| 2013 | VATTIDTIHTATTTLQNKVRPAHVELGLEVTKAVLLTANPASTAKELDA |
| SEQ ID NO: 164 | EGAALKARLEKVSQYPDLTPNDVATVYVRTNFSKTIWQVRANRDRYIL |
| | GHKSAAVYKTLNHAITKAVGVRLNPKTTVGNIQAARTELLAAYQTAFN |
| | SPDVKKAA |
| Camp2_SS_Rabies- | MVPQALLFVPLLVFPLCFGVEPTTTISATSTHELSASDARNSIQLLNAHIA |
| G_Pasteur-vaccins | TLQSVQKSVPGSDYSDQIRDLLKAAFDLRGLIETLAHGGIPFYDPSTIMP |
| SEQ ID NO: 165 | RIKLVATTIDTIHTATTTLQNKVRPAHVELGLEVTKAVLLTANPASTAK |
| | ELDAEGAALKARLEKVSQYPDLTPNDVATVYVRTNFSKTIWQVRANR |
| | DRYILGHKSAAVYKTLNHAITKAVGVRLNPKTTVGNIQAARTELLAAY |
| | QTAFNSPDVKKAA |
| Camp2_SS_VZV- | MFLIQCLISAVIFYIQVTNAVEPTTTISATSTHELSASDARNSIQLLNAHIA |
| gI_Oka-vaccine | TLQSVQKSVPGSDYSDQIRDLLKAAFDLRGLIETLAHGGIPFYDPSTIMP |
| SEQ ID NO: 166 | RIKLVATTIDTIHTATTTLQNKVRPAHVELGLEVTKAVLLTANPASTAK |
| | ELDAEGAALKARLEKVSQYPDLTPNDVATVYVRTNFSKTIWQVRANR |
| | DRYILGHKSAAVYKTLNHAITKAVGVRLNPKTTVGNIQAARTELLAAY |
| | QTAFNSPDVKKAA |
| Camp2_SS_Ebola- | MGVTGILQLPRDRFKRTSFFLWVIILFQRTFSVEPTTTISATSTHELSASD |
| GP_Mayinga-76 | ARNSIQLLNAHIATLQSVQKSVPGSDYSDQIRDLLKAAFDLRGLIETLAH |
| SEQ ID NO: 167 | GGIPFYDPSTIMPRIKLVATTIDTIHTATTTLQNKVRPAHVELGLEVTKA |
| | VLLTANPASTAKELDAEGAALKARLEKVSQYPDLTPNDVATVYVRTNF |
| | SKTIWQVRANRDRYILGHKSAAVYKTLNHAITKAVGVRLNPKTTVGNI |
| | QAARTELLAAYQTAFNSPDVKKAA |
| Camp2_SS_Flu- | MKAILVVLLYTFATANAVEPTTTISATSTHELSASDARNSIQLLNAHIAT |
| HA_A/California/7/ | LQSVQKSVPGSDYSDQIRDLLKAAFDLRGLIETLAHGGIPFYDPSTIMPRI |
| 2009_TMB | KLVATTIDTIHTATTTLQNKVRPAHVELGLEVTKAVLLTANPASTAKEL |
| SEQ ID NO: 168 | DAEGAALKARLEKVSQYPDLTPNDVATVYVRTNFSKTIWQVRANRDR |
| | YILGHKSAAVYKTLNHAITKAVGVRLNPKTTVGNIQAARTELLAAYQT |
| | AFNSPDVKKAASGSILAIYSTVASSLVLVVSLGAISFGSG |
| Camp2_SS_Flu- | MKAIIVLLMVVTSNAVEPTTTISATSTHELSASDARNSIQLLNAHIATLQ |
| HA_B/Phuket/3073/ | SVQKSVPGSDYSDQIRDLLKAAFDLRGLIETLAHGGIPFYDPSTIMPRIKL |
| 2013_TMB | VATTIDTIHTATTTLQNKVRPAHVELGLEVTKAVLLTANPASTAKELDA |
| SEQ ID NO: 169 | EGAALKARLEKVSQYPDLTPNDVATVYVRTNFSKTIWQVRANRDRYIL |
| | GHKSAAVYKTLNHAITKAVGVRLNPKTTVGNIQAARTELLAAYQTAFN |
| | SPDVKKAASGSSTAASSLAVTLMLAIFIVYMVGSG |
| Camp2_SS_Rabies- | MVPQALLFVPLLVFPLCFGVEPTTTISATSTHELSASDARNSIQLLNAHIA |
| G_Pasteur- | TLQSVQKSVPGSDYSDQIRDLLKAAFDLRGLIETLAHGGIPFYDPSTIMP |
| vaccins_TMB | RIKLVATTIDTIHTATTTLQNKVRPAHVELGLEVTKAVLLTANPASTAK |
| SEQ ID NO: 170 | ELDAEGAALKARLEKVSQYPDLTPNDVATVYVRTNFSKTIWQVRANR |
| | DRYILGHKSAAVYKTLNHAITKAVGVRLNPKTTVGNIQAARTELLAAY |
| | QTAFNSPDVKKAASGSVLLSAGALTALMLIIFLMTCWGSG |
| Camp2_SS_VZV- | MFLIQCLISAVIFYIQVTNAVEPTTTISATSTHELSASDARNSIQLLNAHIA |
| gI_Oka- | TLQSVQKSVPGSDYSDQIRDLLKAAFDLRGLIETLAHGGIPFYDPSTIMP |
| vaccine_TMB | RIKLVATTIDTIHTATTTLQNKVRPAHVELGLEVTKAVLLTANPASTAK |
| SEQ ID NO: 171 | ELDAEGAALKARLEKVSQYPDLTPNDVATVYVRTNFSKTIWQVRANR |
| | DRYILGHKSAAVYKTLNHAITKAVGVRLNPKTTVGNIQAARTELLAAY |
| | QTAFNSPDVKKAASGSIIIPIVASVMILTAMVIVIVIGSG |
| Camp2_SS_Ebola- | MGVTGILQLPRDRFKRTSFFLWVIILFQRTFSVEPTTTISATSTHELSASD |
| GP_Mayinga- | ARNSIQLLNAHIATLQSVQKSVPGSDYSDQIRDLLKAAFDLRGLIETLAH |
| 76_TMB | GGIPFYDPSTIMPRIKLVATTIDTIHTATTTLQNKVRPAHVELGLEVTKA |
| SEQ ID NO: 172 | VLLTANPASTAKELDAEGAALKARLEKVSQYPDLTPNDVATVYVRTNF |
| | SKTIWQVRANRDRYILGHKSAAVYKTLNHAITKAVGVRLNPKTTVGNI |
| | QAARTELLAAYQTAFNSPDVKKAASGSWIPAGIGVTGVIIAVIALFCIGS |
| | G |
| PITP | MAGPTVTVIPVGREGGDITISGKGFSTTGFGVYVAVAPASVPEFYGNSD |
| SEQ ID NO: 173 | KFYGYDPSKDTTESPSTIWVYTPSQKAIGSRFAQGRPMNNDGSFTITMK |
| | APPFEQGKDFVVLTTKAHGVGKTDHSDDTRTPVTYREATPAPTGPKTPI |
| | APSKQPSKQAAPSKQVKPSKQAGPNKQSTTPQQKTAEHRSQTPAAHRT |
| | MTKQVCTIGASKVTSGSLTWGIRTSFTSYLRGPIANGSWKLSGGANWN |
| | GSAFTFPLTSGSFDPATKSGSLKYSGSVHMTGHHGILDMTLAEPSLQIKG |
| | STGHLYLDVKSSSMDGKKTNYGRVDFATFGVSVSGNAAIKGSPVKLTA |
| | TGAKAFAGFYRAGEPMNPLSTNLTLSAEKVCHNVTVDAVTGKVIGDDS |
| | GKGAGRGLPVT |
| PITP_SS_Flu- | MKAILVVLLYTFATANAAGPTVTVIPVGREGGDITISGKGFSTTGFGVY |
| HA_A/California/7/ | VAVAPASVPEFYGNSDKFYGYDPSKDTTESPSTIWVYTPSQKAIGSRFA |
| 2009 | QGRPMNNDGSFTITMKAPPFEQGKDFVVLTTKAHGVGKTDHSDDTRTP |
| SEQ ID NO: 174 | VTYREATPAPTGPKTPIAPSKQPSKQAAPSKQVKPSKQAGPNKQSTTPQ |
| | QKTAEHRSQTPAAHRTMTKQVCTIGASKVTSGSLTWGIRTSFTSYLRGP |
| | IANGSWKLSGGANWNGSAFTFPLTSGSFDPATKSGSLKYSGSVHMTGH |
| | HGILDMTLAEPSLQIKGSTGHLYLDVKSSSMDGKKTNYGRVDFATFGV |
| | SVSGNAAIKGSPVKLTATGAKAFAGFYRAGEPMNPLSTNLTLSAEKVC |
| | HNVTVDAVTGKVIGDDSGKGAGRGLPVT |
| PITP_SS_Flu- | MKAIIVLLMVVTSNAAGPTVTVIPVGREGGDITISGKGFSTTGFGVYVA |
| HA_B/Phuket/3073/ | VAPASVPEFYGNSDKFYGYDPSKDTTESPSTIWVYTPSQKAIGSRFAQG |
| 2013 | RPMNNDGSFTITMKAPPFEQGKDFVVLTTKAHGVGKTDHSDDTRTPVT |
| SEQ ID NO: 175 | YREATPAPTGPKTPIAPSKQPSKQAAPSKQVKPSKQAGPNKQSTTPQQK |
| | TAEHRSQTPAAHRTMTKQVCTIGASKVTSGSLTWGIRTSFTSYLRGPIA |
| | NGSWKLSGGANWNGSAFTFPLTSGSFDPATKSGSLKYSGSVHMTGHH |
| | GILDMTLAEPSLQIKGSTGHLYLDVKSSSMDGKKTNYGRVDFATFGVS |
| | VSGNAAIKGSPVKLTATGAKAFAGFYRAGEPMNPLSTNLTLSAEKVCH |
| | NVTVDAVTGKVIGDDSGKGAGRGLPVT |
| PITP_SS_Rabies- | MVPQALLFVPLLVFPLCFGAGPTVTVIPVGREGGDITISGKGFSTTGFGV |
| G_Pasteur-vaccins | YVAVAPASVPEFYGNSDKFYGYDPSKDTTESPSTIWVYTPSQKAIGSRF |
| SEQ ID NO: 176 | AQGRPMNNDGSFTITMKAPPFEQGKDFVVLTTKAHGVGKTDHSDDTR |
| | TPVTYREATPAPTGPKTPIAPSKQPSKQAAPSKQVKPSKQAGPNKQSTTP |
| | QQKTAEHRSQTPAAHRTMTKQVCTIGASKVTSGSLTWGIRTSFTSYLRG |
| | PIANGSWKLSGGANWNGSAFTFPLTSGSFDPATKSGSLKYSGSVHMTG |
| | HHGILDMTLAEPSLQIKGSTGHLYLDVKSSSMDGKKTNYGRVDFATFG |
| | VSVSGNAAIKGSPVKLTATGAKAFAGFYRAGEPMNPLSTNLTLSAEKV |
| | CHNVTVDAVTGKVIGDDSGKGAGRGLPVT |
| PITP_SS_VZV- | MFLIQCLISAVIFYIQVTNAAGPTVTVIPVGREGGDITISGKGFSTTGFGV |
| gI_Oka-vaccine | YVAVAPASVPEFYGNSDKFYGYDPSKDTTESPSTIWVYTPSQKAIGSRF |
| SEQ ID NO: 177 | AQGRPMNNDGSFTITMKAPPFEQGKDFVVLTTKAHGVGKTDHSDDTR |
| | TPVTYREATPAPTGPKTPIAPSKQPSKQAAPSKQVKPSKQAGPNKQSTTP |
| | QQKTAEHRSQTPAAHRTMTKQVCTIGASKVTSGSLTWGIRTSFTSYLRG |
| | PIANGSWKLSGGANWNGSAFTFPLTSGSFDPATKSGSLKYSGSVHMTG |
| | HHGILDMTLAEPSLQIKGSTGHLYLDVKSSSMDGKKTNYGRVDFATFG |
| | VSVSGNAAIKGSPVKLTATGAKAFAGFYRAGEPMNPLSTNLTLSAEKV |
| | CHNVTVDAVTGKVIGDDSGKGAGRGLPVT |
| PITP_SS_Ebola- | MGVTGILQLPRDRFKRTSFFLWVIILFQRTFSAGPTVTVIPVGREGGDITI |
| GP_Mayinga-76 | SGKGFSTTGFGVYVAVAPASVPEFYGNSDKFYGYDPSKDTTESPSTIWV |
| SEQ ID NO: 178 | YTPSQKAIGSRFAQGRPMNNDGSFTITMKAPPFEQGKDFVVLTTKAHG |
| | VGKTDHSDDTRTPVTYREATPAPTGPKTPIAPSKQPSKQAAPSKQVKPS |
| | KQAGPNKQSTTPQQKTAEHRSQTPAAHRTMTKQVCTIGASKVTSGSLT |
| | WGIRTSFTSYLRGPIANGSWKLSGGANWNGSAFTFPLTSGSFDPATKSG |
| | SLKYSGSVHMTGHHGILDMTLAEPSLQIKGSTGHLYLDVKSSSMDGKK |
| | TNYGRVDFATFGVSVSGNAAIKGSPVKLTATGAKAFAGFYRAGEPMNP |
| | LSTNLTLSAEKVCHNVTVDAVTGKVIGDDSGKGAGRGLPVT |
| PITP_SS_Flu- | MKAILVVLLYTFATANAAGPTVTVIPVGREGGDITISGKGFSTTGFGVY |
| HA_A/California/7/ | VAVAPASVPEFYGNSDKFYGYDPSKDTTESPSTIWVYTPSQKAIGSRFA |
| 2009_TMB | QGRPMNNDGSFTITMKAPPFEQGKDFVVLTTKAHGVGKTDHSDDTRTP |
| SEQ ID NO: 179 | VTYREATPAPTGPKTPIAPSKQPSKQAAPSKQVKPSKQAGPNKQSTTPQ |
| | QKTAEHRSQTPAAHRTMTKQVCTIGASKVTSGSLTWGIRTSFTSYLRGP |
| | IANGSWKLSGGANWNGSAFTFPLTSGSFDPATKSGSLKYSGSVHMTGH |
| | HGILDMTLAEPSLQIKGSTGHLYLDVKSSSMDGKKTNYGRVDFATFGV |
| | SVSGNAAIKGSPVKLTATGAKAFAGFYRAGEPMNPLSTNLTLSAEKVC |
| | HNVTVDAVTGKVIGDDSGKGAGRGLPVTSGSILAIYSTVASSLVLVVSL |
| | GAISFGSG |
| PITP_SS_Flu- | MKAIIVLLMVVTSNAAGPTVTVIPVGREGGDITISGKGFSTTGFGVYVA |
| HA_B/Phuket/3073/ | VAPASVPEFYGNSDKFYGYDPSKDTTESPSTIWVYTPSQKAIGSRFAQG |
| 2013_TMB | RPMNNDGSFTITMKAPPFEQGKDFVVLTTKAHGVGKTDHSDDTRTPVT |
| SEQ ID NO: 180 | YREATPAPTGPKTPIAPSKQPSKQAAPSKQVKPSKQAGPNKQSTTPQQK |
| | TAEHRSQTPAAHRTMTKQVCTIGASKVTSGSLTWGIRTSFTSYLRGPIA |
| | NGSWKLSGGANWNGSAFTFPLTSGSFDPATKSGSLKYSGSVHMTGHH |
| | GILDMTLAEPSLQIKGSTGHLYLDVKSSSMDGKKTNYGRVDFATFGVS |
| | VSGNAAIKGSPVKLTATGAKAFAGFYRAGEPMNPLSTNLTLSAEKVCH |
| | NVTVDAVTGKVIGDDSGKGAGRGLPVTSGSSTAASSLAVTLMLAIFIVY |
| | MVGSG |
| PITP_SS_Rabies- | MVPQALLFVPLLVFPLCFGAGPTVTVIPVGREGGDITISGKGFSTTGFGV |
| G_Pasteur- | YVAVAPASVPEFYGNSDKFYGYDPSKDTTESPSTIWVYTPSQKAIGSRF |
| vaccins_TMB | AQGRPMNNDGSFTITMKAPPFEQGKDFVVLTTKAHGVGKTDHSDDTR |
| SEQ ID NO: 181 | TPVTYREATPAPTGPKTPIAPSKQPSKQAAPSKQVKPSKQAGPNKQSTTP |
| | QQKTAEHRSQTPAAHRTMTKQVCTIGASKVTSGSLTWGIRTSFTSYLRG |
| | PIANGSWKLSGGANWNGSAFTFPLTSGSFDPATKSGSLKYSGSVHMTG |
| | HHGILDMTLAEPSLQIKGSTGHLYLDVKSSSMDGKKTNYGRVDFATFG |
| | VSVSGNAAIKGSPVKLTATGAKAFAGFYRAGEPMNPLSTNLTLSAEKV |
| | CHNVTVDAVTGKVIGDDSGKGAGRGLPVTSGSVLLSAGALTALMLIIFL |
| | MTCWGSG |
| PITP_SS_VZV- | MFLIQCLISAVIFYIQVTNAAGPTVTVIPVGREGGDITISGKGFSTTGFGV |
| gI_Oka- | YVAVAPASVPEFYGNSDKFYGYDPSKDTTESPSTIWVYTPSQKAIGSRF |
| vaccine_TMB | AQGRPMNNDGSFTITMKAPPFEQGKDFVVLTTKAHGVGKTDHSDDTR |
| SEQ ID NO: 182 | TPVTYREATPAPTGPKTPIAPSKQPSKQAAPSKQVKPSKQAGPNKQSTTP |
| | QQKTAEHRSQTPAAHRTMTKQVCTIGASKVTSGSLTWGIRTSFTSYLRG |
| | PIANGSWKLSGGANWNGSAFTFPLTSGSFDPATKSGSLKYSGSVHMTG |
| | HHGILDMTLAEPSLQIKGSTGHLYLDVKSSSMDGKKTNYGRVDFATFG |
| | VSVSGNAAIKGSPVKLTATGAKAFAGFYRAGEPMNPLSTNLTLSAEKV |
| | CHNVTVDAVTGKVIGDDSGKGAGRGLPVTSGSIIIPIVASVMILTAMVIV |
| | IVIGSG |
| PITP_SS_Ebola- | MGVTGILQLPRDRFKRTSFFLWVIILFQRTFSAGPTVTVIPVGREGGDITI |
| GP_Mayinga- | SGKGFSTTGFGVYVAVAPASVPEFYGNSDKFYGYDPSKDTTESPSTIWV |
| 76_TMB | YTPSQKAIGSRFAQGRPMNNDGSFTITMKAPPFEQGKDFVVLTTKAHG |
| SEQ ID NO: 183 | VGKTDHSDDTRTPVTYREATPAPTGPKTPIAPSKQPSKQAAPSKQVKPS |
| | KQAGPNKQSTTPQQKTAEHRSQTPAAHRTMTKQVCTIGASKVTSGSLT |
| | WGIRTSFTSYLRGPIANGSWKLSGGANWNGSAFTFPLTSGSFDPATKSG |
| | SLKYSGSVHMTGHHGILDMTLAEPSLQIKGSTGHLYLDVKSSSMDGKK |
| | TNYGRVDFATFGVSVSGNAAIKGSPVKLTATGAKAFAGFYRAGEPMNP |
| | LSTNLTLSAEKVCHNVTVDAVTGKVIGDDSGKGAGRGLPVTSGSWIPA |
| | GIGVTGVIIAVIALFCIGSG |

### Example 5: In Vitro Antigen Expression from Expanded mRNA Design Panel

This Example outlines the analysis of cell viability, protein expression, and localization following the transfection of the mRNA panel (described in Example 4) into HEK293T cells. The transfection and Western blot analysis were previously described in Example 1. The antigens in the Western blot analyzed were OspA ST1, CAMP2, and PITP.

HEK Expi293F cell counts and cell percent viability resulting from two expression tests with the mRNA construct panel post-transfection were measured. All cell viability values exceeded 80% at 24 and 48 hours post transfection, indicating that the conditions were normal and that none of the mRNA constructs produced off-target cell cytotoxicity.

### OspA

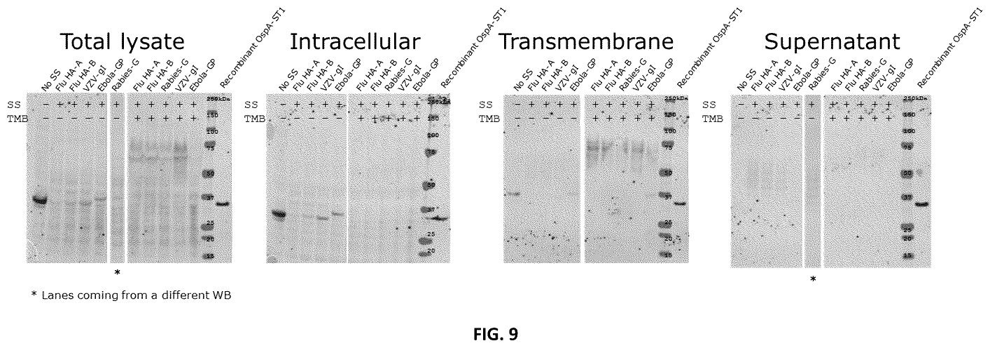

As shown in FIG. 9, in vitro expression of OspA ST1 fused to signal sequences (labeled “SS” on gels) derived from Influenza A, Influenza B, Rabies, VZV, and Ebola glycoproteins with or without their respective transmembrane domains (“TMB”) was achieved at 48-hours post-transfection in HEK Expi293F cells. OspA ST1 (expected size about 28-34 kDa) was detected using a rabbit polyclonal antibody to OspA at a 1:2000 dilution (ABCAM ab106081). Controls included an OspA ST1 construct without any SS or any lipidation sequence (first lane on all gels, labeled “No SS”) as well as recombinant OspA ST1 (last lane on all gels). With respect to testing OspA localization, samples were collected from crude extracts (total lysate), cell supernatants, and fractionated cell samples containing either intracellular or transmembrane compartments. These results demonstrate that adding a transmembrane domain induces localization of OspA ST1 at the cell membrane and reduces secretion and intracellular localization.

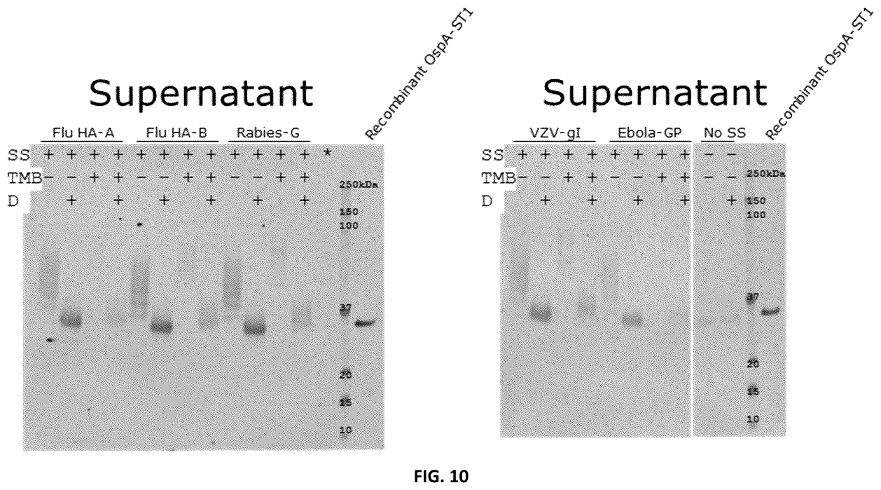

Furthermore, since the representative gels of the supernatant fraction in FIG. 9 exhibited faint signal (last column in panel), the supernatant OspA fractions were concentrated 7-fold and half of the resulting volume was deglycosylated (represented by a “D” on the gels) to analyze the protein by Western blot before and after enzymatic treatment as shown in FIG. 10. OspA ST1 was present in the supernatant fraction for all the OspA constructs tested but with varied expression levels. For instance, the OspA ST1 construct fused to the Ebola glycoprotein SS, which also had a lower SignalP cleavage score compared to the other constructs, had reduced expression in the supernatant fraction relative to the other OspA constructs (see FIG. 10, second gel, lanes 5 and 6). Additionally, fusing the constructs to the transmembrane domain reduced (but did not completely abolish) OspA supernatant detection in some of the samples.

### CAMP2

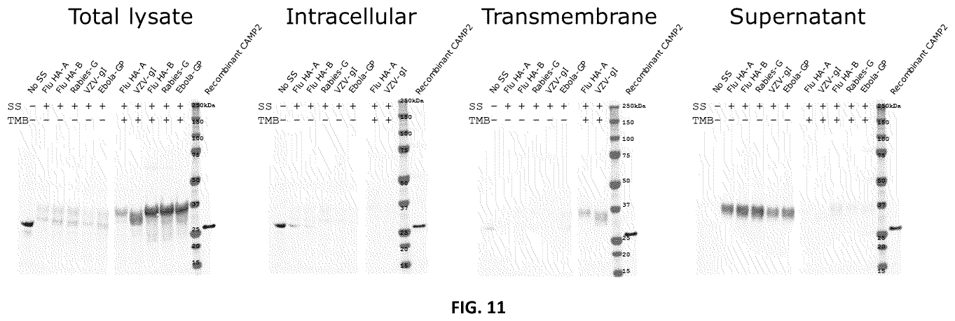

As shown in FIG. 11, in vitro expression of CAMP2 fused to signal sequences (labeled “SS” on gels) derived from Influenza A, Influenza B, Rabies, VZV, and Ebola glycoproteins with or without their respective transmembrane domains (“TMB”) was achieved at 48-hours post-transfection in HEK Expi293F cells. CAMP2 (expected size about 26-32 kDa) was detected using a rabbit polyclonal antibody to CAMP2 (generated in-house) at a 1:1500 dilution. Controls included a CAMP2 construct without any SS (first lane on all gels, labeled “No SS”) as well as recombinant CAMP2 (last lane on all gels). With respect to testing CAMP2 localization, samples were collected from crude extracts (total lysate), cell supernatants, and fractionated cell samples containing either intracellular or transmembrane compartments. These results demonstrate that adding a transmembrane domain induces localization of CAMP2 at the cell membrane and reduces secretion and intracellular localization (compare constructs with or without transmembrane domains in last column labeled “supernatant”). The Western blot analysis demonstrated that CAMP2 is well expressed and localizes in the expected fractions depending on the presence of a secretion signal peptide or a transmembrane domain.

### PITP

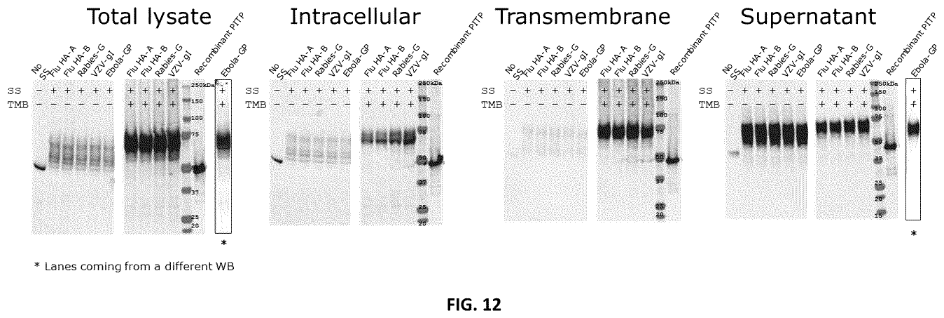

As shown in FIG. 12, in vitro expression of PITP fused to signal sequences (labeled “SS” on gels) derived from Influenza A, Influenza B, Rabies, VZV, and Ebola glycoproteins with or without their respective transmembrane domains (“TMB”) was achieved at and 48-hours post-transfection in HEK Expi293F cells. PITP (expected size about 42-48 kDa) was detected using a mouse polyclonal to PITP (generated in-house) at a 1:1000 dilution. Controls included an PITP construct without any SS or any TMB (first lane on all gels, labeled “No SS”) as well as recombinant PITP (last lane on all gels). With respect to testing PITP localization, samples were collected from crude extracts (total lysate), cell supernatants, and fractionated cell samples containing either intracellular or transmembrane compartments. Like the OspA and the CAMP2 antigen localization analysis, these results demonstrate that adding a transmembrane domain induces localization of PITP at the cell membrane and reduces secretion and intracellular localization (compare constructs with or without transmembrane domains in last column labeled “supernatant”). Notwithstanding, PITP transmembrane containing constructs did show some escape into the supernatant fraction.

This Western blot analysis demonstrated that PITP is well expressed and localizes in the expected fractions depending on the presence of a secretion signal peptide or a transmembrane domain.

### SUMMARY

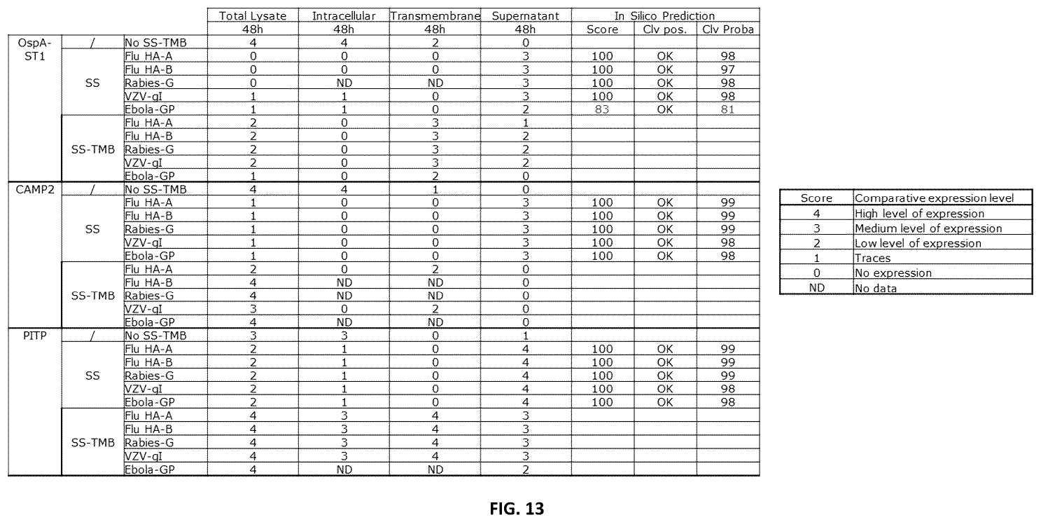

To compare the Western blot analysis for the OspA ST1, CAMP2, and PITP the protein expression and localization results were tabulated as shown in FIG. 13. In general, OspA ST1 antigen containing constructs are not well expressed compared to CAMP2 or PITP containing constructs. All three protein antigens were expressed in their expected locations but with varying degrees of expression and displayed some escape to the supernatant at varying degrees when the transmembrane domain was introduced. The construct, OspA ST1_SS-Ebola-GP, which had a low SignalP cleavage score, showed lower expression in the supernatant fraction indicating that it was likely accumulating in the intracellular fraction.

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All patents and publications cited herein are incorporated by reference herein in their entirety.

1. A nucleic acid comprising an open reading frame (ORF), wherein the ORF comprises:
a polynucleotide sequence encoding at least one antigenic prokaryotic polypeptide and
a polynucleotide sequence encoding at least one viral secretion signal peptide.

2. The nucleic acid of claim 1, wherein the ORF further comprises a polynucleotide sequence encoding at least one transmembrane domain (TMB).

3. The nucleic acid of claim 1, wherein:
the viral secretion signal peptide is derived from a viral sequence in a virus able to infect humans,
the viral secretion signal peptide is derived from a viral sequence selected from the group consisting of: an influenza secretion signal peptide sequence, and a non-influenza secretion signal peptide sequence selected from the group consisting of a SARS CoV-2 secretion signal peptide sequence, a varicella-zoster virus (VZV) secretion signal peptide sequence, a measles secretion signal peptide sequence, a rubella secretion signal peptide sequence, a mumps secretion signal peptide sequence, an Ebola secretion signal peptide sequence, a smallpox secretion signal peptide sequence, and a rabies secretion signal peptide sequence; and/or
the viral secretion signal peptide is selected from the group consisting of: an influenza hemagglutinin (HA) secretion signal peptide sequence, a SARS CoV-2 spike secretion signal peptide sequence, a VZV gB secretion signal peptide sequence, a VZV gE secretion signal peptide sequence, a VZV gI secretion signal peptide sequence, a VZV gK secretion signal peptide sequence, a measles F-protein secretion signal peptide sequence, a rubella E1 protein secretion signal peptide sequence, a rubella E2 protein secretion signal peptide sequence, a mumps F-protein secretion signal peptide sequence, an Ebola GP protein secretion signal peptide sequence, a smallpox 6 kDa IC protein secretion signal peptide sequence, and a rabies G protein secretion signal peptide sequence, preferably wherein the viral secretion signal peptide comprises an HA secretion signal peptide sequence from influenza A or influenza B, more preferably from influenza A.

4-5. (canceled)

6. The nucleic acid of claim 3, wherein the HA secretion signal peptide sequence comprises an amino acid sequence MKX₁X₂LX₃VX₄LX₅TFX₆X₇X₈X₉A (SEQ ID NO: 145) wherein
X₁ is selected from A and V;
X₂ is selected from I and K;
X₃ is selected from V and L;
X₄ is selected from L and M;
X₅ is selected from Y and C;
X₆ is selected from T and A;
X₇ is selected from T and A;
X₈ is selected from A and T; and
X₉ is selected from N and Y, optionally wherein the HA secretion signal peptide sequence comprises an amino acid sequence selected from SEQ ID NO: 95-109.

7. (canceled)

8. The nucleic acid of claim 3, wherein the HA secretion signal peptide sequence comprises an amino acid sequence MKX₁IIALSX₂ILCLVFX₃ (SEQ ID NO: 146) wherein
X₁ is selected from T and A;
X₂ is selected from Y, N, C, and H; and
X₃ is selected from T and A, optionally wherein the HA secretion signal peptide sequence comprises an amino acid sequence selected from SEQ ID NOs: 110-131.

9. (canceled)

10. The nucleic acid of claim 3, wherein:
the HA secretion signal peptide sequence comprises an amino acid sequence MKAIIVLLMVVTSXiA (SEQ ID NO: 147) wherein X₁ is selected from S and N; or
the HA secretion signal peptide sequence comprises an amino acid sequence MXiAIIVLLMVVTSNA (SEQ ID NO: 148) wherein X₁ is selected from K and E, optionally wherein the HA secretion signal peptide sequence comprises an amino acid sequence selected from SEQ ID NOs: 132-144.

11-12. (canceled)

13. The nucleic acid of any one of claim 1, wherein the viral secretion signal peptide comprises an amino acid sequence selected from the group consisting of:
(SEQ ID NO: 1)
MKAKLLVLLCTFTATYA;
(SEQ ID NO: 2)
MKAILVVLLYTFATANA;
(SEQ ID NO: 3)
MKTIIALSYILCLVFA;
(SEQ ID NO: 4)
MKAIIVLLMVVTSNA;
(SEQ ID NO: 5)
MFVFLVLLPLVS;
(SEQ ID NO: 6)
MFLLTTKRTMFVFLVLLPLVS;
(SEQ ID NO: 7)
MSPCGYYSKWRNRDRPEYRRNLRFRRFFSSIHPNAAAGSGFNGPGVFIT
SVTGVWLCFLCIFSMFVTAVVS;
(SEQ ID NO: 8)
MGTVNKPVVGVLMGFGIITGTLRITNPVRA;
(SEQ ID NO: 9)
MFLIQCLISAVIFYIQVTNA;
(SEQ ID NO: 10)
MQALGIKTEHFIIMCLLSGHA;
(SEQ ID NO: 11)
MGLKVNVSAIFMAVLLTLQTPTG;
(SEQ ID NO: 12)
MGAAAALTAVVLOGYNPPAYG;
(SEQ ID NO: 13)
MGAPQAFLAGLLLAAVAVGTARA;
(SEQ ID NO: 14)
MKVFLVTCLGFAVFSSSVC;
(SEQ ID NO: 15)
MGVTGILQLPRDRFKRTSFFLWVIILFQRTFS;
(SEQ ID NO: 16)
MRSLIIFLLFPSIIYS;
and
(SEQ ID NO: 184)
MVPQALLFVPLLVFPLCFG.

14. (canceled)

15. The nucleic acid of claim 1, wherein the viral secretion signal peptide is positioned at the N-terminus or C-terminus of the antigenic prokaryotic polypeptide, optionally wherein the viral secretion signal peptide is attached to the antigenic prokaryotic polypeptide with a linker.

16-17. (canceled)

18. The nucleic acid of claim 2, wherein the TMB:
(a) comprises or consists of 15 to 50 amino acid residues, preferably 15 to 30 amino acid residues, more preferably 18 to 25 amino acid residues; and/or
(b) comprises at least 50% of hydrophobic amino acid residues, preferably selected in the group consisting of: alanine, isoleucine, leucine, valine, phenylalanine, tryptophane and tyrosine; and/or
(c) comprises at least one alpha helix.

19. The nucleic acid of claim 2, wherein:
the TMB is derived from an integral membrane protein, preferably from a single pass membrane protein, more preferably from a bitopic membrane protein, even more preferably from a bitopic membrane protein of Type I; and/or
the TMB is derived from a non-human sequence, optionally wherein the antigenic prokaryotic polypeptide is derived from a prokaryotic transmembrane protein, and wherein the TMB is the TMB of the prokaryotic transmembrane protein or the antigenic prokaryotic polypeptide is not derived from a prokaryotic transmembrane protein, optionally wherein the TMB is derived from a viral sequence, optionally wherein:
the TMB is derived from a viral transmembrane domain sequence selected from the group consisting of: an influenza transmembrane domain sequence, and a non-influenza transmembrane domain sequence selected from the group consisting of a SARS CoV-2 transmembrane domain sequence, a varicella-zoster virus (VZV) transmembrane domain sequence, a measles transmembrane domain sequence, a rubella transmembrane domain sequence, a mumps transmembrane domain sequence, an Ebola transmembrane domain sequence, and a rabies transmembrane domain sequence:
the TMB is selected from the group consisting of: an influenza hemagglutinin (HA) transmembrane domain sequence, a SARS CoV-2 spike transmembrane domain sequence, a VZV gB transmembrane domain sequence, a VZV gE transmembrane domain sequence, a VZV gI transmembrane domain sequence, a VZV gK transmembrane domain sequence, a measles F-protein transmembrane domain sequence, a rubella E1 protein transmembrane domain sequence, a rubella E2 protein transmembrane domain sequence, a mumps F-protein transmembrane domain sequence, an Ebola GP protein transmembrane domain sequence and a rabies G protein transmembrane domain sequence, preferably wherein the TMB comprises an HA transmembrane domain sequence from influenza A or influenza B, more preferably from influenza A.

20-25. (canceled)

26. The nucleic acid of claim 2, wherein the TMB comprises an amino acid sequence selected from the group consisting of:
(SEQ ID NO: 17)
ILAIYSTVASSLVLLVSLGAISF;
(SEQ ID NO: 18)
ILAIYSTVASSLVLVVSLGAISF;
(SEQ ID NO: 19)
ILWISFAISCFLLCVVLLGFI;
(SEQ ID NO: 20)
STAASSLAVTLMLAIFIVYMV;
(SEQ ID NO: 21)
WYIWLGFIAGLIAIVMVTIML;
(SEQ ID NO: 22)
FGALAVGLLVLAGLVAAFFAY;
(SEQ ID NO: 23)
AAWTGGLAAVVLLCLVIFLIC;
(SEQ ID NO: 24)
IIIPIVASVMILTAMVIVIVI;
(SEQ ID NO: 25)
YFWCVQLKMIFFAWFVYGMYL;
(SEQ ID NO: 26)
IVYILIAVCLGGLIGIPALIC;
(SEQ ID NO: 27)
LDHAFAAFVLLVPWVLIFMVC;
(SEQ ID NO: 28)
WWQLTLGAICALLLAGLLACC;
(SEQ ID NO: 29)
IVAALVLSILSIIISLLFCCW;
(SEQ ID NO: 30)
WIPAGIGVTGVIIAVIALFCI;
and
(SEQ ID NO: 185)
VLLSAGALTALMLIIFLMTCW.

27-30. (canceled)

31. A nucleic acid comprising an open reading frame (ORF), wherein the ORF comprises:
a polynucleotide sequence encoding at least one antigenic polypeptide, preferably an antigenic prokaryotic polypeptide, and
a polynucleotide sequence encoding at least one transmembrane domain (TMB), wherein the TMB is heterologous to the antigenic polypeptide,
wherein optionally the ORF further comprises a polynucleotide sequence encoding at least one secretion signal peptide.

32. A nucleic acid comprising an open reading frame (ORF), wherein the ORF comprises:
a polynucleotide sequence encoding at least one antigenic prokaryotic polypeptide and
a polynucleotide sequence encoding at least one transmembrane domain (TMB).

33-44. (canceled)

45. The nucleic acid of claim 32, wherein the antigenic prokaryotic polypeptide is derived from a bacteria of a genus selected from the group consisting of Acetobacter, Acinetobacter, Actinomyces, Aerococcus, Agrobacterium, Anaplasma, Azorhizobia, Azotobacter, Bacillus, Bacteroides, Bartonella, Bordetella, Borrelia, Brucella, Burkkolderia, Calymmatobacterium, Campylobacter, Chlamydia, Chlamydophila, Clostridium, Corynebacterium, Coxiella, Cutibacterium, Ehrlichia, Enterobacter, Enterococcus, Escherichia, Francisella, Fusobacterium, Gardnerella, Haemophilus, Helicobacter, Klebsiella, Lactobacillus, Lactococcus, Legionella, Listeria, Methanobacterium, Microbacterium, Micrococcus, Moraxella, Mycobacterium, Mycoplasma, Neisseria, Pasteurella, Pediococcus, Peptostreptococcus, Porphyromonas, Prevotella, Propionibacterium, Pseudomonas, Rhizobium, Rickettsia, Rochalimaea, Rothia, Salmonella, Serratia, Shigella, Sarcina, Spirillum, Spirochaetes, Staphylococcus, Stenotrophomonas, Streptobacillus, Streptococcus, Tetragenococcus, Treponema, Vibrio, Viridans, Walbachia, and Yersinia, preferably from a bacteria of a species selected from the group consisting of Acetobacter aurantius, Acinetobacter baumannii, Actinomyces israelii, Agrobacterium radiobacter, Agrobacterium tumefaciens, Anaplasma phagocytophilum, Azorhizobium caulinodans, Azotobacter vinelandii, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus Thuringiensis, Bacteroides fragilis, Bacteroides gingivalis, Bacteroides melaninogenicus, Bartonella henselae, Bartonella Quintana, Bordetella bronchiseptica, Bordetella pertussis, Borrelia burgdorferi. Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Campylobacter pylori, Chlamydia trachomatis, Chlamydophila pneumoniae, Chlamydophila psittaci, Clostridium botulinum, Clostridium difficile, Clostridium perfringens, Clostridium tetani, Corynebacterium diphtheriae, Corynebacterium fusiforme, Coxiella burnetii, Cutibacterium acnes, Cutibacterium avidum, Cutibacterium granulosum, Cutibacterium namnetense, Cutibacterium humerusii, Ehrlichia chaffeensis, Enterobacter cloacae, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus bulgaricus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Neisseria gonorrhoeae, Neisseria meningitidis, Pasteurella multocida, Pasteurella tularensis, Peptostreptococcus, Porphyromonas gingivalis, Prevotella melaninogenica, Propionibacterium acnes, Pseudomonas aeruginosa, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus, Staphylococcus epidermidis, Stenotrophomonas maltophilia, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Viridans streptococci, Wolbachia, Yersinia enterocolitica, Yersinia pestis and Yersinia pseudotuberculosis.

46. The nucleic acid of claim 1, wherein:
the antigenic prokaryotic polypeptide is derived from a bacteria of the genus Borrelia, preferably selected from the species B. burgdorferi, afzelii, garinii, bavariensis, mayonii, spielmanii, lusitaniae, bissettii and/or valaisiana; and/or
the antigenic prokarvotic polypeptide is OspA or a fragment or variant thereof, wherein the OspA or fragment or variant thereof comprises at least 5 amino acids, preferably wherein the antigenic prokarvotic polypeptide comprises:
(a) an amino acid sequence derived from OspA ST1, preferably with at least 85% identity to the sequence
(SEQ ID NO: 31)
KQNVSSLDEKNSVSVDLPGEMKVLVSKEKNKDGKYDLIATVDKLELKGT
SDKNNGSGVLEGVKADKSKVKLTISDDLGQTTLEVFKEDGKTLVSKKVT
SKDKSSTEEKFNEKGEVSEKIITRADGTRLEYTGIKSDGSGKAKEVLKG
YDLKGELSSEKTTLVVKEGTVTLSKNISKSGEVSVELNDTDSSAATKKT
AAWNSGTSTLTITVNSKKTKDLVFTKENTITVQQYDSNGTKLEGSAVEI
TKLDEIKNALK;
(b) an amino acid sequence derived from OspA ST2, preferably with at least 85% identity to the sequence
(SEQ ID NO: 32)
KQNVSSLDEKNSASVDLPGEMKVLVSKEKDKDGKYSLKATVDKIELKGT
SDKDNGSGVLEGTKDDKSKAKLTIADDLSKTTFELFKEDGKTLVSRKVS
SKDKTSTDEMFNEKGELSAKTMTRENGTKLEYTEMKSDGTGKAKEVLKN
FTLEGKVANDKVTLEVKEGTVTLSKEIAKSGEVTVALNDTNTTQATKKT
GAWDSKTSTLTISVNSKKTTQLVFTKQDTITVQKYDSAGTNLEGTAVEI
KTLDELKNALK;
(c) an amino acid sequence derived from OspA ST3, preferably with at least 85% identity to the sequence
SEQ ID NO: 186)
KQNVSSLDEKNSVSVDLPGGMKVLVSKEKDKDGKYSLMATVEKLELKGT
SDKSNGSGVLEGEKADKSKAKLTISQDLNQTTFEIFKEDGKTLVSRKVN
SKDKSSTEEKFNDKGKLSEKVVTRANGTRLEYTEIKNDGSGKAKEVLKG
FALEGTLTDGGETKLTVTEGTVTLSKNISKSGEITVALNDTETTPADKK
TGEWKSDTSTLTISKNSQKPKQLVFTKENTITVQNYNRAGNALEGSPAE
IKDLAELKAALK;
(d) an amino acid sequence derived from OspA ST4, preferably with at least 85% identity to the sequence
(SEQ ID NO: 187)
KQNVSSLDEKNSVSVDLPGEMKVLVSKEKDKDGKYSLMATVDKLELKGT
SDKSNGSGTLEGEKSDKSKAKLTISEDLSKTTFEIFKEDGKTLVSKKVN
SKDKSSIEEKFNAKGELSEKTILRANGTRLEYTEIKSDGTGKAKEVLKD
FALEGTLAADKTTLKVTEGTVVLSKHIPNSGEITVELNDSNSTQATKKT
GKWDSNTSTLTISVNSKKTKNIVFTKEDTITVQKYDSAGTNLEGNAVEI
KTLDELKNALK;
(e) an amino acid sequence derived from OspA ST5, preferably with at least 85% identity to the sequence
(SEQ ID NO: 188)
KQNVSSLDEKNSVSVDLPGGMKVLVSKEKDKDGKYSLMATVEKLELKGT
SDKNNGSGTLEGEKTDKSKVKLTIAEDLSKTTFEIFKEDGKTLVSKKVT
LKDKSSTEEKFNEKGEISEKTIVRANGTRLEYTDIKSDGSGKAKEVLKD
FTLEGTLAADGKTTLKVTEGTVVLSKNILKSGEITVALDDSDTTQATKK
TGKWDSKTSTLTISVNSQKTKNLVFTKEDTITVQKYDSAGTNLEGKAVE
ITTLEKLKDALK;
(f) an amino acid sequence derived from OspA ST6, preferably with at least 85% identity to the sequence
(SEQ ID NO: 189)
KQNVSSLDEKNSVSVDLPGGMTVLVSKEKDKDGKYSLEATVDKLELKGT
SDKNNGSGTLEGEKTDKSKVKSTIADDLSQTKFEIFKEDGKTLVSKKVT
LKDKSSTEEKFNGKGETSEKTIVRANGTRLEYTDIKSDGSGKAKEVLKD
FTLEGTLAADGKTTLKVTEGTVVLSKNILKSGEITAALDDSDTTRATKK
TGKWDSKTSTLTISVNSQKTKNLVFTKEDTITVQRYDSAGTNLEGKAVE
ITTLKELKNALK;
(g) an amino acid sequence derived from OspA ST7, preferably with at least 85% identity to the sequence
(SEQ ID NO: 190)
KQNVSSLDEKNSVSVDLPGEMKVLVSKEKDKDGKYSLEATVDKLELKGT
SDKNNGSGVLEGVKAAKSKAKLTIADDLSQTKFEIFKEDGKTLVSKKVT
LKDKSSTEEKFNDKGKLSEKVVTRANGTRLEYTEIQNDGSGKAKEVLKS
LTLEGTLTADGETKLTVEAGTVTLSKNISESGEITVELKDTETTPADKK
SGTWDSKTSTLTISKNSQKTKQLVFTKENTITVQKYNTAGTKLEGSPAE
IKDLEALKAALK;
(h) any combination of (a)-(g);
(i) a sequence derived from OspA ST1 according to (a) and a sequence derived from OspA ST2 according to (b), or
(i) a sequence derived from OspA ST1 according to (a), a sequence derived from OspA ST2 according to (b), a sequence derived from OspA ST3 according to (c), a sequence derived from OspA ST4 according to (d), a sequence derived from OspA ST5 according to (e), a sequence derived from OspA ST6 according to (f) and a sequence derived from OspA ST7 according to (g).

47-52. (canceled)

53. The nucleic acid of claim 1, wherein the nucleic acid is DNA or wherein the nucleic acid is messenger RNA (mRNA), wherein in particular the mRNA may be non-replicating mRNA, self-replicating mRNA or trans-replicating mRNA, optionally wherein the mRNA comprises at least one 5′ untranslated region (5′ UTR), at least one 3′ untranslated region (3′ UTR), and/or at least one polyadenylation (poly(A)) sequence, optionally wherein the mRNA comprises at least one chemical modification.

54-61. (canceled)

62. A composition comprising at least one nucleic acid of claim 1, optionally further comprising a lipid nanoparticle (LNP), optionally wherein the nucleic acid is encapsulated in the LNP.

63-88. (canceled)

89. A method of eliciting an immune response in a subject in need thereof, comprising administering to the subject the nucleic acid of claim 1.

90. A method of treating or preventing a prokaryotic infection in a subject in need thereof, comprising administering to the subject the nucleic acid of claim 1.

91. A method of secreting an antigenic prokaryotic polypeptide in a host cell, the method comprising administering to the host cell the nucleic acid of claim 1.

92. A method of displaying an antigenic prokaryotic polypeptide on the surface of a host cell, the method comprising administering to the host the nucleic acid of claim 1.

93. (canceled)


SEQUENCE LISTING (239 sequences)

SEQ ID NO: 1 - protein, 17 aa, unidentified
  source 1..17: note = Influenza virus
MKAKLLVLLCTFTATYA

SEQ ID NO: 2 - protein, 17 aa, unidentified
  source 1..17: note = Influenza virus
MKAILVVLLYTFATANA

SEQ ID NO: 3 - protein, 16 aa, unidentified
  source 1..16: note = Influenza virus
MKTIIALSYILCLVFA

SEQ ID NO: 4 - protein, 15 aa, unidentified
  source 1..15: note = Influenza virus
MKAIIVLLMVVTSNA

SEQ ID NO: 5 - protein, 12 aa, Severe acute respiratory syndrome-related coronavirus
  source 1..12: note = SARS CoV-2
MFVFLVLLPLVS

SEQ ID NO: 6 - protein, 21 aa, Severe acute respiratory syndrome-related coronavirus
  source 1..21: note = SARS CoV-2
MFLLTTKRTMFVFLVLLPLVS

SEQ ID NO: 7 - protein, 71 aa, Human alphaherpesvirus 3
MSPCGYYSKWRNRDRPEYRRNLRFRRFFSSIHPNAAAGSGFNGPGVFITSVTGVWLCFLCIFSMFVTAVVS

SEQ ID NO: 8 - protein, 30 aa, Human alphaherpesvirus 3
MGTVNKPVVGVLMGFGIITGTLRITNPVRA

SEQ ID NO: 9 - protein, 20 aa, Human alphaherpesvirus 3
MFLIQCLISAVIFYIQVTNA

SEQ ID NO: 10 - protein, 21 aa, Human alphaherpesvirus 3
MQALGIKTEHFIIMCLLSGHA

SEQ ID NO: 11 - protein, 23 aa, Measles morbillivirus
MGLKVNVSAIFMAVLLTLQTPTG

SEQ ID NO: 12 - protein, 21 aa, Rubivirus rubellae
MGAAAALTAVVLQGYNPPAYG

SEQ ID NO: 13 - protein, 23 aa, Rubivirus rubellae
MGAPQAFLAGLLLAAVAVGTARA

SEQ ID NO: 14 - protein, 19 aa, Mumps orthorubulavirus
MKVFLVTCLGFAVFSSSVC

SEQ ID NO: 15 - protein, 32 aa, unidentified
  source 1..32: note = Ebolavirus
MGVTGILQLPRDRFKRTSFFLWVIILFQRTFS

SEQ ID NO: 16 - protein, 16 aa, Human alphaherpesvirus 3
MRSLIIFLLFPSIIYS

SEQ ID NO: 17 - protein, 23 aa, unidentified
  source 1..23: note = Influenza virus
ILAIYSTVASSLVLLVSLGAISF

SEQ ID NO: 18 - protein, 23 aa, unidentified
  source 1..23: note = Influenza virus
ILAIYSTVASSLVLVVSLGAISF

SEQ ID NO: 19 - protein, 21 aa, unidentified
  source 1..21: note = Influenza virus
ILWISFAISCFLLCVVLLGFI

SEQ ID NO: 20 - protein, 21 aa, unidentified
  source 1..21: note = Influenza virus
STAASSLAVTLMLAIFIVYMV

SEQ ID NO: 21 - protein, 21 aa, Severe acute respiratory syndrome-related coronavirus
  source 1..21: note = SARS CoV-2
WYIWLGFIAGLIAIVMVTIML

SEQ ID NO: 22 - protein, 21 aa, Human alphaherpesvirus 3
FGALAVGLLVLAGLVAAFFAY

SEQ ID NO: 23 - protein, 21 aa, Human alphaherpesvirus 3
AAWTGGLAAVVLLCLVIFLIC

SEQ ID NO: 24 - protein, 21 aa, Human alphaherpesvirus 3
IIIPIVASVMILTAMVIVIVI

SEQ ID NO: 25 - protein, 21 aa, Human alphaherpesvirus 3
YFWCVQLKMIFFAWFVYGMYL

SEQ ID NO: 26 - protein, 21 aa, Measles morbillivirus
IVYILIAVCLGGLIGIPALIC

SEQ ID NO: 27 - protein, 21 aa, Rubivirus rubellae
LDHAFAAFVLLVPWVLIFMVC

SEQ ID NO: 28 - protein, 21 aa, Rubivirus rubellae
WWQLTLGAICALLLAGLLACC

SEQ ID NO: 29 - protein, 21 aa, Mumps orthorubulavirus
IVAALVLSILSIIISLLFCCW

SEQ ID NO: 30 - protein, 21 aa, unidentified
  source 1..21: note = Ebolavirus
WIPAGIGVTGVIIAVIALFCI

SEQ ID NO: 31 - protein, 257 aa, Borrelia burgdorferi
MKQNVSSLDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 32 - protein, 257 aa, Borrelia burgdorferi
MKQNVSSLDEKNSASVDLPGEMKVLVSKEKDKDGKYSLKATVDKIELKGTSDKDNGSGVLEGTKDDKSKAKLTIADDLSKTTFELFKEDGKTLVSRKVSSKDKTSTDEMFNEKGELSAKTMTRENGTKLEYTEMKSDGTGKAKEVLKNFTLEGKVANDKVTLEVKEGTVTLSKEIAKSGEVTVALNDTNTTQATKKTGAWDSKTSTLTISVNSKKTTQLVFTKQDTITVQKYDSAGTNLEGTAVEIKTLDELKNALK

SEQ ID NO: 33 - protein, 273 aa, synthetic construct
MKAKLLVLLCTFTATYAKQNVSSLDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 34 - protein, 273 aa, synthetic construct
MKAKLLVLLCTFTATYAKQNVSSLDEKNSASVDLPGEMKVLVSKEKDKDGKYSLKATVDKIELKGTSDKDNGSGVLEGTKDDKSKAKLTIADDLSKTTFELFKEDGKTLVSRKVSSKDKTSTDEMFNEKGELSAKTMTRENGTKLEYTEMKSDGTGKAKEVLKNFTLEGKVANDKVTLEVKEGTVTLSKEIAKSGEVTVALNDTNTTQATKKTGAWDSKTSTLTISVNSKKTTQLVFTKQDTITVQKYDSAGTNLEGTAVEIKTLDELKNALK

SEQ ID NO: 35 - protein, 302 aa, synthetic construct
MKAKLLVLLCTFTATYAKQNVSSLDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKSGSILAIYSTVASSLVLLVSLGAISFGSG

SEQ ID NO: 36 - protein, 302 aa, synthetic construct
MKAKLLVLLCTFTATYAKQNVSSLDEKNSASVDLPGEMKVLVSKEKDKDGKYSLKATVDKIELKGTSDKDNGSGVLEGTKDDKSKAKLTIADDLSKTTFELFKEDGKTLVSRKVSSKDKTSTDEMFNEKGELSAKTMTRENGTKLEYTEMKSDGTGKAKEVLKNFTLEGKVANDKVTLEVKEGTVTLSKEIAKSGEVTVALNDTNTTQATKKTGAWDSKTSTLTISVNSKKTTQLVFTKQDTITVQKYDSAGTNLEGTAVEIKTLDELKNALKSGSILAIYSTVASSLVLLVSLGAISFGSG

SEQ ID NO: 37 - protein, 273 aa, synthetic construct
MKAKLLVLLCTFTATYAKQNVGSLDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNIDKSGEVSVELDDTDSSAATKKTAAWNSGTSTLTITVNSKKTKDLVFTKENTITVQQYDSAGTKLEGSAVEITKLDEIKNALK

SEQ ID NO: 38 - protein, 273 aa, synthetic construct
MKAKLLVLLCTFTATYAKQNVGSLDEKNSASVDLPGEMKVLVSKEKDKDGKYSLKATVDKIELKGTSDKDDGSGVLEGTKDDKSKAKLTIADDLSKTTFELFKEDGKTLVSRKVSSKDKTSTDEMFNEKGELSAKTMTREDGTKLEYTEMKSDGTGKAKEVLKHFTLEGKVANDKVTLEVKEGTVTLSKEIAKSGEVTVALKDTSTTQATKKTGAWDSKTSTLTISVNSKKTTQLVFTKQDTITVQKYDSAGTNLEGTAVEIKTLDELKNALK

SEQ ID NO: 39 - protein, 302 aa, synthetic construct
MKAKLLVLLCTFTATYAKQNVGSLDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNIDKSGEVSVELDDTDSSAATKKTAAWNSGTSTLTITVNSKKTKDLVFTKENTITVQQYDSAGTKLEGSAVEITKLDEIKNALKSGSILAIYSTVASSLVLLVSLGAISFGSG

SEQ ID NO: 40 - protein, 302 aa, synthetic construct
MKAKLLVLLCTFTATYAKQNVGSLDEKNSASVDLPGEMKVLVSKEKDKDGKYSLKATVDKIELKGTSDKDDGSGVLEGTKDDKSKAKLTIADDLSKTTFELFKEDGKTLVSRKVSSKDKTSTDEMFNEKGELSAKTMTREDGTKLEYTEMKSDGTGKAKEVLKHFTLEGKVANDKVTLEVKEGTVTLSKEIAKSGEVTVALKDTSTTQATKKTGAWDSKTSTLTISVNSKKTTQLVFTKQDTITVQKYDSAGTNLEGTAVEIKTLDELKNALKSGSILAIYSTVASSLVLLVSLGAISFGSG

SEQ ID NO: 41 - RNA, 1019 nt, Borrelia burgdorferi
  source 1..1019: mol_type = other RNA
  mRNA 1..1019: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaagcaaaatgtgagtagtctggacgagaaaaatagcgtgagtgtggatctgccaggagagatgaaggtgctggtgtctaaagagaaaaacaaggacggaaagtacgacctgatcgccactgtggacaagctggagctgaaaggcacctctgataagaacaatggctctggcgtgctggaaggggtgaaggcagacaagagcaaagtgaagctgactatctctgacgacctggggcaaaccacactggaggtcttcaaggaagacggcaaaacactggtctctaagaaagtcaccagcaaggataagtcctccacagaggagaaattcaacgagaaaggcgaagtgtctgagaagatcatcaccagggccgacggaaccaggctggagtacaccggcatcaaaagcgacgggtcagggaaggctaaggaagtgctgaaaggatacgatctgaaaggagagctgtcctccgagaagacaactctggtggtgaaagagggaaccgttacactgtctaagaacatctccaagtctggggaagtcagcgtggagctgaacgacaccgacagctctgctgctacaaagaagactgctgcttggaacagcggcacctctaccctgacaatcaccgtgaattctaagaagaccaaggacctggtgttcactaaggagaataccattactgtccagcagtatgattccaatgggactaaactggaaggatccgccgtggagattactaagctggacgagatcaagaatgccctgaagtgacgggtggcatccctgtgacccctccccagtgcctctcctggccctggaagttgccactccagtgcccaccagccttgtcctaataaaattaagttgcatcaagct

SEQ ID NO: 42 - RNA, 1019 nt, Borrelia burgdorferi
  source 1..1019: mol_type = other RNA
  mRNA 1..1019: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaagcaaaacgtgtccagcctggacgagaagaattccgttagcgtggatctgccaggggagatgaaagtgctggtgtccaaggaaaagaacaaagatggcaagtatgacctgattgcaactgtggacaaactggagctgaagggcacttctgataaaaataacggcagcggcgttctggagggcgttaaggccgacaagtccaaggtgaagctgacaatctccgacgacctggggcagaccacactggaggtgtttaaagaggatggcaagaccctggtctctaaaaaagtgacttccaaagacaagtccagcacagaagagaaattcaatgaaaagggagaggtgtccgagaagatcatcactcgggccgacgggactaggctggagtacaccgggatcaagagcgacggctccggaaaggcaaaggaggtgctgaaaggctatgacctgaaaggcgagctgagcagcgagaaaactaccctggtggtgaaggaaggcacagtgactctgtcaaaaaatatctctaagtccggcgaggtgagcgtggaactgaatgacactgacagctccgctgcaactaagaaaacagccgcttggaactctggcacatctactctcactattaccgtgaactccaagaaaaccaaggacctggtgtttaccaaggagaatacaatcacagtgcaacagtacgacagcaacggcactaagctggagggctccgccgttgagattactaagctcgacgagatcaaaaacgccctgaaatgacgggtggcatccctgtgacccctccccagtgcctctcctggccctggaagttgccactccagtgcccaccagccttgtcctaataaaattaagttgcatcaagct

SEQ ID NO: 43 - RNA, 1019 nt, Borrelia burgdorferi
  source 1..1019: mol_type = other RNA
  mRNA 1..1019: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaacagaacgtgtcttccctggacgagaaaaattctgcaagcgtggacctgcctggcgaaatgaaggtgctggtgagcaaggaaaaagacaaggacggcaagtattctctgaaggcaacagtggataagattgaactgaagggaacatctgacaaggataatggcagtggggtgctggaagggactaaggacgacaagtctaaggccaagctgaccatcgcagatgacctgtccaagacaacctttgagctttttaaagaggacggcaagaccctggtgagccggaaagtgtcttctaaggacaagacatctactgacgagatgttcaatgaaaaaggggagctgagcgccaagacaatgaccagagagaacggaacaaagctggagtacactgagatgaagtccgacgggaccggaaaagctaaggaagtcctgaagaatttcactctggagggcaaggttgccaacgataaggtgacactggaggtgaaagagggaacagtgaccctgtccaaggaaattgccaagagtggcgaagtgaccgtggctctgaacgataccaatacaactcaggccacaaagaagaccggagcatgggactccaaaacatccaccctgaccattagcgttaattcaaagaaaaccacccagctggtgtttactaagcaggacaccatcaccgtgcagaagtatgattccgcaggcactaatctggaagggacagctgtggagatcaagaccctggatgagctgaaaaatgccctgaaatgacgggtggcatccctgtgacccctccccagtgcctctcctggccctggaagttgccactccagtgcccaccagccttgtcctaataaaattaagttgcatcaagct

SEQ ID NO: 44 - RNA, 1019 nt, Borrelia burgdorferi
  source 1..1019: mol_type = other RNA
  mRNA 1..1019: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaagcagaacgtgagtagcctggatgaaaagaatagcgcctctgtggacctgcccggggagatgaaggtgctggtctctaaagagaaagataaggatggaaaatacagcctgaaggcaacagtggacaaaatcgagctcaaaggaacatccgataaggataacgggagcggagtcctggagggcaccaaagacgacaagagcaaggccaagctgacaatcgccgatgatctgagtaagacaactttcgagctctttaaggaggacggcaagaccctcgtgagcagaaaggtgagcagcaaagacaagacctctaccgacgagatgttcaatgagaagggggaactctctgctaagacaatgacacgggagaatggaactaaactggagtacacagaaatgaagtctgacggcactgggaaggcaaaagaggtgctgaagaatttcacactggaagggaaggtggctaacgataaagtgacactggaggttaaggagggaaccgtgactctgtctaaggagattgcaaaaagcggagaggtgacagtcgccctgaacgacactaacaccacccaggccactaagaagacaggagcctgggacagtaaaacctccacactgaccatttccgtgaacagcaagaagacaacccagctggtcttcactaaacaggatactatcacagtccagaagtacgactccgccggaaccaatctggagggaactgccgtggagatcaaaaccctggacgagctcaaaaacgccctgaagtgacgggtggcatccctgtgacccctccccagtgcctctcctggccctggaagttgccactccagtgcccaccagccttgtcctaataaaattaagttgcatcaagct

SEQ ID NO: 45 - RNA, 1067 nt, synthetic construct
  source 1..1067: mol_type = other RNA
  mRNA 1..1067: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaggccaagctgctggtcctgctctgtacctttacagccacttacgccaagcagaacgtgtcttccctggacgaaaagaacagcgtctctgtggacctgcctggagagatgaaggtcctggtgtctaaagagaagaataaggacggcaagtacgacctcattgcaactgtggacaagctggagctgaagggaacatccgacaagaataatggcagcggcgttctggagggcgtgaaggccgacaaaagcaaagtgaagctgactatctctgatgacctggggcaaactacactggaagtgttcaaggaggatgggaagacactggtgagcaaaaaagtgaccagcaaggacaaatcctctacagaggaaaagtttaatgagaaaggcgaggtgtctgaaaagatcatcactcgcgccgacgggactcggctggagtataccgggatcaagagcgacgggtccgggaaagccaaggaggtgctgaaaggatatgatctgaagggagagctctcttccgagaagacaacactggtggtcaaggagggaacagttaccctgtccaaaaacatcagcaagtccggggaggtctccgtcgaactgaatgacaccgactcctctgccgccaccaagaagaccgccgcatggaactctggaacaagcacactgacaatcacagtgaattccaagaagactaaggacctcgtgttcacaaaggagaacacaatcacagtgcagcagtacgacagcaacggaaccaaactggagggctccgcagtggaaatcacaaaactggacgagattaagaacgctctgaagtgacgggtggcatccctgtgacccctccccagtgcctctcctggccctggaagttgccactccagtgcccaccagccttgtcctaataaaattaagttgcatcaagct

SEQ ID NO: 46 - RNA, 1067 nt, synthetic construct
  source 1..1067: mol_type = other RNA
  mRNA 1..1067: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaggccaaactgctcgtgctcttatgcacattcacagcaacctacgccaaacagaatgtcagttccctcgatgagaagaattccgtgagcgtggacctgccaggcgagatgaaggtgctggtgtccaaggagaagaacaaagacgggaaatacgacctgatcgccaccgtggataagctggagctgaaaggcacaagcgacaagaataacggatccggcgtgctggagggagtcaaggccgataagagcaaggtgaagctgaccatcagcgacgatctggggcagactaccctggaagtctttaaggaggacgggaaaacactggtgtcaaaaaaggtgacatccaaagataaatccagcaccgaagagaagtttaacgagaagggggaggtctccgagaagattattacacgcgcagacggcacaaggctggagtataccggaatcaagtccgatggatccggcaaagctaaggaggtgctgaagggatacgatctgaaaggagagctgtcttctgagaagactactctggtggtgaaagaaggcactgtgactctgtctaagaacatctccaagtccggcgaagtttccgtggaacttaacgacacagattcatccgccgccacaaagaagaccgccgcctggaattccggcactagtacactgaccattaccgtcaacagcaagaagacaaaggatctcgtgttcaccaaagaaaatacaattaccgtgcagcagtacgactccaatggcacaaagctggagggctctgcagtggagatcactaagctggacgagatcaaaaatgccctgaaatgacgggtggcatccctgtgacccctccccagtgcctctcctggccctggaagttgccactccagtgcccaccagccttgtcctaataaaattaagttgcatcaagct

SEQ ID NO: 47 - RNA, 1067 nt, synthetic construct
  source 1..1067: mol_type = other RNA
  mRNA 1..1067: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaggcaaagctgctggtgctgctgtgtaccttcactgccacctacgccaagcagaacgtctccagcctggacgagaagaattctgcctccgtcgacctgcccggcgaaatgaaggtcctggtgagcaaggaaaaagacaaagacggcaagtattccctgaaggccacagtggacaaaatcgaactgaaagggacctctgataaggataacggaagcggagtgctggaaggaactaaggatgataagagcaaagccaagctgactattgctgatgatctctctaagaccacctttgaactcttcaaggaggacggcaagacactggtgtcaagaaaggtgtccagcaaggacaagacttctactgatgaaatgtttaatgagaagggagaactgtccgctaagaccatgacacgggagaacggcactaagctggagtataccgagatgaaaagcgatggcactggaaaggccaaggaggtgctgaaaaattttaccctggaaggaaaagtggccaatgataaggtgacactggaggtgaaggagggcacagtcactctgtccaaagagatcgccaagtccggcgaagtgaccgtggctctcaatgacaccaacacaactcaagccacaaaaaagaccggagcatgggattctaaaacatccactctgaccattagcgttaactccaagaagacaacccagctggtctttacaaaacaggacactatcactgtgcagaagtatgattctgccggaaccaatctcgagggcacagccgtggaaatcaagaccctggacgagctgaagaacgccctgaagtgacgggtggcatccctgtgacccctccccagtgcctctcctggccctggaagttgccactccagtgcccaccagccttgtcctaataaaattaagttgcatcaagct

SEQ ID NO: 48 - RNA, 1154 nt, synthetic construct
  source 1..1154: mol_type = other RNA
  mRNA 1..1154: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaggccaagctgctggtgctgctgtgtacattcacagcaacctatgccaagcagaacgtgtctagcctggatgagaagaacagcgtgtccgtggacctcccaggagagatgaaggttctggtgtctaaggagaagaacaaggatgggaagtacgacctgattgcaaccgtcgacaagctggagctgaagggaaccagcgacaaaaacaatggaagcggagtgctggagggagtcaaagcagacaaaagcaaggttaaactgaccatcagcgacgacctgggacagaccacactggaggtctttaaggaagatggaaaaacactcgtgagcaaaaaggtgacctccaaggataagagctctacagaggagaagttcaatgaaaagggagaggtctccgagaagattatcacaagagccgacggcacaagactggagtataccggcattaagtccgacggctctgggaaggccaaagaggtcctgaagggatatgacctgaagggcgaactgtcctccgaaaagacaaccctggtggtgaaagaggggaccgtgaccctgagcaagaacatctcaaaatctggggaggtgtccgtggagctgaacgacactgactcttctgccgctactaagaagaccgcagcatggaattccggcaccagcactctgaccatcaccgtgaacagcaagaagactaaggacctggtgtttactaaggaaaatactatcaccgtccagcagtacgacagcaacgggaccaagctggagggctccgcagtggaaatcactaagctggacgagatcaaaaacgctctgaagtccgggagcatcctggcaatctatagcacagtcgccagctccctggttctcctggtgagcctgggggcaatttccttcggatctggctgacgggtggcatccctgtgacccctccccagtgcctctcctggccctggaagttgccactccagtgcccaccagccttgtcctaataaaattaagttgcatcaagct

SEQ ID NO: 49 - RNA, 1154 nt, synthetic construct
  source 1..1154: mol_type = other RNA
  mRNA 1..1154: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaagcaaagctgctggtgctgctgtgcacattcaccgcaacatacgccaaacagaatgtgtcttccctggacgagaaaaattccgtctctgtggacctgcccggcgaaatgaaggtcctggtgtctaaggaaaagaacaaagacgggaagtacgacctgatcgcaaccgtggataagctggaactgaaggggactagcgacaagaataatggatccggcgtgctggaaggggtgaaagctgacaagtccaaggtcaagctgaccatttctgacgacctggggcagacaaccctggaggtcttcaaagaggacggaaagacactggtgagcaagaaggtgacatccaaggataagtcttccaccgaagaaaaattcaatgagaaaggggaagtgtccgagaaaatcattactcgggccgatggcacaagactggagtacacaggcatcaaatccgacggaagcgggaaggccaaggaggttctgaagggctacgacctgaaaggggagctgtccagcgaaaagacaacactggtggtgaaagaaggaactgtcacactgtctaagaatatcagcaagtcaggcgaggtgagcgtggagctgaacgacaccgactcctccgctgcaacaaaaaagaccgccgcttggaactccggaactagcaccctgactattactgtgaattccaaaaagaccaaagacctggtgtttactaaggagaacaccattaccgtccagcagtatgatagcaatggcaccaaactggaaggaagcgcagttgagatcaccaaactggacgagatcaagaacgcactgaaatccggatccatcctggccatctatagcaccgtcgccagctctctggtgctgctggtgtccctcggggctatctcattcgggtccggctgacgggtggcatccctgtgacccctccccagtgcctctcctggccctggaagttgccactccagtgcccaccagccttgtcctaataaaattaagttgcatcaagct

SEQ ID NO: 50 - RNA, 1154 nt, synthetic construct
  source 1..1154: mol_type = other RNA
  mRNA 1..1154: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaggctaaactgctggtcctgctgtgtaccttcaccgctacatacgccaagcagaatgtgagctccctggacgagaaaaattccgcttccgtggacctgcccggggagatgaaggtgctggtgtccaaggagaaggataaggacggaaagtacagtctgaaggccactgtggacaagatcgagctgaaggggacatctgacaaagataatggatcaggagtgctggaagggacaaaggatgacaagagcaaagccaagctgaccattgccgacgacctgagcaaaactacttttgaactcttcaaagaagacgggaagaccctggtcagccgcaaggtgtcctctaaggacaagactagcactgatgagatgttcaacgagaagggggagctgtctgcaaagacaatgacaagggagaacgggacaaagctggagtacactgagatgaagtccgacggcacagggaaagcaaaagaggttctgaagaacttcaccctggaaggcaaagtggcaaacgacaaggtgacactggaggtgaaggagggaacagtgactctgtccaaggagatcgctaaaagcggcgaggtgactgtggctctgaatgacactaatacaacccaggctacaaagaagaccggagcttgggattctaagactagcactctgactatctccgtgaattctaaaaagacaacccagctggtgttcaccaagcaggataccattacagtgcagaagtacgattccgccggcactaatctggaggggactgctgtggagatcaagaccctggacgaactgaagaacgcactcaagtccggatcaatcctggctatctatagcactgtggcttcctctctggtgctgctggtttccctgggggccatttccttcggcagtggctgacgggtggcatccctgtgacccctccccagtgcctctcctggccctggaagttgccactccagtgcccaccagccttgtcctaataaaattaagttgcatcaagct

SEQ ID NO: 51 - RNA, 1154 nt, synthetic construct
  source 1..1154: mol_type = other RNA
  mRNA 1..1154: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaggcaaaactgctggtgctgctgtgtacattcacagctacttatgcaaagcagaacgtgtcctctctggacgagaagaactccgcctccgtcgatctcccaggagagatgaaagttcttgtgagcaaggagaaggacaaggatggcaaatatagcctgaaggccaccgtggacaagatcgagctgaaaggcacctccgacaaggacaatggctccggagtgctggaaggaaccaaggacgataagtctaaggctaagctgaccatcgctgacgacctgagcaagactacatttgaactcttcaaggaggacggcaagacactggtgtccagaaaggtgagctctaaggacaagactagcactgacgaaatgttcaatgagaagggggagctgagcgctaagacaatgactcgcgagaatggcacaaagctcgaatatacagagatgaagtccgacgggactggcaaggccaaggaagtgctgaagaactttactctggaagggaaagtcgctaatgacaaggtgacactcgaggtgaaggagggcacagtgaccctgtctaaagagatcgctaagtccggggaggtgaccgtcgccctgaacgatacaaacacaacccaggccacaaagaaaaccggcgcatgggatagcaagacatccactctgacaatttctgtgaacagcaagaagacaacccagctggtctttaccaagcaggatacaatcacagtccagaaatatgactctgccggaactaatctggagggaactgcagtggagatcaagacactcgatgaactgaagaatgcactgaagtccgggagcatcctggctatctatagcaccgtcgcctccagcctcgttctgctggtgagcctgggcgccatttccttcggcagcggctgacgggtggcatccctgtgacccctccccagtgcctctcctggccctggaagttgccactccagtgcccaccagccttgtcctaataaaattaagttgcatcaagct

SEQ ID NO: 52 - RNA, 1067 nt, synthetic construct
  source 1..1067: mol_type = other RNA
  mRNA 1..1067: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaggctaagctgctggttctgctgtgtacttttaccgccacatacgctaagcagaacgtgggaagcctggacgagaaaaactccgtgtctgtggatctgcctggggagatgaaggtgctcgttagcaaggagaagaataaggacgggaagtatgacctcattgccacagtggacaagctggagctgaaaggcaccagcgataagaataacggctccggcgtgctggagggggtcaaagccgacaagtccaaggtgaaactgacaatttctgacgacctggggcagaccactctggaggtctttaaagaggacggcaagacactcgtgtccaagaaagtgacctctaaagataagtcctccactgaggagaagtttaatgaaaagggggaagttagcgagaagattattacaagagctgatgggactcgcctggagtataccggcatcaagtccgacggctctggcaaagcaaaggaggtgctgaaaggctatgatctgaagggagagctgtcttccgagaagaccacactggtggtgaaagagggcaccgtgacactgagtaagaacattgacaagtccggcgaagtgagcgtcgaactggacgacactgatagctccgccgccaccaagaaaacagccgcatggaatagcgggacaagcactctgaccatcacagttaactctaaaaagacaaaggacctggtttttactaaagagaataccattaccgttcagcagtatgacagcgccggaaccaagctggagggaagcgcagtggaaatcactaagctggacgagattaagaacgcactgaagtgacgggtggcatccctgtgacccctccccagtgcctctcctggccctggaagttgccactccagtgcccaccagccttgtcctaataaaattaagttgcatcaagct

SEQ ID NO: 53 - RNA, 1067 nt, synthetic construct
  source 1..1067: mol_type = other RNA
  mRNA 1..1067: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaggccaaactcctggtgctcctgtgtaccttcaccgctacctacgccaagcagaacgtgggaagcctggacgaaaagaattccgtgagcgtcgacctgcccggcgaaatgaaggtgctcgtgtccaaggagaagaataaggatggaaagtatgacctgattgccactgtcgataagctggaactgaaggggacctccgacaagaataacggctcaggcgtgctggagggcgttaaggcagataagagcaaagtgaagctgacaatttctgatgatctgggacagactacactcgaggtctttaaggaagacggcaagaccctggtgagcaagaaagtgactagcaaggataagtcctccacagaggagaaattcaacgaaaaaggagaggtgtctgaaaaaattattacacgggctgatggaaccaggctggagtacacagggattaagtccgacgggtctggaaaggctaaagaggtgctgaaaggatatgacctgaagggggaactgtcttccgaaaagaccacactggtcgtgaaggagggcacagtgacactgtctaaaaacatcgacaagagcggggaggtgagtgtcgagctggacgacactgattcttccgctgccacaaagaagaccgcagcctggaatagcgggacctccacactgactatcaccgtgaactctaagaagacaaaggacctggtgtttacaaaggagaataccatcactgtgcagcagtacgattccgccggcactaagctggagggatccgcagtggaaatcacaaagctggatgagatcaagaatgcactaaagtgacgggtggcatccctgtgacccctccccagtgcctctcctggccctggaagttgccactccagtgcccaccagccttgtcctaataaaattaagttgcatcaagct

SEQ ID NO: 54 - RNA, 1067 nt, synthetic construct
  source 1..1067: mol_type = other RNA
  mRNA 1..1067: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaggccaaactgctggtgctgctgtgcactttcactgcaacttacgccaagcagaacgtgggatctctggatgaaaagaattctgcaagcgtggatctgccaggcgagatgaaggttctggtctccaaggagaaggacaaggacggcaaatattctctgaaagccacagtggataagattgagctgaagggcacatccgataaggacgacggctctggggtgctggagggaaccaaggacgacaaaagcaaggccaagctgacaatcgccgacgacctgtctaaaactactttcgaactctttaaggaggatggcaaaaccctggtgtccaggaaagtgtcctccaaagacaagacctctacagatgagatgttcaatgaaaagggggagctgtccgccaaaaccatgaccagagaggacggaaccaagctggaatataccgaaatgaagtccgacggcaccggaaaagccaaggaggtcctgaaacacttcacactggaggggaaggtggccaatgacaaggtgacactggaggtgaaagagggcacagtgactctgagcaaagagatcgccaaatctggcgaggttactgtggcactgaaggatacaagcaccacacaggccaccaagaaaacaggcgcctgggatagcaagacatccactctgacaatttccgtcaattctaagaagaccacccagctcgtgttcactaagcaggacactatcaccgtgcagaagtatgactctgcaggaacaaacctggaaggcacagctgtggaaatcaaaactctggatgaactgaaaaacgccctgaagtgacgggtggcatccctgtgacccctccccagtgcctctcctggccctggaagttgccactccagtgcccaccagccttgtcctaataaaattaagttgcatcaagct

SEQ ID NO: 55 - RNA, 1067 nt, synthetic construct
  source 1..1067: mol_type = other RNA
  mRNA 1..1067: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaagccaaacttctggtcctgctctgtaccttcactgcaacctacgccaagcagaacgtgggctctctggacgagaagaacagtgctagcgtcgacctgccaggggaaatgaaggtgctggtctctaaagagaaggacaaagacggcaagtatagcctgaaggccacagtggacaagattgaactgaagggaacttccgacaaggacgacgggagcggcgtcctggaaggcacaaaggatgacaagtccaaagccaagctgaccattgctgacgacctgtctaagaccaccttcgaactcttcaaggaagatggaaagacactggtgagcaggaaggtgtctagcaaggacaagacctcaacagacgaaatgtttaatgaaaaaggcgagctgtctgccaagacgatgacccgcgaggacggaaccaagctggaatacaccgaaatgaagtccgatgggactggcaaggccaaggaggtcctgaagcacttcacactggaggggaaggtggccaatgataaggtgactctcgaggtcaaggagggcacagttaccctgtctaaggagatcgccaaatccggcgaggtgaccgtggcactgaaggatacaagcactactcaggcaactaagaagacaggcgcttgggactctaagacaagcacactgactatctctgtgaattccaaaaagacaactcagctggtgttcacaaagcaggatacaattaccgtgcagaagtatgattccgctggaacaaatctggagggaacagccgtggagattaagacactggacgagctgaagaatgctctgaaatgacgggtggcatccctgtgacccctccccagtgcctctcctggccctggaagttgccactccagtgcccaccagccttgtcctaataaaattaagttgcatcaagct

SEQ ID NO: 56 - RNA, 1154 nt, synthetic construct
  source 1..1154: mol_type = other RNA
  mRNA 1..1154: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaagcaaagctgctggtcctgctgtgtactttcacagcaacttatgcaaaacagaatgtgggctctctggacgagaaaaacagcgtgtccgtggacctgcccggagagatgaaagtgctggtgtccaaggagaagaacaaggatgggaagtacgacctgatcgctaccgttgacaagctggagctgaaggggactagcgataaaaacaacggctccggcgtcctggaaggagtgaaggccgacaagagcaaggtgaaactgactatttccgacgatctgggacagacaactctggaagtgtttaaggaagacggaaaaactctcgtttccaagaaggtgacatccaaggacaagtcctccaccgaggagaagttcaatgagaagggagaggtttccgagaagatcatcacaagagcagacggcaccagactggagtacaccggaatcaagagcgacggctctggcaaggccaaagaggtgctgaagggctatgacctgaagggggaactgtcctctgagaaaaccactctggtggtgaaagagggcacagtgacactgtccaaaaacatcgacaagagtggggaggtgtccgtggagctggacgatacagacagctccgccgcaaccaaaaagactgcagcctggaactccggaacatctaccctgactatcactgttaactccaagaaaactaaggacctggtgtttactaaggagaatacaatcacagtgcagcagtatgatagcgccggaaccaagctggagggctccgccgtggagatcacaaagctggatgagattaagaacgccctgaagtctgggtccattctggcaatctacagcacagtggcctcttctctggtgctgctggtttccctgggcgccattagttttggctctggctgacgggtggcatccctgtgacccctccccagtgcctctcctggccctggaagttgccactccagtgcccaccagccttgtcctaataaaattaagttgcatcaagct

SEQ ID NO: 57 - RNA, 1154 nt, synthetic construct
  source 1..1154: mol_type = other RNA
  mRNA 1..1154: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaagccaagctcctggtgctcctgtgcacattcactgcaacttacgccaagcagaacgtgggaagcctggacgagaagaacagcgtgagcgtggatctgcccggagagatgaaggtcctggtgtccaaggagaagaataaggacggaaaatacgacctcattgctaccgtcgataaactggagctgaagggaaccagcgataagaacaatggatctggagtgctggagggggtcaaggctgataagtcaaaggtgaaactgacaatcagtgatgacctgggccagacaaccctggaggtgttcaaggaagacgggaaaactctggtgagtaagaaggtcacctccaaagataagagcagcacagaagagaagttcaacgaaaagggagaggtgagcgagaagatcatcacacgcgctgacggcacccgcctggagtataccggcatcaagtccgacgggtccggcaaagctaaagaggtgctgaaaggctacgatctgaagggagagctgagctccgagaaaactaccctggtggtgaaggagggaacagtgacactgtccaaaaacatcgataagagtggggaggtgagcgttgagctggacgacacagacagcagcgcagccacaaagaagacagccgcatggaatagcgggaccagcacactgaccatcacagtgaattctaaaaagactaaagacctcgtctttactaaagaaaacactattactgtccagcaatacgactctgccggaactaagctggagggatctgccgtggagattaccaagctggacgagatcaagaacgccctgaagtccggcagcatcctcgccatctactccaccgtggcctctagcctggttctgctggtgagcctgggcgccatttcttttgggagcggatgacgggtggcatccctgtgacccctccccagtgcctctcctggccctggaagttgccactccagtgcccaccagccttgtcctaataaaattaagttgcatcaagct

SEQ ID NO: 58 - RNA, 1154 nt, synthetic construct
  source 1..1154: mol_type = other RNA
  mRNA 1..1154: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaggctaagctgctggtgctgctgtgcaccttcactgcaacctacgccaagcaaaatgtcggctccctggatgaaaagaatagcgcctccgtggacctgcctggggagatgaaagtgctggtgtccaaggaaaaagacaaagacgggaagtacagcctgaaggccactgtggacaagattgagctgaaagggacaagcgacaaggatgacgggtctggagtgctggagggcactaaggacgataagagcaaggctaagctgactatcgccgatgacctgagcaagaccacttttgaactcttcaaggaagacggcaaaacactggtatcccgcaaggtgtctagcaaggataagacatccacagacgagatgttcaatgagaaaggagaactgagcgccaagaccatgaccagggaagatggaaccaagctggaatacaccgagatgaagtctgacggcaccggcaaggccaaggaggtgctgaagcactttactttggagggcaaagtggccaacgacaaggtgacactggaggtgaaagaggggaccgtgaccctgtctaaagagattgcaaagtccggcgaagtgaccgtggccctcaaggacacttctaccacccaggctacaaagaagaccggcgcttgggattccaaaacttctaccctgactatctccgtgaattctaagaagacaacccagctggtgtttaccaagcaggataccattaccgtgcagaaatatgatagcgccggcactaatctggagggcaccgctgtggagattaagaccctggacgagctgaaaaacgcactgaaatccggctctattctggcaatctactccacagtggcttcaagcctggtgctgctcgtgtccctcggggcaatctccttcggatctgggtgacgggtggcatccctgtgacccctccccagtgcctctcctggccctggaagttgccactccagtgcccaccagccttgtcctaataaaattaagttgcatcaagct

SEQ ID NO: 59 - RNA, 1154 nt, synthetic construct
  source 1..1154: mol_type = other RNA
  mRNA 1..1154: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaggcaaagctgctggtgctcctgtgcacctttacagctacatacgcaaaacagaacgtgggcagcctggacgagaagaatagcgccagcgtggatctgccaggcgagatgaaggtgctggtgagcaaagagaaggataaagatgggaagtacagcctgaaggctactgtggacaagatcgaactgaagggaactagcgataaagacgatggaagcggcgtgctggagggaactaaggacgataaaagcaaagccaagctgacaattgccgacgacctgtccaaaactacatttgaactctttaaggaggatggaaaaacactggtgagcagaaaggtgtcttctaaggacaagacctccaccgatgagatgttcaacgagaagggcgagctgtctgcaaaaactatgacacgcgaggacggaacaaagctggagtatactgagatgaagtctgacggcaccgggaaggccaaagaggtgctgaaacacttcaccctggaggggaaggtggctaacgataaggttaccctggaggtcaaggagggaactgtgactctgagcaaggagatcgccaagtctggcgaggtcacagtggccctcaaggacacaagcaccacccaggctacaaagaagacaggcgcctgggattctaaaacaagcaccctgactatcagtgtcaactccaagaagacaacccagctggtgttcaccaagcaggacaccatcaccgtgcagaaatatgactctgctggaacaaacctggaagggaccgctgtcgaaatcaagactctggacgaactgaagaatgccctgaagtccgggagcatcctggcaatctactctacagtggcttcctcccttgttctgctggtcagcctgggcgccatcagctttggctctggctgacgggtggcatccctgtgacccctccccagtgcctctcctggccctggaagttgccactccagtgcccaccagccttgtcctaataaaattaagttgcatcaagct

SEQ ID NO: 60 - RNA, 51 nt, synthetic construct
  source 1..51: mol_type = other RNA
atgaaggccaagctgctggtcctgctctgtacctttacagccacttacgcc

SEQ ID NO: 61 - RNA, 51 nt, synthetic construct
  source 1..51: mol_type = other RNA
atgaaggccaaactgctcgtgctcttatgcacattcacagcaacctacgcc

SEQ ID NO: 62 - RNA, 51 nt, synthetic construct
  source 1..51: mol_type = other RNA
atgaaggctaagctgctggttctgctgtgtacttttaccgccacatacgct

SEQ ID NO: 63 - RNA, 51 nt, synthetic construct
  source 1..51: mol_type = other RNA
atgaaggccaaactcctggtgctcctgtgtaccttcaccgctacctacgcc

SEQ ID NO: 64 - RNA, 51 nt, synthetic construct
  source 1..51: mol_type = other RNA
atgaaggcaaagctgctggtgctgctgtgtaccttcactgccacctacgcc

SEQ ID NO: 65 - RNA, 51 nt, synthetic construct
  source 1..51: mol_type = other RNA
atgaaggccaaactgctggtgctgctgtgcactttcactgcaacttacgcc

SEQ ID NO: 66 - RNA, 51 nt, synthetic construct
  source 1..51: mol_type = other RNA
atgaaagccaaacttctggtcctgctctgtaccttcactgcaacctacgcc

SEQ ID NO: 67 - RNA, 51 nt, synthetic construct
  source 1..51: mol_type = other RNA
atgaaggccaagctgctggtgctgctgtgtacattcacagcaacctatgcc

SEQ ID NO: 68 - RNA, 51 nt, synthetic construct
  source 1..51: mol_type = other RNA
atgaaagcaaagctgctggtgctgctgtgcacattcaccgcaacatacgcc

SEQ ID NO: 69 - RNA, 51 nt, synthetic construct
  source 1..51: mol_type = other RNA
atgaaagcaaagctgctggtcctgctgtgtactttcacagcaacttatgca

SEQ ID NO: 70 - RNA, 51 nt, synthetic construct
  source 1..51: mol_type = other RNA
atgaaagccaagctcctggtgctcctgtgcacattcactgcaacttacgcc

SEQ ID NO: 71 - RNA, 51 nt, synthetic construct
  source 1..51: mol_type = other RNA
atgaaggctaaactgctggtcctgctgtgtaccttcaccgctacatacgcc

SEQ ID NO: 72 - RNA, 51 nt, synthetic construct
  source 1..51: mol_type = other RNA
atgaaggcaaaactgctggtgctgctgtgtacattcacagctacttatgca

SEQ ID NO: 73 - RNA, 69 nt, synthetic construct
  source 1..69: mol_type = other RNA
atcctggcaatctatagcacagtcgccagctccctggttctcctggtgagcctgggggcaatttccttc

SEQ ID NO: 74 - RNA, 69 nt, synthetic construct
  source 1..69: mol_type = other RNA
atcctggccatctatagcaccgtcgccagctctctggtgctgctggtgtccctcggggctatctcattc

SEQ ID NO: 75 - RNA, 69 nt, synthetic construct
  source 1..69: mol_type = other RNA
attctggcaatctacagcacagtggcctcttctctggtgctgctggtttccctgggcgccattagtttt

SEQ ID NO: 76 - RNA, 69 nt, synthetic construct
  source 1..69: mol_type = other RNA
atcctcgccatctactccaccgtggcctctagcctggttctgctggtgagcctgggcgccatttctttt

SEQ ID NO: 77 - RNA, 69 nt, synthetic construct
  source 1..69: mol_type = other RNA
atcctggctatctatagcactgtggcttcctctctggtgctgctggtttccctgggggccatttccttc

SEQ ID NO: 78 - RNA, 69 nt, synthetic construct
  source 1..69: mol_type = other RNA
atcctggctatctatagcaccgtcgcctccagcctcgttctgctggtgagcctgggcgccatttccttc

SEQ ID NO: 79 - RNA, 69 nt, synthetic construct
  source 1..69: mol_type = other RNA
attctggcaatctactccacagtggcttcaagcctggtgctgctcgtgtccctcggggcaatctccttc

SEQ ID NO: 80 - RNA, 69 nt, synthetic construct
  source 1..69: mol_type = other RNA
atcctggcaatctactctacagtggcttcctcccttgttctgctggtcagcctgggcgccatcagcttt

SEQ ID NO: 81 - protein, 5 aa, synthetic construct
DGGGS

SEQ ID NO: 82 - protein, 5 aa, synthetic construct
TGEKP

SEQ ID NO: 83 - protein, 14 aa, synthetic construct
EGKSSGSGSESKVD

SEQ ID NO: 84 - protein, 18 aa, synthetic construct
KESGSVSSEQLAQFRSLD

SEQ ID NO: 85 - protein, 8 aa, synthetic construct
GGRRGGGS

SEQ ID NO: 86 - protein, 9 aa, synthetic construct
LRQRDGERP

SEQ ID NO: 87 - protein, 12 aa, synthetic construct
LRQKDGGGSERP

SEQ ID NO: 88 - protein, 18 aa, synthetic construct
GSTSGSGKPGSGEGSTKG

SEQ ID NO: 89 - RNA, 140 nt, synthetic construct
  source 1..140: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 90 - RNA, 100 nt, synthetic construct
  source 1..100: mol_type = other RNA
cgggtggcatccctgtgacccctccccagtgcctctcctggccctggaagttgccactccagtgcccaccagccttgtcctaataaaattaagttgcatc

SEQ ID NO: 91 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 92 - protein, 4 aa, synthetic construct
GGRR

SEQ ID NO: 93 - protein, 5 aa, synthetic construct
  REGION 1..5: note = The entire sequence of amino acids 1-5 can be repeated one to five times.
GGGGS

SEQ ID NO: 94 - RNA, 11 nt, synthetic construct
  source 1..11: mol_type = other RNA
gggatcctacc

SEQ ID NO: 95 - protein, 17 aa, synthetic construct
MKAKLLVLLCTFTATYA

SEQ ID NO: 96 - protein, 17 aa, synthetic construct
MKAILVVLLYTFTTANA

SEQ ID NO: 97 - protein, 17 aa, synthetic construct
MKVKLLVLLCTFTATYA

SEQ ID NO: 98 - protein, 17 aa, synthetic construct
MKAILVVLLYTFATANA

SEQ ID NO: 99 - protein, 17 aa, synthetic construct
MKAILVVLLYTFTTANA

SEQ ID NO: 100 - protein, 17 aa, synthetic construct
MKAILVVLLYTFTTANA

SEQ ID NO: 101 - protein, 17 aa, synthetic construct
MKAILVVLLYTFTTANA

SEQ ID NO: 102 - protein, 17 aa, synthetic construct
MKAKLLVLLCTFTATYA

SEQ ID NO: 103 - protein, 17 aa, synthetic construct
MKVKLLVLLCTFTATYA

SEQ ID NO: 104 - protein, 17 aa, synthetic construct
MKAILVVMLYTFTTANA

SEQ ID NO: 105 - protein, 17 aa, synthetic construct
MKAILVVMLYTFTTANA

SEQ ID NO: 106 - protein, 17 aa, synthetic construct
MKAILVVMLYTFTTANA

SEQ ID NO: 107 - protein, 17 aa, synthetic construct
MKAILVVMLYTFTTANA

SEQ ID NO: 108 - protein, 17 aa, synthetic construct
MKAILVVMLYTFTTANA

SEQ ID NO: 109 - protein, 17 aa, synthetic construct
MKAILVVLLYTFTTANA

SEQ ID NO: 110 - protein, 16 aa, synthetic construct
MKTIIALSYILCLVFT

SEQ ID NO: 111 - protein, 16 aa, synthetic construct
MKTIIALSYILCLVFA

SEQ ID NO: 112 - protein, 16 aa, synthetic construct
MKTIIALSYILCLVFA

SEQ ID NO: 113 - protein, 16 aa, synthetic construct
MKTIIALSNILCLVFA

SEQ ID NO: 114 - protein, 16 aa, synthetic construct
MKTIIALSNILCLVFA

SEQ ID NO: 115 - protein, 16 aa, synthetic construct
MKTIIALSYILCLVFA

SEQ ID NO: 116 - protein, 16 aa, synthetic construct
MKAIIALSNILCLVFA

SEQ ID NO: 117 - protein, 16 aa, synthetic construct
MKAIIALSNILCLVFA

SEQ ID NO: 118 - protein, 16 aa, synthetic construct
MKTIIALSYILCLVFA

SEQ ID NO: 119 - protein, 16 aa, synthetic construct
MKTIIALSCILCLVFA

SEQ ID NO: 120 - protein, 16 aa, synthetic construct
MKTIIALSYILCLVFA

SEQ ID NO: 121 - protein, 16 aa, synthetic construct
MKTIIALSYILCLVFA

SEQ ID NO: 122 - protein, 16 aa, synthetic construct
MKTIIALSYILCLVFA

SEQ ID NO: 123 - protein, 16 aa, synthetic construct
MKTIIALSYILCLVFA

SEQ ID NO: 124 - protein, 16 aa, synthetic construct
MKTIIALSYILCLVFA

SEQ ID NO: 125 - protein, 16 aa, synthetic construct
MKTIIALSYILCLVFA

SEQ ID NO: 126 - protein, 16 aa, synthetic construct
MKTIIALSYILCLVFA

SEQ ID NO: 127 - protein, 16 aa, synthetic construct
MKTIIALSYILCLVFA

SEQ ID NO: 128 - protein, 16 aa, synthetic construct
MKTIIALSHILCLVFA

SEQ ID NO: 129 - protein, 16 aa, synthetic construct
MKTIIALSYILCLVFA

SEQ ID NO: 130 - protein, 16 aa, synthetic construct
MKTIIALSYILCLVFA

SEQ ID NO: 131 - protein, 16 aa, synthetic construct
MKTIIALSYILCLVFA

SEQ ID NO: 132 - protein, 15 aa, synthetic construct
MKAIIVLLMVVTSNA

SEQ ID NO: 133 - skipped

SEQ ID NO: 134 - protein, 15 aa, synthetic construct
MKAIIVLLMVVTSNA

SEQ ID NO: 135 - protein, 15 aa, synthetic construct
MKAIIVLLMVVTSSA

SEQ ID NO: 136 - protein, 15 aa, synthetic construct
MKAIIVLLMVVTSNA

SEQ ID NO: 137 - protein, 15 aa, synthetic construct
MKAIIVLLMVVTSNA

SEQ ID NO: 138 - protein, 15 aa, synthetic construct
MKAIIVLLMVVTSNA

SEQ ID NO: 139 - protein, 15 aa, synthetic construct
MKAIIVLLMVVTSNA

SEQ ID NO: 140 - protein, 15 aa, synthetic construct
MKAIIVLLMVVTSNA

SEQ ID NO: 141 - protein, 15 aa, synthetic construct
MKAIIVLLMVVTSNA

SEQ ID NO: 142 - protein, 15 aa, synthetic construct
MKAIIVLLMVVTSNA

SEQ ID NO: 143 - protein, 15 aa, synthetic construct
MEAIIVLLMVVTSNA

SEQ ID NO: 144 - protein, 15 aa, synthetic construct
MKAIIVLLMVVTSNA

SEQ ID NO: 145 - protein, 17 aa, synthetic construct
  VARIANT 3: note = X can be A or V
  VARIANT 4: note = X can be I or K
  VARIANT 6: note = X can be L or V
  VARIANT 8: note = X can be L or M
  VARIANT 10: note = X can be Y or C
  VARIANT 13..15: note = X can be T or A
  VARIANT 16: note = X can be N or Y
MKXXLXVXLXTFXXXXA

SEQ ID NO: 146 - protein, 16 aa, synthetic construct
  VARIANT 3: note = X can be A or T
  VARIANT 9: note = X can be Y, N, C or H
  VARIANT 16: note = X can be A or T
MKXIIALSXILCLVFX

SEQ ID NO: 147 - protein, 15 aa, synthetic construct
  VARIANT 14: note = X can be S or N
MKAIIVLLMVVTSXA

SEQ ID NO: 148 - protein, 15 aa, synthetic construct
  VARIANT 2: note = X can be K or E
MXAIIVLLMVVTSNA

SEQ ID NO: 149 - protein, 240 aa, Propionibacterium acnes
MVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 150 - protein, 400 aa, Propionibacterium acnes
MAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 151 - protein, 257 aa, Borrelia burgdorferi
MKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 152 - protein, 273 aa, synthetic construct
MKAILVVLLYTFATANAKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 153 - protein, 271 aa, synthetic construct
MKAIIVLLMVVTSNAKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 154 - protein, 275 aa, synthetic construct
MVPQALLFVPLLVFPLCFGKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 155 - protein, 276 aa, synthetic construct
MFLIQCLISAVIFYIQVTNAKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 156 - protein, 288 aa, synthetic construct
MGVTGILQLPRDRFKRTSFFLWVIILFQRTFSKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 157 - protein, 302 aa, synthetic construct
MKAILVVLLYTFATANAKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKSGSILAIYSTVASSLVLVVSLGAISFGSG

SEQ ID NO: 158 - protein, 298 aa, synthetic construct
MKAIIVLLMVVTSNAKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKSGSSTAASSLAVTLMLAIFIVYMVGSG

SEQ ID NO: 159 - protein, 302 aa, synthetic construct
MVPQALLFVPLLVFPLCFGKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKSGSVLLSAGALTALMLIIFLMTCWGSG

SEQ ID NO: 160 - protein, 303 aa, synthetic construct
MFLIQCLISAVIFYIQVTNAKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKSGSIIIPIVASVMILTAMVIVIVIGSG

SEQ ID NO: 161 - protein, 315 aa, synthetic construct
MGVTGILQLPRDRFKRTSFFLWVIILFQRTFSKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKSGSWIPAGIGVTGVIIAVIALFCIGSG

SEQ ID NO: 162 - protein, 240 aa, synthetic construct; identical to SEQ ID NO: 149

SEQ ID NO: 163 - protein, 256 aa, synthetic construct
MKAILVVLLYTFATANAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 164 - protein, 254 aa, synthetic construct
MKAIIVLLMVVTSNAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 165 - protein, 258 aa, synthetic construct
MVPQALLFVPLLVFPLCFGVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 166 - protein, 259 aa, synthetic construct
MFLIQCLISAVIFYIQVTNAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 167 - protein, 271 aa, synthetic construct
MGVTGILQLPRDRFKRTSFFLWVIILFQRTFSVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 168 - protein, 285 aa, synthetic construct
MKAILVVLLYTFATANAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAASGSILAIYSTVASSLVLVVSLGAISFGSG

SEQ ID NO: 169 - protein, 281 aa, synthetic construct
MKAIIVLLMVVTSNAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAASGSSTAASSLAVTLMLAIFIVYMVGSG

SEQ ID NO: 170 - protein, 285 aa, synthetic construct
MVPQALLFVPLLVFPLCFGVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAASGSVLLSAGALTALMLIIFLMTCWGSG

SEQ ID NO: 171 - protein, 286 aa, synthetic construct
MFLIQCLISAVIFYIQVTNAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAASGSIIIPIVASVMILTAMVIVIVIGSG

SEQ ID NO: 172 - protein, 298 aa, synthetic construct
MGVTGILQLPRDRFKRTSFFLWVIILFQRTFSVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAASGSWIPAGIGVTGVIIAVIALFCIGSG

SEQ ID NO: 173 - protein, 400 aa, synthetic construct; identical to SEQ ID NO: 150

SEQ ID NO: 174 - protein, 416 aa, synthetic construct
MKAILVVLLYTFATANAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 175 - protein, 414 aa, synthetic construct
MKAIIVLLMVVTSNAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 176 - protein, 418 aa, synthetic construct
MVPQALLFVPLLVFPLCFGAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 177 - protein, 419 aa, synthetic construct
MFLIQCLISAVIFYIQVTNAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 178 - protein, 431 aa, synthetic construct
MGVTGILQLPRDRFKRTSFFLWVIILFQRTFSAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 179 - protein, 445 aa, synthetic construct
MKAILVVLLYTFATANAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVTSGSILAIYSTVASSLVLVVSLGAISFGSG

SEQ ID NO: 180 - protein, 441 aa, synthetic construct
MKAIIVLLMVVTSNAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVTSGSSTAASSLAVTLMLAIFIVYMVGSG

SEQ ID NO: 181 - protein, 445 aa, synthetic construct
MVPQALLFVPLLVFPLCFGAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVTSGSVLLSAGALTALMLIIFLMTCWGSG

SEQ ID NO: 182 - protein, 446 aa, synthetic construct
MFLIQCLISAVIFYIQVTNAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVTSGSIIIPIVASVMILTAMVIVIVIGSG

SEQ ID NO: 183 - protein, 458 aa, synthetic construct
MGVTGILQLPRDRFKRTSFFLWVIILFQRTFSAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVTSGSWIPAGIGVTGVIIAVIALFCIGSG

SEQ ID NO: 184 - protein, 19 aa, unidentified
  source 1..19: note = Rabies virus
MVPQALLFVPLLVFPLCFG

SEQ ID NO: 185 - protein, 20 aa, unidentified
  source 1..20: note = Rabies virus
VLLSAGALTALMLIIFLMTC

SEQ ID NO: 186 - protein, 257 aa, synthetic construct
KQNVSSLDEKNSVSVDLPGGMKVLVSKEKDKDGKYSLMATVEKLELKGTSDKSNGSGVLEGEKADKSKAKLTISQDLNQTTFEIFKEDGKTLVSRKVNSKDKSSTEEKFNDKGKLSEKVVTRANGTRLEYTEIKNDGSGKAKEVLKGFALEGTLTDGGETKLTVTEGTVTLSKNISKSGEITVALNDTETTPADKKTGEWKSDTSTLTISKNSQKPKQLVFTKENTITVQNYNRAGNALEGSPAEIKDLAELKAALK

SEQ ID NO: 187 - protein, 256 aa, synthetic construct
KQNVSSLDEKNSVSVDLPGEMKVLVSKEKDKDGKYSLMATVDKLELKGTSDKSNGSGTLEGEKSDKSKAKLTISEDLSKTTFEIFKEDGKTLVSKKVNSKDKSSIEEKFNAKGELSEKTILRANGTRLEYTEIKSDGTGKAKEVLKDFALEGTLAADKTTLKVTEGTVVLSKHIPNSGEITVELNDSNSTQATKKTGKWDSNTSTLTISVNSKKTKNIVFTKEDTITVQKYDSAGTNLEGNAVEIKTLDELKNALK

SEQ ID NO: 188 - protein, 257 aa, synthetic construct
KQNVSSLDEKNSVSVDLPGGMKVLVSKEKDKDGKYSLMATVEKLELKGTSDKNNGSGTLEGEKTDKSKVKLTIAEDLSKTTFEIFKEDGKTLVSKKVTLKDKSSTEEKFNEKGEISEKTIVRANGTRLEYTDIKSDGSGKAKEVLKDFTLEGTLAADGKTTLKVTEGTVVLSKNILKSGEITVALDDSDTTQATKKTGKWDSKTSTLTISVNSQKTKNLVFTKEDTITVQKYDSAGTNLEGKAVEITTLEKLKDALK

SEQ ID NO: 189 - protein, 257 aa, synthetic construct
KQNVSSLDEKNSVSVDLPGGMTVLVSKEKDKDGKYSLEATVDKLELKGTSDKNNGSGTLEGEKTDKSKVKSTIADDLSQTKFEIFKEDGKTLVSKKVTLKDKSSTEEKFNGKGETSEKTIVRANGTRLEYTDIKSDGSGKAKEVLKDFTLEGTLAADGKTTLKVTEGTVVLSKNILKSGEITAALDDSDTTRATKKTGKWDSKTSTLTISVNSQKTKNLVFTKEDTITVQRYDSAGTNLEGKAVEITTLKELKNALK

SEQ ID NO: 190 - protein, 257 aa, synthetic construct
KQNVSSLDEKNSVSVDLPGEMKVLVSKEKDKDGKYSLEATVDKLELKGTSDKNNGSGVLEGVKAAKSKAKLTIADDLSQTKFEIFKEDGKTLVSKKVTLKDKSSTEEKFNDKGKLSEKVVTRANGTRLEYTEIQNDGSGKAKEVLKSLTLEGTLTADGETKLTVEAGTVTLSKNISESGEITVELKDTETTPADKKSGTWDSKTSTLTISKNSQKTKQLVFTKENTITVQKYNTAGTKLEGSPAEIKDLEALKAALK

SEQ ID NO: 191 - protein, 4 aa, synthetic construct
  REGION 1..4: note = The entire sequence of amino acids 1-4 can be repeated one to eight times.
GGGG

SEQ ID NO: 192 - protein, 4 aa, synthetic construct
  REGION 1..4: note = The entire sequence of amino acids 1-4 can be repeated one to eight times.
GSGS

SEQ ID NO: 193 - protein, 273 aa, synthetic construct
MKAKLLVLLCTFTATYAKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 194 - protein, 273 aa, synthetic construct; identical to SEQ ID NO: 152

SEQ ID NO: 195 - protein, 272 aa, synthetic construct
MKTIIALSYILCLVFAKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 196 - protein, 271 aa, synthetic construct; identical to SEQ ID NO: 153

SEQ ID NO: 197 - protein, 268 aa, synthetic construct
MFVFLVLLPLVSKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 198 - protein, 277 aa, synthetic construct
MFLLTTKRTMFVFLVLLPLVSKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 199 - protein, 275 aa, synthetic construct; identical to SEQ ID NO: 154

SEQ ID NO: 200 - protein, 286 aa, synthetic construct
MGTVNKPVVGVLMGFGIITGTLRITNPVRAKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 201 - protein, 276 aa, synthetic construct; identical to SEQ ID NO: 155

SEQ ID NO: 202 - protein, 277 aa, synthetic construct
MQALGIKTEHFIIMCLLSGHAKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 203 - protein, 279 aa, synthetic construct
MGLKVNVSAIFMAVLLTLQTPTGKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 204 - protein, 277 aa, synthetic construct
MGAAAALTAVVLQGYNPPAYGKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 205 - protein, 279 aa, synthetic construct
MGAPQAFLAGLLLAAVAVGTARAKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 206 - protein, 275 aa, synthetic construct
MKVFLVTCLGFAVFSSSVCKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 207 - protein, 288 aa, synthetic construct; identical to SEQ ID NO: 156

SEQ ID NO: 208 - protein, 272 aa, synthetic construct
MRSLIIFLLFPSIIYSKQNVSSLDEKNSVSVDLPGEMKVLVSKEKNKDGKYDLIATVDKLELKGTSDKSNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 209 - protein, 256 aa, synthetic construct
MKAKLLVLLCTFTATYAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 210 - protein, 256 aa, synthetic construct; identical to SEQ ID NO: 163

SEQ ID NO: 211 - protein, 255 aa, synthetic construct
MKTIIALSYILCLVFAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 212 - protein, 251 aa, synthetic construct
MFVFLVLLPLVSVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 213 - protein, 260 aa, synthetic construct
MFLLTTKRTMFVFLVLLPLVSVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 214 - protein, 258 aa, synthetic construct; identical to SEQ ID NO: 165

SEQ ID NO: 215 - protein, 269 aa, synthetic construct
MGTVNKPVVGVLMGFGIITGTLRITNPVRAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 216 - protein, 259 aa, synthetic construct; identical to SEQ ID NO: 166

SEQ ID NO: 217 - protein, 260 aa, synthetic construct
MQALGIKTEHFIIMCLLSGHAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 218 - protein, 262 aa, synthetic construct
MGLKVNVSAIFMAVLLTLQTPTGVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 219 - protein, 260 aa, synthetic construct
MGAAAALTAVVLQGYNPPAYGVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 220 - protein, 262 aa, synthetic construct
MGAPQAFLAGLLLAAVAVGTARAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 221 - protein, 258 aa, synthetic construct
MKVFLVTCLGFAVFSSSVCVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 222 - protein, 271 aa, synthetic construct; identical to SEQ ID NO: 167

SEQ ID NO: 223 - protein, 255 aa, synthetic construct
MRSLIIFLLFPSIIYSVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAA

SEQ ID NO: 224 - protein, 416 aa, synthetic construct
MKAKLLVLLCTFTATYAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 225 - protein, 416 aa, synthetic construct; identical to SEQ ID NO: 174

SEQ ID NO: 226 - protein, 415 aa, synthetic construct
MKTIIALSYILCLVFAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 227 - protein, 414 aa, synthetic construct; identical to SEQ ID NO: 175

SEQ ID NO: 228 - protein, 411 aa, synthetic construct
MFVFLVLLPLVSAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 229 - protein, 420 aa, synthetic construct
MFLLTTKRTMFVFLVLLPLVSAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 230 - protein, 418 aa, synthetic construct; identical to SEQ ID NO: 176

SEQ ID NO: 231 - protein, 429 aa, synthetic construct
MGTVNKPVVGVLMGFGIITGTLRITNPVRAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 232 - protein, 419 aa, synthetic construct; identical to SEQ ID NO: 177

SEQ ID NO: 233 - protein, 420 aa, synthetic construct
MQALGIKTEHFIIMCLLSGHAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 234 - protein, 422 aa, synthetic construct
MGLKVNVSAIFMAVLLTLQTPTGAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 235 - protein, 420 aa, synthetic construct
MGAAAALTAVVLQGYNPPAYGAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 236 - protein, 422 aa, synthetic construct
MGAPQAFLAGLLLAAVAVGTARAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 237 - protein, 418 aa, synthetic construct
MKVFLVTCLGFAVFSSSVCAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 238 - protein, 431 aa, synthetic construct; identical to SEQ ID NO: 178

SEQ ID NO: 239 - protein, 415 aa, synthetic construct
MRSLIIFLLFPSIIYSAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT